(19) United States
(12) Patent Application Publication
(10) Pub. No.: US 2026/0125644 A1
(43) Pub. Date: May 7, 2026

(54) ENGINEERED IMMUNE CELLS EXPRESSING A CAR AND A PLURALITY OF PROTEIN EXPRESSION BLOCKERS AND USES THEREOF

(71) Applicant: MediSix Therapeutics, Inc., Helios (SG)

(72) Inventors: Dario CAMPANA, Singapore (SG); Ying Xim TAN, Singapore (SG); Alex Xing Fah WONG, Singapore (SG); Simin ZHENG, Singapore (SG)

(21) Appl. No.: 19/101,833

(22) PCT Filed: Aug. 10, 2023
(86) PCT No.: PCT/US2023/072042

§ 371 Date: Feb. 6, 2025

Related U.S. Application Data
(60) Provisional application No. 63/397,067, filed on Aug. 11, 2022.
(60) Provisional application No. 63/408,228, filed on Sep. 20, 2022.
(60) Provisional application No. 63/425,174, filed on Nov. 14, 2022.

(51) Int. Cl. C12N 5/0783; A61K 35/17; A61K 40/11; A61K 40/15; A61K 40/31; A61K 40/42; A61K 40/50; C12N 5/10; C12N 15/85

(52) U.S. Cl. CPC C12N 5/0636; A61K 35/17; A61K 40/11; A61K 40/15; A61K 40/31; A61K 40/421; A61K 40/50; C12N 5/0646; C12N 5/10; C12N 15/85; C12N 2510/00

(57) ABSTRACT

The present invention provides one or more expression vectors encoding one or more of an anti-CD7 chimeric antigen receptor (CAR), an anti-CD7 protein expression blocker, a kill gene or a suicide gene, and an anti-CD3 protein expression blocker. Also provided are immune cells comprising the one or more expression vectors. Also provided are methods of producing such one or more expression vectors or immune cells. Also provided are methods of using such one or more expression vectors or immune cells for treating cancer while reducing symptoms of Graft-versus-host disease (GvHD).

### CROSS-REFERENCE TO RELATED APPLICATIONS

This application is a U.S. National Stage Entry of PCT/US2023/072042, filed Aug. 10, 2023, which claims the benefit of U.S. Provisional Application Ser. No. 63/397,067, filed Aug. 11, 2022, U.S. Provisional Application Ser. No. 63/408,228, filed Sep. 20, 2022, and U.S. Provisional Application Ser. No. 63/425,174, filed Nov. 14, 2022, the entire content of each of which is incorporated herein by reference in their entirety.

### SEQUENCE LISTING

The instant application contains a Sequence Listing which has been submitted electronically in XML format and is hereby incorporated by reference in its entirety. Said XML copy, created on Nov. 18, 2025, is named 62190_725_831_SL.txt and is 207,046 bytes in size.

### BACKGROUND OF THE INVENTION

Chimeric antigen receptors (CARs) can redirect immune cells to specifically recognize and kill tumor cells. CARs are artificial multi-domain proteins constituted by a single-chain variable region (scFv) of an antibody linked to a signaling molecule via a transmembrane domain. When the scFv ligates its cognate antigen, signal transduction is triggered, resulting in tumor cell killing by CAR-expressing cytotoxic T lymphocytes (Eshhar Z, Waks T, et al. PNAS USA. 90(2):720-724, 1993; Geiger T L, et al. J Immunol. 162(10):5931-5939, 1999; Brentjens R J, et al. Nat Med. 9(3):279-286, 2003; Cooper L J, et al. Blood 101(4):1637-1644, 2003; Imai C, et al. Leukemia. 18:676-684, 2004). Clinical trials with CAR-expressing autologous T lymphocytes have shown positive responses in patients with B-cell refractory leukemia and lymphoma (see, e.g., Till B G, et al. Blood 119(17):3940-3950, 2012; Maude S L, et al. N Engl J Med. 371(16):1507-1517, 2014).

The development of CAR technology to target T cell malignancies has lagged far behind the progress made for their B-cell counterparts. Novel therapies for T-cell malignancies are needed but progress to date has been slow. In particular, effective immunotherapeutic options are lacking and treatment of T-cell acute lymphocytic leukemia (T-ALL) relies on intensive chemotherapy and hematopoietic stem cell transplant. Despite aggressive treatment regimen associated with significant morbidity, results with these approaches are far from satisfactory.

CAR-T cells have recently been developed in which the target antigen of the CAR-T is itself expressed in the CAR-T cell (Png et al., Blood, 2017, 1(25):2348-2360, WO 2018/098306). To avoid self-killing (e.g., fratricide), the CAR-T cells also express a PEBL that serves to reduce the expression of the target antigen on the cell surface of the CAR-T. To produce viable CAR-T cells, a protein expression blocker (PEBL) protein can be expressed to bind and sequester the target protein prior to the subsequent expression of the CAR. Due to the pre-existing presence of the target antigen on the cell surface of the resulting engineered T cells, simultaneous expression of the CAR and the PEBL may result in fratricide. In particular, the pre-existing cell surface target antigens may not be susceptible to sequestration by the newly expressed PEBL proteins, and may be recognized and targeted by the newly expressed CAR proteins.

An alternative to simultaneous expression can be sequential expression. However, sequential expression of a PEBL and then a CAR in a T-cell creates several challenges for the clinical implementation of PEBL CAR-T cells. First, sequential engineering of the T cells requires the separate manufacture and administration of distinct viral vectors, one for the PEBL and a second for the CAR. This increases cost and time, as well as the complexity of experimental manipulation to produce the engineered CAR-T cells. In addition, sequential engineering of the T cells results in a complex mix of engineered cells in the final clinical product, creating challenges with product characterization, uniformity and efficacy. Because only a fraction of the T cells integrates the introduced gene at each engineering step, the final product (the engineered T cells) will comprise some cells that only received the PEBL gene, some cells that only received the CAR gene, and some cells that received both genes.

In summary, there is a significant unmet need for new therapeutic options for patients with T-cell malignancies. There is a need for methods for producing an engineered CAR-T cell and eliminating CAR-mediated self-killing or fratricide of the T cell. Additionally, expression of endogenous T-cell receptors (TCRs) carries the risk for graft-versus-host-disease (GvHD), and a method for reducing risk of developing GvHD is needed for effective allogeneic CAR-T cell therapies.

### SUMMARY OF THE INVENTION

Recognized herein is a need for improved CAR-T cell therapies and methods of producing the engineered CAR-T cells. The compositions and methods provided herein can produce engineered CAR-T cells and eliminate CAR-mediated self-killing or fratricide of the T cells. The compositions and methods provided herein can also reduce the risk of developing graft-versus-host-disease (GvHD).

In an aspect, the present disclosure provides an engineered immune cell, comprising: (a) a first nucleic acid sequence encoding (i) a domain that binds to a subunit of a T cell receptor (TCR) complex linked to (ii) a synthetic localizing domain; (b) a second nucleic acid sequence encoding a chimeric antigen receptor (CAR); (c) a third nucleic acid sequence encoding a surface polypeptide binding domain linked to a synthetic surface polypeptide localizing domain; and wherein the engineered immune cell comprises at least twice as much of the first nucleic acid sequence as the second nucleic acid sequence or the third nucleic acid sequence.

In some embodiments, the subunit of the TCR complex is CD3ε. In some embodiments, the synthetic localizing domain comprises an ER retention signal. In some embodiments, the ER retention signal comprises the amino acid sequence KDEL (SEQ ID NO: 73). In some embodiments, the synthetic localizing domain further comprises a Myc tag.

In some embodiments, the synthetic localizing domain further comprises a linker sequence. In some embodiments, the first nucleic acid sequence comprises, in 5′ to 3′ direction, a sequence encoding the domain that binds to a subunit of the TCR complex, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73). In some embodiments, the first nucleic acid sequence comprises, in 5′ to 3′ direction, a sequence encoding an anti-CD3ε antibody, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73).

In some embodiments, the second nucleic acid sequence and the third nucleic acid sequence are on the same nucleic acid molecule.

In some embodiments, the same nucleic acid molecule is a vector.

In some embodiments, the first nucleic acid sequence is on a nucleic acid molecule separate from the second or the third nucleic acid sequence, and wherein the nucleic acid molecule having the first nucleic acid sequence is a first expression vector.

In some embodiments, the engineered immune cell described herein further comprises a fourth nucleic acid sequence encoding a kill gene. In some embodiments, the kill gene comprises CD20 or a derivative thereof.

In some embodiments, the first expression vector further comprises the fourth nucleic acid sequence. In some embodiments, the first expression vector further comprises a ribosome codon skipping site between the first nucleic acid sequence and the fourth nucleic acid sequence.

In some embodiments, the first expression vector comprises, from 5′ to 3′, a promoter, the fourth nucleic acid sequence, the ribosome codon skipping site, and the first nucleic acid sequence. In some embodiments, the ribosomal codon skipping site comprises a 2A self-cleaving peptide. In some embodiments, the first expression vector is a lentiviral vector.

In some embodiments, the CAR comprises a target binding domain that binds to the surface polypeptide. In some embodiments, the surface polypeptide is CD7.

In some embodiments, the surface polypeptide binding domain is a first antibody or antigen binding domain thereof, and the target binding domain is a second antibody or antigen binding domain thereof, and wherein the amino acid sequence of the first antibody and the amino acid sequence of the second antibody are at least 80% identical.

In some embodiments, the amino acid sequences of HC CDRs and/or LC CDRs of the first antibody and the second antibody are at least 90% identical.

In some embodiments, the first antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46. In some embodiments, the second antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, the domain that binds to the subunit of the TCR complex comprises an antibody or an antigen binding domain thereof. In some embodiments, the antibody is an anti-CD3ε antibody. In some embodiments, the antibody is a single chain Fv (scFv) or a single domain antibody (sdAb).

In some embodiments, the anti-CD3ε antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 90% sequence identity to SEQ ID NO: 114. In some embodiments, the anti-CD36 antibody comprises a light chain variable domain having an amino acid sequence with at least 90% sequence identity to SEQ ID NO: 115. In some embodiments, the anti-CD36 antibody comprises an amino acid sequence having at least 90% sequence identity to SEQ ID NO: 104.

In some embodiments, the vector is a second expression vector, and wherein the second expression vector is a bicistronic lentiviral expression vector. In some embodiments, the second nucleic acid sequence and the third nucleic acid sequence are operably linked by an Internal Ribosome Entry Site (IRES) or a ribosomal codon skipping site. In some embodiments, the ribosomal codon skipping site comprises a 2A self-cleaving peptide.

In some embodiments, the synthetic localizing domain or the synthetic surface polypeptide localizing domain comprises an endoplasmic reticulum (ER) retention sequence, a Golgi retention sequence, a proteasome localizing sequence, or a transmembrane domain. In some embodiments, the ER retention sequence comprises the amino acid sequence KDEL (SEQ ID NO: 73) or KKXX, where X is any amino acid. In some embodiments, the ER retention sequence comprises the amino acid sequence KDEL (SEQ ID NO: 73) and the third nucleic acid sequence further comprises a sequence encoding a linker that couples the surface polypeptide binding domain and the synthetic surface polypeptide localizing domain. In some embodiments, the synthetic localizing domain and the synthetic surface polypeptide localizing domain comprise the same ER retention sequence.

In some embodiments, the CAR further comprises a transmembrane domain, a 4-1BB intracellular signaling domain, and a CD3ζ intracellular signaling domain. In some embodiments, surface expression of the TCR complex and the surface polypeptide are downregulated in the engineered immune cell.

In some embodiments, the engineered immune cell is a T cell. In some embodiments, the engineered immune cell is a natural killer (NK) cell.

In an aspect, the present disclosure provides an engineered immune cell, comprising: (a) a first nucleic acid sequence encoding a CD3ε binding domain linked to a synthetic localizing domain, wherein the CD3ε binding domain comprises a heavy chain complementarity-determining region (HC CDR1) of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223; (b) a second nucleic acid sequence encoding a chimeric antigen receptor (CAR); and (c) a third nucleic acid sequence encoding a surface polypeptide binding domain linked to a synthetic surface polypeptide localizing domain.

In some embodiments, the CD3ε binding domain comprises a heavy chain variable domain having an amino acid sequence with at least 90% sequence identity to SEQ ID NO: 114. In some embodiments, the CD3ε binding domain comprises a light chain variable domain having an amino acid sequence with at least 90% sequence identity to SEQ ID NO: 115. In some embodiments, the CD3ε binding domain comprises an amino acid sequence having at least 90% sequence identity to SEQ ID NO: 104.

In some embodiments, the synthetic localizing domain comprises an ER retention signal. In some embodiments, the ER retention signal comprises the amino acid sequence KDEL (SEQ ID NO: 73). In some embodiments, the synthetic localizing domain further comprises a Myc tag. In some embodiments, the synthetic localizing domain further comprises a linker sequence.

In some embodiments, the first nucleic acid sequence comprises, in 5′ to 3′ direction, a sequence encoding the domain that binds to a subunit of the TCR complex, a sequence encoding a Myc tag, a sequence encoding a (GGGGS)4 sequence (SEQ ID NO: 79), and a sequence encoding the ER retention signal comprising the amino acid sequence KDEL (SEQ ID NO: 73). In some embodiments, the first nucleic acid sequence comprises, in 5′ to 3′ direction, a sequence encoding an anti-CD3ε antibody, a sequence encoding a Myc tag, a sequence encoding a (GGGGS)4 sequence (SEQ ID NO: 79), and a sequence encoding the ER retention signal comprising the amino acid sequence KDEL (SEQ ID NO: 73).

In some embodiments, the second nucleic acid sequence and the third nucleic acid sequence are on the same nucleic acid molecule. In some embodiments, the same nucleic acid molecule is a vector. In some embodiments, the first nucleic acid sequence is on a nucleic acid molecule separate from the second or the third nucleic acid sequence, and wherein the nucleic acid molecule having the first nucleic acid sequence is a first expression vector.

In some embodiments, the engineered immune cell described herein further comprises a fourth nucleic acid sequence encoding a kill gene. In some embodiments, the kill gene comprises CD20 or a derivative thereof. In some embodiments, the first expression vector further comprises the fourth nucleic acid sequence. In some embodiments, the first expression vector further comprises a ribosome codon skipping site between the first nucleic acid sequence and the fourth nucleic acid sequence. In some embodiments, the first expression vector comprises, from 5′ to 3′, a promoter, the fourth nucleic acid sequence, the ribosome codon skipping site, and the first nucleic acid sequence. In some embodiments, the ribosomal codon skipping site comprises a 2A self-cleaving peptide. In some embodiments, the first expression vector is a lentiviral vector.

In some embodiments, the CAR comprises a target binding domain that binds to the surface polypeptide. In some embodiments, the surface polypeptide is CD7. In some embodiments, the surface polypeptide binding domain is a first antibody or antigen binding domain thereof, and the target binding domain is a second antibody or antigen binding domain thereof, and wherein the amino acid sequence of the first antibody and the amino acid sequence of the second antibody are at least 80% identical.

In some embodiments, the amino acid sequences of HC CDRs and/or LC CDRs of the first antibody and the second antibody are at least 90% identical. In some embodiments, the first antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46. In some embodiments, the second antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, the vector is a second expression vector, and wherein the second expression vector is a bicistronic lentiviral expression vector. In some embodiments, the second nucleic acid sequence and the third nucleic acid sequence are operably linked by an Internal Ribosome Entry Site (IRES) or a ribosomal codon skipping site. In some embodiments, the ribosomal codon skipping site comprises a 2A self-cleaving peptide.

In some embodiments, the synthetic localizing domain or the synthetic surface polypeptide localizing domain comprises an endoplasmic reticulum (ER) retention sequence, a Golgi retention sequence, a proteasome localizing sequence, or a transmembrane domain.

In some embodiments, the ER retention sequence comprises the amino acid sequence KDEL (SEQ ID NO: 73) or KKXX, where X is any amino acid. In some embodiments, the ER retention sequence comprises the amino acid sequence KDEL (SEQ ID NO: 73) and the third nucleic acid sequence further comprises a sequence encoding a linker that couples the surface polypeptide binding domain and the synthetic surface polypeptide localizing domain. In some embodiments, the synthetic localizing domain and the synthetic surface polypeptide localizing domain comprise the same ER retention sequence.

In some embodiments, the CAR further comprises a transmembrane domain, a 4-1BB intracellular signaling domain, and a CD3ζ intracellular signaling domain. In some embodiments, surface expression of the TCR complex and the surface polypeptide are downregulated in the engineered immune cell.

In some embodiments, the engineered immune cell is a T cell. In some embodiments, the engineered immune cell is a natural killer (NK) cell.

In an aspect, the present disclosure provides a cell population comprising engineered immune cells, wherein the engineered immune cells comprise: (a) a first nucleic acid sequence encoding a domain that binds to a subunit of a T cell receptor (TCR) complex linked to a synthetic localizing domain; and (b) a second nucleic acid sequence encoding a chimeric antigen receptor (CAR); and wherein less than 2% of the cells express the TCR complex on their surface.

In some embodiments, the engineered immune cells further comprise a third nucleic acid sequence encoding a surface polypeptide binding domain linked to a synthetic surface polypeptide localizing domain.

In some embodiments, each of the engineered immune cells comprises at least twice as much of the first nucleic acid sequence as the second nucleic acid sequence or the third nucleic acid sequence.

In some embodiments, the second nucleic acid sequence and the third nucleic acid sequence are on the same nucleic acid molecule. In some embodiments, the same nucleic acid molecule is a vector. In some embodiments, the first nucleic acid sequence is on a nucleic acid molecule separate from the second or the third nucleic acid sequence, and wherein the nucleic acid molecule having the first nucleic acid sequence is a first expression vector.

In some embodiments, the cell population described herein further comprises a fourth nucleic acid sequence encoding a kill gene. In some embodiments, the kill gene comprises CD20 or a derivative thereof.

In some embodiments, the first expression vector further comprises the fourth nucleic acid sequence. In some embodiments, the first expression vector further comprises a ribosome codon skipping site between the first nucleic acid sequence and the fourth nucleic acid sequence. In some embodiments, the first expression vector comprises, from 5′ to 3′, a promoter, the fourth nucleic acid sequence, the ribosome codon skipping site, and the first nucleic acid sequence. In some embodiments, the ribosomal codon skipping site comprises a 2A self-cleaving peptide.

In some embodiments, the first expression vector is a lentiviral vector.

In some embodiments, the subunit of the TCR complex is CD38. In some embodiments, the domain that binds to the subunit of the TCR complex comprises an antibody or an antigen binding domain thereof. In some embodiments, the antibody is an anti-CD3ε antibody. In some embodiments, the antibody is a single chain Fv (scFv) or a single domain antibody (sdAb).

In some embodiments, the anti-CD36 antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 90% sequence identity to SEQ ID NO: 114. In some embodiments, the anti-CD36 antibody comprises a light chain variable domain having an amino acid sequence with at least 90% sequence identity to SEQ ID NO: 115. In some embodiments, the anti-CD36 antibody comprises an amino acid sequence having at least 90% sequence identity to SEQ ID NO: 104.

In some embodiments, the engineered immune cells are depleted of CD3+ cells. In some embodiments, the engineered immune cells are subjected to two or more rounds of depletion of CD3+ cells.

In some embodiments, the synthetic localizing domain comprises an ER retention signal. In some embodiments, the ER retention signal comprises the amino acid sequence KDEL (SEQ ID NO: 73). In some embodiments, the synthetic localizing domain further comprises a Myc tag. In some embodiments, the synthetic localizing domain further comprises a linker sequence.

In some embodiments, the first nucleic acid sequence comprises, in 5′ to 3′ direction, a sequence encoding the domain that binds to a subunit of the TCR complex, a sequence encoding a Myc tag, a sequence encoding a (GGGGS)4 sequence (SEQ ID NO: 79), and a sequence encoding the ER retention signal comprising the amino acid sequence KDEL (SEQ ID NO: 73). In some embodiments, the first nucleic acid sequence comprises, in 5′ to 3′ direction, a sequence encoding an anti-CD3ε antibody, a sequence encoding a Myc tag, a sequence encoding a (GGGGS)4 sequence (SEQ ID NO: 79), and a sequence encoding the ER retention signal comprising the amino acid sequence KDEL (SEQ ID NO: 73).

In some embodiments, the CAR comprises a target binding domain that binds to the surface polypeptide. In some embodiments, the surface polypeptide is CD7.

In some embodiments, the surface polypeptide binding domain is a first antibody or antigen binding domain thereof, and the target binding domain is a second antibody or antigen binding domain thereof, and wherein the amino acid sequence of the first antibody and the amino acid sequence of the second antibody are at least 80% identical. In some embodiments, the amino acid sequences of HC CDRs and/or LC CDRs of the first antibody and the second antibody are at least 90% identical.

In some embodiments, the first antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46. In some embodiments, the second antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, the vector is a second expression vector, and wherein the second expression vector is a bicistronic lentiviral expression vector. In some embodiments, the second nucleic acid sequence and the third nucleic acid sequence are operably linked by an Internal Ribosome Entry Site (IRES) or a ribosomal codon skipping site. In some embodiments, the ribosomal codon skipping site comprises a 2A self-cleaving peptide.

In some embodiments, the synthetic localizing domain or the synthetic surface polypeptide localizing domain comprises an endoplasmic reticulum (ER) retention sequence, a Golgi retention sequence, a proteasome localizing sequence, or a transmembrane domain.

In some embodiments, the ER retention sequence comprises the amino acid sequence KDEL (SEQ ID NO: 73) or KKXX, where X is any amino acid. In some embodiments, the ER retention sequence comprises the amino acid sequence KDEL (SEQ ID NO: 73) and the third nucleic acid sequence further comprises a sequence encoding a linker that couples the surface polypeptide binding domain and the synthetic surface polypeptide localizing domain. In some embodiments, the synthetic localizing domain and the synthetic surface polypeptide localizing domain comprise the same ER retention sequence.

In some embodiments, the CAR further comprises a transmembrane domain, a 4-1BB intracellular signaling domain, and a CD3ζ intracellular signaling domain. In some embodiments, surface expression of the TCR complex and the surface polypeptide are downregulated in the engineered immune cell.

In some embodiments, the engineered immune cells are T cells. In some embodiments, the engineered immune cells are natural killer (NK) cells.

In an aspect, the present disclosure provides a bicistronic vector comprising: (a) a first nucleotide sequence encoding a kill gene; and (b) a second nucleotide sequence encoding a surface polypeptide binding domain linked to a localizing domain.

In some embodiments, the bicistronic vector comprises, in a 5′ to 3′ direction, a transcriptional start site, the first nucleotide sequence encoding the kill gene, a ribosomal codon skipping site, and the second nucleotide sequence encoding the surface polypeptide binding domain linked to the localizing domain. In some embodiments, the kill gene encodes a cell surface antigen. In some embodiments, the cell surface antigen is CD20 or a derivative thereof. In some embodiments, the surface polypeptide binding domain binds to a subunit of a TCR complex. In some embodiments, the subunit is CD3ε.

In some embodiments, the localizing domain comprises an ER retention signal. In some embodiments, the ER retention signal comprises the amino acid sequence KDEL (SEQ ID NO: 73). In some embodiments, the localizing domain further comprises a Myc tag. In some embodiments, the localizing domain further comprises a linker sequence.

In some embodiments, the second nucleotide sequence comprises, in 5′ to 3′ direction, a sequence encoding the surface polypeptide binding domain, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73).

In some embodiments, the surface polypeptide binding domain that binds to the subunit of the TCR complex comprises an antibody or an antigen binding domain thereof. In some embodiments, the antibody is an anti-CD3ε antibody. In some embodiments, the antibody is a single chain Fv (scFv) or a single domain antibody (sdAb). In some embodiments, the ribosomal codon skipping site comprises a 2A self-cleaving peptide.

In some embodiments, the bicistronic vector is a lentiviral vector.

In some embodiments, the first nucleotide sequence and the second nucleotide sequence are operably linked by an Internal Ribosome Entry Site (IRES). In some embodiments, the localizing domain comprises an endoplasmic reticulum (ER) retention sequence, a Golgi retention sequence, a proteasome localizing sequence, or a transmembrane domain.

In some embodiments, the surface polypeptide binding domain is an anti-CD3ε antibody or antigen binding domain thereof. In some embodiments, the anti-CD3ε antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 90% sequence identity to SEQ ID NO: 114. In some embodiments, the anti-CD3ε antibody comprises a light chain variable domain having an amino acid sequence with at least 90% sequence identity to SEQ ID NO: 115. In some embodiments, the anti-CD3ε antibody comprises an amino acid sequence having at least 90% sequence identity to SEQ ID NO: 104.

In an aspect, the present disclosure provides a recombinant nucleic acid molecule encoding a CD3ε binding domain linked to a synthetic localizing domain, wherein the CD3ε binding domain comprises a heavy chain complementarity-determining region (HC CDR1) of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223, and wherein the synthetic localizing domain comprises a linker sequence having at least 5 amino acids in length and an amino acid sequence KDEL (SEQ ID NO: 73).

In some embodiments, the linker sequence comprises (GGGGS)n (SEQ ID NO: 224), where n is any integer from 1 to 10. In some embodiments, the linker sequence comprises (GGGGS)4 (SEQ ID NO: 79). In some embodiments, the synthetic localizing domain further comprises a Myc tag. In some embodiments, the recombinant nucleic acid molecule comprises a sequence of SEQ ID NO: 99. In some embodiments, provided herein is an engineered immune cell comprising the recombinant nucleic acid molecule described herein.

In an aspect, the present disclosure provides a method of producing a population of engineered immune cells, the method comprising: (a) introducing into immune cells: (i) a first nucleic acid sequence encoding (i) a domain that binds to a subunit of a T cell receptor (TCR) complex linked to (ii) a synthetic localizing domain; (ii) a second nucleic acid sequence encoding a chimeric antigen receptor (CAR); and (iii) a third nucleic acid sequence encoding a surface polypeptide binding domain linked to a synthetic surface polypeptide localizing domain, thereby producing the population of engineered immune cells; and (b) culturing the population of engineered immune cells; thereby expressing the CAR and downregulating surface expression of the TCR complex and the surface polypeptide in the population of engineered immune cell; wherein the amount of the first nucleic acid sequence is at least twice as high as the amount of the second nucleic acid sequence or the third nucleic acid sequence.

In some embodiments, the CAR comprises a target binding domain that binds to the surface polypeptide. In some embodiments, the downregulation of the surface polypeptide prevents fratricide of the population of engineered immune cells by the CAR. In some embodiments, the surface polypeptide comprises CD7.

In some embodiments, the second nucleic acid sequence and the third nucleic acid sequence are on a same nucleic acid molecule. In some embodiments, the same nucleic acid molecule is a bicistronic vector. In some embodiments, the first nucleic acid sequence is on a nucleic acid molecule separate from the second nucleic acid sequence or the third nucleic acid sequence, and wherein the nucleic acid molecule is a first vector.

In some embodiments, introducing into immune cells comprises co-transducing the bicistronic vector comprising the second nucleic acid sequence and the third nucleic acid sequence and the first vector comprising the first nucleic acid sequence. In some embodiments, introducing into immune cells comprises transducing the first vector comprising the first nucleic acid sequence from 0 to 2 days prior to transducing the bicistronic vector comprising the second nucleic acid sequence and the third nucleic acid sequence.

In some embodiments, a multiplicity of infection (MOI) of the bicistronic vector is about 10. In some embodiments, an MOI of the first vector is at least about 20. In some embodiments, an MOI of the first vector is from 20 to 40. In some embodiments, a ratio of an MOI of the bicistronic vector and an MOI of the first vector is at most about 1:8.

In some embodiments, the method described herein further comprises introducing into the immune cells a fourth nucleic acid sequence encoding a kill gene. In some embodiments, the kill gene encodes a surface antigen. In some embodiments, the surface antigen is CD20 or a derivative thereof.

In some embodiments, the method described herein further comprises depleting cells that express the TCR complex from the population of engineered immune cells.

In some embodiments, the method described herein further comprises enriching cells that express the surface antigen, thereby depleting cells that express the TCR complex.

In some embodiments, the method described herein further comprises, prior to depleting, enriching cells that express the surface antigen, thereby producing a population of CD20+ TCR− engineered immune cells.

In some embodiments, culturing the population of engineered immune cells comprising expanding the population of engineered immune cells for at least 5 days between the introducing and the depleting.

In some embodiments, at least 95% of the population of CD20+TCR− engineered immune cells do not express CD3. In some embodiments, the subunit of the TCR complex is CD3ε. In some embodiments, the domain that binds to the subunit of the TCR complex comprises an antibody or an antigen binding domain thereof. In some embodiments, the antibody is an anti-CD3ε antibody. In some embodiments, the antibody is a single chain Fv (scFv) or a single domain antibody (sdAb).

In some embodiments, the anti-CD36 antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 90% sequence identity to SEQ ID NO: 104. In some embodiments, the anti-CD36 antibody comprises a light chain variable domain having an amino acid sequence with at least 90% sequence identity to SEQ ID NO: 105. In some embodiments, the anti-CD36 antibody comprises an amino acid sequence having at least 90% sequence identity to SEQ ID NO: 104.

In some embodiments, the synthetic localizing domain comprises an ER retention signal. In some embodiments, the ER retention signal comprises the amino acid sequence KDEL (SEQ ID NO: 73). In some embodiments, the synthetic localizing domain further comprises a Myc tag. In some embodiments, the synthetic localizing domain further comprises a linker sequence.

In some embodiments, the first vector further comprises the fourth nucleic acid sequence. In some embodiments, the first vector comprises, from 5′ to 3′, a promoter, the fourth nucleic acid sequence, the ribosome codon skipping site, and the first nucleic acid sequence. In some embodiments, the ribosomal codon skipping site comprises a 2A self-cleaving peptide

In some embodiments, the first vector is a lentiviral vector.

In some embodiments, the surface polypeptide binding domain is a first antibody or antigen binding domain thereof, and the target binding domain is a second antibody or antigen binding domain thereof, and wherein the amino acid sequence of the first antibody and the amino acid sequence of the second antibody are at least 80% identical. In some embodiments, the amino acid sequences of HC CDRs and/or LC CDRs of the first antibody and the second antibody are at least 90% identical.

In some embodiments, the first antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46. In some embodiments, the second antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, the second nucleic acid sequence and the third nucleic acid sequence are operably linked by an Internal Ribosome Entry Site (IRES) or a ribosomal codon skipping site. In some embodiments, the ribosomal codon skipping site comprises a 2A self-cleaving peptide. In some embodiments, the synthetic localizing domain or the synthetic surface polypeptide localizing domain comprises an endoplasmic reticulum (ER) retention sequence, a Golgi retention sequence, a proteasome localizing sequence, or a transmembrane domain. In some embodiments, the ER retention sequence comprises the amino acid sequence KDEL (SEQ ID NO: 73) or KKXX, where X is any amino acid. In some embodiments, the ER retention sequence comprises the amino acid sequence KDEL (SEQ ID NO: 73) and the third nucleic acid sequence further comprises a sequence encoding a linker that couples the surface polypeptide binding domain and the synthetic surface polypeptide localizing domain. In some embodiments, the synthetic localizing domain and the synthetic surface polypeptide localizing domain comprise the same ER retention sequence.

In some embodiments, the CAR further comprises a transmembrane domain, a 4-1BB intracellular signaling domain, and a CD3ζ intracellular signaling domain. In some embodiments, surface expression of the TCR complex and the surface polypeptide are downregulated in the engineered immune cell.

In some embodiments, the engineered immune cells are T cells. In some embodiments, the engineered immune cells are natural killer (NK) cells.

In an aspect, the present disclosure provides a method of treating a disease in a subject in need thereof, the method comprising: administering a therapeutically effective amount of engineered immune cells, wherein said engineered immune cells comprise: (a) a first nucleic acid sequence encoding (i) a domain that binds to a subunit of a T cell receptor (TCR) complex linked to (ii) a synthetic localizing domain; (b) a second nucleic acid sequence encoding a chimeric antigen receptor (CAR); and (c) a third nucleic acid sequence encoding a surface polypeptide binding domain linked to a synthetic surface polypeptide localizing domain; wherein the domain that binds to the subunit of a TCR complex linked to the synthetic localizing domain downregulates surface expression of the TCR complex, thereby treating the disease in the subject in need thereof.

In some embodiments, a risk of developing a graft-versus-host disease (GvHD) response in the subject after administering the engineered immune cells is reduced compared to a risk associated with administration of otherwise identical immune cells comprising the second or the third nucleic acid sequence but not the first nucleic acid sequence. In some embodiments, the subject has cancer.

In some embodiments, the CAR comprises a target binding domain that binds to CD7.

In some embodiments, the subunit of the TCR complex is CD38.

In some embodiments, the engineered immune cells are T cells or natural killer (NK) cells.

In some embodiments, the cancer comprises CD7 positive cancer.

In some embodiments, the cancer comprises acute lymphoblastic leukemia (T-ALL), early T-cell progenitor acute lymphoblastic leukemia (ETP-ALL), acute myeloid leukemia, or T-cell lymphoblastic lymphoma.

In some embodiments, the engineered immune cells suppress tumor cell growth in the subject.

In some embodiments, the domain that binds to the subunit of the TCR complex comprises an antibody or an antigen binding domain thereof. In some embodiments, the antibody is an anti-CD3ε antibody. In some embodiments, the antibody is a single chain Fv (scFv) or a single domain antibody (sdAb).

In some embodiments, the anti-CD3ε antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 90% sequence identity to SEQ ID NO: 114. In some embodiments, the anti-CD36 antibody comprises a light chain variable domain having an amino acid sequence with at least 90% sequence identity to SEQ ID NO: 115. In some embodiments, the anti-CD36 antibody comprises an amino acid sequence having at least 90% sequence identity to SEQ ID NO: 104.

In some embodiments, the synthetic localizing domain comprises an ER retention signal. In some embodiments, the ER retention signal comprises the amino acid sequence KDEL (SEQ ID NO: 73). In some embodiments, the synthetic localizing domain further comprises a Myc tag. In some embodiments, the synthetic localizing domain further comprises a linker sequence.

In some embodiments, the first nucleic acid sequence comprises, in 5′ to 3′ direction, a sequence encoding the domain that binds to a subunit of the TCR complex, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73). In some embodiments, the first nucleic acid sequence comprises, in 5′ to 3′ direction, a sequence encoding an anti-CD3ε antibody, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73).

In some embodiments, the second nucleic acid sequence and the third nucleic acid sequence are on the same nucleic acid molecule. In some embodiments, the same nucleic acid molecule is a vector. In some embodiments, the first nucleic acid sequence is on a nucleic acid molecule separate from the second or the third nucleic acid sequence, and wherein the nucleic acid molecule having the first nucleic acid sequence is a first expression vector.

In some embodiments, the method described herein further comprises a fourth nucleic acid sequence encoding a kill gene. In some embodiments, the kill gene comprises CD20 or a derivative thereof. In some embodiments, the first expression vector further comprises the fourth nucleic acid sequence.

In some embodiments, the first expression vector further comprises a ribosome codon skipping site between the first nucleic acid sequence and the fourth nucleic acid sequence. In some embodiments, the first expression vector comprises, from 5′ to 3′, a promoter, the fourth nucleic acid sequence, the ribosome codon skipping site, and the first nucleic acid sequence. In some embodiments, the ribosomal codon skipping site comprises a 2A self-cleaving peptide

In some embodiments, the first expression vector is a lentiviral vector.

In some embodiments, the surface polypeptide is CD7.

In some embodiments, the surface polypeptide binding domain is a first antibody or antigen binding domain thereof, and the target binding domain is a second antibody or antigen binding domain thereof, and wherein the amino acid sequence of the first antibody and the amino acid sequence of the second antibody are at least 80% identical. In some embodiments, the amino acid sequences of HC CDRs and/or LC CDRs of the first antibody and the second antibody are at least 90% identical.

In some embodiments, the first antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46. In some embodiments, the second antibody comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, the vector is a second expression vector, and wherein the second expression vector is a bicistronic lentiviral expression vector.

In some embodiments, the second nucleic acid sequence and the third nucleic acid sequence are operably linked by an Internal Ribosome Entry Site (IRES) or a ribosomal codon skipping site. In some embodiments, the ribosomal codon skipping site comprises a 2A self-cleaving peptide. In some embodiments, the synthetic localizing domain or the synthetic surface polypeptide localizing domain comprises an endoplasmic reticulum (ER) retention sequence, a Golgi retention sequence, a proteasome localizing sequence, or a transmembrane domain. In some embodiments, the ER retention sequence comprises the amino acid sequence KDEL (SEQ ID NO: 73) or KKXX, where X is any amino acid. In some embodiments, the ER retention sequence comprises the amino acid sequence KDEL (SEQ ID NO: 73) and the third nucleic acid sequence further comprises a sequence encoding a linker that couples the surface polypeptide binding domain and the synthetic surface polypeptide localizing domain. In some embodiments, wherein the synthetic localizing domain and the synthetic surface polypeptide localizing domain comprise the same ER retention sequence.

In some embodiments, the CAR further comprises a transmembrane domain, a 4-1BB intracellular signaling domain, and a CD3ζ intracellular signaling domain. In some embodiments, the engineered immune cells are T cells. In some embodiments, the engineered immune cells are natural killer (NK) cells.

In an aspect, the present disclosure provides a method of depleting engineered immune cells after administration in a subject in need thereof, the method comprising administering to the subject a population of engineered immune cells that express: (a) a chimeric antigen receptor (CAR) specific for a T cell surface antigen; (b) a first protein expression blocker (PEBL) that downregulates cell surface expression of the T cell surface antigen; (c) a second PEBL that downregulates cell surface expression of a subunit of a T cell receptor (TCR) complex; and (d) a kill protein.

In some embodiments, the kill protein is a cell surface antigen. In some embodiments, the cell surface antigen is CD20 or a derivative thereof. In some embodiments, the kill protein is a truncated CD20 (CD20t). In some embodiments, the CD20t comprises an amino acid sequence of SEQ ID NO: 106.

In some embodiments, the method described herein further comprises administering rituximab or ofatumumab to the subject to induce elimination of the population of engineered immune cells, thereby depleting the population of engineered immune cells.

In some embodiments, the subject has been diagnosed with an immune condition. In some embodiments, the subject has been diagnosed with a T cell malignancy.

In some embodiments, the T cell surface antigen is CD7. In some embodiments, the subunit of the TCR complex is CD38.

In some embodiments, the population of engineered immune cells comprises T cells. In some embodiments, the population of engineered immune cells comprises natural killer (NK) cells.

In an aspect, the present disclosure provides a kit comprising: (a) a first expression vector comprising a nucleotide sequence encoding (i) a domain that binds to a subunit of a T cell receptor (TCR) complex linked to (ii) a synthetic localizing domain and a nucleotide sequence encoding a kill gene; and (b) a second expression vector comprising a nucleotide sequence encoding a chimeric antigen receptor (CAR) and a nucleotide sequence encoding a surface polypeptide binding domain linked to a synthetic surface polypeptide localizing domain.

In some embodiments, the first expression vector or the second expression vector is a retroviral vector. In some embodiments, the first expression vector or the second expression vector is a lentiviral vector.

In some embodiments, the CAR comprises a target binding domain that binds to CD7. In some embodiments, the surface polypeptide is CD7. In some embodiments, the subunit of the TCR complex is CD38.

In some embodiments, the nucleotide sequence encoding (i) the domain that binds to a subunit of a TCR complex linked to (ii) a synthetic localizing domain and the nucleotide sequence encoding the kill gene are operably linked by an Internal Ribosome Entry Site (IRES) or a ribosomal codon skipping site. In some embodiments, the nucleotide sequence encoding the CAR and the nucleotide sequence encoding the surface polypeptide binding domain linked to the synthetic surface polypeptide localizing domain are operably linked by an Internal Ribosome Entry Site (IRES) or a ribosomal codon skipping site. In some embodiments, the ribosomal codon skipping site comprises a 2A self-cleaving peptide.

In some embodiments, the first expression vector and the second expression vector are mixed at a ratio of at least 2:1.

### BRIEF DESCRIPTION OF THE DRAWINGS

FIG. 1 depicts scheme of allogeneic PCART7-CD3PEBL cells (allo-PCART7) using healthy donor T cells.

FIG. 2 depicts phenotypical validation of allogeneic PCART7-CD3PEBL cells (allo-PCART7). T cells were thawed and activated with TransAct before transducing with PCART7 LVV alone or in combination with CD3 PEBL LVV. Three days later, PCART7-transduced cells were electroporated with TRAC RNPs to generate PCART7-TRAC KO cells. Cells were expanded for another 10 days before CD3 depletion. Cells were stained with respective antibodies and analyzed using flow cytometry.

FIGS. 3A-3C depict phenotypic characteristics of allo-PCART7. FIG. 3A shows flow cytometric evaluation of the expression of CAR, CD7 and CD3 15 days after the start of the manufacturing process, with and without CD3 depletion.CD3-depletion was able to increase the quality of allo-PCART7. FIG. 3B top panel shows that % of CAR+ cells at time of harvest between PCART7 and allo-PCART7 were similar between the two groups. However, as depicted in FIG. 3B bottom panel, allo-PCART7 shows depletion of TCRαβ and CD3 markers as analyzed by flow cytometry. FIG. 3C shows that the engineered T cells, PCART7 and allo-PCART7, had similar expansion and viability across the manufacturing duration.

FIG. 4 depicts that allogeneic PCART7-CD3PEBL cells were not responsive to TransAct mediated TCR stimulation. Cells were treated for 72 hours with or without TransAct before detecting CD25 activation marker by flow cytometry.

FIGS. 5A-5C depict that allo-PCART7 cells show effective sustained CD3 downregulation. FIG. 5A shows the experimental timeline. Allo-PCART7 cells were stimulated with target cells or TransAct (CD3 and CD28 activation of T cells) every 3-4 days to validate the effectiveness of CD3 PEBL in retaining CD3 inside the cells. FIG. 5B shows that allo-PCART7 cells did not express CD3 and are distinct from CD3+ PCART7 cells both before and after stimulation with CD3-KO Jurkat cells (solid line). After stimulation with CD3-KO Jurkat cells, the number allo-PCART7 and PCART7 cells were increased, but CD3 level remained unchanged, suggesting that in allo-PCART7 cells, CD3 was effectively retained inside the cells by CD3 PEBL. Solid line indicates co-culture of CD3-KO Jurkat cells with either allo-PCART7 cells or PCART7 cells. Dashed line indicates either allo-PCART7 cells only or PCART7 cells only. FIG. 5C shows that at Day 10 post-stimulation with TransAct, % expression level of CD25, which is a cell surface marker for activated lymphocytes during active immune response such as in GvHD, remained unchanged in allo-PCART7 cells. However, PCART7 cells showed a large increase in % CD25 expression after stimulation.

FIG. 6 depicts that allo-PCART7 cells can effectively and specifically kill T-cell acute lymphoblastic leukemia (T-ALL) cells in vitro. In this experiment, the antitumor potential of allo-PCART7 cells was evaluated in vitro using co-culture of allo-PCART7 cells with T-ALL cells at a 1:1 effector-to-target (E:T) ratio. CD7+ and CD7− T-ALL cells were engineered to express green fluorescent protein (GFP), and the co-culture experiment was performed using an IncuCyte® system to monitor the amount of T-ALL target cells in experimental wells over time. On the y-axis is the GFP signal from target cells in the well and the x-axis is the duration of co-culture. As shown on the left panel, Jurkat-GFP cells (CD7+T-ALL cell line) alone were expanding. However, once these cells were co-cultured with PCART7 cells or allo-PCART7 cells, there was a decrease in CD7+ target cells, reaching an undetectable level. As shown on the right panel, Nalm6-GFP (B cell precursor leukemia cell line) was used as CD7− target cells. Co-culture of engineered T cells, either PCART7 cells or allo-PCART7 cells, did not affect CD7− cells, suggesting that there was no non-specific targeting of CD7 by these engineered T cells.

FIG. 7 depicts in vivo efficacy of allogeneic PCART7-CD3PEBL (allo-PCART7). NSG mice were intravenously injected with 1×10⁶ FLuc-GFP expressing CCRF-CEM leukemic cells. Three days later, tumor-bearing mice were treated with PBS (vehicle), non-transduced T cells, PCART7 or PCART7-CD3PEBL. Bioluminescence from tumor cells were detected using IVIS imaging.

FIGS. 8A-8B depict that allo-PCART7 cells effectively kill T-ALL cells in NSG mouse model. FIG. 8A shows experimental scheme and timeline. Briefly, on Day 0, NSG mice were infused with CCRF-CEM FLuc-GFP cells derived from the CCRF-CEM T-ALL cell line, via intravenous injection (i.v.) at 1×10⁶ cells per mice. On Day 5, these mice received either PBS (vehicle), non-transduced T cells, or allo-PCART7 cells via i.v. injection. Tumor growth was then monitored by IVIS imaging. As shown in FIG. 8B, mice treated with PBS (vehicle group) and mice treated with non-transduced T-cells (T-cells group) showed high tumor burden at Day 21. However, mice treated with allo-PCART7 cells revealed a dose-dependent suppression of tumor cells. Total flux [p/s] is shown below each group and the highest dose treatment showed the lowest level of tumor. These results showed that allo-PCART7 cells are effective in killing leukemic cells in mice.

FIG. 9 depicts that allo-PCART7 cells do not trigger GvHD in a mouse model. In this experiment, cells were injected into irradiated NSG mice and the weight of these mice were tracked. The result shows that the weight of mice that were injected with non-transduced T cells dropped substantially and had to be euthanized. However, the weight of mice that were injected with allo-PCART7 cells remained similar to the irradiated control mice, suggesting that allo-PCART7 cells are not xenoreactive.

FIG. 10 depicts scheme of manufacturing of allogeneic PCART7-CD3PEBL with truncated CD20 or allo-PCART7^{KG}. These allo-PCART7^{KG} cells comprise anti-CD7 CAR that recognize and kill CD7+ target cells. An anti-CD7 PEBL is present in these cells to remove surface CD7 to prevent self-killing or fratricide. Anti-CD3 PEBL is present to minimize risk of graft vs host disease (GVHD). Further, additional safety mechanism is engineered in these cells by expressing a kill gene or a suicide gene such as truncated CD20 to enable elimination of the allo-PCART7^{KG} cells from the patients with an anti-CD20 antibody during adverse events.

FIG. 11 depicts detailed workflow of allo-PCART7^{KG} manufacturing process. Briefly, cells comprising immune cells, e.g., T cells, are collected from healthy donors on Day 0. On Day 1, T cells are then isolated and activated using TransAct, which is a ready-to-use reagent for in vitro activation and expansion of human T cells. On Day 2, T cells are co-transduced using lentiviral vectors to introduce 2 PEBLs (CD3 PEBL and CD7 PEBL), CD7 CAR, and optionally a suicide gene or a kill gene. Following the transduction, cells are expanded from Day 3 to Day 13 in vitro. Between Day 13 to Day 15, CD3 depletion of engineered T cells is performed using CD3 microbeads. These cells are then cryopreserved as a final cell product.

FIGS. 12A-12B depict phenotypic characteristics of allo-PCART7 cells with a suicide gene (or kill gene or KG), and these cells are termed allo-PCART7^{KG}(PCART7^{TCRnegKG}) In this example, allo-PCART7 were transduced to express truncated CD20, which is used as a suicide or a kill gene. FIG. 12A shows that 15 day after the start of manufacturing, allo-PCART7^{KG} cells (PCART7^{TCRnegKG}) had desired characteristics (CD7 CAR+/CD7−/CD3−/CD20+) when analyzed by flow cytometry. FIG. 12B shows the cytotoxic activity of allo-PCART7^{KG} cells (PCART7^{TCRnegKG}) compared to allo-PCART7 cells (PCART7^{TCRneg}) and PCART7 cells. This result shows that there was no difference in the killing capacity and targeting of CD7+ cells (Jurkat cells) by PCART7 cells, allo-PCART7 cells (PCART7^{TCRneg}) and allo-PCART7^{KG} cells (PCART7^{TCRnegKG}) Suggesting that expression of a suicide gene or a kill gene (e.g., CD20, CD20t) does not significantly affect the function of the allo-PCART7^{KG} cells.

FIGS. 13A-13B depict the function of the kill gene in allo-PCART7^{KG} cells. In order to validate the function of the kill gene, a complement-dependent cytotoxicity (CDC) assay and an antibody-dependent cellular cytotoxicity (ADCC) assay were performed. As shown in FIGS. 13A-13B, result from CDC assay and ADCC assay show that allo-PCART7^{KG} cells, which express CD20t, were recognized by rituximab, an anti-CD20 monoclonal antibody, resulting in triggering of CDC or ADCC, thereby specifically eliminating allo-PCART7^{KG} cells. In contrast, addition of rituximab did not induce cytotoxicity in allo-PCART7 cells that did not express CD20. Trastuzumab, which is a monoclonal antibody targeting HER2, was used as a negative control.

FIGS. 14A-14B depict the outcome of sequential transduction of PCART7 and CD3 PEBL. FIG. 14A shows the experimental timeline. Following activation of PBMCs, a first lentiviral vector transduction occurred on Day 1 with a second lentiviral vector transduction occurring on Day 6. FACS analysis was then performed on transduced cells. FIG. 14B shows cells were not viable with transduction of CD3 PEBL followed by PCART7. For sequential transduction of PCART7 followed by CD3 PEBL, the CD3 downregulation was sub-optimal.

FIG. 15 depicts a comparison of OKT3 and UCHT1 PEBLs. UCHT1 PEBL showed superior performance for downregulating surface CD3 expression in allo-PCART7 cells.

FIGS. 16A-16C depict phenotypic characteristics of allo-PCART7 with varying multiplicity of infection (MOI) for CD3 PEBL. FIG. 16A shows the experimental timeline. Following T cell activation, cells were co-transduced with PCART7 and CD3 PEBL. FIG. 16B shows cells stained with respective antibodies to confirm the expression of CD3, CD7, and/or CAR by flow cytometry 15 days after manufacturing. There was a MOI-dependent downregulation of CD3 by CD3 PEBL. Cotransduction of PCART7 with CD3 PEBL MOI of greater than 5 resulted in >95% CAR+CD7−CD3− population. FIG. 16C shows cotransduction of PCART7 with CD3 PEBL MOI of 5 (10+5) show robust phenotype and cell expansion.

FIGS. 17A-17B depict results to identify a minimum copy number ratio of CD3PEBL/PCART7 for CD3 retention. FIG. 17A shows T cells were co-transduced with PCART7 and CD3 PEBL at the indicated MOIs, followed by CD3 depletion on Day 15 of manufacturing. CD3 expression was examined by flow cytometry. FIG. 17B shows percentage of CD3-negative cells plotted against CD3PEBL/PCART7 VCN ratio to determine a minimum VCN ratio to maintain >95% CD3-negative cells at Day 14.

FIGS. 18A-18B depict comparisons of different kill gene constructs for CD3 PEBL vector optimization. FIG. 18A shows schematic representations of the tested constructs. The CD3 PEBL was separated from the kill gene (CD20t) by a P2A sequence (e.g., KG(P2A)) or a IRES sequence (e.g., KG(IRES)). FIG. 18B shows flow cytometry plots demonstrating that KG(P2A) cotransduced with PCART7 led to higher CD20 expression and better CD3 downregulation than KG(IRES).

FIGS. 19A-19B depict optimization of MOI ratio for PCART7:CD3 PEBL cotransduction. FIG. 19A shows results following co-transduction with PCART7 lentiviral vector and CD3 PEBL lentiviral vector without a kill gene. 15 days after manufacturing, cells were assessed for CAR and CD3 expression by flow cytometry. FIG. 19B shows similar co-transduction as FIG. 19A but with a kill gene. PCART7 at MOI 10 and CD3 PEBL at MOI 40 showed optimal ratio for co-transduction.

FIGS. 20A-20B depict addition of a CD20 enrichment step to the cell manufacturing process. FIG. 20A shows flow cytometry plots examining CD20 and CD3 expression. Enrichment of CD20 prior to CD3 depletion led to recovery of CD20 high-expressing cells (Day 0). FIG. 20B shows addition of CD20 enrichment step to cell manufacturing led to higher CD20 MFI.

FIGS. 21A-21G depict functional evaluations of CD20-enriched allo-PCART7^{KG(P2A)} cells. FIG. 21A shows cytotoxicity of non-transduced control T cells and CD20-enriched allo-PCART7^{KG(P2A)} cells induced by Trastuzumab. FIG. 21B shows cytotoxicity of non-transduced control T cells and CD20-enriched allo-PCART7^{KG(P2A)} cells induced by Rituximab. FIG. 21C shows CD3 and CD20 expression 14 days post CD3 depletion in allo-PCART7^{KG(P2A)} cells and CD20-enriched allo-PCART7^{KG(P2A)} cells by flow cytometry. FIGS. 21D-21F show cytotoxic activity of allo-PCART7 and allo-PCART7^{KG(P2A)} cells against CD7+ and CD7− target cells assessed using IncuCyte® live-cell analysis. FIG. 21G shows an experimental protocol for production of allo-PCART7^{KG(P2A)} cells.

### DETAILED DESCRIPTION OF THE INVENTION

### Introduction

CAR-T cells have been developed to target T cell malignancies. Patients with T cell malignancies such as T-cell acute lymphoblastic leukemia (T-ALL) with refractory/relapse T-ALL have low or dysfunctional T cells that are unable to produce autologous T cells for effectively engineered CAR-T cells. Engineered allogeneic CAR-T cells provide an alternative source of cells, allowing CAR-T cell infusion in such patients, with the aim to induce remission in these patients prior to stem cell transplantation or autologous CAR-T cell infusion, and these can improve the survival rate. There are several advantages of allogeneic CAR-T cells, for examples, T cells used for engineering can be selected from healthy donor, which provides healthy T cells; the manufacturing process can be standardized to provide high-quality engineered CAR-T cell product; and the resulting engineered CAR-T cell product can be readily available and distributed around the world for patients in need. One concern regarding an allogeneic transplant is a condition called graft versus host disease (or GvHD). In GvHD, the donated cells view the recipient's body as foreign, thereby attacking the recipient's body, and this can be life-threatening. GvHD is mediated by T cell receptor (TCR)/CD3 on the T cell surface.

The present disclosure provides engineered immune cells comprising anti-CD7 CAR that recognize and kill CD7+ target cells; anti-CD7 protein expression blocker (CD7 PEBL) which removes surface CD7 expression of the engineered immune cells in order to prevent fratricide or self-killing; and anti-CD3 protein expression blocker (CD3 PEBL) to avoid graft vs host disease (GvHD) or symptoms thereof. Additionally, these engineered immune cells can further be incorporated with an additional safety mechanism by expressing a kill gene to eliminate the engineered immune cells from the patients during situation of adverse events.

The present disclosure provides engineered immune cell compositions and methods for co-expression of a fratricide-inducing chimeric antigen receptor (e.g., CAR) and a first fratricide-preventing protein (e.g., protein expression blocker (PEBL)) in T cells that downregulate a first immune cell surface protein, and a second PEBL that downregulate a second immune cell surface protein that result in viable CAR-expressing cytotoxic T lymphocytes (CAR-T) that target T cell antigens. In some aspects, the CAR and the first PEBL target the same molecule, while the second PEBL targets a different molecule than the CAR and the first PEBL. In some aspects, a single expression vector comprises a nucleic acid encoding the CAR and a nucleic acid encoding the first PEBL. In some instances, the nucleic acid encoding the CAR and the nucleic acid encoding the first PEBL are located in a bicistronic vector (e.g., bicistronic retroviral vector, bicistronic lentiviral vector) such that the CAR and the first PEBL are expressed simultaneously. In some aspects, the nucleic acid encoding the CAR and the nucleic acid encoding the first PEBL are operably linked by Internal Ribosome Entry Site (IRES) or a ribosomal codon skipping site. In some embodiments, the ribosomal codon skipping site comprises a 2A self-cleaving peptide. In some instances, the nucleic acid encoding the CAR and the nucleic acid encoding the first PEBL are located under the same promoter. In some instances, the nucleic acid encoding the CAR and the nucleic acid encoding the first PEBL are located under different promoters. In some instances, the nucleic acid encoding the second PEBL is located in a separate vector from the vector comprising the nucleic acids encoding the CAR and the first PEBL. In some instances, the nucleic acid encoding the second PEBL is located in the same vector that comprises the nucleic acids encoding the CAR and the first PEBL. In some instances, the nucleic acid encoding the CAR, the first PEBL, and the second PEBL are located in different vectors. In some instances, the nucleic acid encoding the second PEBL is located in a second retroviral vector or a lentiviral vector. In some embodiments, the second retroviral vector or lentiviral vector further comprises a kill gene. In some embodiments, the nucleic acids encoding the second PEBL and the kill gene are located in the same vector that comprises the nucleic acids encoding the CAR and the first PEBL.

In some aspects, the nucleic acid encoding the second PEBL and the nucleic acid encoding the kill gene are operably linked by Internal Ribosome Entry Site (IRES) or a ribosomal codon skipping site. In some embodiments, the ribosomal codon skipping site comprises a 2A self-cleaving peptide. In some instances, the nucleic acids encoding the second PEBL and the nucleic acid encoding the kill gene are located under the same promoter. In some instances, the nucleic acids encoding the second PEBL and the nucleic acid encoding the kill gene are located under different promoters.

In some instances, wherein the nucleic acid encoding the second PEBL is located in a separate vector from the vector comprising the nucleic acids encoding the CAR and the first PEBL, the two vectors can be introduced simultaneously. In some instances, wherein the nucleic acid encoding the second PEBL is located in a separate vector from the vector comprising the nucleic acids encoding the CAR and the first PEBL, the two vectors can be introduced sequentially. In some instances, wherein the nucleic acid encoding the second PEBL is located in a separate vector from the vector comprising the nucleic acids encoding the CAR and the first PEBL, the vector encoding the CAR and the first PEBL is introduced before the vector encoding the second PEBL. In some instances, wherein the nucleic acid encoding the second PEBL is located in a separate vector from the vector comprising the nucleic acids encoding the CAR and the first PEBL, the vector encoding the CAR and the first PEBL is introduced after the vector encoding the second PEBL.

In some instances, wherein the nucleic acid encoding the second PEBL and the kill gene are located in a separate vector from the vector comprising the nucleic acids encoding the CAR and the first PEBL, the two vectors can be introduced simultaneously. In some instances, wherein the nucleic acid encoding the second PEBL and the kill gene are located in a separate vector from the vector comprising the nucleic acids encoding the CAR and the first PEBL, the two vectors can be introduced sequentially. In some instances, wherein the nucleic acid encoding the second PEBL and the kill gene are located in a separate vector from the vector comprising the nucleic acids encoding the CAR and the first PEBL, the vector encoding the CAR and the first PEBL is introduced before the vector encoding the second PEBL and the kill gene. In some instances, wherein the nucleic acid encoding the second PEBL and the kill gene are located in a separate vector from the vector comprising the nucleic acids encoding the CAR and the first PEBL, the vector encoding the CAR and the first PEBL is introduced after the vector encoding the second PEBL and the kill gene.

In some instances, wherein the nucleic acid encoding the CAR, the first PEBL, and the second PEBL are located in different vectors, the different vectors can be introduced simultaneously. In some instances, wherein the nucleic acid encoding the CAR, the first PEBL, and the second PEBL are located in different vectors, the different vectors can be introduced sequentially.

In some instances, where the nucleic acid encoding the CAR, the first PEBL, the kill gene, and the second PEBL are located in different vectors, the different vectors can be introduced simultaneously. In some instances, where the nucleic acid encoding the CAR, the first PEBL, the kill gene, and the second PEBL are located in different vectors, the different vectors can be introduced sequentially.

In some instances, the engineered immune cell comprises a CAR and a first PEBL targeting an immune cell marker polypeptide (e.g., CD7, etc.) and a second PEBL targeting an immune cell receptor polypeptide (e.g., T cell receptor complex, CD3, etc.). Thus, described herein includes fratricide-resistant CAR-T cells expressing a CAR directed against CD7 and such CAR-T cell has reduced or no surface expression of CD7 as well as T cell receptor complex protein (e.g., CD3). In some instances, the present invention is based, in part, on co-expression of a chimeric antigen receptor (CAR) directed against CD7 and a protein expression blocker (PEBL) directed against CD7 using a bicistronic construct, such as a bicistronic viral vector, and another protein expression blocker (PEBL) directed against CD3 in immune cells (e.g., T cells) in a separate vector. In some instances, the co-expression of a chimeric antigen receptor (CAR) directed against CD7, a protein expression blocker (PEBL) directed against CD7, and a protein expression blocker (PEBL) directed against the immune cell receptor (e.g., CD3) in immune cells (e.g., T cells) can be achieved by using a vector that has triple transgenes (CAR, the first PEBL, and the second PEBL). In some instances, expressions of the three transgenes are driven by separate promoters coupled to the transgenes. In some instances, expressions of CAR and the first PEBL are driven by a first promoter and the expression of the second PEBL is driven by a second promoter.

In some instances, the engineered immune cell comprises a CAR, a first PEBL targeting an immune cell marker polypeptide (e.g., CD7, etc.), a second PEBL targeting an immune cell receptor polypeptide (e.g., T cell receptor complex, CD3, etc.), and a kill gene (e.g., CD20 or truncated CD20). Thus, described herein includes fratricide-resistant CAR-T cells expressing a CAR and CD20 or truncated CD20 directed against CD7 and such CAR-T cell has reduced or no surface expression of CD7 as well as T cell receptor complex protein (e.g., CD3). In some instances, the present invention is based, in part, on co-expression of a chimeric antigen receptor (CAR) directed against CD7 and a protein expression blocker (PEBL) directed against CD7 using a bicistronic construct, such as a bicistronic viral vector, and another protein expression blocker (PEBL) directed against CD3 in immune cells (e.g., T cells) along with a kill gene (e.g., CD20 or truncated CD20 (CD20t)) in another separate bicistronic vector. In some instances, the co-expression of a chimeric antigen receptor (CAR) directed against CD7, a protein expression blocker (PEBL) directed against CD7, a protein expression blocker (PEBL) directed against the immune cell receptor (e.g., CD3) in immune cells (e.g., T cells), and a kill gene (e.g., CD20 or truncated CD20 (CD20t)) can be achieved by using a vector that has quadruple transgenes (CAR, the first PEBL, the kill gene, and the second PEBL). In some instances, expressions of the four transgenes are driven by separate promoters coupled to the transgenes. In some instances, expressions of CAR and the first PEBL are driven by a first promoter and the expression of the kill gene and the second PEBL is driven by a second promoter.

In one aspect, the present invention relates to an engineered immune cell (e.g., an engineered T cell (PCART7-CD3PEBL(allo-PCART7)) comprising i) a bicistronic construct comprising a polynucleotide sequence encoding an anti-CD7 CAR and a polynucleotide sequence encoding an anti-CD7 PEBL, and ii) another construct comprising a polynucleotide sequence encoding an anti-CD3 PEBL. In some embodiments, the second construct is another bicistronic construct comprising a polynucleotide sequence encoding a kill gene or a suicide gene and a polynucleotide sequence encoding a CD3 PEBL, and these engineered immune cells can be an engineered T cell (allo-PCART7^{KG}). In some embodiments, the CAR comprises intracellular signaling domains of 4-1BB and CD3ζ, and an antibody (e.g., a single chain variable fragment or scFv) that specifically binds CD7. The CD7 CAR of the present invention is sometimes referred to herein as “anti-CD7-41BB-CD3ζ”. In some embodiments, the CAR also includes a CD8a hinge and transmembrane domain. In some embodiments, the anti-CD7 PEBL comprises an antibody (e.g., an scFv) that specifically binds CD7 and an intracellular localization sequence. In certain embodiments, the anti-CD7 PEBL comprises an antibody (e.g., an scFv) that specifically binds CD7, CD8a hinge and transmembrane domains, and an intracellular localization sequence.

CD7 is a 40 kDa type I transmembrane glycoprotein which is the primary marker for T cell malignancies, and which is highly expressed in all cases of T cell ALL, including early T-cell progenitor acute lymphoblastic leukemia (ETP-ALL). An anti-CD7 CAR can induce T cells to exert specific cytotoxicity against T cell malignancies. Further, T cell cytotoxicity has been shown to be markedly increased when an anti-CD7 CAR was used in combination with downregulation of CD7 expression on the effector T cells. Downregulation (e.g., elimination, reduction, and/or relocalization) of CD7 in a T cell via expression of anti-CD7 PEBL prevented the fratricidal effect exerted by the corresponding anti-CD7 CAR. This led to greater T cell recovery after CAR expression as compared to cells that retained the target antigen (e.g., CD7), and a more effective cytotoxicity against T leukemia/lymphoma cells. In some instances, the anti-CD3 PEBL comprises an antibody (e.g., an scFv) that specifically binds CD3 and an intracellular localization sequence. In certain embodiments, the anti-CD3 PEBL comprises an antibody (e.g., an scFv) that specifically binds CD3. In some instances, the CD3 binding domain comprises a portion or a fragment of an anti-CD3 antibody derived from Clone OKT3 or Clone UCHT1. In some instances, the CD3 binding domain comprises binding sequences derived from an anti-CD3 antibody derived from Clone OKT3 or Clone UCHT1.

T-cell receptor (TCR) is a protein complex found on the surface of T cells. Human T cells have four TCR genes: TCRα, TCRβ, TCRγ, and TCRδ, which form two distinct heterodimers: TCRα/TCRβ or TCRγ/TCRδ. The majority of mature T cells expresses TCRα and TCRβ isoforms, and these type of T cells are generally referred to as αβ T cells. In contrast, a small portion (0.5-5%) of T cells express TCRγ and TCRδ isoforms, and they are referred to as γδ T cells. Both TCRα/TCRβ or TCRγ/TCRδ heterodimers form multiprotein complexes with CD3 δ, γ, ε, and ζ chains (TCR-CD3 complex). These CD3 proteins associate with TCR via non-covalent hydrophobic interactions and may be required for a complete TCR localization on the cell surface. This TCR-CD3 complex of T cells plays an important role in coupling antigen recognition to several intracellular signal-transduction pathways, and it is the primary determinant of T cell development and activation of immune responses to foreign antigens, which can trigger GvHD. (see e.g., Shah K, et al. Signal Transduction and Targeted Therapy. 6:412, 2021; Kamiya T, et al. Blood Adv. 2(5):517-528, 2018).

Since the expression of endogenous TCR-CD3 complex carries the risk for GvHD, removing surface TCRαβ by using protein expression blockers (PEBL) on CD3 can block surface CD3 and TCRαβ expression, thereby reducing risk of GvHD. Anti-CD7 CAR T cells with double expression of anti-CD7 PEBL and anti-CD3 PEBL (sometimes referred herein as “PCART7-CD3PEBL” or “allo-PCART7”) not only have less the fratricidal effect but also reduce risk of mediating GvHD.

A kill gene or a suicide gene is a gene that, upon activation, induce cell death by killing itself via cellular process such as apoptosis or necrosis or by other mechanisms such as activation of effector cells in an immune response. Activation of a suicide gene can be done using a specific agent, e.g., antibody or pharmaceutical compound, which eventually leads to cell death. In some instances, a kill gene comprises CD20, p53 protein, inducible Caspase 9 (iCasp9), herpes simplex virus tyrosine kinase (HSV-TK), human thymidylate kinase (TMPK), epidermal growth factor receptor (EGFR), or derivative thereof. In some embodiments, the suicide gene comprises CD20, a derivative thereof, or a portion thereof (e.g., a functional fragment thereof). In some embodiments, the suicide gene comprises a modified CD20. In some embodiments, the suicide gene comprises a truncated CD20. In some embodiments, the CD20 is a human CD20. In some instances, the human CD20 is in a truncated CD20 form. In some instances, the engineered immune cells, e.g., allo-PCART7 cells, further express the kill gene, e.g., a truncated CD20, on the cell surface.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule. Nucleic acids and polynucleotides as used herein are interchangeable.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “sequence identity” means that two nucleotide sequences or two amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

As will be appreciated by those of skill in the art, in some aspects, the nucleic acid further comprises a plasmid sequence. The plasmid sequence can include, for example, one or more sequences of a promoter sequence, a selection marker sequence, or a locus-targeting sequence.

The term “promoter” or “promoter element” as used herein is defined as a DNA sequence recognized by the synthetic machinery of the cell, or introduced synthetic machinery, that may assist to initiate the specific transcription of a polynucleotide sequence.

The term retroviral vector” can refer to a gammaretroviral vector. A retroviral vector may include, e.g., a promoter, a packaging signal, a primer binding site (PBS), one or more (e.g., two) long terminal repeats (LTR), and polynucleotides of interest, e.g., a polynucleotide encoding a CAR and a polynucleotide encoding a PEBL. A retroviral vector may lack viral structural genes such as gag, pol, and env. Exemplary retroviral (e.g., gammaretroviral) vectors include Murine Embryonic Stem Cell Virus (MESV), Murine Stem Cell Virus (MSCV), Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Maetzig et al., Viruses, 2011; 3(6): 677-713.

The term “bicistronic expression” is typically achieved by operably linking the polynucleotides described herein to a promoter, and incorporating the bicistronic construct into an expression vector. The vectors can be suitable for replication and integration eukaryotes. Typical cloning vectors contain transcription and translation terminators, initiation sequences, and promoters useful for regulation of the expression of the desired nucleic acid sequence. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the immediate early cytomegalovirus (CMV), EF-1α, ubiquitin C, or phosphoglycerokinase (PGK) promoters. A strong constitutive promoter sequence capable of driving high levels of expression of any polynucleotide sequence operatively linked thereto can be used. Other constitutive promoter sequences may be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1 Ovian leukemia virus promoter, an Epstein-Barr virus immediate early promoter, a Rous sarcoma virus promoter, as well as human gene promoters such as, but not limited to, the actin promoter, the myosin promoter, and the like. In some embodiments, the promoter is an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

As used herein, “antibody” means an intact antibody or antigen-binding fragment of an antibody, including an intact antibody or antigen-binding fragment modified or engineered, or that is a human antibody. Examples of antibodies modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)₂, Fv, single chain antibodies (e.g., scFv), minibodies and diabodies.

The term “specifically (or selectively) binds” or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

In certain embodiments, the antibody that binds CD7 is a single-chain variable fragment antibody (“scFv antibody”). scFv refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun (1994) The Pharmacology Of Monoclonal Antibodies, vol. 113, Rosenburg and Moore eds. Springer-Verlag, New York, pp. 269-315. See also, PCT Publication No. WO 88/01649 and U.S. Pat. Nos. 4,946,778 and 5,260,203. As would be appreciated by those of skill in the art, various suitable linkers can be designed and tested for optimal function, as provided in the art, and as disclosed herein.

As used herein, an “engineered” immune cell includes an immune cell that has been genetically modified as compared to a naturally-occurring immune cell. For example, an engineered T cell produced according to the present methods carries a nucleic acid comprising a nucleotide sequence that does not naturally occur in a T cell from which it was derived, such as the nucleic acids exemplified herein.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, a “CD7 CAR+/CD7-negative” T cell refers to a T cell expressing a chimeric antigen receptor against human CD7 and having low or no surface expression of endogenous CD7. In some instances, a “CD7 CAR+/CD7-negative” T cell refers to “PCART7” cells. In some embodiments, the low or no surface expression of endogenous CD7 is due to expression of a PEBL against human CD7 which prevents or hinders endogenous CD7 protein to translocated to the surface of the T cell. In some instances, surface expression of CD7 can be determined using standard methods known to those in the art such as but not limited to immunocytochemistry, flow cytometry, or FACS.

As used herein, a “PCART7-CD3PEBL” T cell refers to a T cell expressing a chimeric antigen receptor against human CD7 and having low or no surface expression of endogenous CD7 and CD3. In some instances, the “PCART7-CD3PEBL” T cell refers to “allogeneic PCART7” or “allo-PCART7” cell. In some embodiments, the low or no surface expression of endogenous CD7 and CD3 is due to expression of a PEBL against human CD7 and another PEBL against human CD3, which prevents or hinders endogenous CD7 protein and endogenous CD3 protein, respectively, to translocate to the surface of the T cells. In some instances, surface expression of CD7 and CD3 can be determined using standard methods known to those in the art such as but not limited to immunocytochemistry, flow cytometry, or FACS.

As used herein, an “allo-PCART7^{KG}” T cell refers to a T cell expressing a chimeric antigen receptor against human CD7, having low or no surface expression of endogenous CD7 and CD3, and expressing a kill gene or suicide gene. In some embodiments, the low or no surface expression of endogenous CD7 and CD3 is due to expression of a PEBL against human CD7 and another PEBL against human CD3, which prevents or hinders endogenous CD7 protein and endogenous CD3 protein, respectively, to translocate to the surface of the T cells. In some embodiments, the kill gene is CD20. In some instances, surface expression of CD7, CD3, and CD20 can be determined using standard methods known to those in the art such as but not limited to immunocytochemistry, flow cytometry, or FACS.

The term “autologous” and its grammatical equivalents as used herein can refer to as originating from the same being. For example, a sample (e.g., cells) can be removed, processed, and given back to the same subject (e.g., patient) at a later time. An autologous process is distinguished from an allogenic process where the donor and the recipient are different subjects.

“Allogeneic” refers to a graft derived from a different animal of the same species.

As used herein, the terms “treat,” “treating,” or “treatment,” refer to counteracting a medical condition (e.g., a condition related to a T cell malignancy) to the extent that the medical condition is improved according to a clinically-acceptable standard.

As used herein, “subject” refers to a mammal (e.g., human, non-human primate, cow, sheep, goat, horse, dog, cat, rabbit, guinea pig, rat, mouse). In certain embodiments, the subject is a human. A “subject in need thereof” refers to a subject (e.g., patient) who has, or is at risk for developing, a disease or condition that can be treated (e.g., improved, ameliorated, prevented) by inducing T cells to exert specific cytotoxicity against malignant T cells.

As defined herein, a “therapeutic amount” refers to an amount that, when administered to a subject, is sufficient to achieve a desired therapeutic effect (treats a condition related to a T cell malignancy) in the subject under the conditions of administration. An effective amount of the agent to be administered can be determined by a clinician of ordinary skill using the guidance provided herein and other methods known in the art, and is dependent on several factors including, for example, the particular agent chosen, the subject's age, sensitivity, tolerance to drugs and overall well-being.

As used herein, a “kill gene” or a “suicide gene” refers to a gene that, upon activation, will induce cell death either by itself (e.g., self-induced apoptosis) or by other mechanism (e.g., as in effector cell mediated immune response, complement-dependent cytotoxicity, antibody-dependent cellular cytotoxicity, etc.). The activation of the kill gene can be done using an agent, e.g., antibody or drug, and this results in cell death.

As used herein, “multiplicity of infection (MOI)” refers to the ratio of the number of agents (e.g., viral particles) to the number of infection targets (e.g., host cells) in an infection medium. A MOI may affect transduction or infection. For a construct, a higher MOI can achieve a higher transduction rate.

As used herein, “vector copy number (VCN)” refers to the number of agents (e.g., viral particles) within a host cell. For example, a VCN of 1 can refer to a single viral particle transduced and integrated into the genome of a single host cell.

### DETAILED DESCRIPTION OF THE EMBODIMENTS

As described in detail below, the anti-CD7 CAR (also referred to as “CD7 CAR”) can comprise an antigen binding domain targeting CD7 based on the TH69 antibody. In some embodiments, the antigen binding domain of the CD7 CAR is based on the 3A1F antibody. In some embodiments, the antigen binding domain of the CD7 CAR is based on the T3-3A1 antibody. In some embodiments, the CD7 CAR of the present invention comprises an amino acid sequence selected from the group consisting of SEQ ID NO:28, SEQ ID NO:29, SEQ ID NO:30, and SEQ ID NO:31. In some embodiments, the CD7 CAR comprises an amino acid sequence having at least 90% sequence identity to one selected from the group consisting of SEQ ID NO:28, SEQ ID NO:29, SEQ ID NO:30, and SEQ ID NO:31. In some cases, the engineered immune cell of the present invention comprises the CD7 CAR of SEQ ID NO:28. In some cases, the engineered immune cell comprises the CD7 CAR having at least 90% sequence identity to SEQ ID NO:28. In some cases, the engineered immune cell comprises the CD7 CAR of SEQ ID NO: 29. In some cases, the engineered immune cell comprises the CD7 CAR having at least 90% sequence identity to SEQ ID NO:30. In some cases, the engineered immune cell comprises the CD7 CAR having at least 90% sequence identity to SEQ ID NO:30. In some cases, the engineered immune cell comprises the CD7 CAR having at least 90% sequence identity to SEQ ID NO:31. In some cases, the engineered immune cell comprises the CD7 CAR having at least 90% sequence identity to SEQ ID NO:31.

In some embodiments, the CD7 PEBL of the present disclosure comprises an amino acid sequence selected from the group consisting of SEQ ID NO:24, SEQ ID NO:25, SEQ ID NO:26, and SEQ ID NO:27. In some embodiments, the CD7 PEBL of the present invention comprises an amino acid sequence having at least 90% sequence identity to one selected from the group consisting of SEQ ID NO:24, SEQ ID NO:25, SEQ ID NO:26, and SEQ ID NO:27. In some instances, the engineered immune cell of the present invention comprises the CD7 PEBL of SEQ ID NO: 24. In some instances, the engineered immune cell of the present invention comprises the CD7 PEBL having at least 90% sequence identity to SEQ ID NO:24. In some instances, the engineered immune cell comprises the CD7 PEBL of SEQ ID NO: 25. In some instances, the engineered immune cell comprises the CD7 PEBL having at least 90% sequence identity to SEQ ID NO: 25. In some instances, the engineered immune cell comprises the CD7 PEBL of SEQ ID NO: 26. In some instances, the engineered immune cell comprises the CD7 PEBL having at least 90% sequence identity to SEQ ID NO: 26. In some instances, the engineered immune cell comprises the CD7 PEBL of SEQ ID NO: 27. In some instances, the engineered immune cell comprises the CD7 PEBL having at least 90% sequence identity to SEQ ID NO: 27.

In some embodiments, the engineered immune cell or population of engineered immune cells of the present invention comprises a CD7 PEBL of SEQ ID NO: 24 and a CD7 CAR of SEQ ID NO:28. In some embodiments, the engineered immune cell or population of engineered immune cells comprises a CD7 PEBL having at least 90% sequence identity to SEQ ID NO: 24 and a CD7 CAR having at least 90% sequence identity to SEQ ID NO:28. In some embodiments, the engineered immune cell or population of engineered immune cells comprises a CD7 PEBL of SEQ ID NO: 24 and a CD7 CAR of SEQ ID NO:30. In some embodiments, the engineered immune cell or population of engineered immune cells comprises a CD7 PEBL having at least 90% sequence identity SEQ ID NO: 24 and a CD7 CAR having at least 90% sequence identity SEQ ID NO:30. In some embodiments, the engineered immune cell or population of engineered immune cells comprises a CD7 PEBL of SEQ ID NO: 26 and a CD7 CAR of SEQ ID NO:28. In some embodiments, the engineered immune cell or population of engineered immune cells comprises a CD7 PEBL having at least 90% sequence identity SEQ ID NO: 26 and a CD7 CAR having at least 90% sequence identity SEQ ID NO:28. In some embodiments, the engineered immune cell or population of engineered immune cells comprises a CD7 PEBL of SEQ ID NO: 26 and a CD7 CAR of SEQ ID NO:30. In some embodiments, the engineered immune cell or population of engineered immune cells comprises a CD7 PEBL having at least 90% sequence identity SEQ ID NO: 26 and a CD7 CAR having at least 90% sequence identity SEQ ID NO:30. In some embodiments, the engineered immune cell or population of engineered immune cells comprises a CD7 PEBL of SEQ ID NO: 25 and a CD7 CAR of SEQ ID NO:29. In some embodiments, the engineered immune cell or population of engineered immune cells comprises a CD7 PEBL having at least 90% sequence identity SEQ ID NO: 25 and a CD7 CAR having at least 90% sequence identity SEQ ID NO:29. In some embodiments, the engineered immune cell or population of engineered immune cells comprises a CD7 PEBL of SEQ ID NO: 25 and a CD7 CAR of SEQ ID NO:31. In some embodiments, the engineered immune cell or population of engineered immune cells comprises a CD7 PEBL having at least 90% sequence identity SEQ ID NO: 25 and a CD7 CAR having at least 90% sequence identity SEQ ID NO:31. In some embodiments, the engineered immune cell or population of engineered immune cells comprises a CD7 PEBL of SEQ ID NO: 27 and a CD7 CAR of SEQ ID NO:29. In some embodiments, the engineered immune cell or population of engineered immune cells comprises a CD7 PEBL having at least 90% sequence identity SEQ ID NO: 27 and a CD7 CAR having at least 90% sequence identity SEQ ID NO:29. In some embodiments, the engineered immune cell or population of engineered immune cells comprises a CD7 PEBL of SEQ ID NO: 27 and a CD7 CAR of SEQ ID NO:31. In some embodiments, the engineered immune cell or population of engineered immune cells comprises a CD7 PEBL having at least 90% sequence identity SEQ ID NO: 27 and a CD7 CAR having at least 90% sequence identity SEQ ID NO:31.

In some instances, the engineered immune cell is an engineered T cell. In some embodiments, the engineered immune cell is an engineered CD4+ T cell. In some embodiments, the engineered immune cell is an engineered CD8+ T cell. In some embodiments, the engineered immune cell harboring the bicistronic construct or dual-promoter construct is generated from PBMCs. In some embodiments, the engineered immune cell harboring the bicistronic construct or dual-promoter construct is generated from purified CD4+ T cells. In some embodiments, the engineered immune cell harboring the bicistronic construct or dual-promoter construct is generated from purified CD8+ T cells. In some embodiments, the engineered immune cell harboring the bicistronic construct or dual-promoter construct is generated from a population of cells comprising purified CD4+ T cells and purified CD8+ T cells. In some embodiments, the engineered immune cell harboring the bicistronic construct or dual-promoter construct is generated from a population of cells comprising purified CD3+ T cells.

In some embodiments, the CD3 PEBL of the present disclosure comprises a CD3 binding domain and an intracellular localization sequence. In some instances, the CD3 binding domain comprises an antibody that binds a CD3/TCRαβcomplex protein. In some instances, the antibody is a single chain variable fragment (scFv) that binds the CD3/TCRαβcomplex protein selected from the group consisting of TCRα, TCRβ, CD3δ, CD3ε, CD3γ, and CD3ζ. The localizing domain can also include a transmembrane domain selected from a transmembrane domain derived from CD8a, CD8P, 4-1BB, CD28, CD34, CD4, FcεRI γ, CD16, OX40, CD3ζ, CD3 δ, CD3 ε, CD3γ, TCRα, CD32, CD64, VEGFR2, FAS, or FGFR2B. In some embodiments, the ER retention sequence comprises an amino acid sequence selected from KDEL (SEQ ID NO: 73), KDEL-like motif, KKMP (SEQ ID NO: 225), KKTN (SEQ ID NO: 226), KKXX, XXXKTN, KXKXX, or [HKR][DE][ED][LF] wherein X is any amino acid. In some embodiments, the ER retention sequences are described in, for example, Pelham, 1988, EMBO J; Raykhel, 2007, JCB; Robbi, 1991, JBC; Raykhel, 2007, JCB; Alanen, 2011, JMB; Schindler, 1993, Eur J Cell Biol; Jackson, 1990, EMBO J; Nilsson, 1989, Cell; Itin, 1995, EMBO J; Neve, 2003, Exp Cell Research; Nufer, 2003, JBC; Zerangue, 2000, PNAS; and Gao, 2014, Trends Plant Sci., each of which is incorporated by reference in its entirety.

In some instances, the CD3 binding domain comprises an antibody comprising sequences as described in the table below. In some instances, the anti-CD3 PEBL comprises an antibody (e.g., a scFv) that specifically binds CD3 and an intracellular localization sequence. In certain embodiments, the anti-CD3 PEBL comprises an antibody (e.g., a scFv) that specifically binds CD3. In some instances, the CD3 binding domain comprises a portion or a fragment of an anti-CD3 antibody derived from Clone OKT3 or Clone UCHT1. In some instances, the CD3 binding domain comprises binding sequences derived from an anti-CD3 antibody derived from Clone OKT3 or Clone UCHT1.

In some embodiments, the PEBL against CD3 comprises an amino acid sequence of SEQ ID NO: 101. In some embodiments, the CD3 binding domain comprises binding sequences derived from an anti-CD3 antibody derived from Clone OKT3 as in SEQ ID NO: 102. In some embodiments, the PEBL against CD3 comprises an amino acid sequence of SEQ ID NO: 103. In some embodiments, the CD3 binding domain comprises binding sequences derived from an anti-CD3 antibody derived from Clone UCHT1 as in SEQ ID NO: 104.

TABLE 1
Exemplary antibody sequences for CD3 binding domains

| SEQ ID NO | Name | Sequence |
|---|---|---|
| 101 | OKT3 PEBL5 | MALPVTALLLPLALLLHAARPQIVLTQSPAIMSASPGEKVTMTCSASSSVSYMNWYQQKSGTS |
| | sequence | PKRWIYDTSKLASGVPAHFRGSGSGTSYSLTISGMEAEDAATYYCQQWSSNPFTFGSGTKLEI |
| | | NRGGGGSGGGGSGGGGSGGGGSEVQLQQSGAELARPGASVKMSCKASGYTFTRYTMHWVKQRP |
| | | GQGLEWIGYINPSRGYTNYNQKFKDKATLTTDKSSSTAYMQLSSLTSEDSAVYYCARYYDDHY |
| | | CLDYWGQGTTLTVSSAGGGGSGGGGSGGGGSGGGGSAEKDEL |
| 102 | OKT3 scFv | QIVLTQSPAIMSASPGEKVTMTCSASSSVSYMNWYQQKSGTSPKRWIYDTSKLASGVPAHFRG |
| | | SGSGTSYSLTISGMEAEDAATYYCQQWSSNPFTFGSGTKLEINRGGGGSGGGGSGGGGSGGGG |
| | | SEVQLQQSGAELARPGASVKMSCKASGYTFTRYTMHWVKQRPGQGLEWIGYINPSRGYTNYNQ |
| | | KFKDKATLTTDKSSSTAYMQLSSLTSEDSAVYYCARYYDDHYCLDYWGQGTTLTVSSA |
| 110 | OKT3 VH | EVQLQQSGAELARPGASVKMSCKASGYTFTRYTMHWVKQRPGQGLEWIGYINPSRGYTNYNQK |
| | amino acid | FKDKATLTTDKSSSTAYMQLSSLTSEDSAVYYCARYYDDHYCLDYWGQGTTLTVSSA |
| | sequence | |
| 111 | OKT3 VL | QIVLTQSPAIMSASPGEKVTMTCSASSSVSYMNWYQQKSGTSPKRWIYDTSKLASGVPAHFRG |
| | amino acid | SGSGTSYSLTISGMEAEDAATYYCQQWSSNPFTFGSGTKLEINR |
| | sequence | |
| 112 | OKT3 VH | GAGGTCCAGCTGCAGCAGTCTGGGGCTGAACTGGCAAGACCTGGGGCCTCAGTGAAGATGTCC |
| | nucleotide | TGCAAGGCTTCTGGCTACACCTTTACTAGGTACACGATGCACTGGGTAAAACAGAGGCCTGGA |
| | sequence | CAGGGTCTGGAATGGATTGGATACATTAATCCTAGCCGTGGTTATACTAATTACAATCAGAAG |
| | | TTCAAGGACAAGGCCACATTGACTACAGACAAATCCTCCAGCACAGCCTACATGCAACTGAGC |
| | | AGCCTGACATCTGAGGACTCTGCAGTCTATTACTGTGCAAGATATTATGATGATCATTACTGC |
| | | CTTGACTACTGGGGCCAAGGCACCACTCTCACAGTCTCCTCAGCC |
| 113 | OKT3 VL | CAAATTGTTCTCACCCAGTCTCCAGCAATCATGTCTGCATCTCCAGGGGAGAAGGTCACCATG |
| | nucleotide | ACCTGCAGTGCCAGCTCAAGTGTAAGTTACATGAACTGGTACCAGCAGAAGTCAGGCACCTCC |
| | sequence | CCCAAAAGATGGATTTATGACACATCCAAACTGGCTTCTGGAGTCCCTGCTCACTTCAGGGGC |
| | | AGTGGGTCTGGGACCTCTTACTCTCTCACAATCAGCGGCATGGAGGCTGAAGATGCTGCCACT |
| | | TATTACTGCCAGCAGTGGAGTAGTAACCCATTCACGTTCGGCTCGGGGACAAAGTTGGAAATA |
| | | AACCGG |
| 200 | OCKT3 | RYTMH |
| | HC CDR1 | |
| | (Kabat) | |
| 201 | OCKT3 | YINPSRGYTNYNQKFKD |
| | HC CDR2 | |
| | (Kabat) | |
| 202 | OCKT3 | YYDDHYCLDY |
| | HC CDR3 | |
| | (Kabat) | |
| 203 | OCKT3 | GYTFTRY |
| | HC CDR1 | |
| | (Chothia) | |
| 204 | OCKT3 | NPSRGY |
| | HC CDR2 | |
| | (Chothia) | |
| 205 | OCKT3 | YYDDHYCLDY |
| | HC CDR3 | |
| | (Chothia) | |
| 206 | OCKT3 | SASSSVSYMN |
| | LC CDR1 | |
| | (Kabat) | |
| 207 | OCKT3 | DTSKLAS |
| | LC CDR2 | |
| | (Kabat) | |
| 208 | OCKT3 | QQWSSNPFT |
| | LC CDR3 | |
| | (Kabat) | |
| 209 | OCKT3 | SASSSVSYMN |
| | LC CDR1 | |
| | (Chothia) | |
| 210 | OCKT3 | DTSKLAS |
| | LC CDR2 | |
| | (Chothia) | |
| 211 | OCKT3 | QQWSSNPFT |
| | LC CDR3 | |
| | (Chothia) | |
| 103 | UCHT1 | MALPVTALLLPLALLLHAARPDIQMTQTTSSLSASLGDRVTISCRASQDIRNYLNWYQQKPDG |
| | PEBL22 | TVKLLIYYTSRLHSGVPSKFSGSGSGTDYSLTISNLEQEDIATYFCQQGNTLPWTFAGGTKLE |
| | sequence | IKGGGGSGGGGSGGGGSGGGGSEVQLQQSGPELVKPGASMKISCKASGYSFTGYTMNWVKQSH |
| | | GKNLEWMGLINPYKGVSTYNQKFKDKATLTVDKSSSTAYMELLSLTSEDSAVYYCARSGYYGD |
| | | SDWYFDVWGQGTTLTVFSEQKLISEEDLGGGGSGGGGSGGGGSGGGGSAEKDEL |
| 104 | UCHT1 scFv | DIQMTQTTSSLSASLGDRVTISCRASQDIRNYLNWYQQKPDGTVKLLIYYTSRLHSGVPSKFS |
| | | GSGSGTDYSLTISNLEQEDIATYFCQQGNTLPWTFAGGTKLEIKGGGGSGGGGSGGGGSGGGG |
| | | SEVQLQQSGPELVKPGASMKISCKASGYSFTGYTMNWVKQSHGKNLEWMGLINPYKGVSTYNQ |
| | | KFKDKATLTVDKSSSTAYMELLSLTSEDSAVYYCARSGYYGDSDWYFDVWGQGTTLTVFS |
| 114 | UCHT1 VH | EVQLQQSGPELVKPGASMKISCKASGYSFTGYTMNWVKQSHGKNLEWMGLINPYKGVSTYNQK |
| | amino acid | FKDKATLTVDKSSSTAYMELLSLTSEDSAVYYCARSGYYGDSDWYFDVWGQGTTLTVFS |
| | sequence | |
| 115 | UCHT1 VL | DIQMTQTTSSLSASLGDRVTISCRASQDIRNYLNWYQQKPDGTVKLLIYYTSRLHSGVPSKFS |
| | amino acid | GSGSGTDYSLTISNLEQEDIATYFCQQGNTLPWTFAGGTKLEIK |
| | sequence | |
| 116 | UCHT1 VH | GAGGTGCAGCTGCAGCAGAGCGGCCCCGAGCTGGTGAAGCCCGGCGCCAGCATGAAGATC |
| | nucleotide | AGCTGCAAGGCCAGCGGCTACAGCTTCACCGGCTACACCATGAACTGGGTGAAGCAGAGC |
| | sequence | CACGGCAAGAACCTGGAGTGGATGGGCCTGATCAACCCCTACAAGGGCGTGAGCACCTAC |
| | | AACCAGAAGTTCAAGGACAAGGCCACCCTGACCGTGGACAAGAGCAGCAGCACCGCCTAC |
| | | ATGGAGCTGCTGAGCCTGACCAGCGAGGACAGCGCCGTGTACTACTGCGCCAGGAGCGGC |
| | | TACTACGGCGACAGCGACTGGTACTTCGACGTGTGGGGCCAGGGCACCACCCTGACCGTG |
| | | TTCAGC |
| 117 | UCHT1 VL | GACATCCAGATGACCCAGACCACCAGCAGCCTGAGCGCCAGCCTGGGCGACAGGGTGACC |
| | nucleotide | ATCAGCTGCAGGGCCAGCCAGGACATCAGGAACTACCTGAACTGGTACCAGCAGAAGCCC |
| | sequence | GACGGCACCGTGAAGCTGCTGATCTACTACACCAGCAGGCTGCACAGCGGCGTGCCCAGC |
| | | AAGTTCAGCGGCAGCGGCAGCGGCACCGACTACAGCCTGACCATCAGCAACCTGGAGCAG |
| | | GAGGACATCGCCACCTACTTCTGCCAGCAGGGCAACACCCTGCCCTGGACCTTCGCCGGC |
| | | GGCACCAAGCTGGAGATCAAG |
| 212 | UCHT1 HC | GYTMN |
| | CDR1 (Kabat) | |
| 213 | UCHT1 HC | LINPYKGVSTYNQKFKD |
| | CDR2 (Kabat) | |
| 214 | UCHT1 HC | SGYYGDSDWYFDV |
| | CDR3 (Kabat) | |
| 215 | UCHT1 HC | GYSFTGY |
| | CDR1 | |
| | (Chothia) | |
| 216 | UCHT1 HC | NPYKGV |
| | CDR2 | |
| | (Chothia) | |
| 217 | UCHT1 HC | SGYYGDSDWYFDV |
| | CDR3 | |
| | (Chothia) | |
| 218 | UCHT1 LC | RASQDIRNYLN |
| | CDR1 (Kabat) | |
| 219 | UCHT1 LC | YTSRLHS |
| | CDR2 (Kabat) | |
| 220 | UCHT1 LC | QQGNTLPWT |
| | CDR3 (Kabat) | |
| 221 | UCHT1 LC | RASQDIRNYLN |
| | CDR1 | |
| | (Chothia) | |
| 222 | UCHT1 LC | YTSRLHS |
| | CDR2 | |
| | (Chothia) | |
| 223 | UCHT1 LC | QQGNTLPWT |
| | CDR3 | |
| | (Chothia) | |

In some instances, the engineered immune cells such as CD7 CAR+/CD7-negative T cell or PCART7-CD3PEBL T cell can further comprises a fourth nucleic acid comprising a suicide gene or a kill gene. In some embodiments, the suicide gene or a kill gene comprises CD20 or a derivative thereof. In some embodiments, the suicide gene or a kill gene comprises CD20, a derivative thereof, or a portion thereof. In some embodiments, the suicide gene or a kill gene comprises a modified CD20. In some embodiments, the suicide gene comprises a truncated CD20. In some embodiments, the suicide gene or a kill gene comprises CD20, p53 protein, inducible Caspase 9 (iCasp9), herpes simplex virus tyrosine kinase (HSV-TK), human thymidylate kinase (TMPK), epidermal growth factor receptor (EGFR), or derivative thereof. In some embodiments, the fourth nucleic acid is located in the second expression vector, wherein the second expression vector encodes the second PEBL. In some embodiments, the fourth nucleic acid is located in the first expression vector, wherein the first expression vector encodes the CAR and the first PEBL. In some embodiments, the fourth nucleic acid is located in a separate expression vector, e.g., a third expression vector.

In some instances, the nucleic acid encoding the second PEBL and the nucleic acid encoding the fourth nucleic acid comprising a suicide gene are located in a bicistronic vector (e.g., bicistronic retroviral vector, bicistronic lentiviral vector) such that the second PEBL and the fourth nucleic acid encoding the suicide gene are expressed simultaneously. In some aspects, the nucleic acid encoding the second PEBL and the nucleic acid encoding the suicide gene are the operably linked by Internal Ribosome Entry Site (IRES) or a ribosomal codon skipping site. In some embodiments, the ribosomal codon skipping site comprises a 2A self-cleaving peptide. In some instances, the nucleic acid encoding the second PEBL and the nucleic acid encoding the suicide gene is located in a second retroviral vector or a lentiviral vector.

In some instances, wherein the nucleic acid encoding the second PEBL is located in a separate vector from the vector comprising the nucleic acids encoding the CAR and the first PEBL, the two vectors can be introduced simultaneously. In some instances, wherein the nucleic acid encoding the second PEBL is located in a separate vector from the vector comprising the nucleic acids encoding the CAR and the first PEBL, the two vectors can be introduced sequentially. In some instances, wherein the nucleic acid encoding the second PEBL is located in a separate vector from the vector comprising the nucleic acids encoding the CAR and the first PEBL, the vector encoding the CAR and the first PEBL is introduced before the vector encoding the second PEBL. In some instances, wherein the nucleic acid encoding the second PEBL is located in a separate vector from the vector comprising the nucleic acids encoding the CAR and the first PEBL, the vector encoding the CAR and the first PEBL is introduced after the vector encoding the second PEBL.

In some instances, wherein the nucleic acid encoding the suicide gene or the kill gene and the second PEBL is located in a separate vector from the vector comprising the nucleic acids encoding the CAR and the first PEBL, the two vectors can be introduced simultaneously. In some instances, wherein the nucleic acid encoding the suicide gene or the kill gene and the second PEBL is located in a separate vector from the vector comprising the nucleic acids encoding the CAR and the first PEBL, the two vectors can be introduced sequentially. In some instances, wherein the nucleic acid encoding the suicide gene or the kill gene and the second PEBL is located in a separate vector from the vector comprising the nucleic acids encoding the CAR and the first PEBL, the vector encoding the CAR and the first PEBL is introduced before the vector encoding the suicide gene or the kill gene and the second PEBL. In some instances, wherein the nucleic acid encoding the suicide gene or the kill gene and the second PEBL is located in a separate vector from the vector comprising the nucleic acids encoding the CAR and the first PEBL, the vector encoding the CAR and the first PEBL is introduced after the vector encoding the suicide gene or the kill gene and the second PEBL.

In some instances, the nucleic acids encoding the CAR, the first PEBL, and the second PEBL are located in different vectors. In some instances, the nucleic acids encoding the CAR, the first PEBL, and the second PEBL are located in the same vector. In some instances, wherein the nucleic acid encoding the CAR, the first PEBL, and the second PEBL is located in different vectors, the different vectors can be introduced simultaneously. In some instances, wherein the nucleic acid encoding the CAR, the first PEBL, and the second PEBL is located in different vectors, the different vectors can be introduced sequentially.

In some instances, the nucleic acids encoding the CAR, the first PEBL, the suicide gene, and the second PEBL are located in different vectors. In some instances, the nucleic acids encoding the CAR, the first PEBL, the suicide gene, and the second PEBL are located in the same vector. In some instances, wherein the nucleic acids encoding the CAR, the first PEBL, the suicide gene or the kill gene, and the second PEBL are located in different vectors, the different vectors can be introduced simultaneously. In some instances, wherein the nucleic acids encoding the CAR, the first PEBL, the suicide gene or the kill gene, and the second PEBL are located in different vectors, the different vectors can be introduced sequentially.

In some instances, wherein the nucleic acid encoding the second PEBL and the suicide gene is located in a separate vector from the vector comprising the nucleic acids encoding the CAR and the first PEBL, the two vectors can be introduced simultaneously. In some instances, wherein the nucleic acid encoding the second PEBL and the suicide gene is located in a separate vector from the vector comprising the nucleic acids encoding the CAR and the first PEBL, the two vectors can be introduced sequentially. In some instances, wherein the nucleic acid encoding the second PEBL and the suicide gene is located in a separate vector from the vector comprising the nucleic acids encoding the CAR and the first PEBL, the vector encoding the CAR and the first PEBL is introduced before the vector encoding the second PEBL and the suicide gene. In some instances, wherein the nucleic acid encoding the second PEBL and the suicide gene is located in a separate vector from the vector comprising the nucleic acids encoding the CAR and the first PEBL, the vector encoding the CAR and the first PEBL is introduced after the vector encoding the second PEBL and the suicide gene.

In some instances, wherein the nucleic acid encoding the CAR, the first PEBL, the second PEBL, and the suicide gene is located in different vectors, the different vectors can be introduced simultaneously. In some instances, wherein the nucleic acid encoding the CAR, the first PEBL, the second PEBL, and the suicide gene is located in different vectors, the different vectors can be introduced sequentially.

In some instances, the engineered immune cell is an NK cell. In some instances, the engineered immune cell is CD56, CD161, CD16, CD94 or CD57 positive NK cell.

In some aspects, provided herein is an engineered immune cell comprising: (a) a first nucleic acid sequence encoding (i) a domain that binds to a subunit of a T cell receptor (TCR) complex linked to (ii) a synthetic localizing domain. The engineered immune cell can further comprise (b) a second nucleic acid sequence encoding a chimeric antigen receptor (CAR).

The engineered immune cell can further comprise (c) a third nucleic acid sequence encoding a surface polypeptide binding domain linked to a synthetic surface polypeptide localizing domain. The engineered immune cell can comprise at least twice as much of the first nucleic acid sequence as the second nucleic acid sequence or the third nucleic acid sequence.

The engineered immune cell can comprise a first nucleic acid sequence encoding a CD3ε binding domain linked to a synthetic localizing domain, wherein the CD36 binding domain comprises a heavy chain complementarity-determining region (HC CDR1) of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223; a second nucleic acid sequence encoding a chimeric antigen receptor (CAR); and a third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is at least about 1.5-fold, at least about 2.0-fold, at least about 2.5-fold, at least about 3.0-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 14.0-fold, at least about 15.0-fold, at least about 20.0-fold, at least about 25.0-fold, at least about 30.0-fold, at least about 35.0-fold, at least about 40.0-fold, at least about 45.0-fold, or at least about 50.0-fold more than the amount of the second nucleic acid sequence encoding the CAR or the third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is from about 1.5-fold to about 50-fold more than the amount of the second nucleic acid sequence encoding the CAR or the third nucleic acid sequence encoding a surface polypeptide binding domain linked to a synthetic surface polypeptide localizing domain. In some embodiments, the amount of the first nucleic acid sequence encoding (i) a domain that binds to a subunit of a T cell receptor (TCR) complex linked to (ii) a synthetic localizing domain in the engineered immune cell is from about 1.5 fold to about 2 fold, from about 1.5 fold to about 5 fold, from about 1.5 fold to about 7.5 fold, from about 1.5 fold to about 10 fold, from about 1.5 fold to about 15 fold, from about 1.5 fold to about 20 fold, from about 1.5 fold to about 25 fold, from about 1.5 fold to about 30 fold, from about 1.5 fold to about 35 fold, from about 1.5 fold to about 40 fold, from about 1.5 fold to about 50 fold, from about 2 fold to about 5 fold, from about 2 fold to about 7.5 fold, from about 2 fold to about 10 fold, from about 2 fold to about 15 fold, from about 2 fold to about 20 fold, from about 2 fold to about 25 fold, from about 2 fold to about 30 fold, from about 2 fold to about 35 fold, from about 2 fold to about 40 fold, from about 2 fold to about 50 fold, from about 5 fold to about 7.5 fold, from about 5 fold to about 10 fold, from about 5 fold to about 15 fold, from about 5 fold to about 20 fold, from about 5 fold to about 25 fold, from about 5 fold to about 30 fold, from about 5 fold to about 35 fold, from about 5 fold to about 40 fold, from about 5 fold to about 50 fold, from about 7.5 fold to about 10 fold, from about 7.5 fold to about 15 fold, from about 7.5 fold to about 20 fold, from about 7.5 fold to about 25 fold, from about 7.5 fold to about 30 fold, from about 7.5 fold to about 35 fold, from about 7.5 fold to about 40 fold, from about 7.5 fold to about 50 fold, from about 10 fold to about 15 fold, from about 10 fold to about 20 fold, from about 10 fold to about 25 fold, from about 10 fold to about 30 fold, from about 10 fold to about 35 fold, from about 10 fold to about 40 fold, from about 10 fold to about 50 fold, from about 15 fold to about 20 fold, from about 15 fold to about 25 fold, from about 15 fold to about 30 fold, from about 15 fold to about 35 fold, from about 15 fold to about 40 fold, from about 15 fold to about 50 fold, from about 20 fold to about 25 fold, from about 20 fold to about 30 fold, from about 20 fold to about 35 fold, from about 20 fold to about 40 fold, from about 20 fold to about 50 fold, from about 25 fold to about 30 fold, from about 25 fold to about 35 fold, from about 25 fold to about 40 fold, from about 25 fold to about 50 fold, from about 30 fold to about 35 fold, from about 30 fold to about 40 fold, from about 30 fold to about 50 fold, from about 35 fold to about 40 fold, from about 35 fold to about 50 fold, or about 40 fold to about 50 fold more than the amount of the second nucleic acid sequence encoding the CAR or the third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 20 times, at least about 25 times, at least about 30 times, at least about 35 times, or at least about 40 times more than the amount of the second nucleic acid sequence encoding the CAR or the third nucleic acid sequence encoding a surface polypeptide binding domain linked to a synthetic surface polypeptide localizing domain. In some embodiments, the amount of the first nucleic acid sequence encoding (i) a domain that binds to a subunit of a T cell receptor (TCR) complex linked to (ii) a synthetic localizing domain in the engineered immune cell is at most about 40 times, at most about 35 times, at most about 30 times, at most about 25 times, at most about 20 times, at most about 15 times, at most about 10 times, at most about 9 times, at most about 8 times, at most about 7 times, at most about 6 times, at most about 5 times, at most about 4 times, at most about 3 times, or at most about 2 times more than the amount of the second nucleic acid sequence encoding the CAR or the third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is from about 2 times to about 20 times more than the amount of the second nucleic acid sequence encoding the CAR or the third nucleic acid sequence encoding a surface polypeptide binding domain linked to a synthetic surface polypeptide localizing domain. In some embodiments, the amount of the first nucleic acid sequence encoding (i) a domain that binds to a subunit of a T cell receptor (TCR) complex linked to (ii) a synthetic localizing domain in the engineered immune cell is from about 2 times to about 3 times, from about 2 times to about 4 times, from about 2 times to about 5 times, from about 2 times to about 6 times, from about 2 times to about 7 times, from about 2 times to about 8 times, from about 2 times to about 9 times, from about 2 times to about 10 times, from about 2 times to about 15 times, from about 2 times to about 7 times, from about 2 times to about 20 times, from about 3 times to about 4 times, from about 3 times to about 5 times, from about 3 times to about 6 times, from about 3 times to about 7 times, from about 3 times to about 8 times, from about 3 times to about 9 times, from about 3 times to about 10 times, from about 3 times to about 15 times, from about 3 times to about 7 times, from about 3 times to about 20 times, from about 4 times to about 5 times, from about 4 times to about 6 times, from about 4 times to about 7 times, from about 4 times to about 8 times, from about 4 times to about 9 times, from about 4 times to about 10 times, from about 4 times to about 15 times, from about 4 times to about 7 times, from about 4 times to about 20 times, from about 5 times to about 6 times, from about 5 times to about 7 times, from about 5 times to about 8 times, from about 5 times to about 9 times, from about 5 times to about 10 times, from about 5 times to about 15 times, from about 5 times to about 7 times, from about 5 times to about 20 times, from about 6 times to about 7 times, from about 6 times to about 8 times, from about 6 times to about 9 times, from about 6 times to about 10 times, from about 6 times to about 15 times, from about 6 times to about 7 times, from about 6 times to about 20 times, from about 7 times to about 8 times, from about 7 times to about 9 times, from about 7 times to about 10 times, from about 7 times to about 15 times, from about 7 times to about 7 times, from about 7 times to about 20 times, from about 8 times to about 9 times, from about 8 times to about 10 times, from about 8 times to about 15 times, from about 8 times to about 7 times, from about 8 times to about 20 times, from about 9 times to about 10 times, from about 9 times to about 15 times, from about 9 times to about 7 times, from about 9 times to about 20 times, from about 10 times to about 15 times, from about 10 times to about 7 times, from about 10 times to about 20 times, from about 15 times to about 7 times, from about 15 times to about 20 times, or about 7 times to about 20 times more than the amount of the second nucleic acid sequence encoding the CAR or the third nucleic acid sequence encoding a surface polypeptide binding domain linked to a synthetic surface polypeptide localizing domain.

The domain linked to a synthetic localizing domain can bind to a subunit of a TCR complex. In some embodiments, the subunit of the TCR complex is CD38, CD3γ, or CD36. In some embodiments, the subunit of the TCR complex is CD3ε. In some embodiments, the domain binding to the subunit of the TCR complex (e.g., CD38) can be an antibody or an antigen binding domain. In some embodiments, the antibody binds to CD3ε (e.g., an anti-CD3ε antibody). In some embodiments, the antibody is a single chain Fv (scFv) or a single domain antibody (sdAb).

The anti-CD3ε antibody can be any anti-CD3ε antibody described herein or known in the art. In some embodiments, the anti-CD3ε antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 70%, at least 75%, at least 80%, at least 85%, at least 90%, at least 95%, at least 96%, at least 97%, at least 98%, at least 99%, or 100% sequence identity to SEQ ID NO: 114. In some embodiments, the anti-CD3, antibody comprises a light chain variable domain having an amino acid sequence with at least 70%, at least 75%, at least 80%, at least 85%, at least 90%, at least 95%, at least 96%, at least 97%, at least 98%, at least 99%, or 100% sequence identity to SEQ ID NO: 115. In some embodiments, the anti-CD3, antibody comprises an amino acid sequence having at least 70%, at least 75%, at least 80%, at least 85%, at least 90%, at least 95%, at least 96%, at least 97%, at least 98%, at least 99%, or 100% sequence identity to SEQ ID NO: 104.

In some embodiments, the engineered immune cell described herein comprises a synthetic localizing domain. In some embodiments, the synthetic localizing domain comprises an ER retention signal (e.g., ER retention sequence), a Golgi retention sequence, and/or a proteasome localizing sequence. In some embodiments, the ER retention signal comprises an amino acid sequence of KDEL (SEQ ID NO: 73) or KKXX, where X can be any amino acid. In some embodiments, the synthetic localizing domain can comprise a Myc tag and/or a linker sequence. In some embodiments, the Myc tag comprises the amino acid sequence EQKLISEEDL (SEQ ID NO: 227). In some embodiments, the linker sequence can be (GGGGS)n (SEQ ID NO: 232), where n is any integer from 1 to 12.

In some embodiments, the first nucleic acid sequence of an engineered immune cell described herein comprises, in 5′ to 3′ direction, a sequence encoding the domain that binds to a subunit of the TCR complex, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73). In some embodiments, the first nucleic acid sequence of an engineered immune cell described herein comprises, in 5′ to 3′ direction, a sequence encoding an anti-CD3, antibody, a sequence encoding a Myc tag, a sequence encoding a (GGGGS)₄ sequence (SEQ ID NO: 79), and a sequence encoding the ER retention signal comprising the amino acid sequence KDEL (SEQ ID NO: 73).

In some embodiments, a second nucleic acid sequence and a third nucleic acid sequence are on the same nucleic acid molecule. In some embodiments, a second nucleic acid sequence and a third nucleic acid sequence are on different nucleic acid molecules. In some embodiments, the same nucleic acid molecule may be a vector.

In some embodiments, the first nucleic acid sequence may be on a nucleic acid molecule separate from the second or the third nucleic acid sequence. The nucleic acid molecule having the first nucleic acid sequence may be an expression vector.

In some embodiments, the engineered immune cell described herein may comprise a nucleic acid sequence encoding a kill gene (e.g., a suicide gene). In some embodiments, an expression vector with a first nucleic acid sequence can also comprise a nucleic acid sequence encoding a kill gene. The kill gene can be CD20 or a fragment thereof. In some embodiments, an expression vector can comprise a ribosome codon skipping site between a first nucleic acid sequence and another nucleic acid. In some embodiments, an expression vector can comprise a ribosome codon skipping site between a first nucleic acid sequence and a fourth nucleic acid sequence that encodes a kill gene. A ribosome codon skipping site can refer to a mechanism of translation in which a specific viral peptide prevents a ribosome from covalently linking a new inserted amino acid. A ribosome skipping event can be induced by a “2A-like”, or CHYSEL (cis-acting hydrolase element) sequence. In some embodiments, the ribosome codon skipping site can comprise a 2A self-cleaving peptide.

In some embodiments, an expression vector can be a lentiviral vector, a retroviral vector, an adenoviral vector, or an adeno-associated viral vector. In some embodiments, an expression vector can be a lentiviral vector.

In some embodiments, a CAR described herein comprises a target binding domain that binds to the surface polypeptide. In some embodiments, the surface polypeptide can be CD7. In some embodiments, a surface polypeptide binding domain and a target binding domain may comprise a first antibody or antigen binding domain and a second antibody or antigen binding domain respectively. In some embodiments, an amino acid sequence of the surface polypeptide binding domain (e.g., binding domain of a CAR) and an amino acid sequence of a target binding domain (e.g., binding domain of a PEBL) can be at least 60%, at least 65%, at least 70%, at least 75%, at least 80%, at least 85%, at least 90%, at least 95%, at least 96%, at least 97%, at least 98%, or at least 99%, identical. In some embodiments, an amino acid sequence of the surface polypeptide binding domain (e.g., binding domain of a CAR) and an amino acid sequence of a target binding domain (e.g., binding domain of a PEBL) can be at least 80% identical. In some embodiments, an amino acid sequence of the surface polypeptide binding domain (e.g., binding domain of a CAR) and an amino acid sequence of a target binding domain (e.g., binding domain of a PEBL) can be at least 90% identical. In some embodiments, an amino acid sequence of the surface polypeptide binding domain (e.g., binding domain of a CAR) and an amino acid sequence of a target binding domain (e.g., binding domain of a PEBL) can be identical.

In some embodiments, an antibody of an engineered immune cell described herein comprises a HC CDR1 of SEQ ID NO: 47, a HC CDR2 of SEQ ID NO: 48, a HC CDR3 of SEQ ID NO: 49, and a light chain (LC) CDR1 of SEQ ID NO: 44, a LC CDR2 of SEQ ID NO: 45, a LC CDR3 of SEQ ID NO: 46.

In some embodiments, an expression vector described herein can be a bicistronic lentiviral expression vector. In some embodiments, the nucleic acid sequence encoding the CAR and the nucleic acid sequence encoding the PEBL can be operably linked in a bicistronic lentiviral expression vector. In some embodiments, the nucleic acid sequence encoding the CAR and the nucleic acid sequence encoding the PEBL can be operably linked by an Internal Ribosome Entry Site (IRES) or a ribosomal codon skipping site.

In some embodiments, the synthetic localizing domain or the synthetic surface polypeptide localizing domain described herein may comprise an endoplasmic reticulum (ER) retention sequence, a Golgi retention sequence, a proteasome localizing sequence, or a transmembrane domain. In some embodiments, the ER retention sequence comprises the amino acid sequence KDEL (SEQ ID NO: 73) and an additional nucleic acid sequence further comprises a sequence encoding a linker that couples the surface polypeptide binding domain and the synthetic surface polypeptide localizing domain. In some embodiments, the synthetic localizing domain and the synthetic surface polypeptide localizing domain comprise the same ER retention sequence. In some embodiments, the synthetic localizing domain and the synthetic surface polypeptide localizing domain comprise different ER retention sequences.

In some embodiments, a synthetic surface polypeptide localizing domain may refer to a localizing domain that directs the surface polypeptide to an intracellular compartment. In some embodiments, the synthetic surface polypeptide localizing domain can direct the surface polypeptide to an intracellular compartment indirectly via a binding domain. For example, the synthetic surface polypeptide localizing domain can be linked to a binding domain that targets the surface polypeptide. The surface polypeptide can be CD7. In some embodiments, the binding domain can be an antibody or fragment thereof.

In some embodiments, the CAR comprises a transmembrane domain and an intracellular signaling domain. In some embodiments, the intracellular signaling domain can be a 4-1BB intracellular signaling domain, a CD3ζ intracellular signaling domain, or a CD28 intracellular signaling domain. In some embodiments, the CAR may comprise a costimulatory domain. In some embodiments, a costimulatory domain of a CAR described herein may comprise inducible T cell co-stimulator (ICOS), CD27, MYD88, CD40, CD30, CD84, CRTAM, DR3, GITR, HVEM, ICOS, SLAMF1, TIM1, or OX40 (CD134).

In some embodiments, in an engineered immune cell described herein, expression of the TCR complex and the surface polypeptide may be downregulated. In some embodiments, the engineered immune cell may be a T cell or a natural killer (NK) cell.

Also provided herein is a cell population comprising such engineered immune cells. In some aspects, the present disclosure provides a cell population of engineered immune cells described herein comprising (i) a nucleic acid sequence encoding a domain that binds to a subunit of a T cell receptor (TCR) complex linked to a synthetic localizing domain; and (ii) a nucleic acid sequence encoding a chimeric antigen receptor (CAR).

In some embodiments, less than 0.5%, less than 1%, less than 1.5%, less than 1.6%, less than 1.7%, less than 1.8%, less than 1.9%, less than 2%, less than 2.1%, less than 2.2%, less than 2.3%, less than 2.4%, less than 2.5%, less than 2.6%, less than 2.7%, less than 2.8%, less than 2.9%, less than 3%, less than 3.5%, or less than 4% of the cells in the cell population express the TCR complex on their surface. In some embodiments, no more than 4%, no more than 3.5%, no more than 3%, no more than 2.9%, no more than 2.8%, no more than 2.7%, no more than 2.6%, no more than 2.5%, no more than 2.4%, no more than 2.3%, no more than 2.2%, no more than 2.1%, no more than 2%, no more than 1.9%, no more than 1.8%, no more than 1.7%, no more than 1.6%, no more than 1.5%, no more than 1%, or no more than 0.5% of the cells in the cell population express the TCR complex on their surface. In some embodiments, the TCR complex comprises a subunit, wherein the subunit may be a CD3.

In some embodiments, the percentage of cells in the cell population expressing CD3 may be determined subsequent to a CD3 depletion step. In some embodiments, the percentage of cells in the cell population expressing CD3 is less than 0.5%, less than 1%, less than 1.5%, less than 1.6%, less than 1.7%, less than 1.8%, less than 1.9%, less than 2%, less than 2.1%, less than 2.2%, less than 2.3%, less than 2.4%, less than 2.5%, less than 2.6%, less than 2.7%, less than 2.8%, less than 2.9%, less than 3%, less than 3.5%, or less than 4% following CD3 depletion. In some embodiments, the percentage of cells in the cell population expressing CD3 is no more than 4%, no more than 3.5%, no more than 3%, no more than 2.9%, no more than 2.8%, no more than 2.7%, no more than 2.6%, no more than 2.5%, no more than 2.4%, no more than 2.3%, no more than 2.2%, no more than 2.1%, no more than 2%, no more than 1.9%, no more than 1.8%, no more than 1.7%, no more than 1.6%, no more than 1.5%, no more than 1%, or no more than 0.5% following CD3 depletion.

In some embodiments, the engineered immune cells in the cell population may undergo at least 1 round, at least 2 rounds, at least 3 rounds, at least 4 rounds, at least 5 rounds, at least 6 rounds, at least 7 rounds, at least 8 rounds, at least 9 rounds, or at least 10 rounds of CD3 depletion. In some embodiments, the engineered immune cells in the cell population may undergo at most 10 rounds, at most 9 rounds, at most 8 rounds, at most 7 rounds, at most 6 rounds, at most 5 rounds, at most 4 rounds, at most 3 rounds, at most 2 rounds, or at most 1 round of CD3 depletion. In some embodiments, the engineered immune cells in the cell population may undergo about 1 round to about 5 rounds of CD3 depletion. In some embodiments, the engineered immune cells in the cell population may undergo about 1 round to about 2 rounds, about 1 round to about 3 rounds, about 1 round to about 4 rounds, about 1 round to about 5 rounds, about 2 rounds to about 3 rounds, about 2 rounds to about 4 rounds, about 2 rounds to about 5 rounds, about 3 rounds to about 4 rounds, about 3 rounds to about 5 rounds, or about 4 rounds to about 5 rounds of CD3 depletion.

### Bicistronic Expression Constructs of a CAR and a First PEBL

Provided herein are recombinant bicistronic viral constructs or vectors that contain a polynucleotide encoding a CAR (e.g., a CAR comprising a binding domain specific for CD7) and a polynucleotide encoding a PEBL (e.g., a PEBL comprising a binding domain specific for CD7), as described herein. In some embodiments, the recombinant bicistronic viral construct includes an internal ribosomal entry site (IRES) sequence between the nucleic acid sequence of the CAR and the nucleic acid sequence of the PEBL. In some embodiments, the recombinant bicistronic viral construct includes a ribosomal codon skipping site sequence (also referred to as a sequence encoding a 2A self-cleaving peptide) between the nucleic acid sequence of the CAR and the nucleic acid sequence of the PEBL. In some embodiments of a bicistronic construct, a polynucleotide encoding a CAR is located upstream (at the 5′ end) of an IRES sequence, and a polynucleotide encoding a PEBL is located downstream (at the 3′ end) of the IRES. In some cases, a nucleic acid sequence encoding a CAR is operably linked to an IRES sequence and an IRES sequence is operably linked to a nucleic acid sequence encoding a PEBL. In some cases, a nucleic acid sequence encoding a PEBL is operably linked to an IRES sequence and an IRES sequence is operably linked to a nucleic acid sequence encoding a CAR.

In some embodiments of a bicistronic construct, a polynucleotide encoding a CAR is located upstream (at the 5′ end) of a polynucleotide encoding 2A self-cleaving peptide, and a polynucleotide encoding a PEBL is located downstream (at the 3′ end) of the polynucleotide encoding 2A self-cleaving peptide. In some cases, a nucleic acid sequence encoding a CAR is operably linked to a nucleic acid sequence encoding a 2A self-cleaving peptide, which is operably linked to a nucleic acid sequence encoding a PEBL. In some cases, a nucleic acid sequence encoding a PEBL is operably linked to a nucleic acid sequence encoding a 2A self-cleaving peptide, which is operably linked to a nucleic acid sequence encoding a CAR.

The mechanism of ribosomal codon skipping via a 2A peptide sequence is useful for generating two proteins from one transcript; a normal peptide bond is impaired at the 2A sequence, resulting in two discontinuous protein fragments from one translation event. Self-cleaving 2A peptides (e.g., 2A cleavage sites) are described in Kim et al., PLoS One, 2011, 6(4):e18556.

In some embodiments, the IRES is from an Encephalomyocarditis virus. In some embodiments, the IRES is from an Enterovirus. In some embodiments, the nucleic acid sequence of the IRES sequence is set forth in SEQ ID NO:62 (see, e.g., Table 2).

In some embodiments, the ribosomal codon skipping site is based on a 2A self-cleaving peptide (see, e.g., Table 3). In some embodiments, the 2A self-cleaving peptide is selected from the group consisting of P2A, E2A, F2A, and T2A. In some instances, the amino acid sequence of the P2A peptide comprises the amino acid sequence of SEQ ID NO:67, or an amino acid sequence having at least 90% sequence identify thereto. In some instances, the amino acid sequence of the E2A peptide comprises the amino acid sequence of SEQ ID NO:68, or an amino acid sequence having at least 90% sequence identify thereto. In some instances, the amino acid sequence of the F2A peptide comprises the amino acid sequence of SEQ ID NO:69, or an amino acid sequence having at least 90% sequence identify thereto. In some instances, the amino acid sequence of the T2A peptide comprises the amino acid sequence of SEQ ID NO:70, or an amino acid sequence having at least 90% sequence identify thereto.

In some embodiments, the viral construct (e.g., retroviral construct) comprises a nucleic acid sequence encoding a 2A self-cleaving peptide (e.g., 2A peptide cleavage site) selected from the group consisting of P2A, E2A, F2A, and T2A, wherein the polynucleotide encoding 2A self-cleaving peptide links the nucleic acid sequence encoding the CAR and the nucleic acid sequence encoding the PEBL. In other words, the polynucleotide encoding 2A self-cleaving peptide is between the nucleic acid sequence encoding the CAR and the nucleic acid sequence encoding the PEBL. As described above, in some embodiments, the construct comprises or consists of from 5′ end to 3′ end: a nucleic acid sequence encoding a CAR, a nucleic acid sequence encoding a P2A self-cleaving peptide, and a nucleic acid sequence encoding a PEBL. In some embodiments, the construct comprises or consists of from 5′ end to 3′ end: a nucleic acid sequence encoding any CD7 CAR described herein, a nucleic acid sequence encoding a P2A self-cleaving peptide, and a nucleic acid sequence encoding any CD7 PEBL described herein. In some embodiments, the construct comprises or consists of from 5′ end to 3′ end: a nucleic acid sequence encoding any CD7 CAR described herein, a nucleic acid sequence encoding an E2A self-cleaving peptide, and a nucleic acid sequence encoding any CD7 PEBL described herein. In some embodiments, the construct comprises or consists of from 5′ end to 3′ end: a nucleic acid sequence encoding any CD7 CAR described herein, a nucleic acid sequence encoding an F2A self-cleaving peptide, and a nucleic acid sequence encoding any CD7 PEBL described herein. In some embodiments, the construct comprises or consists of from 5′ end to 3′ end: a nucleic acid sequence encoding any CD7 CAR described herein, a nucleic acid sequence encoding a T2A self-cleaving peptide, and a nucleic acid sequence encoding any CD7 PEBL described herein.

In some embodiments, the construct comprises or consists of from 5′ end to 3′ end: a nucleic acid sequence encoding a PEBL, a nucleic acid sequence encoding a P2A self-cleaving peptide, and a nucleic acid sequence encoding a CAR. In some embodiments, the construct comprises or consists of from 5′ end to 3′ end: a nucleic acid sequence encoding a PEBL, a nucleic acid sequence encoding an E2A self-cleaving peptide, and a nucleic acid sequence encoding a CAR. In some embodiments, the construct comprises or consists of from 5′ end to 3′ end: a nucleic acid sequence encoding a PEBL, a nucleic acid sequence encoding an F2A self-cleaving peptide, and a nucleic acid sequence encoding a CAR. In some embodiments, the construct comprises or consists of from 5′ end to 3′ end: a nucleic acid sequence encoding a PEBL, a nucleic acid sequence encoding a T2A self-cleaving peptide, and a nucleic acid sequence encoding a CAR.

In some embodiments, the nucleic acid sequence encoding the P2A comprises or consists of a nucleic acid having at least 90% sequence identity to SEQ ID NO:63. In some embodiments, the nucleic acid sequence encoding the P2A comprises or consisting of a nucleic acid of SEQ ID NO:63. In some embodiments, the nucleic acid sequence encoding the E2A comprises or consists of a nucleic acid having at least 90% sequence identity to SEQ ID NO:64. In some embodiments, the nucleic acid sequence encoding the E2A comprises or consists of a nucleic acid of SEQ ID NO:64. In some embodiments, the nucleic acid sequence encoding the F2A comprises or consists of a nucleic acid having at least 90% sequence identity to SEQ ID NO:65. In some embodiments, the nucleic acid sequence encoding the F2A comprises or consists of a nucleic acid of SEQ ID NO:65. In some embodiments, the nucleic acid sequence encoding the T2A comprises or consists of a nucleic acid sequence having at least 90% sequence identity to SEQ ID NO:66. In some embodiments, the nucleic acid sequence encoding the T2A comprises or consists of a nucleic acid of SEQ ID NO:66.

In some embodiments, the nucleic acid sequence encoding the PEBL is disposed (e.g., located) 5′ to the nucleic acid sequence encoding the CAR. In some embodiments, the nucleic acid sequence encoding the CAR is disposed 5′ to the nucleic acid sequence encoding the PEBL. In some embodiments, a bicistronic construct comprises or consists of from 5′ to 3′ end: (a1) SEQ ID NO:4, SEQ ID NO:63, and SEQ ID NO:2; (a2) SEQ ID NO:4, SEQ ID NO:63, and SEQ ID NO:3; (a3) SEQ ID NO:5, SEQ ID NO:63, and SEQ ID NO:2; (a4) SEQ ID NO:5, SEQ ID NO:63, and SEQ ID NO:3; (b1) SEQ ID NO:4, SEQ ID NO:64, and SEQ ID NO: 2; (b2) SEQ ID NO:4, SEQ ID NO: 64, and SEQ ID NO:3; (b3) SEQ ID NO:5, SEQ ID NO: 64, and SEQ ID NO: 2; (b4) SEQ ID NO:5, SEQ ID NO: 64, and SEQ ID NO:3; (c1) SEQ ID NO:4, SEQ ID NO:65, and SEQ ID NO:2; (c2) SEQ ID NO:4, SEQ ID NO: 65, and SEQ ID NO:3; (c3) SEQ ID NO:5, SEQ ID NO: 65, and SEQ ID NO:2; (c4) SEQ ID NO:5, SEQ ID NO: 65, and SEQ ID NO:3; (d1) SEQ ID NO:4, SEQ ID NO:66, and SEQ ID NO:2; (d2) SEQ ID NO:4, SEQ ID NO: 66, and SEQ ID NO:3; (d3) SEQ ID NO:5, SEQ ID NO: 66, and SEQ ID NO:2; (d4) SEQ ID NO:5, SEQ ID NO: 66, and SEQ ID NO:3; (e1) SEQ ID NO:2, SEQ ID NO:63, and SEQ ID NO:4; (e2) SEQ ID NO:3, SEQ ID NO:63, and SEQ ID NO:4; (e3) SEQ ID NO:2, SEQ ID NO:63, and SEQ ID NO:5; (e4) SEQ ID NO:3, SEQ ID NO:63, and SEQ ID NO:5; (f1) SEQ ID NO:2, SEQ ID NO:64, and SEQ ID NO:4; (f2) SEQ ID NO:3, SEQ ID NO:64, and SEQ ID NO:4; (f3) SEQ ID NO:2, SEQ ID NO:63, and SEQ ID NO: 5; (f4) SEQ ID NO:3, SEQ ID NO:64, and SEQ ID NO:5; (g1) SEQ ID NO:2, SEQ ID NO:65, and SEQ ID NO: 4; (g2) SEQ ID NO:3, SEQ ID NO:65, and SEQ ID NO:4; (g3) SEQ ID NO:2, SEQ ID NO:65, and SEQ ID NO:5; (g4) SEQ ID NO:3, SEQ ID NO:65, and SEQ ID NO:5; (h1) SEQ ID NO:2, SEQ ID NO:66, and SEQ ID NO:4; (h2) SEQ ID NO:3, SEQ ID NO:66, and SEQ ID NO:4; (h3) SEQ ID NO:2, SEQ ID NO:66, and SEQ ID NO:5; or (h4) SEQ ID NO:3, SEQ ID NO: 66, and SEQ ID NO:5.

In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:67, and a polynucleotide encoding a CD7 (TH69) PEBL of SEQ ID NO:24. In some embodiments, a bicistronic construct comprises or consists of from 5′ to 3′ end: a polynucleotide encoding a CD7 (TH69) CAR, a polynucleotide encoding a P2A peptide of SEQ ID NO:67, and a polynucleotide encoding a CD7 (TH69) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:67, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH69) CAR, a polynucleotide encoding a P2A peptide of SEQ ID NO:67, and a polynucleotide encoding a CD7 (3A1F) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:67, and a polynucleotide encoding a CD7 (TH69) PEBL of SEQ ID NO:24. In some embodiments, a bicistronic construct comprises or consists of from 5′ to 3′ end: a polynucleotide encoding a CD7 (3A1F) CAR, a polynucleotide encoding a P2A peptide of SEQ ID NO:67, and a polynucleotide encoding a CD7 (TH69) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:67, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR, a polynucleotide encoding a P2A peptide of SEQ ID NO:67, and a polynucleotide encoding a CD7 (3A1F) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:68, and a polynucleotide encoding a CD7 (TH69) PEBL of SEQ ID NO:24. In some embodiments, a bicistronic construct comprises or consists of from 5′ to 3′ end: a polynucleotide encoding a CD7 (TH69) CAR, a polynucleotide encoding a P2A peptide of SEQ ID NO:68, and a polynucleotide encoding a CD7 (TH69) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:68, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH69) CAR, a polynucleotide encoding a P2A peptide of SEQ ID NO:68, and a polynucleotide encoding a CD7 (3A1F) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:68, and a polynucleotide encoding a CD7 (TH69) PEBL of SEQ ID NO:24. In some embodiments, a bicistronic construct comprises or consists of from 5′ to 3′ end: a polynucleotide encoding a CD7 (3A1F) CAR, a polynucleotide encoding a P2A peptide of SEQ ID NO:68, and a polynucleotide encoding a CD7 (TH69) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:68, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR, a polynucleotide encoding a P2A peptide of SEQ ID NO:68, and a polynucleotide encoding a CD7 (3A1F) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:69, and a polynucleotide encoding a CD7 (TH69) PEBL of SEQ ID NO:24. In some embodiments, a bicistronic construct comprises or consists of from 5′ to 3′ end: a polynucleotide encoding a CD7 (TH69) CAR, a polynucleotide encoding a P2A peptide of SEQ ID NO:69, and a polynucleotide encoding a CD7 (TH69) PEBL of SEQ ID NO: 24 or SEQ ID NO: 25. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:69, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH69) CAR, a polynucleotide encoding a P2A peptide of SEQ ID NO:69, and a polynucleotide encoding a CD7 (3A1F) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:69, and a polynucleotide encoding a CD7 (TH69) PEBL of SEQ ID NO:24. In some embodiments, a bicistronic construct comprises or consists of from 5′ to 3′ end: a polynucleotide encoding a CD7 (3A1F) CAR, a polynucleotide encoding a P2A peptide of SEQ ID NO:69, and a polynucleotide encoding a CD7 (TH69) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:69, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR, a polynucleotide encoding a P2A peptide of SEQ ID NO:69, and a polynucleotide encoding a CD7 (3A1F) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:70, and a polynucleotide encoding a CD7 (TH69) PEBL of SEQ ID NO:24. In some embodiments, a bicistronic construct comprises or consists of from 5′ to 3′ end: a polynucleotide encoding a CD7 (TH69) CAR, a polynucleotide encoding a P2A peptide of SEQ ID NO:70, and a polynucleotide encoding a CD7 (TH69) PEBL. In some embodiments, a bicistronic construct comprises or consists of from 5′ to 3′ end: a polynucleotide encoding a CD7 (TH69) CAR of SEQ ID NO:28, a polynucleotide encoding a P2A peptide of SEQ ID NO:70, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH69) CAR, a polynucleotide encoding a P2A peptide of SEQ ID NO:70, and a polynucleotide encoding a CD7 (3A1F) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:70, and a polynucleotide encoding a CD7 (TH69) PEBL of SEQ ID NO:24. In some embodiments, a bicistronic construct comprises or consists of from 5′ to 3′ end: a polynucleotide encoding a CD7 (3A1F) CAR, a polynucleotide encoding a P2A peptide of SEQ ID NO:70, and a polynucleotide encoding a CD7 (TH69) PEBL. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:70, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR, a polynucleotide encoding a P2A peptide of SEQ ID NO:70, and a polynucleotide encoding a CD7 (3A1F) PEBL.

In some embodiments, the polynucleotide sequence encoding the PEBL is disposed 5′ (upstream) of an IRES site and the IRES site is disposed 5′ to the polynucleotide sequence encoding the CAR. In some embodiments, the polynucleotide sequence encoding the CAR is disposed 5′ of an IRES site and the IRES site is disposed 5′ to the polynucleotide sequence encoding the PEBL.

In some embodiments, the polynucleotide sequence encoding the PEBL is disposed 5′ (upstream) of the ribosomal codon skipping site and the ribosomal codon skipping site is disposed 5′ to the polynucleotide sequence encoding the CAR. In some embodiments, the polynucleotide sequence encoding the CAR is disposed 5′ of the ribosomal codon skipping site and the ribosomal codon skipping site is disposed 5′ to the polynucleotide sequence encoding the PEBL. In some embodiments, the polynucleotide sequence encoding the PEBL is not disposed 5′ (upstream) to the polynucleotide sequence encoding the CAR. For example, the polynucleotide sequence encoding the PEBL may not be disposed 5′ (upstream) of the ribosomal codon skipping site, which is in turn disposed 5′ to the polynucleotide sequence encoding the CAR.

In some aspects, provided herein is a recombinant bicistronic construct comprising at least 900 sequence identity to a nucleic acid sequence of one or more selected from the group consisting of SEQ ID NO:63, SEQ ID NO:64, SEQ ID NO:65, and SEQ ID NO:66. In some embodiments, the recombinant bicistronic construct comprises at least 90% sequence identity to the nucleic acid sequence of SEQ ID NO: 63. In some embodiments, the recombinant bicistronic construct comprises at least 90% sequence identity to the nucleic acid sequence of SEQ ID NO: 64. In some embodiments, the recombinant bicistronic construct comprises at least 90% sequence identity to the nucleic acid sequence of SEQ ID NO:65. In some embodiments, the recombinant bicistronic construct comprises at least 90% sequence identity to the nucleic acid sequence of SEQ ID NO: 66. In some embodiments, the recombinant bicistronic construct comprises a nucleic acid sequence of one selected from the group consisting of SEQ ID NO: 63, SEQ ID NO: 64, SEQ ID NO: 65, and SEQ ID NO: 66.

TABLE 2
Nucleic acid sequences of ribosomal codon skipping peptides and IRES

| Name | SEQ ID NO | Nucleic Acid Sequence |
|---|---|---|
| IRES | SEQ ID NO: 62 | CGGGATCAATTCCGCCCCCCCCCTAACGTTACTGGCCGAAGCCGCT |
| | | TGGAATAAGGCCGGTGTGCGTTTGTCTATATGTTATTTTCCACCAT |
| | | ATTGCCGTCTTTTGGCAATGTGAGGGCCCGGAAACCTGGCCCTGTC |
| | | TTCTTGACGAGCATTCCTAGGGGTCTTTCCCCTCTCGCCAAAGGAA |
| | | TGCAAGGTCTGTTGAATGTCGTGAAGGAAGCAGTTCCTCTGGAAGC |
| | | TTCTTGAAGACAAACAACGTCTGTAGCGACCCTTTGCAGGCAGCGG |
| | | AACCCCCCACCTGGCGACAGGTGCCTCTGCGGCCAAAAGCCACGTG |
| | | TATAAGATACACCTGCAAAGGCGGCACAACCCCAGTGCCACGTTGT |
| | | GAGTTGGATAGTTGTGGAAAGAGTCAAATGGCTCTCCTCAAGCGTA |
| | | TTCAACAAGGGGCTGAAGGATGCCCAGAAGGTACCCCATTGTATGG |
| | | GATCTGATCTGGGGCCTCGGTGCACATGCTTTACATGTGTTTAGTC |
| | | GAGGTTAAAAAAACGTCTAGGCCCCCCGAACCACGGGGACGTGGTT |
| | | TTCCTTTGAAAAACACGATAATACC |
| P2A | SEQ ID NO: 63 | GCCACAAACTTCTCTCTGCTAAAGCAAGCAGGTGATGTTGAAGAAA |
| | | ACCCCGGGCCT |
| E2A | SEQ ID NO: 64 | CAGTGTACTAATTATGCTCTCTTGAAATTGGCTGGAGATGTTGAGA |
| | | GCAACGGAGGTCCC |
| F2A | SEQ ID NO: 65 | GTGAAACAGACTTTGAATTTTGACCTTCTCAAGTTGGCGGGAGACG |
| | | TGGAGTCCAACCCTGGACCT |
| T2A | SEQ ID NO: 66 | GAGGGCAGGGGAAGTCTTCTAACATGCGGGGACGTGGAGGAAAATC |
| | | CCGGCCCA |

TABLE 3
Amino acid sequences of ribosomal
codon skipping sites

| Name | SEQ ID NO | Amino Acid Sequence |
|---|---|---|
| P2A | SEQ ID NO: 67 | ATNFSLLKQAGDVEENPGP |
| E2A | SEQ ID NO: 68 | QCTNYALLKLAGDVESNPGP |
| F2A | SEQ ID NO: 69 | VKQTLNFDLLKLAGDVESNPGP |
| T2A | SEQ ID NO: 70 | EGRGSLLTCGDVEENPGP |

The present invention provides vectors such as expression vectors in which any of the polynucleotides described herein is inserted. In some embodiments, the vector is derived from retroviruses such as lentiviruses. Such vectors are suitable tools to achieve long-term gene transfer since they allow long-term, stable integration of an exogenous polynucleotide (e.g., transgene) and its propagation in daughter cells. Unlike vectors derived from onco-retroviruses such as murine leukemia viruses, lentiviral vectors can transduce non-proliferating cells. Lentiviral vectors also have low immunogenicity. In other embodiments, the vector is an adenoviral vector. In certain embodiments, the vector is a plasmid.

In some embodiments, the promoter comprises at least 90%, e.g., 90%, 91%, 92%, 93%, 94%, 95%, 96%, 97%, 98%, 99%, 99.9%, or more sequence identity to a CMV promoter. In some embodiments, the promoter comprises a CMV promoter. In some embodiments, the CMV promoter comprises the sequence of SEQ ID NO:6. In some embodiments, any of the constructs described herein comprises or consists of a CMV promoter of SEQ ID NO:6.

In some embodiments, the promoter comprises at least 90%, e.g., 90%, 91%, 92%, 93%, 94%, 95%, 96%, 97%, 98%, 99%, 99.9%, or more sequence identity to an EF1α promoter. In some embodiments, the promoter comprises an EF1α promoter. In some embodiments, the EF1α promoter comprises the sequence of SEQ ID NO:7. In some embodiments, the EF1α promoter comprises the sequence of SEQ ID NO:7. In some embodiments, any of the constructs described herein comprises or consists of an EF1α promoter of SEQ ID NO:7.

In some embodiments, the promoter comprises at least 90%, e.g., 90%, 91%, 92%, 93%, 94%, 95%, 96%, 97%, 98%, 99%, 99.9%, or more sequence identity to an EFS promoter. In some embodiments, the promoter comprises an EFS promoter. In some embodiments, the EFS promoter comprises the sequence of SEQ ID NO:8. In some embodiments, the EFS promoter comprises the sequence of SEQ ID NO: 8. In some embodiments, any of the constructs described herein comprises or consists of an EFS promoter of SEQ ID NO:8.

In some embodiments, the promoter comprises at least 90%, e.g., 90%, 91%, 92%, 93%, 94%, 95%, 96%, 97%, 98%, 99%, 99.9%, or more sequence identity to a murine stem cell virus (MSCV) promoter. In some embodiments, the promoter comprises a MSCV promoter. In some embodiments, the MSCV promoter comprises the sequence of SEQ ID NO:9. In some embodiments, any of the constructs described herein comprises or consists of a MSCV promoter of SEQ ID NO:9.

In some embodiments, the promoter comprises at least 90%, e.g., 90%, 91%, 92%, 93%, 94%, 95%, 96%, 97%, 98%, 99%, 99.9%, or more sequence identity to a phosphoglycerate kinase (PGK) promoter. In some embodiments, the promoter comprises a PGK promoter. In some embodiments, the PGK promoter comprises the sequence of SEQ ID NO:10. In some embodiments, any of the constructs described herein comprises or consists of a PGK promoter of SEQ ID NO:10.

In some embodiments, the bicistronic vector comprises or consists of the nucleic acid sequence of SEQ ID NO:11. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:11. The bicistronic vector comprises a nucleic acid sequence comprising from 5′ end to 3′ end: a nucleic acid sequence encoding a CD7 PEBL, an IRES sequence, and a nucleic acid sequence encoding a CD7 CAR, and optionally at the 5′ end, a promoter selected from the group consisting of a CMV promoter (e.g., SEQ ID NO:6), EF1α promoter (e.g., SEQ ID NO:7), EFS promoter (e.g., SEQ ID NO:8), MSCV promoter (e.g., SEQ ID NO:9), and PGK promoter (e.g., SEQ ID NO:10).

In some embodiments, the bicistronic vector comprises the nucleic acid sequence of SEQ ID NO:12. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO: 12. The bicistronic vector comprises a nucleic acid sequence comprising from 5′ end to 3′ end: a nucleic acid sequence encoding a CD7 CAR, an IRES sequence, and a nucleic acid sequence encoding a CD7 PEBL, optionally at the 5′ end, a promoter selected from the group consisting of a CMV promoter, EF1α promoter, EFS promoter, MSCV promoter, and PGK promoter.

In some embodiments, the bicistronic vector comprises the nucleic acid sequence of SEQ ID NO:13. The bicistronic vector comprises at least 90%, e.g., 90%, 91%, 92%, 93%, 94%, 95%, 96%, 97%, 98%, 99%, 99.9%, or more sequence identity to the sequence of SEQ ID NO:13. The bicistronic vector comprises a nucleic acid sequence comprising from 5′ end to 3′ end: a nucleic acid sequence encoding a CD7 CAR, a nucleic acid sequence encoding a P2A peptide, and a nucleic acid sequence encoding a CD7 PEBL, optionally at the 5′ end, a promoter selected from the group consisting of a CMV promoter, EF1α promoter, EFS promoter, MSCV promoter, and PGK promoter.

In some embodiments, the bicistronic vector comprises the nucleic acid sequence of SEQ ID NO:14. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO: 14. The bicistronic vector comprises a nucleic acid sequence comprising from 5′ end to 3′ end: a promoter, a nucleic acid sequence encoding a CD7 CAR, a P2A sequence, and a nucleic acid sequence encoding a CD7 PEBL. In some instances the bicistronic vector comprises a nucleic acid sequence comprising from 5′ end to 3′ end: a MSCV promoter, a nucleic acid sequence encoding a CD7 CAR, a nucleic acid sequence encoding a P2A peptide, and a nucleic acid sequence encoding a CD7 PEBL.

In some embodiments, the bicistronic vector comprises the nucleic acid sequence of SEQ ID NO:15. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:15. In some instances the bicistronic vector comprises a nucleic acid sequence comprising from 5′ end to 3′ end: an EF1α promoter, a nucleic acid sequence encoding a CD7 CAR, a nucleic acid sequence encoding a P2A peptide, and a nucleic acid sequence encoding a CD7 PEBL.

In some embodiments, the bicistronic vector comprises the nucleic acid sequence of SEQ ID NO:16. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:16. In some instances the bicistronic vector comprises a nucleic acid sequence comprising from 5′ end to 3′ end: an EFS promoter, a nucleic acid sequence encoding a CD7 CAR, a nucleic acid sequence encoding a P2A peptide, and a nucleic acid sequence encoding a CD7 PEBL.

### Dual Promoter Retroviral Constructs of a CAR and a First PEBL

Provided herein are recombinant retroviral constructs (or vectors) for simultaneous expression of a CAR and a PEBL in a cell such as a T cell. In some embodiments, the retroviral constructs include a promoter operably linked to a polynucleotide encoding any of the CARs described herein and a promoter operably linked to a polynucleotide encoding any of the PEBLs described herein. In some embodiments, the promoter for the CAR and the promoter for the PEBL share less than 90% sequence identity, e.g., less than 90% identity, less than 80% identity, less than 75% sequence identity, less 70% sequence identity, less than 65% sequence identity, less than 60% sequence identity, less than 55% sequence identity, and the like. In some embodiments, the promoter for the CAR and the promoter for the PEBL share 80% sequence identity or less, e.g., 80% identity, 75% sequence identity, 70% sequence identity, 65% sequence identity, 60% sequence identity, 55% sequence identity, and the like. In some embodiments, the promoter for the CAR and the promoter for the PEBL share at least 50% sequence identity, e.g., 50% sequence identity, 55% sequence identity, 60% sequence identity, 65% sequence identity, 70% sequence identity, 75% sequence identity, 80% sequence identity, 85% sequence identity, 90% sequence identity, 95% sequence identity, or more sequence identity.

In some embodiments, the promoter for the CAR (referred to as the first promoter) is different than the promoter for the PEBL (referred to as the second promoter). The first promoter and the second promoter can have the same sequence. In other instances, the first promoter and the second promoter have different sequences.

In some embodiments, the first promoter and/or second promoter comprises at least 90%, e.g., 90%, 91%, 92%, 93%, 94%, 95%, 96%, 97%, 98%, 99%, 99.9%, or more sequence identity to a CMV promoter. In some embodiments, the first promoter and/or second promoter comprises a CMV promoter. In some embodiments, the CMV promoter comprises the sequence of SEQ ID NO:6.

In some embodiments, the first promoter and/or second promoter comprises at least 90%, e.g., 90%, 91%, 92%, 93%, 94%, 95%, 96%, 97%, 98%, 99%, 99.9%, or more sequence identity to an EF1α promoter. In some embodiments, the first promoter and/or second promoter comprises an EF1α promoter. In some embodiments, the EF1α promoter comprises the sequence of SEQ ID NO:7.

In some embodiments, the first promoter and/or second promoter comprises at least 90%, e.g., 90%, 91%, 92%, 93%, 94%, 95%, 96%, 97%, 98%, 99%, 99.9%, or more sequence identity to an EFS promoter. In some embodiments, the first promoter and/or second promoter comprises an EFS promoter. In some embodiments, the EFS promoter comprises the sequence of SEQ ID NO:8.

In some embodiments, the first promoter and/or second promoter comprises at least 90%, e.g., 90%, 91%, 92%, 93%, 94%, 95%, 96%, 97%, 98%, 99%, 99.9%, or more sequence identity to a murine stem cell virus (MSCV) promoter. In some embodiments, the first promoter and/or second promoter comprises a MSCV promoter. In some embodiments, the MSCV promoter comprises the sequence of SEQ ID NO:9.

In some embodiments, the first promoter and/or second promoter comprises at least 90%, e.g., 90%, 91%, 92%, 93%, 94%, 95%, 96%, 97%, 98%, 99%, 99.9%, or more sequence identity to a phosphoglycerate kinase (PGK) promoter. In some embodiments, the first promoter and/or second promoter comprises a PGK promoter. In some embodiments, the PGK promoter comprises the sequence of SEQ ID NO:10.

In some embodiments, the retroviral constructs from 5′ to 3′ include the first promoter operably linked to the polynucleotide encoding the CAR and the second promoter operably linked to the polynucleotide encoding the PEBL. In various embodiments, the retroviral constructs from 5′ to 3′ include the second promoter operably linked to the polynucleotide encoding the PEBL and the first promoter operably linked to the polynucleotide encoding the CAR.

In some embodiments, the first promoter is located upstream of the second promoter. In some embodiments, the first promoter is a CMV promoter and the second promoter is an EFS promoter. In some embodiments, the first promoter is a CMV promoter and the second promoter is an EF1α promoter. In some embodiments, the first promoter is a CMV promoter and the second promoter is a PGK promoter. In some embodiments, the first promoter is a CMV promoter and the second promoter is a MSCV promoter. In some embodiments, the first promoter is a CMV promoter and the second promoter is a CMV promoter. In some embodiments, the first promoter is a MSCV promoter and the second promoter is an EFS promoter. In some embodiments, the first promoter is a MSCV promoter and the second promoter is an EF1α promoter. In some embodiments, the first promoter is a MSCV promoter and the second promoter is a PGK promoter. In some embodiments, the first promoter is a MSCV promoter and the second promoter is a CMV promoter. In some embodiments, the first promoter is a MSCV promoter and the second promoter is a MSCV promoter. In some embodiments, the first promoter is a PGK promoter and the second promoter is an EFS promoter. In some embodiments, the first promoter is a PGK promoter and the second promoter is an EF1α promoter. In some embodiments, the first promoter is a PGK promoter and the second promoter is a MSCV promoter. In some embodiments, the first promoter is a PGK promoter and the second promoter is a CMV promoter. In some embodiments, the first promoter is a PGK promoter and the second promoter is a PGK promoter. In some embodiments, the first promoter is an EF1α promoter and the second promoter is a MSCV promoter. In some embodiments, the first promoter is an EF1α promoter and the second promoter is a PGK promoter. In some embodiments, the first promoter is an EF1α promoter and the second promoter is an EFS promoter. In some embodiments, the first promoter is an EF1α promoter and the second promoter is a CMV promoter. In some embodiments, the first promoter is an EF1α promoter and the second promoter is an EF1α promoter. In some embodiments, the first promoter is an EFS promoter and the second promoter is a MSCV promoter. In some embodiments, the first promoter is an EFS promoter and the second promoter is an EF1α promoter. In some embodiments, the first promoter is an EFS promoter and the second promoter is a PGK promoter. In some embodiments, the first promoter is an EFS promoter and the second promoter is a CMV promoter. In some embodiments, the first promoter is an EFS promoter and the second promoter is an EFS promoter.

In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO: 17. In some embodiments, the retroviral construct of the present invention comprises the nucleic acid sequence of SEQ ID NO:17. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:18. In some embodiments, the retroviral construct of the present invention comprises the nucleic acid sequence of SEQ ID NO:18. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:19. In some embodiments, the retroviral construct of the present invention comprises the nucleic acid sequence of SEQ ID NO:19. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:20. In some embodiments, the retroviral construct of the present invention comprises the nucleic acid sequence of SEQ ID NO:20. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:21. In some embodiments, the retroviral construct of the present invention comprises the nucleic acid sequence of SEQ ID NO:21. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:22. In some embodiments, the retroviral construct of the present invention comprises the nucleic acid sequence of SEQ ID NO:22. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:23. In some embodiments, the retroviral construct of the present invention comprises the nucleic acid sequence of SEQ ID NO:23.

Antibodies that Bind CD7

In certain embodiments, the anti-CD7 scFv based on the TH69 antibody comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:32 and 33, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:32. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:33. In some instances, the heavy chain variable region comprises at least one (e.g., 1, 2, 3, 4, 5, 6, 7, 8, 9, 10, or more) amino acid substitution in the sequence set forth in SEQ ID NO:32. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO:32. In some instances, the light chain variable region comprises at least one (e.g., 1, 2, 3, 4, 5, 6, 7, 8, 9, 10, or more) amino acid substitution in the sequence set forth in SEQ ID NO:33. In certain instances, the light chain variable region comprises 10 or fewer amino acid (e.g., 0, 1, 2, 3, 4, 5, 6, 7, 8, 9, or 10) substitutions in the sequence set forth in SEQ ID NO:33. Any of the amino acid substitutions described herein can be conservative or non-conservative substitutions.

In some embodiments, the anti-CD7 scFv comprises a VL CDR1 of SEQ ID NO:44 (SASQGISNYLN), a VL CDR2 of SEQ ID NO:45 (YTSSLHS), and a VL CDR3 of SEQ ID NO:46 (QQYSKLPYT). In some embodiments, the anti-CD7 scFv comprises a VH CDR1 of SEQ ID NO:47 (SYAMS), a VH CDR2 of SEQ ID NO:48 (SISSGGFTYYPDSVKG), and a VH CDR3 of SEQ ID NO:49 (DEVRGYLDV). In some embodiments, the anti-CD7 scFv comprises a VL CDR1 of SEQ ID NO:44, a VL CDR2 of SEQ ID NO:45, a VL CDR3 of SEQ ID NO:46, a VH CDR1 of SEQ ID NO:47, a VH CDR2 of SEQ ID NO:48, and a VH CDR3 of SEQ ID NO:49.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:38. In other embodiments, the nucleic acid sequence encoding the VL comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:39.

In certain embodiments, the anti-CD7 scFv based on the 3A1F antibody comprises a variable heavy chain (heavy chain variable region or VH) and a variable light chain (light chain variable region or VL) having a sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:34 and 35, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:34. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:35. In some instances, the heavy chain variable region comprises at least one (e.g., 1, 2, 3, 4, 5, 6, 7, 8, 9, 10, or more) amino acid substitution in the sequence set forth in SEQ ID NO:34. In certain instances, the heavy chain variable region comprises 10 or fewer amino acid (e.g., 0, 1, 2, 3, 4, 5, 6, 7, 8, 9, 10) substitutions in the sequence set forth in SEQ ID NO:34. In some cases, the light chain variable region comprises at least one (e.g., 1, 2, 3, 4, 5, 6, 7, 8, 9, or more) amino acid substitution in the sequence set forth in SEQ ID NO:35. In certain cases, the heavy chain variable region comprises 10 or fewer amino acid (e.g., 0, 1, 2, 3, 4, 5, 6, 7, 8, 9, 10) substitutions in the sequence set forth in SEQ ID NO:35. Any of the amino acid substitutions described herein can be conservative or non-conservative substitutions.

In some embodiments, the anti-CD7 scFv comprises a VL CDR1 of SEQ ID NO:50 (RASQSISNNLH), a VL CDR2 of SEQ ID NO:51 (SASQSIS), and a VL CDR3 of SEQ ID NO:52 (QQSNSWPYT). In some embodiments, the anti-CD7 scFv comprises a VH CDR1 of SEQ ID NO:53 (SYWMH), a VH CDR2 of SEQ ID NO:54 (KINPSNGRTNYNEKFKS), and a VH CDR3 of SEQ ID NO:55 (GGVYYDLYYYALDY). In various embodiments, the anti-CD7 scFv comprises a VL CDR1 of SEQ ID NO:50, a VL CDR2 of SEQ ID NO:51, a VL CDR3 of SEQ ID NO:52, a VH CDR1 of SEQ ID NO:53, a VH CDR2 of SEQ ID NO:54, and a VH CDR3 of SEQ ID NO:55.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:40. In other embodiments, the nucleic acid sequence encoding a VL comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:41.

In certain embodiments, the anti-CD7 scFv based on the T3-3A1 antibody comprises a variable heavy chain (heavy chain variable region or VH) and a variable light chain (light chain variable region or VL) having a sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:36 and 37, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:36. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:37. In some instances, the heavy chain variable region comprises at least one (e.g., 1, 2, 3, 4, 5, 6, 7, 8, 9, 10, or more) amino acid substitution in the sequence set forth in SEQ ID NO:36. In certain instances, the heavy chain variable region comprises 13 or fewer amino acid (e.g., 0, 1, 2, 3, 4, 5, 6, 7, 8, 9, 10, 11, 12, or 13) substitutions in the sequence set forth in SEQ ID NO:36. In some cases, the light chain variable region comprises at least one (e.g., 1, 2, 3, 4, 5, 6, 7, 8, 9, 10, or more) amino acid substitution in the sequence set forth in SEQ ID NO:37. In certain cases, the heavy chain variable region comprises 11 or fewer amino acid (e.g., 0, 1, 2, 3, 4, 5, 6, 7, 8, 9, 10, or 11) substitutions in the sequence set forth in SEQ ID NO:37. Any of the amino acid substitutions described herein can be conservative or non-conservative substitutions.

In some embodiments, the anti-CD7 scFv comprises a VL CDR1 of SEQ ID NO:56 (RASKSVSASGYSYMH), a VL CDR2 of SEQ ID NO:57 (LASNLES), and a VL CDR3 of SEQ ID NO:58 (QHSRELPYT). In some embodiments, the anti-CD7 scFv comprises a VH CDR1 of SEQ ID NO:59 (SFGMH), a VH CDR2 of SEQ ID NO:60(YISSGSSTLHYADTVKG), and a VH CDR3 of SEQ ID NO:61 (WGNYPHYAMDY). In various embodiments, the anti-CD7 scFv comprises a VL CDR1 of SEQ ID NO:56, a VL CDR2 of SEQ ID NO:57, a VL CDR3 of SEQ ID NO:58, a VH CDR1 of SEQ ID NO:59, a VH CDR2 of SEQ ID NO:60, and a VH CDR3 of SEQ ID NO:61.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:42. In other embodiments, the nucleic acid sequence encoding the VL comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:43.

In some embodiments, the scFv of the present invention comprises a variable heavy chain sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to a variable heavy chain sequence of an anti-CD7 antibody. In some embodiments, the scFv of the present invention comprises a variable light chain sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to a variable light chain sequence of an anti-CD7 antibody. For instance, the anti-CD7 antibody can be any such recognized by one skilled in the art.

TABLE 4
Amino acid sequences of VH regions and VL regions of anti-CD7 scFvs

| Antibody | Component | Amino Acid Sequence |
|---|---|---|
| TH69 | VH | EVQLVESGGGLVKPGGSLKLSCAASGLTFSSYAMSWVRQTPEKRLE |
| | | WVASISSGGFTYYPDSVKGRFTISRDNARNILYLQMSSLRSEDTAM |
| | | YYCARDEVRGYLDVWGAGTTVTVSS (SEQ ID NO: 32) |
| | VL | AAYKDIQMTQTTSSLSASLGDRVTISCSASQGISNYLNWYQQKPDG |
| | | TVKLLIYYTSSLHSGVPSRFSGSGSGTDYSLTISNLEPEDIATYYC |
| | | QQYSKLPYTFGGGTKLEIKR (SEQ ID NO: 33) |
| 3A1F | VH | QVQLQESGAELVKPGASVKLSCKASGYTFTSYWMHWVKQRPGQGLE |
| | | WIGKINPSNGRTNYNEKFKSKATLTVDKSSSTAYMQLSSLTSEDSA |
| | | VYYCARGGVYYDLYYYALDYWGQGTTVTVSS (SEQ ID NO: 34) |
| | VL | DIELTQSPATLSVTPGDSVSLSCRASQSISNNLHWYQQKSHESPRL |
| | | LIKSASQSISGIPSRFSGSGSGTDFTLSINSVETEDFGMYFCQQSN |
| | | SWPYTFGGGTKLEIKR (SEQ ID NO: 35) |
| T3-3A1 | VH | DVQLVESGGGLVQPGGSRKLSCAASGFTFSSFGMHWVRQAPEKGLE |
| | | WVAYISSGSSTLHYADTVKGRFTISRDNPKNTLFLQMTSLRSEDTA |
| | | MYYCARWGNYPHYAMDYWGQGTSVTVSS (SEQ ID NO: 36) |
| | VL | DIVMTQSPASLAVSLGQRATISCRASKSVSASGYSYMHWYQQKPGQ |
| | | PPKLLIYLASNLESGVPARFSGSGSGTDFTLNIHPVEEEDAVTYYC |
| | | QHSRELPYTFGGGTKLEIK (SEQ ID NO: 37) |

TABLE 5
Nucleic acid sequences of VH regions and VL regions of anti-CD7 scFvs

| Antibody | Component | Nucleic Acid Sequence |
|---|---|---|
| TH69 | VH | GAGGTGCAGCTGGTCGAATCTGGAGGAGGACTGGTGAAGCCAGGAG |
| | | GATCTCTGAAACTGAGTTGTGCCGCTTCAGGCCTGACCTTCTCAAG |
| | | CTACGCCATGAGCTGGGTGCGACAGACACCTGAGAAGCGGCTGGAA |
| | | TGGGTCGCTAGCATCTCCTCTGGCGGGTTCACATACTATCCAGACT |
| | | CCGTGAAAGGCAGATTTACTATCTCTCGGGATAACGCAAGAAATAT |
| | | TCTGTACCTGCAGATGAGTTCACTGAGGAGCGAGGACACCGCAATG |
| | | TACTATTGTGCCAGGGACGAAGTGCGCGGCTATCTGGATGTCTGGG |
| | | GAGCTGGCACTACCGTCACCGTCTCCAGC (SEQ ID NO: 38) |
| | VL | GCCGCATACAAGGATATTCAGATGACTCAGACCACAAGCTCCCTGA |
| | | GCGCCTCCCTGGGAGACCGAGTGACAATCTCTTGCAGTGCATCACA |
| | | GGGAATTAGCAACTACCTGAATTGGTATCAGCAGAAGCCAGATGGC |
| | | ACTGTGAAACTGCTGATCTACTATACCTCTAGTCTGCACAGTGGGG |
| | | TCCCCTCACGATTCAGCGGATCCGGCTCTGGGACAGACTACAGCCT |
| | | GACTATCTCCAACCTGGAGCCCGAAGATATTGCCACCTACTATTGC |
| | | CAGCAGTACTCCAAGCTGCCTTATACCTTTGGGGGGGGAACAAAGC |
| | | TGGAGATTAAAAGG (SEQ ID NO: 39) |
| 3A1F | VH | CAGGTCCAGCTGCAGGAGTCAGGAGCTGAGCTGGTGAAGCCAGGGG |
| | | CAAGCGTCAAACTGTCCTGCAAGGCCTCTGGATATACATTCACTAG |
| | | CTACTGGATGCACTGGGTGAAACAGAGACCCGGACAGGGCCTGGAG |
| | | TGGATCGGAAAGATTAACCCTAGCAATGGCAGGACCAACTACAACG |
| | | AAAAGTTTAAATCCAAGGCAACCCTGACAGTGGACAAGAGCTCCTC |
| | | TACAGCCTACATGCAGCTGAGTTCACTGACTTCAGAGGATAGCGCA |
| | | GTGTACTATTGCGCCAGAGGCGGGGTCTACTATGACCTGTACTATT |
| | | ACGCCCTGGATTATTGGGGGCAGGGAACCACAGTGACTGTCAGCTC |
| | | C (SEQ ID NO: 40) |
| | VL | GACATCGAGCTGACCCAGAGTCCTGCTACACTGAGCGTGACTCCAG |
| | | GCGATTCTGTCAGTCTGTCATGTCGGGCAAGCCAGTCCATCTCTAA |
| | | CAATCTGCACTGGTACCAGCAGAAATCCCATGAATCTCCACGACTG |
| | | CTGATTAAGAGTGCCTCACAGAGCATCTCCGGCATTCCCTCCCGGT |
| | | TCTCTGGCAGTGGGTCAGGAACTGACTTTACCCTGAGTATTAACTC |
| | | AGTGGAGACAGAAGATTTCGGCATGTATTTTTGCCAGCAGAGCAAT |
| | | TCCTGGCCCTACACTTTCGGAGGGGGGACCAAACTGGAGATCAAGC |
| | | GG (SEQ ID NO: 41) |
| T3-3A1 | VH | GATGTGCAGCTGGTGGAGTCTGGGGGAGGCTTAGTGCAGCCTGGAG |
| | | GGTCCCGGAAACTCTCCTGTGCAGCCTCTGGATTCACTTTCAGTAG |
| | | CTTTGGAATGCACTGGGTTCGTCAGGCTCCAGAGAAGGGGCTGGAG |
| | | TGGGTCGCATACATTAGTAGTGGCAGTAGTACCCTCCACTATGCAG |
| | | ACACAGTGAAGGGCCGATTCACCATCTCCAGAGACAATCCCAAGAA |
| | | CACCCTGTTCCTGCAAATGACCAGTCTAAGGTCTGAGGACACGGCC |
| | | ATGTATTACTGTGCAAGATGGGGTAACTACCCTCACTATGCTATGG |
| | | ACTACTGGGGTCAAGGAACCTCAGTCACCGTCTCCTCA |
| | | SEQ ID NO: 42) |
| | VL | GACATTGTGATGACCCAGTCTCCTGCTTCCTTAGCTGTATCTCTGG |
| | | GGCAGAGGGCCACCATCTCATGCAGGGCCAGCAAAAGTGTCAGTGC |
| | | ATCTGGCTATAGTTATATGCACTGGTACCAACAGAAACCAGGACAG |
| | | CCACCCAAACTCCTCATCTATCTTGCATCCAACCTAGAATCTGGGG |
| | | TCCCTGCCAGGTTCAGTGGCAGTGGGTCTGGGACAGACTTCACCCT |
| | | CAACATCCATCCTGTGGAGGAGGAGGATGCTGTAACCTATTACTGT |
| | | CAGCACAGTAGGGAGCTTCCGTACACGTTCGGAGGGGGGACCAAGC |
| | | TGGAAATAAAA (SEQ ID NO: 43) |

TABLE 15
Exemplary amino acid sequences of VH, VL, and CDR regions of anti-CD7 scFvs

| | | | LC | LC | LC | HC | HC | HC |
|---|---|---|---|---|---|---|---|---|
| Name | VL | VH | CDR1 | CDR2 | CDR3 | CDR1 | CDR2 | CDR3 |
| TH69 | AAYKDIQMT | EVQLVESGG | SASQGI | YTSSLH | QQYSKL | SYAMS | SISSGG | DEVRGY |
| | QTTSSLSAS | GLVKPGGSL | SNYLN | S (SEQ | PYT | (SEQ | FTYYPD | LDV |
| | LGDRVTISC | KLSCAASGL | (SEQ | ID NO: | (SEQ | ID NO: | SVKG | (SEQ |
| | SASQGISNY | TFSSYAMSW | ID NO: | 45) | ID NO: | 47) | (SEQ | ID NO: |
| | LNWYQQKPD | VRQTPEKRL | 44) | | 46) | | ID NO: | 49) |
| | GTVKLLIYY | EWVASISSG | | | | | 48) | |
| | TSSLHSGVP | GFTYYPDSV | | | | | | |
| | SRFSGSGSG | KGRFTISRD | | | | | | |
| | TDYSLTISN | NARNILYLQ | | | | | | |
| | LEPEDIATY | MSSLRSEDT | | | | | | |
| | YCQQYSKLP | AMYYCARDE | | | | | | |
| | YTFGGGTKL | VRGYLDVWG | | | | | | |
| | EIKR (SEQ | AGTTVTVSS | | | | | | |
| | ID NO: 33) | (SEQ ID | | | | | | |
| | | NO: 32) | | | | | | |
| 3A1F | DIELTQSPA | QVQLQESGA | RASQSI | SASQSI | QQSNSW | SYWMH | KINPSN | GGVYYD |
| | TLSVTPGDS | ELVKPGASV | SNNLH | S (SEQ | PYT | (SEQ | GRTNYN | LYYYAL |
| | VSLSCRASQ | KLSCKASGY | (SEQ ID | ID NO: | (SEQ | ID NO: | EKFKS | DY |
| | SISNNLHWY | TFTSYWMHW | NO: 50) | 51) | ID NO: | 53) | (SEQ | (SEQ |
| | QQKSHESPR | VKQRPGQGL | | | 52) | | ID NO: | ID NO: |
| | LLIKSASQS | EWIGKINPS | | | | | 54) | 55) |
| | ISGIPSRFS | NGRTNYNEK | | | | | | |
| | GSGSGTDFT | FKSKATLTV | | | | | | |
| | LSINSVETE | DKSSSTAYM | | | | | | |
| | DFGMYFCQQ | QLSSLTSED | | | | | | |
| | SNSWPYTFG | SAVYYCARG | | | | | | |
| | GGTKLEIKR | GVYYDLYYY | | | | | | |
| | (SEQ ID | ALDYWGQGT | | | | | | |
| | NO: 35) | TVTVSS | | | | | | |
| | | (SEQ ID | | | | | | |
| | | NO: 34) | | | | | | |
| T3-3A1 | DIVMTQSPA | DVQLVESGG | RASKSV | LASNLE | QHSREL | SFGMH | YISSGS | WGNYPH |
| | SLAVSLGQR | GLVQPGGSR | SASGYS | S (SEQ | PYT | (SEQ | STLHYA | YAMDY |
| | ATISCRASK | KLSCAASGF | YMH | ID NO: | (SEQ | ID NO: | DTVKG | (SEQ |
| | SVSASGYSY | TFSSFGMHW | (SEQ ID | 57) | ID NO: | 59) | (SEQ | ID NO: |
| | MHWYQQKPG | VRQAPEKGL | NO: 56) | | 58) | | ID NO: | 61) |
| | QPPKLLIYL | EWVAYISSG | | | | | 60) | |
| | ASNLESGVP | SSTLHYADT | | | | | | |
| | ARFSGSGSG | VKGRFTISR | | | | | | |
| | TDFTLNIHP | DNPKNTLFL | | | | | | |
| | VEEEDAVTY | QMTSLRSED | | | | | | |
| | YCQHSRELP | TAMYYCARW | | | | | | |
| | YTFGGGTKL | GNYPHYAMD | | | | | | |
| | EIK (SEQ | YWGQGTSVT | | | | | | |
| | ID NO: 37) | VSS (SEQ | | | | | | |
| | | ID NO: 36) | | | | | | |

### Downregulation of Intracellular CD7 Via CD7 PEBL

As described herein, T cell cytotoxicity was shown to be markedly increased when anti-CD7 CAR was used in combination with downregulation of CD7 expression on the effector T cells. As demonstrated herein, downregulation (e.g., elimination, reduction, and/or relocalization) of CD7 prevented the fratricidal effect exerted by the corresponding anti-CD7 CAR, allowing greater T cell recovery after CAR expression as compared to cells that retained the target antigen (e.g., CD7), and a more effective cytotoxicity against T leukemia/lymphoma cells. As those of skill in the art would appreciate, downregulation of CD7 expression on the effector T cells can be achieved according to a variety of known methods including, for example, protein expression blockers (PEBLs) against CD7 (as described in WO2016/126213), RNAi against CD7, or gene editing methods such as, e.g., meganucleases, TALEN, CRISPR/Cas9, and zinc finger nucleases. Gene disruption may also be possible through base editing technologies. Base editors are capable of making single base pair changes at defined genetic loci to alter gene expression. Adenine base editors (ABEs) and cytosine base editors (CBEs) combine a deaminase enzyme with a Cas nickase to mediate gene editing without double-stranded breaks in DNA. Without wishing to be bound by theory, the methods provided herein may provide for an efficient targeted multiplexed editing system. In some embodiments, a base editor system comprises a nucleotide binding domain, a deaminase domain for deaminating nucleobases in a target nucleotide sequence; and one or more guide RNA molecules (gRNAs) targeting Cas to a specific locus. Adenine base editors make A to G (or T to C) point mutations at a target site and cytosine base editors make C to T (or G to A) point mutations at a target site. In some cases, cytosine base editors can be fused with an inhibitor of uracil DNA glycosylase (UGI) to prevent base excision repair. In some embodiments, the deaminase is an adenosine deaminase. In some embodiments, the adenosine deaminase catalyzes the hydrolytic deamination of adenine or adenosine in deoxyribonucleic acid (DNA). In some embodiments, the deaminase may be AID, CDA1, or APOBEC3G. In some embodiments, ADE or CBE may create 1, 2, 3, 4, 5, 6, 7, 8, 9, 10, or more simultaneous edits at a genomic target site. In some embodiments, base editors can disrupt gene expression by making point mutations in splicing motifs or start codons. In some embodiments, base editors can disrupt gene expression by making point mutations to create termination codons. In some embodiments, a base editing system described herein may be used to create a CD3 CAR T cell. In some embodiments, the base editor system can comprise a dual base editor (e.g., a fusion of adenine and cytosine base editing components). A dual base editor (e.g., combinatorial base editor or multifunctional base editor) can comprise an adenosine deaminase domain and a cytidine deaminase domain. A dual base editor may further comprise one or more UGIs.

The present disclosure describes PEBLs that bind target antigens and sequester the target antigens to the cytoplasm of a cell. The present disclosure describes PEBLs that bind target antigens and sequester the target antigens to intracellular compartments of a cell. The target antigens may be synthesized and bind to the PEBLs intracellularly.

In certain embodiments, provided herein is a polynucleotide comprising a nucleic acid sequence encoding a PEBL comprising a target-binding molecule (e.g., a CD7 antigen binding domain) linked to a localizing domain. In some instances, the PEBL comprises from the N-terminus to the C-terminus: a CD7 antigen binding domain, an optional domain linker, and a cellular localizing domain. In some embodiments, the PEBL further comprises a signal peptide fused N-terminal to the CD7 antigen binding domain. In some embodiments, the CD7 antigen binding domain comprises a VL domain, a domain linker, and a VH domain.

As used herein, “linked” in the context of the protein expression blocker refers to a gene encoding a target-binding molecule directly in frame (e.g., without a linker) adjacent to one or more genes encoding one or more localizing domains. Alternatively, the gene encoding a target-binding molecule may be connected to one or more genes encoding one or more localizing domains through a linker sequence, e.g., as described in WO2016/126213. As would be appreciated by those of skill in the art, such linker sequences as well as variants of such linker sequences are known in the art. Methods of designing constructs that incorporate linker sequences as well as methods of assessing functionality are readily available to those of skill in the art.

In some embodiments, the localizing domain of the PEBL comprises an endoplasmic reticulum (ER) or Golgi retention sequence; or a proteosome localizing sequence. In certain embodiments, the localizing domain comprises an endoplasmic reticulum (ER) retention peptide of Table 6. In certain embodiments, the localizing domain comprises a proteasome localizing sequence set forth in Table 6. The localizing domain can direct the PEBL to a specific cellular compartment, such as the Golgi or endoplasmic reticulum, the proteasome, or the cell membrane, depending on the application.

In some embodiments, proteasome localization is achieved by linking the scFv sequence to a tripartite motif containing 21 (TRIM21) targeting domain sequence and coexpressing the sequence encoding the human TRIM21 E3 ubiquitin ligase protein. TRIM21 binds with high affinity to the Fc domains of antibodies and can recruit the ubiquitin-proteosome complex to degrade molecules (e.g., proteins and peptides) bound to the antibodies. The TRIM21 targeting domain sequence encodes amino acid sequences selected from the group of human immunoglobulin G (IgG) constant regions (Fc) genes such as IgG1, IgG2, or IgG4 and is used to form a fusion protein comprising scFv and Fc domains. In this embodiment, the exogenously expressed TRIM21 protein binds the scFv-Fc fusion protein bound to the target protein (e.g., CD7) and directs the complex to the proteasome for degradation.

Details of the amino acid sequence of the human TRIM21 E3 ligase protein can be found, for example, in NCBI Protein database under NCBI Ref. Seq. No. NP_003132.2. Details of the nucleic acid sequence encoding the human TRIM21 E3 ligase protein can be found, for example, in NCBI Protein database under NCBI Ref. Seq. No. NM_003141.3.

In some embodiments, the PEBL also includes a hinge domain and transmembrane domain sequence derived from CD8a, CD8P, 4-1BB, CD28, CD34, CD4, FcεRIγ, CD16, OX40, CD3ζ, CD3ε, CD3γ, CD3δ, TCRα, CD32, CD64, VEGFR2, FAS, or FGFR2B. In some embodiments, the PEBL comprises a hinge and transmembrane domain selected from the group consisting of a hinge and transmembrane domain of CD8a, a hinge and transmembrane domain of CD8β, a hinge and transmembrane domain of 4-1BB, a hinge and transmembrane domain of CD28, a hinge and transmembrane domain of CD34, a hinge and transmembrane domain of CD4, a hinge and transmembrane domain of FcεRIγ, a hinge domain and transmembrane domain of CD16, a hinge and transmembrane domain of OX40, a hinge and transmembrane domain of CD3ζ, a hinge and transmembrane domain of CD3ε, a hinge and transmembrane domain of CD3γ, a hinge and transmembrane domain of CD3δ, a hinge and transmembrane domain of TCRα, a hinge and transmembrane domain of CD32, a hinge and transmembrane domain of CD64, a hinge and transmembrane domain of VEGFR2, a hinge and transmembrane domain of FAS, and a hinge and transmembrane domain of FGFR2B.

In certain embodiments, the PEBL comprises one or more of the components set forth in Table 6.

TABLE 6
Amino acid sequence information for select components of a CD7 PEBL

| Component | SEQ ID NO | Amino Acid Sequence |
|---|---|---|
| ER localization domain | SEQ ID | EQKLISEEDLKDEL |
| KDEL (SEQ ID NO: 73) | NO: 71 | |
| tethered to scFv with myc | | |
| (“myc KDEL”) | | |
| Localization domain | SEQ ID | GGGGSGGGGSGGGGSGGGGSAEKDEL |
| “link(20)AEKDEL” | NO: 72 | |
| KDEL domain | SEQ ID | KDEL |
| | NO: 73 | |
| KKXX domain | | KKXX where X is any amino acid |
| KXD/E domain | | KXD or KXE where X is any amino |
| | | acid |
| YQRL domain | SEQ ID | YQRL |
| | NO: 75 | |
| PEST motif | SEQ ID | PEST |
| | NO: 76 | |
| Localization domain | SEQ ID | TTTPAPRPPTPAPTIASQPLSLRPEACRPAAGGAV |
| “mb DEKKMP” domain | NO: 77 | HTRGLDFACDIYIWAPLAGTCGVLLLSLVITLYKY |
| | | KSRRSFIDEKKMP |
| CD8α hinge and | SEQ ID | TTTPAPRPPTPAPTIASQPLSLRPEACRPAAGGAV |
| transmembrane domain | NO: 78 | HTRGLDFACDIYIWAPLAGTCGVLLLSLVITLY |
| VH-VL linker | SEQ ID | GGGGSGGGGSGGGGSGGGGS |
| | NO: 79 | |
| CD8α signal peptide | SEQ ID | MALPVTALLLPLALLLHAARP |
| | NO: 80 | |
| PEBL22 retention | SEQ ID | EQKLISEEDLGGGGSGGGGSGGGGSGGGGSAEKDE |
| sequence | NO: 99 | L |

In some embodiments, the CD7 PEBL contains CD7 antigen binding domain comprising an amino acid sequence of SEQ ID NO:32, an amino acid sequence of SEQ ID NO:33, and a VH-VL linker. The VH-VL linker can be a (G₄S)ₙ linker (SEQ ID NO: 228) where n can range from 1 to 6, e.g., 1, 2, 3, 4, 5, or 6. In one embodiment, the CD7 PEBL comprises an amino acid sequence of SEQ ID NO:32, an amino acid sequence of SEQ ID NO:33, and an amino acid sequence of SEQ ID NO:79. In some embodiments, the CD7 PEBL comprises an amino acid sequence having at least 90% sequence identity or at least 95% sequence identity to SEQ ID NO:32, the amino acid sequence of SEQ ID NO:33, and the amino acid sequence of SEQ ID NO:79. In certain embodiments, the CD7 PEBL comprises an amino acid sequence of SEQ ID NO:32, an amino acid sequence having at least 90% sequence identity or at least 95% sequence identity to SEQ ID NO: 33, and an amino acid sequence of SEQ ID NO:79. In other embodiments, the anti-CD7 protein expression blocker comprises an amino acid sequence having at least 90% sequence identity or at least 95% sequence identity to SEQ ID NO:32, an amino acid sequence having at least 90% sequence identity or at least 95% sequence identity to SEQ ID NO:33, and an amino acid sequence of SEQ ID NO:79.

In some embodiments, the CD7 PEBL contains CD7 antigen binding domain comprising an amino acid sequence of SEQ ID NO:34, an amino acid sequence of SEQ ID NO:35, and a VH-VL linker. The VH-VL linker can be a (G₄S)ₙ linker (SEQ ID NO: 228) where n can range from 1 to 6, e.g., 1, 2, 3, 4, 5, or 6. In one embodiment, the CD7 PEBL comprises an amino acid sequence of SEQ ID NO:34, an amino acid sequence of SEQ ID NO:35, and an amino acid sequence of SEQ ID NO:79. In some embodiments, the CD7 PEBL comprises an amino acid sequence having at least 95% sequence identity to SEQ ID NO:34, the amino acid sequence of SEQ ID NO:35, and the amino acid sequence of SEQ ID NO:79. In certain embodiments, the CD7 PEBL comprises an amino acid sequence of SEQ ID NO:34, an amino acid sequence having at least 95% sequence identity to SEQ ID NO:35, and an amino acid sequence of SEQ ID NO:79. In other embodiments, the CD7 PEBL comprises an amino acid sequence having at least 90% sequence identity or at least 95% sequence identity to SEQ ID NO:34, an amino acid sequence having at least 90% sequence identity or at least 95% sequence identity to SEQ ID NO:35, and an amino acid sequence of SEQ ID NO:79.

In some embodiments, the CD7 PEBL contains CD7 antigen binding domain comprising an amino acid sequence of SEQ ID NO:36, an amino acid sequence of SEQ ID NO:37, and a VH-VL linker. The VH-VL linker can be a (G₄S)ₙ linker (SEQ ID NO: 233) where n can range from 1 to 5, e.g., 1, 2, 3, 4, 5, or 6. In one embodiment, the CD7 PEBL comprises an amino acid sequence of SEQ ID NO:36, an amino acid sequence of SEQ ID NO:37, and an amino acid sequence of SEQ ID NO:79. In some embodiments, the CD7 PEBL comprises an amino acid sequence having at least 90% sequence identity or at least 95% sequence identity to SEQ ID NO:36, the amino acid sequence of SEQ ID NO:37, and the amino acid sequence of SEQ ID NO:79. In certain embodiments, the CD7 PEBL comprises an amino acid sequence of SEQ ID NO:36, an amino acid sequence having at least 90% sequence identity or at least 95% sequence identity to SEQ ID NO:37, and an amino acid sequence of SEQ ID NO:79. In other embodiments, the CD7 PEBL comprises an amino acid sequence having at least 90% sequence identity or at least 95% sequence identity to SEQ ID NO:36, an amino acid sequence having at least 90% sequence identity or at least 95% sequence identity to SEQ ID NO:37, and an amino acid sequence of SEQ ID NO:79.

In some instance, CD7 PEBL also comprises a localization domain selected from any one sequence set forth in SEQ ID NOs:72-77. In some cases, the CD7 PEBL also comprises a CD8a signal peptide such as but not limited to the CD8a signal peptide set forth in SEQ ID NO:80. In other cases, the anti-CD7 protein expression blocker also comprises CD8a hinge and transmembrane domains such as but not limited to the CD8a hinge and transmembrane domains set forth in SEQ ID NO:78.

In one embodiment, the CD7 PEBL encoded by the bicistronic vector described herein comprises the sequence of SEQ ID NO:24 and a proline at the N-terminus. In some embodiments, the CD7 PEBL comprises the sequence of SEQ ID NO:25. The N-terminal proline residue arises from the 2A cleavage. In some embodiments, the CD7 PEBL encoded by the bicistronic vector described herein comprises the sequence of SEQ ID NO:26 and a proline at the N-terminus. In some embodiments, the CD7 PEBL comprises the sequence of SEQ ID NO:27.

In some embodiments, an engineered immune cell of the present invention comprises a CD7 PEBL encoded by a bicistronic vector such that the CD7 PEBL comprises the sequence of SEQ ID NO:24 and a proline at the N-terminus or the sequence of SEQ ID NO:25. In some embodiments, the engineered immune cell is a CD4+ T cell comprising a CD7 PEBL encoded by a bicistronic vector such that the CD7 PEBL comprises the sequence of SEQ ID NO:24 and a proline at the N-terminus or the sequence of SEQ ID NO:25. In some embodiments, the engineered immune cell is a CD8+ T cell comprising a CD7 PEBL encoded by the bicistronic vector wherein the CD7 PEBL comprises the sequence of SEQ ID NO:24 and a proline at the N-terminus or the sequence of SEQ ID NO:25. In some embodiments, the engineered immune cell is a CD3+ T cell comprising a CD7 PEBL encoded by the bicistronic vector wherein the CD7 PEBL comprises the sequence of SEQ ID NO:24 and a proline at the N-terminus or the sequence of SEQ ID NO:25.

In some embodiments, an engineered immune cell of the present invention comprises a CD7 PEBL encoded by a bicistronic vector such that the CD7 PEBL comprises the sequence of SEQ ID NO:26 and a proline at the N-terminus or the sequence of SEQ ID NO:27. In some embodiments, the engineered immune cell is a CD4+ T cell comprising a CD7 PEBL encoded by the bicistronic vector wherein the CD7 PEBL comprises the sequence of SEQ ID NO:26 and a proline at the N-terminus or the sequence of SEQ ID NO:27. In some embodiments, the engineered immune cell is a CD8+ T cell comprising a CD7 PEBL encoded by the bicistronic vector wherein the CD7 PEBL comprises the sequence of SEQ ID NO:26 and a proline at the N-terminus or the sequence of SEQ ID NO:27. In some embodiments, the engineered immune cell is a CD3+ T cell comprising a CD7 PEBL encoded by the bicistronic vector wherein the CD7 PEBL comprises the sequence of SEQ ID NO:26 and a proline at the N-terminus or the sequence of SEQ ID NO:27.

In some embodiments, the CD7 PEBL encoded by the dual promoter vector described herein comprises the sequence of SEQ ID NO:24. In some embodiments, the CD7 PEBL encoded by the dual promoter vector described herein binds to CD7 and comprises at least 90% sequence identity to SEQ ID NO:24. In some embodiments, the CD7 PEBL encoded by the dual promoter vector described herein comprises the sequence of SEQ ID NO:26. In some embodiments, the CD7 PEBL encoded by the dual promoter vector described herein binds to CD7 and comprises at least 90% sequence identity to SEQ ID NO:26.

In some embodiments, the polynucleotide encoding the CD7 PEBL comprises one or more nucleic acid sequences set forth in Table 6.

In some embodiments, the VH domain of the anti-CD7 scFv of the PEBL comprises the nucleotide sequence of SEQ ID NO:38 and the VL domain of the anti-CD7 scFv of the PEBL comprises the nucleotide sequence of SEQ ID NO:39. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:38 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:39. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:38 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:39, or a codon optimized variant thereof.

In some embodiments, the VH domain of the anti-CD7 scFv of the PEBL comprises the nucleotide sequence of SEQ ID NO:40 and the VL domain of the anti-CD7 scFv of the PEBL comprises the nucleotide sequence of SEQ ID NO:41. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:40 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:41. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:40 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:41, or a codon optimized variant thereof.

In some embodiments, the VH domain of the anti-CD7 scFv of the PEBL comprises the nucleotide sequence of SEQ ID NO:42 and the VL domain of the anti-CD7 scFv of the PEBL comprises the nucleotide sequence of SEQ ID NO:43. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:42 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:43. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:42 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:43, or a codon optimized variant thereof.

TABLE 7
Nucleic acid sequence information for select
components of a CD7 PEBL

| Component | SEQ ID NO | Sequence |
|---|---|---|
| CD8α signal | SEQ ID | ATGGCTCTGCCTGTGACC |
| peptide | NO: 81 | GCACTGCTGCTGCCCCTG |
| | | GCTCTGCTGCTGCACGCC |
| | | GCAAGACCT |
| VH-VL Linker | SEQ ID | GGAGGAGGAGGAAGCGGA |
| | NO: 82 | GGAGGAGGATCCGGAGGC |
| | | GGGGGATCTGGAGGAGGA |
| | | GGAAGT |
| ER localization | SEQ ID | GAGCAGAAACTGATTAGC |
| domain KDEL | NO: 83 | GAAGAGGACCTGAAAGAT |
| (SEQ ID NO: 73) | | GAACTG |
| tethered to | | |
| scFv with myc | | |
| (“myc KDEL”) | | |

In certain aspects of the present invention, the PEBL can bind to a molecule that is expressed on the surface of a cell including, but not limited to members of the CD1 family of glycoproteins, CD2, CD3, CD4, CD5, CD7, CD8, CD25, CD28, CD38, CD45, CD45RA, CD45RO, CD52, CD56, CD57, CD99, CD127, and CD137.

In some embodiments, the CD7 PEBL comprises a nucleic acid sequence having at least 85% (e.g., 85%, 86%, 87%, 88%, 89%, 90%, or more) sequence identity to SEQ ID NO:2 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:2 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:2.

In some embodiments, the CD7 PEBL comprises a nucleic acid sequence having at least 85% (e.g., 85%, 86%, 87%, 88%, 89%, 90%, or more) sequence identity to SEQ ID NO:3 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:3 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:3.

In some embodiments, an engineered immune cell of the present invention comprises a CD7 PEBL encoded by a bicistronic construct comprising a nucleic acid sequence of the CD7 PEBL having at least 90% (e.g., 90%, 91%, 92%, 93%, 94%, 95%, 96%, 97%, 98%, 99%, or more) sequence identity to SEQ ID NO:2. In some embodiments, the engineered immune cell is a CD4+ T cell comprising a CD7 PEBL encoded by the bicistronic vector construct comprising a nucleic acid sequence of the CD7 PEBL having at least 90% sequence identity to SEQ ID NO:2. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:2. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:2. Also, provided herein is a population comprising such cells.

In some embodiments, an engineered immune cell of the present invention comprises a CD7 PEBL encoded by a bicistronic construct comprising a nucleic acid sequence of the CD7 PEBL having at least 90% (e.g., 90%, 91%, 92%, 93%, 94%, 95%, 96%, 97%, 98%, 99%, or more) sequence identity to SEQ ID NO:3. In some embodiments, the engineered immune cell is a CD4+ T cell comprising a CD7 PEBL encoded by the bicistronic vector construct comprising a nucleic acid sequence of the CD7 PEBL having at least 90% sequence identity to SEQ ID NO:3. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:3. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:3. Also, provided herein is a population comprising such cells.

Chimeric Antigen Receptors that Bind CD7

In some embodiments, the CAR of the present invention comprises intracellular signaling domains of 4-1BB and CD3ζ, and an antigen binding domain (e.g., a single chain variable fragment or scFv) that specifically binds CD7. The CD7 CAR of the present invention is sometimes referred to herein as “anti-CD7-41BB-CD3ζ”. In some embodiments, the CAR also includes a CD8a hinge domain and transmembrane domain, such as but not limited the amino acid sequence of SEQ ID NO:84.

As those skilled in the art would appreciate, in certain embodiments, any of the amino acid sequences of the various components disclosed herein (e.g., scFv, intracellular signaling domain, linker, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein. For example, in certain embodiments, the intracellular signaling domain 4-1BB can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to SEQ ID NO:85, as long as it possesses the desired function. In certain embodiments, the intracellular signaling domain of 4-1BB comprises the amino acid sequence set forth in SEQ ID NO:85.

As another example, in certain embodiments, the intracellular signaling domain 4-1BB can be replaced by another intracellular signaling domain from a co-stimulatory molecule such as CD28, OX40, ICOS, CD27, GITR, HVEM, TIM1, LFA1, CD30, CD84, CRTAM, DR3, SLAMF1, or CD2. In some embodiments, the intracellular signaling domain of the CAR can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2.

As another example, in certain instances, the intracellular signaling domain of 4-1BB can also include another intracellular signaling domain (or a portion thereof) from a co-stimulatory molecule such as CD28, OX40, ICOS, CD27, GITR, HVEM, TIM1, LFA1, CD30, CD84, CRTAM, DR3, SLAMF1, or CD2. In some embodiments, the additional intracellular signaling domain can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2. In other embodiments, the additional intracellular signaling domain comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one or more intracellular signaling domain fragment(s) of CD28, OX40, ICOS, CD27, GITR, HVEM, TIM1, LFA1, or CD2.

As another example, in certain embodiments, the intracellular signaling domain CD3 can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to SEQ ID NO:86, as long as it possesses the desired function. In certain embodiments, the intracellular signaling domain of CD3ζ comprises the amino acid sequence set forth in SEQ ID NO:86.

In some instances, the intracellular signaling domain comprises an immunoreceptor tyrosine-based activation motif (ITAM) or a portion thereof, as long as it possesses the desired function. The intracellular signaling domain of the CAR can include a sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to an ITAM. In some instances, the intracellular signaling domain can have at least 95% sequence identity, at least 96% sequence identity, at least 97% sequence identity, at least 98% sequence identity, at least 99% sequence identity, or 100% sequence identity to FcεRIγ, CD4, CD7, CD8, CD28, OX40, CD79a, CD79b, DAP12, or H2-Kb, as long as it possesses the desired function.

In certain embodiments, the anti-CD7 CAR further comprises a hinge domain and/or a transmembrane domain. Hinge and transmembrane domains suitable for use in the present invention are known in the art, and provided in, e.g., publication WO2016/126213, incorporated by reference in its entirety. In some embodiments, the hinge and transmembrane domains of the anti-CD7 CAR includes a signaling domain (e.g., hinge and transmembrane domains) from CD8P, 4-1BB, CD28, CD34, CD4, FcεRIγ, CD16, OX40, CD3ζ, CD3ε, CD3γ, CD3δ, TCRα, CD32, CD64, VEGFR2, FAS, FGFR2B, or another transmembrane protein.

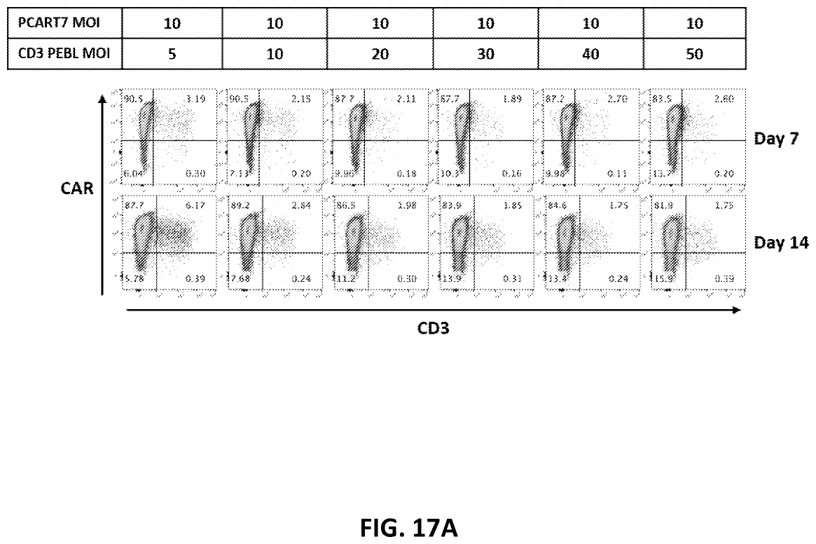

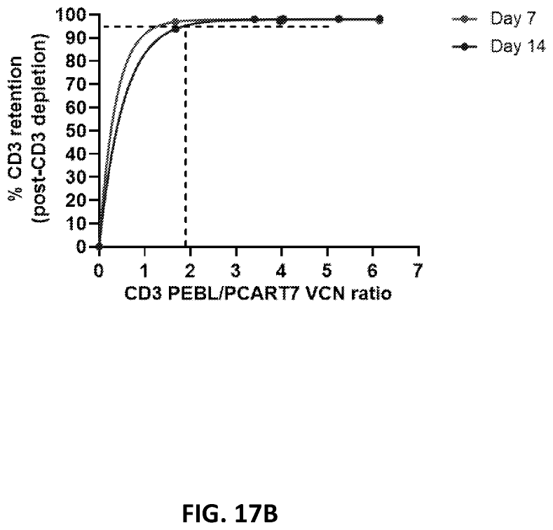

In certain embodiments, the anti-CD7 CAR further comprises a CD8a signal peptide. A schematic of the anti-CD7 CAR comprising the embodiments described herein is shown in FIG. 17 of US 2018/0179280.

In some embodiments, the chimeric antigen receptor (CAR) can bind to a molecule that is expressed on the surface of a cell including, but not limited to members of the CD1 family of glycoproteins, CD2, CD3, CD4, CD5, CD7, CD8, CD25, CD28, CD38, CD45, CD45RA, CD45RO, CD52, CD56, CD57, CD99, CD127, and CD137.

In certain embodiments, an isolated polypeptide of the present invention comprises a amino acid sequence that encodes a CAR according to Table 8. In some embodiments, the polypeptide comprises a amino acid sequence that encodes a component of the CAR according to Table 8.

TABLE 8
Amino acid sequence information for select
components of a CD7 CAR

| Component | SEQ ID NO | Amino Acid Sequence |
|---|---|---|
| CD8α hinge and | SEQ ID | TTTPAPRPPTPAPTIASQPLSLR |
| transmembrane | NO: 84 | PEACRPAAGGAVHTRGLDFACDI |
| domain | | YIWAPLAGTCGVLLLSLVITLY |
| Intracellular | SEQ ID | KRGRKKLLYIFKQPFMRPVQTTQ |
| signaling | NO: 85 | EEDGCSCRFPEEEEGGCEL |
| domain of 4-1BB | | |
| Intracellular | SEQ ID | RVKFSRSADAPAYQQGQNQLYNE |
| signaling | NO: 86 | LNLGRREEYDVLDKRRGRDPEMG |
| domain CDζ | | GKPRRKNPQEGLYNELQKDKMAE |
| | | AYSEIGMKGERRRGKGHDGLYQG |
| | | LSTATKDTYDALHMQALPPR |

In some embodiments, the CD7 CAR comprises a CD7 antigen binding domain, a 4-1BB intracellular signaling domain, a CD3ζ intracellular signaling domain, and CD8 hinge and transmembrane domain. In some embodiments, the CD7 antigen binding domain comprises a VH domain and a VL domain, and a VH-VL linker, such as but not limited to a (G₄S)ₙ linker (SEQ ID NO: 228) where n can range from 1 to 6, e.g., 1, 2, 3, 4, 5, or 6. In some embodiments, the CD7 CAR comprises from N-terminus to C-terminus: a CD8 signal peptide, a CD7 antigen binding domain, aCD8 hinge and transmembrane domain, a 4-1BB intracellular signaling domain, and a CD3ζ intracellular signaling domain.

In some embodiments, the CD7 CAR encoded by the bicistronic vector described herein comprises the amino acid sequence of SEQ ID NO:28. In some embodiments, the CD7 CAR encoded by the bicistronic vector described herein comprises the amino acid sequence of SEQ ID NO:29. In some embodiments, the CD7 CAR encoded by the bicistronic vector described herein comprises the amino acid sequence of SEQ ID NO:30. In some embodiments, the CD7 CAR encoded by the bicistronic vector described herein comprises the amino acid sequence of SEQ ID NO:31.

In some embodiments, an engineered immune cell of the present invention comprises a CD7 CAR encoded by a bicistronic vector such that the CD7 CAR comprises the sequence of SEQ ID NO:28 and additional amino acid residues at the N-terminus produced by cleavage of the 2A self-cleaving peptide, or the CD7 CAR comprises the sequence of SEQ ID NO:29. In some embodiments, the engineered immune cell is a CD4+ T cell comprising a CD7 CAR encoded by a bicistronic vector such that the CD7 CAR comprises the sequence of SEQ ID NO:28 and additional amino acid residues at the C-terminus produced by cleavage of the 2A self-cleaving peptide, or the CD7 CAR comprises the sequence of SEQ ID NO:29. In some embodiments, the engineered immune cell is a CD8+ T cell comprising a CD7 CAR encoded by a bicistronic vector such that the CD7 CAR comprises the sequence of SEQ ID NO:28 and additional amino acid residues at the C-terminus produced by cleavage of the 2A self-cleaving peptide, or the CD7 CAR comprises the sequence of SEQ ID NO:29. In some embodiments, the engineered immune cell is a CD3+ T cell comprising a CD7 CAR encoded by a bicistronic vector such that the CD7 CAR comprises the sequence of SEQ ID NO:28 and additional amino acid residues at the C-terminus produced by cleavage of the 2A self-cleaving peptide, or the CD7 CAR comprises the sequence of SEQ ID NO:29. Also, provided herein are populations comprising such cells.

In some embodiments, an engineered immune cell of the present invention comprises a CD7 CAR encoded by a bicistronic vector such that the CD7 CAR comprises the sequence of SEQ ID NO:30 and additional amino acid residues at the C-terminus produced by cleavage of the 2A self-cleaving peptide, or the CD7 CAR comprises the sequence of SEQ ID NO:31. In some embodiments, the engineered immune cell is a CD4+ T cell comprising a CD7 CAR encoded by a bicistronic vector such that the CD7 CAR comprises the sequence of SEQ ID NO:30 and additional amino acid residues at the C-terminus produced by cleavage of the 2A self-cleaving peptide, or the CD7 CAR comprises the sequence of SEQ ID NO:31. In some embodiments, the engineered immune cell is a CD8+ T cell comprising a CD7 CAR encoded by a bicistronic vector such that the CD7 CAR comprises the sequence of SEQ ID NO:30 and additional amino acid residues at the C-terminus produced by cleavage of the 2A self-cleaving peptide, or the CD7 CAR comprises the sequence of SEQ ID NO:31. In some embodiments, the engineered immune cell is a CD3+ T cell comprising a CD7 CAR encoded by a bicistronic vector such that the CD7 CAR comprises the sequence of SEQ ID NO:30 and additional amino acid residues at the C-terminus produced by cleavage of the 2A self-cleaving peptide, or the CD7 CAR comprises the sequence of SEQ ID NO:31. Also, provided herein are populations of such cells.

In some embodiments, the CD7 CAR encoded by the dual promoter vector described herein comprises the amino acid sequence of SEQ ID NO:28. In some embodiments, an engineered immune cell of the present invention comprises a CD7 CAR encoded by a dual promoter vector such that the CD7 CAR comprises the sequence of SEQ ID NO:28. In some embodiments, the engineered immune cell is a CD4+ T cell comprising a CD7 CAR encoded by a dual promoter vector such that the CD7 CAR comprises the sequence of SEQ ID NO:28. In some embodiments, the engineered immune cell is a CD8+ T cell comprising a CD7 CAR encoded by a dual promoter vector such that the CD7 CAR comprises the sequence of SEQ ID NO:28. In some embodiments, the engineered immune cell is a CD3+ T cell comprising a CD7 CAR encoded by a dual promoter vector such that the CD7 CAR comprises the sequence of SEQ ID NO:28. In some embodiments, the CD7 CAR encoded by the bicistronic vector described herein comprises the amino acid sequence of SEQ ID NO:30. In some embodiments, an engineered immune cell of the present invention comprises a CD7 CAR encoded by a dual promoter vector such that the CD7 CAR comprises the sequence of SEQ ID NO:30. In some embodiments, the engineered immune cell is a CD4+ T cell comprising a CD7 CAR encoded by a dual promoter vector such that the CD7 CAR comprises the sequence of SEQ ID NO:30. In some embodiments, the engineered immune cell is a CD8+ T cell comprising a CD7 CAR encoded by a dual promoter vector such that the CD7 CAR comprises the sequence of SEQ ID NO:30. In some embodiments, the engineered immune cell is a CD3+ T cell comprising a CD7 CAR encoded by a dual promoter vector such that the CD7 CAR comprises the sequence of SEQ ID NO:30. Also, provided herein are populations of such cells.

In certain embodiments, an isolated polynucleotide of a CD7 CAR of the present invention comprises one or more nucleic acid sequences of Table 9?. In some embodiments, the nucleic acid sequence comprises a sequence encoding one or more components of the CAR as set forth in Table 9?.

TABLE 9
Nucleic acid sequence information for select
components of a CD7 CAR

| Component | SEQ ID NO | Nucleic Acid Sequence |
|---|---|---|
| CD8α hinge and | SEQ ID | ACCACTACACCTGCACCAAGGCCTCCCACACCCGC |
| transmembrane | NO: 96 | TCCCACTATCGCTTCCCAGCCACTGTCCCTGAGGC |
| domain | | CCGAGGCCTGCAGGCCAGCAGCTGGCGGAGCCGTG |
| | | CATACTAGGGGGCTGGACTTCGCTTGCGACATCTA |
| | | CATCTGGGCCCCACTGGCAGGGACATGCGGAGTCC |
| | | TGCTGCTGTCCCTGGTCATCACACTGTACTGC |
| Intracellular | SEQ ID | AAGCGGGGGCGCAAAAAACTGCTGTATATCTTTAA |
| signaling | NO: 97 | GCAGCCTTTCATGAGACCAGTGCAGACAACCCAGG |
| domain of | | AGGAAGATGGGTGCTCATGCCGGTTTCCCGAGGAG |
| 4-1BB | | GAGGAAGGCGGCTGCGAGCTG |
| Intracellular | SEQ ID | GGGTGAAGTTTTCCCGCTCAGCAGATGCTCCTGCC |
| signaling | NO: 98 | TACCAGCAGGGCCAGAACCAGCTGTATAATGAGCT |
| domain of CD3ζ | | GAACCTGGGCAGACGCGAAGAGTATGATGTGCTGG |
| | | ACAAAAGGGGGGGAAGAGACCCCGAAATGGGAGGG |
| | | AAGCCAAGGCGGAAAAACCCCCAGGAGGGCCTGTA |
| | | CAATGAGCTGCAGAAGGACAAAATGGCAGAGGCTT |
| | | ACAGTGAGATTGGGATGAAGGGAGAGAGACGGAGG |
| | | GGAAAAGGGCACGATGGCCTGTACCAGGGGCTGAG |
| | | CACAGCAACCAAAGATACTTATGACGCACTGCACA |
| | | TGCAGGCACTGCCACCCAGA |

In some embodiments, the polynucleotide encoding the CD7 CAR comprises a nucleic acid sequence for an antigen binding domain that binds CD7, a nucleic acid sequence for a CD8a hinge and transmembrane domain, a nucleic acid sequence for an intracellular signaling domain of 4-1BB, and a nucleic acid sequence for an intracellular signaling domain of CD3ζ. In certain embodiments, the polynucleotide also includes a nucleic acid sequence for a CD8 signal peptide.

In certain embodiments, the antigen binding domain is an anti-CD7 scFv. In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:38 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:39.

In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:40 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:41. In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:42 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:43.

In some embodiments, the polynucleotide encoding the CD7 CAR comprises from the 5′ end to the 3′ end: a nucleic acid sequence for an antigen binding domain that binds CD7, SEQ ID NO:96, SEQ ID NO:97, and SEQ ID NO:98. In some embodiments, the polynucleotide encoding the CD7 CAR comprises from the 5′ end to the 3′ end: SEQ ID NO:81, a nucleic acid sequence for an antigen binding domain that binds CD7, SEQ ID NO:96, SEQ ID NO:97, and SEQ ID NO:98.

In some embodiments, the CD7 CAR comprises a nucleic acid sequence having at least 85% (e.g., 85%, 86%, 87%, 88%, 89%, 90%, or more) sequence identity to SEQ ID NO:4 and binds to CD7. In some embodiments, the CD7 CAR comprises a nucleic acid sequence having at least 90% (e.g., 90%, 91%, 92%, 93%, 94%, 95%, 96%, 97%, 98%, 99%, or more) sequence identity to SEQ ID NO:4 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:4.

In some embodiments, the CD7 CAR comprises a nucleic acid sequence having at least 85% (e.g., 85%, 86%, 87%, 88%, 89%, 90%, or more) sequence identity to SEQ ID NO:5 and binds to CD7. In some embodiments, the CD7 CAR comprises a nucleic acid sequence having at least 90% (e.g., 90%, 91%, 92%, 93%, 94%, 95%, 96%, 97%, 98%, 99%, or more) sequence identity to SEQ ID NO:5 and binds to CD7. In some embodiments, the CD7 PEBL comprises a nucleic acid sequence having at least 90% (e.g., 90%, 91%, 92%, 93%, 94%, 95%, 96%, 97%, 98%, 99%, or more) sequence identity to SEQ ID NO:5.

In some embodiments, an engineered immune cell of the present invention comprises a CD7 CAR encoded by a bicistronic construct or a dual promoter construct comprising a nucleic acid sequence of the CD7 CAR having at least 90% (e.g., 90%, 91%, 92%, 93%, 94%, 95%, 96%, 97%, 98%, 99%, or more) sequence identity to SEQ ID NO:4. In some embodiments, the engineered immune cell is a CD4+ T cell comprising a CD7 CAR encoded by the bicistronic vector construct or a dual promoter construct comprising a nucleic acid sequence of the CD7 PEBL having at least 90% sequence identity to SEQ ID NO:4. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:4. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:4. Also, provided herein is a population comprising such cells.

In some embodiments, an engineered immune cell of the present invention comprises a CD7 CAR encoded by a bicistronic construct or a dual promoter construct comprising a nucleic acid sequence of the CD7 CAR having at least 90% (e.g., 90%, 91%, 92%, 93%, 94%, 95%, 96%, 97%, 98%, 99%, or more) sequence identity to SEQ ID NO:5. In some embodiments, the engineered immune cell is a CD4+ T cell comprising a CD7 CAR encoded by the bicistronic vector construct comprising a nucleic acid sequence of the CD7 PEBL having at least 90% sequence identity to SEQ ID NO:5. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:5. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:5. Also, provided herein is a population comprising such cells.

### Downregulation of Intracellular CD3 Via CD3 PEBL

In some instances, as described herein, T cell cytotoxicity was shown to be markedly increased when anti-CD7 CAR was used in combination with downregulation of CD7 and CD3 expression on the effector T cells. As demonstrated herein, downregulation (e.g., elimination, reduction, and/or relocalization) of CD7 and CD3 not only prevented the fratricidal effect exerted by the corresponding anti-CD7 CAR, allowing greater T cell recovery after CAR expression as compared to cells that retained the target antigen (e.g., CD7), and a more effective cytotoxicity against T leukemia/lymphoma cells, but also prevented the development of GvHD. As those of skill in the art would appreciate, downregulation of CD7 and CD3 expression on the effector T cells can be achieved according to a variety of known methods including, for example, protein expression blockers (PEBLs) against CD7 (as described in WO2016/126213) and against CD3, RNAi against CD7 and against CD3, or gene editing methods such as, e.g., meganucleases, TALEN, CRISPR/Cas9, and zinc finger nucleases. Gene disruption may also be possible through base editing technologies. Base editors are capable of making single base pair changes at defined genetic loci to alter gene expression. Adenine base editors (ABEs) and cytosine base editors (CBEs) combine a deaminase enzyme with a Cas nickase to mediate gene editing without double-stranded breaks in DNA. Without wishing to be bound by theory, the methods provided herein may provide for an efficient targeted multiplexed editing system. In some embodiments, a base editor system comprises a nucleotide binding domain, a deaminase domain for deaminating nucleobases in a target nucleotide sequence; and one or more guide RNA molecules (gRNAs) targeting Cas to a specific locus. Adenine base editors make A to G (or T to C) point mutations at a target site and cytosine base editors make C to T (or G to A) point mutations at a target site. In some cases, cytosine base editors can be fused with an inhibitor of uracil DNA glycosylase (UGI) to prevent base excision repair. In some embodiments, the deaminase is an adenosine deaminase. In some embodiments, the adenosine deaminase catalyzes the hydrolytic deamination of adenine or adenosine in deoxyribonucleic acid (DNA). In some embodiments, the deaminase may be AID, CDA1, or APOBEC3G. In some embodiments, ADE or CBE may create 1, 2, 3, 4, 5, 6, 7, 8, 9, 10, or more simultaneous edits at a genomic target site. In some embodiments, base editors can disrupt gene expression by making point mutations in splicing motifs or start codons. In some embodiments, base editors can disrupt gene expression by making point mutations to create termination codons. In some embodiments, a base editing system described herein may be used to create a CD3 CAR T cell. In some embodiments, the base editor system can comprise a dual base editor (e.g., a fusion of adenine and cytosine base editing components). A dual base editor (e.g., combinatorial base editor or multifunctional base editor) can comprise an adenosine deaminase domain and a cytidine deaminase domain. A dual base editor may further comprise one or more UGIs.

The present invention describes PEBLs that bind target antigens and sequester the target antigens to a specific cellular compartment of a cell. The target antigens are synthesized and bind to the PEBLs intracellularly.

As those skilled in the art would appreciate, in certain embodiments, any of the amino acid sequences of the various components disclosed herein (e.g., scFv, intracellular signaling domain, linker, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein.

In certain embodiments, provided herein is a polynucleotide comprising a nucleic acid sequence encoding a PEBL comprising a target-binding molecule (e.g., a CD3 antigen binding domain) linked to a localizing domain. In some instances, the PEBL comprises from the N-terminus to the C-terminus: a CD3 antigen binding domain, an optional domain linker, and a cellular localizing domain. In some embodiments, the PEBL further comprises a signal peptide fused N-terminal to the CD3 antigen binding domain. In some embodiments, the CD3 antigen binding domain comprises a VL domain, a domain linker, and a VH domain.

As used herein, “linked” in the context of the protein expression blocker refers to a gene encoding a target-binding molecule directly in frame (e.g., without a linker) adjacent to one or more genes encoding one or more localizing domains. Alternatively, the gene encoding a target-binding molecule may be connected to one or more genes encoding one or more localizing domains through a linker sequence, e.g., as described in WO2016/126213. As would be appreciated by those of skill in the art, such linker sequences as well as variants of such linker sequences are known in the art. Methods of designing constructs that incorporate linker sequences as well as methods of assessing functionality are readily available to those of skill in the art.

In some embodiments, the localizing domain of the PEBL comprises an endoplasmic reticulum (ER) or Golgi retention sequence; or a proteosome localizing sequence. In certain embodiments, the localizing domain comprises an endoplasmic reticulum (ER) retention peptide of Table 10 or Table 11. In certain embodiments, the localizing domain comprises a proteasome localizing sequence set forth in Table 10 or Table 11. The localizing domain can direct the PEBL to a specific cellular compartment, such as the Golgi or endoplasmic reticulum, the proteasome, or the cell membrane, depending on the application.

In some embodiments, proteasome localization is achieved by linking the scFv sequence to a tripartite motif containing 21 (TRIM21) targeting domain sequence and coexpressing the sequence encoding the human TRIM21 E3 ubiquitin ligase protein. TRIM21 binds with high affinity to the Fc domains of antibodies and can recruit the ubiquitin-proteosome complex to degrade molecules (e.g., proteins and peptides) bound to the antibodies. The TRIM21 targeting domain sequence encodes amino acid sequences selected from the group of human immunoglobulin G (IgG) constant regions (Fc) genes such as IgG1, IgG2, or IgG4 and is used to form a fusion protein comprising scFv and Fc domains. In this embodiment, the exogenously expressed TRIM21 protein binds the scFv-Fc fusion protein bound to the target protein (e.g., CD7) and directs the complex to the proteasome for degradation.

Details of the amino acid sequence of the human TRIM21 E3 ligase protein can be found, for example, in NCBI Protein database under NCBI Ref. Seq. No. NP_003132.2. Details of the nucleic acid sequence encoding the human TRIM21 E3 ligase protein can be found, for example, in NCBI Protein database under NCBI Ref. Seq. No. NM_003141.3.

In some embodiments, the PEBL also includes a hinge domain and transmembrane domain sequence derived from CD8α, CD8β, 4-1BB, CD28, CD34, CD4, FcεRIγ, CD16, OX40, CD3ζ, CD3ε, CD3γ, CD3δ, TCRα, CD32, CD64, VEGFR2, FAS, or FGFR2B. In some embodiments, the PEBL comprises a hinge and transmembrane domain selected from the group consisting of a hinge and transmembrane domain of CD8α, a hinge and transmembrane domain of CD8β, a hinge and transmembrane domain of 4-1BB, a hinge and transmembrane domain of CD28, a hinge and transmembrane domain of CD34, a hinge and transmembrane domain of CD4, a hinge and transmembrane domain of FcεRIγ, a hinge domain and transmembrane domain of CD16, a hinge and transmembrane domain of OX40, a hinge and transmembrane domain of CD3ζ, a hinge and transmembrane domain of CD3ε, a hinge and transmembrane domain of CD3γ, a hinge and transmembrane domain of CD3δ, a hinge and transmembrane domain of TCRα, a hinge and transmembrane domain of CD32, a hinge and transmembrane domain of CD64, a hinge and transmembrane domain of VEGFR2, a hinge and transmembrane domain of FAS, and a hinge and transmembrane domain of FGFR2B.

In some embodiments, the PEBL against CD3 comprises one or more of the components set forth in Table 10. In some embodiments, the PEBL against CD3 comprises an amino acid sequence of SEQ ID NO: 101. In some embodiments, the CD3 binding domain comprises binding sequences derived from an anti-CD3 antibody derived from Clone OKT3 as in SEQ ID NO: 102.

OKT3 PEBL5 sequence (SEQ ID NO: 101):
MALPVTALLLPLALLLHAARPQIVLTQSPAIMSASPGEKVTMTCSASSSV
SYMNWYQQKSGTSPKRWIYDTSKLASGVPAHFRGSGSGTSYSLTISGMEA
EDAATYYCQQWSSNPFTFGSGTKLEINRGGGGSGGGGSGGGGSGGGGSEV
QLQQSGAELARPGASVKMSCKASGYTFTRYTMHWVKQRPGQGLEWIGYIN
PSRGYTNYNQKFKDKATLTTDKSSSTAYMQLSSLTSEDSAVYYCARYYDD
HYCLDYWGQGTTLTVSSAGGGGSGGGGSGGGGSGGGGSAEKDEL

TABLE 10
Amino acid sequence information for select
components of a CD3 PEBL

| | |
|---|---|
| CD8 signal | MALPVTALLLPLALLLHAARP |
| peptide | |
| (SEQ ID | |
| NO: 80) | |
| OKT3 scFv | QIVLTQSPAIMSASPGEKVTMTCSASSSVSYMNWYQQK |
| (SEQ ID | SGTSPKRWIYDTSKLASGVPAHFRGSGSGTSYSLTISG |
| NO: 102) | MEAEDAATYYCQQWSSNPFTFGSGTKLEINRGGGGSGG |
| | GGSGGGGSGGGGSEVQLQQSGAELARPGASVKMSCKAS |
| | GYTFTRYTMHWVKQRPGQGLEWIGYINPSRGYTNYNQK |
| | FKDKATLTTDKSSSTAYMQLSSLTSEDSAVYYCARYYD |
| | DHYCLDYWGQGTTLTVSSA |
| GS linker + | GGGGSGGGGSGGGGSGGGGSAE |
| 2 more aa | |
| (SEQ ID | |
| NO: 229) | |
| ER | KDEL |
| retention | |
| (SEQ ID | |
| NO: 73) | |

In some embodiments, the PEBL against CD3 comprises one or more of the components set forth in Table 11. In some embodiments, the PEBL against CD3 comprises an amino acid sequence of SEQ ID NO: 103. In some embodiments, the CD3 binding domain comprises binding sequences derived from an anti-CD3 antibody derived from Clone UCHT1 as in SEQ ID NO: 104.

UCHT1 PEBL22 sequence (SEQ ID NO: 103):
MALPVTALLLPLALLLHAARPDIQMTQTTSSLSASLGDRVTISCRASQDI
RNYLNWYQQKPDGTVKLLIYYTSRLHSGVPSKFSGSGSGTDYSLTISNLE
QEDIATYFCQQGNTLPWTFAGGTKLEIKGGGGSGGGGSGGGGSGGGGSEV
QLQQSGPELVKPGASMKISCKASGYSFTGYTMNWVKQSHGKNLEWMGLIN
PYKGVSTYNQKFKDKATLTVDKSSSTAYMELLSLTSEDSAVYYCARSGYY
GDSDWYFDVWGQGTTLTVFSEQKLISEEDLGGGGSGGGGSGGGGSGGGGS
AEKDEL

TABLE 11
Amino acid sequence information for select
components of a CD3 PEBL

| | |
|---|---|
| CD8 signal | MALPVTALLLPLALLLHAARP |
| peptide | |
| (SEQ ID | |
| NO: 80) | |
| UCHT1 | DIQMTQTTSSLSASLGDRVTISCRASQDIRNYLNWYQQKPDGTVKLLIYYTSRLH |
| scFv | SGVPSKFSGSGSGTDYSLTISNLEQEDIATYFCQQGNTLPWTFAGGTKLEIKGGG |
| (SEQ ID | GSGGGGSGGGGSGGGGSEVQLQQSGPELVKPGASMKISCKASGYSFTGYTMNWVK |
| NO: 104) | QSHGKNLEWMGLINPYKGVSTYNQKFKDKATLTVDKSSSTAYMELLSLTSEDSAV |
| | YYCARSGYYGDSDWYFDVWGQGTTLTVFS |
| Myc tag | EQKLISEEDL |
| (SEQ ID | |
| NO: 227) | |
| GS linker + | GGGGSGGGGSGGGGSGGGGSAE |
| 2 more aa | |
| (SEQ ID | |
| NO: 229) | |
| ER | KDEL |
| retention | |
| (SEQ ID | |
| NO: 73) | |

In some embodiments, the D3 PEBL comprises an amino acid sequence having at least 85% (e.g., 85%, 86%, 87%, 88%, 89%, 90%, or more) sequence identity to SEQ ID NO: 101 and binds to CD3. In some embodiments, the CD3 PEBL comprises an amino acid sequence having at least 90% (e.g., 90%, 91%, 92%, 93%, 94%, 95%, 96%, 97%, 98%, 99%, or more) sequence identity to SEQ ID NO: 101 and binds to CD3. In some embodiments, the CD3 PEBL comprises an amino acid sequence having at least 90% (e.g., 90%, 91%, 92%, 93%, 94%, 95%, 96%, 97%, 98%, 99%, or more) sequence identity to SEQ ID NO: 101.

In some embodiments, an engineered immune cell of the present invention comprises a CD3 PEBL encoded by a construct comprising a nucleic acid sequence encoding an amino acid sequence of the CD3 PEBL having at least 90% (e.g., 90%, 91%, 92%, 93%, 94%, 95%, 96%, 97%, 98%, 99%, or more) sequence identity to SEQ ID NO: 101. In some embodiments, the engineered immune cell is a CD4+ T cell comprising a CD7 PEBL encoded by the construct comprising a nucleic acid sequence encoding an amino acid sequence of the CD3 PEBL having at least 90% sequence identity to SEQ ID NO:101. In some embodiments, the engineered immune cell is a CD8+ T cell comprising a CD3 PEBL encoded by the construct comprising a nucleic acid sequence encoding an amino acid sequence of the CD3 PEBL having at least 90% sequence identity to SEQ ID NO: 101. Also, provided herein is a population comprising such cells.

In some aspects, the CD3 PEBL targets CD3ε. In some aspects, the CD3 PEBL comprises a binding domain that binds to CD3ε. In some aspects, the CD3 PEBL comprises an antibody or its antigen binding domain binding to CD3ε. In some embodiments, the CD3 PEBL comprises an amino acid sequence having at least 85% (e.g., 85%, 86%, 87%, 88%, 89%, 90%, or more) sequence identity to SEQ ID NO:103 and binds to CD3. In some embodiments, the CD3 PEBL comprises an amino acid sequence having at least 90% (e.g., 90%, 91%, 92%, 93%, 94%, 95%, 96%, 97%, 98%, 99%, or more) sequence identity to SEQ ID NO:103 and binds to CD3. In some embodiments, the CD3 PEBL comprises an amino acid sequence having at least 90% (e.g., 90%, 91%, 92%, 93%, 94%, 95%, 96%, 97%, 98%, 99%, or more) sequence identity to SEQ ID NO:103.

In some embodiments, an engineered immune cell of the present invention comprises a CD3 PEBL encoded by a construct comprising a nucleic acid sequence encoding an amino acid sequence of the CD3 PEBL having at least 90% (e.g., 90%, 91%, 92%, 93%, 94%, 95%, 96%, 97%, 98%, 99%, or more) sequence identity to SEQ ID NO:103. In some embodiments, the engineered immune cell is a CD4+ T cell comprising a CD7 PEBL encoded by the construct comprising a nucleic acid sequence encoding an amino acid sequence of the CD3 PEBL having at least 90% sequence identity to SEQ ID NO:103. In some embodiments, the engineered immune cell is a CD8+ T cell comprising a CD3 PEBL encoded by the construct comprising a nucleic acid sequence encoding an amino acid sequence of the CD3 PEBL having at least 90% sequence identity to SEQ ID NO:103. Also, provided herein is a population comprising such cells.

In some aspects, provided herein are recombinant nucleic acid molecules encoding a CD3, binding domain linked to a synthetic localizing domain. In some embodiments, the CD3, binding domain comprises a heavy chain complementarity-determining region (HC CDR1) of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223.

In some embodiments, the synthetic localizing domain comprises a linker sequence having at least 2, at least 3, at least 4, at least 5, at least 6, at least 7, at least 8, at least 9, or at least 10 amino acids in length and an amino acid sequence KDEL (SEQ ID NO: 73). In some embodiments, the linker sequence comprises (GGGGS)n (SEQ ID NO: 224), where n is any integer from 1 to 10. In some embodiments, the linker sequence comprises (GGGGS)₄ (SEQ ID NO: 79). The synthetic localizing domain may also comprise a Myc tag. In some embodiments, the recombinant nucleic acid molecule comprises a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 99. In some embodiments, the recombinant nucleic acid molecule comprises a sequence of SEQ ID NO: 99.

In some embodiments, an engineered immune cell can comprise a recombinant nucleic acid molecule described herein.

### Kill Gene or Suicide Gene

In some embodiments, the engineered immune cell described herein can comprise a kill gene or a suicide gene that induces cell death of the engineered immune cells upon activation. In some embodiments, the construct encoding a CAR of the present disclosure may comprise a kill gene or a suicide gene that induces cell death of the engineered immune cells upon activation. In some cases, the construct encoding the CD3 PEBL may comprise a kill gene.

In some instances, the engineered immune cells such as CD7 CAR+/CD7-negative T cell or PCART7-CD3PEBL T cell can further comprises a fourth nucleic acid comprising a suicide gene. In some embodiments, the suicide gene comprises CD20 or a derivative thereof. In some embodiments, the suicide gene comprises CD20, a derivative thereof, or a portion thereof. A derivative thereof may comprise a functional fragment of CD20, e.g., SEQ ID NO: 106. In some embodiments, the suicide gene comprises a modified CD20. In some embodiments, the suicide gene comprises a truncated CD20. In some embodiments, the suicide gene comprises CD20, p53 protein, inducible Caspase 9 (iCasp9), herpes simplex virus tyrosine kinase (HSV-TK), human thymidylate kinase (TMPK), epidermal growth factor receptor (EGFR), or derivative thereof.

In some embodiments, the fourth nucleic acid is located in the second expression vector, wherein the second expression vector encoding the second PEBL, e.g., the second bicistronic expression vector. In some embodiments, the fourth nucleic acid is located in the first expression vector, wherein the first expression vector encoding the CAR and the first PEBL, e.g., a tricistronic expression vector. In some embodiments, the fourth nucleic acid is located in a separate expression vector, e.g., a third expression vector. In some embodiments, the first nucleic acid encoding the CAR, the second nucleic acid encoding the first PEBL, the third nucleic acid encoding the second PEBL, and the fourth nucleic acid encoding the suicide gene are located in separate expression vectors. In some embodiments, the first nucleic acid encoding the CAR, the second nucleic acid encoding the first PEBL, the third nucleic acid encoding the second PEBL, and the fourth nucleic acid encoding the suicide gene are located in the same expression vector.

### Bicistronic Expression Constructs of a Suicide Gene and a Second PEBL

Provided herein are recombinant bicistronic viral constructs or vectors that contain a polynucleotide encoding a suicide gene and a polynucleotide encoding a second PEBL, as described herein. In some embodiments, the recombinant bicistronic viral construct includes an internal ribosomal entry site (IRES) sequence between the nucleic acid sequence of the suicide gene and the nucleic acid sequence of the second PEBL. In some embodiments, the recombinant bicistronic viral construct includes a ribosomal codon skipping site sequence (also referred to as a sequence encoding a 2A self-cleaving peptide) between the nucleic acid sequence of the suicide gene and the nucleic acid sequence of the second PEBL. In some embodiments of a bicistronic construct, a polynucleotide encoding a suicide gene is located upstream (at the 5′ end) of an IRES sequence, and a polynucleotide encoding a second PEBL is located downstream (at the 3′ end) of the IRES. In some cases, a nucleic acid sequence encoding a suicide gene is operably linked to an IRES sequence and an IRES sequence is operably linked to a nucleic acid sequence encoding a second PEBL. In some cases, a nucleic acid sequence encoding a second PEBL is operably linked to an IRES sequence and an IRES sequence is operably linked to a nucleic acid sequence encoding a suicide gene.

In some embodiments of a bicistronic construct, a polynucleotide encoding a suicide gene is located upstream (at the 5′ end) of a polynucleotide encoding 2A self-cleaving peptide, and a polynucleotide encoding a second PEBL is located downstream (at the 3′ end) of the polynucleotide encoding 2A self-cleaving peptide. In some cases, a nucleic acid sequence encoding a suicide gene is operably linked to a nucleic acid sequence encoding a 2A self-cleaving peptide, which is operably linked to a nucleic acid sequence encoding a second PEBL. In some cases, a nucleic acid sequence encoding a second PEBL is operably linked to a nucleic acid sequence encoding a 2A self-cleaving peptide, which is operably linked to a nucleic acid sequence encoding a suicide gene.

The bicistronic vector provided herein can comprise a first nucleotide sequence encoding a kill gene; and a second nucleotide sequence encoding a surface polypeptide binding domain linked to a localizing domain (e.g., a PEBL).

In some embodiments of a bicistronic construct, a polynucleotide encoding a kill gene is located upstream (at the 5′ end) of a polynucleotide encoding a ribosomal codon skipping site (e.g., a 2A self-cleaving peptide), and a polynucleotide encoding a PEBL is located downstream (at the 3′ end) of the polynucleotide encoding a ribosomal codon skipping site. In some embodiments, the kills gene can encode a cell surface antigen. The cell surface antigen can be CD20. The surface polypeptide binding domain can bind to a subunit of a TCR complex. In some embodiments, the surface polypeptide binding domain can bind to CD3ε. In some embodiments, the surface polypeptide binding domain can bind to CD3ε, CD3γ, or CD3δ. In some embodiments, the surface polypeptide binding domain can be an anti-CD3ε antibody or antigen binding domain thereof.

In some embodiments, the bicistronic construct comprises an anti-CD3ε antibody comprising a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the bicistronic construct comprises an anti-CD3ε antibody comprising a heavy chain variable domain having an amino acid sequence with at least 70%, at least 75%, at least 80%, at least 85%, at least 90%, at least 95%, at least 96%, at least 97%, at least 98%, at least 99%, or at least 100% sequence identity to SEQ ID NO: 114. In some embodiments, the bicistronic construct comprises an anti-CD3ε antibody comprising a light chain variable domain having an amino acid sequence with at least 70%, at least 75%, at least 80%, at least 85%, at least 90%, at least 95%, at least 96%, at least 97%, at least 98%, at least 99%, or at least 100% sequence identity to SEQ ID NO: 115. In some embodiments, the bicistronic construct comprises an anti-CD3ε antibody comprising an amino acid sequence having at least 70%, at least 75%, at least 80%, at least 85%, at least 90%, at least 95%, at least 96%, at least 97%, at least 98%, at least 99%, or at least 100% sequence identity to SEQ ID NO: 104.

The mechanism of ribosomal codon skipping via a 2A peptide sequence is useful for generating two proteins from one transcript; a normal peptide bond is impaired at the 2A sequence, resulting in two discontinuous protein fragments from one translation event. Self-cleaving 2A peptides (e.g., 2A cleavage sites) are described in Kim et al., PLoS One, 2011, 6(4):e18556.

In some embodiments, the IRES is from an Encephalomyocarditis virus. In some embodiments, the IRES is from an Enterovirus. In some embodiments, the nucleic acid sequence of the IRES sequence is set forth in SEQ ID NO: 62 (see, e.g., Table 2).

In some embodiments, the ribosomal codon skipping site is based on a 2A self-cleaving peptide (see, e.g., Table 3). In some embodiments, the 2A self-cleaving peptide is selected from the group consisting of P2A, E2A, F2A, and T2A. In some instances, the amino acid sequence of the P2A peptide comprises the amino acid sequence of SEQ ID NO:67, or an amino acid sequence having at least 90% sequence identify thereto. In some instances, the amino acid sequence of the E2A peptide comprises the amino acid sequence of SEQ ID NO: 68, or an amino acid sequence having at least 90% sequence identify thereto. In some instances, the amino acid sequence of the F2A peptide comprises the amino acid sequence of SEQ ID NO: 69, or an amino acid sequence having at least 90% sequence identify thereto. In some instances, the amino acid sequence of the T2A peptide comprises the amino acid sequence of SEQ ID NO: 70, or an amino acid sequence having at least 90% sequence identify thereto.

In some embodiments, the viral construct (e.g., retroviral construct) comprises a nucleic acid sequence encoding a 2A self-cleaving peptide (e.g., 2A peptide cleavage site) selected from the group consisting of P2A, E2A, F2A, and T2A, wherein the polynucleotide encoding 2A self-cleaving peptide links the nucleic acid sequence encoding the suicide gene and the nucleic acid sequence encoding the second PEBL. In other words, the polynucleotide encoding 2A self-cleaving peptide is between the nucleic acid sequence encoding the suicide gene and the nucleic acid sequence encoding the second PEBL. As described above, in some embodiments, the construct comprises or consisting of from 5′ end to 3′ end: a nucleic acid sequence encoding a suicide gene, a nucleic acid sequence encoding a P2A self-cleaving peptide, and a nucleic acid sequence encoding a second PEBL. In some embodiments, the construct comprises or consisting of from 5′ end to 3′ end: a nucleic acid sequence encoding any suicide gene described herein, a nucleic acid sequence encoding a P2A self-cleaving peptide, and a nucleic acid sequence encoding any CD3 PEBL described herein. In some embodiments, the construct comprises or consisting of from 5′ end to 3′ end: a nucleic acid sequence encoding any suicide gene described herein, a nucleic acid sequence encoding an E2A self-cleaving peptide, and a nucleic acid sequence encoding any CD3 PEBL described herein. In some embodiments, the construct comprises or consisting of from 5′ end to 3′ end: a nucleic acid sequence encoding any suicide gene described herein, a nucleic acid sequence encoding an F2A self-cleaving peptide, and a nucleic acid sequence encoding any CD3 PEBL described herein. In some embodiments, the construct comprises or consisting of from 5′ end to 3′ end: a nucleic acid sequence encoding any suicide gene described herein, a nucleic acid sequence encoding a T2A self-cleaving peptide, and a nucleic acid sequence encoding any CD3 PEBL described herein.

In some embodiments, the construct comprises or consisting of from 5′ end to 3′ end: a nucleic acid sequence encoding a second PEBL, a nucleic acid sequence encoding a P2A self-cleaving peptide, and a nucleic acid sequence encoding a suicide gene. In some embodiments, the construct comprises or consisting of from 5′ end to 3′ end: a nucleic acid sequence encoding a second PEBL, a nucleic acid sequence encoding an E2A self-cleaving peptide, and a nucleic acid sequence encoding a suicide gene. In some embodiments, the construct comprises or consisting of from 5′ end to 3′ end: a nucleic acid sequence encoding a second PEBL, a nucleic acid sequence encoding an F2A self-cleaving peptide, and a nucleic acid sequence encoding a suicide gene. In some embodiments, the construct comprises or consisting of from 5′ end to 3′ end: a nucleic acid sequence encoding a second PEBL, a nucleic acid sequence encoding a T2A self-cleaving peptide, and a nucleic acid sequence encoding a suicide gene.

In some embodiments, the nucleic acid sequence encoding the P2A comprises or consists of a nucleic acid having at least 90% sequence identity to SEQ ID NO: 63. In some embodiments, the nucleic acid sequence encoding the P2A comprises or consists of a nucleic acid of SEQ ID NO: 63. In some embodiments, the nucleic acid sequence encoding the E2A comprises or consists of a nucleic acid having at least 90% sequence identity to SEQ ID NO:64. In some embodiments, the nucleic acid sequence encoding the E2A comprises or consists of a nucleic acid of SEQ ID NO: 64. In some embodiments, the nucleic acid sequence encoding the F2A comprises or consists of a nucleic acid having at least 90% sequence identity to SEQ ID NO: 65. In some embodiments, the nucleic acid sequence encoding the F2A comprises or consists of a nucleic acid of SEQ ID NO: 65. In some embodiments, the nucleic acid sequence encoding the T2A comprises or consists of a nucleic acid sequence having at least 90% sequence identity to SEQ ID NO: 66. In some embodiments, the nucleic acid sequence encoding the T2A comprises or consists of a nucleic acid of SEQ ID NO: 66.

The present invention provides vectors such as expression vectors in which any of the polynucleotides described herein is inserted. In some embodiments, the vector is derived from retroviruses such as lentiviruses. Such vectors are suitable tools to achieve long-term gene transfer since they allow long-term, stable integration of an exogenous polynucleotide (e.g., transgene) and its propagation in daughter cells. Unlike vectors derived from onco-retroviruses such as murine leukemia viruses, lentiviral vectors can transduce non-proliferating cells. Lentiviral vectors also have low immunogenicity. In other embodiments, the vector is an adenoviral vector. In certain embodiments, the vector is a plasmid.

In some embodiments, the promoter comprises at least 90%, e.g., 90%, 91%, 92%, 93%, 94%, 95%, 96%, 97%, 98%, 99%, 99.9%, or more sequence identity to a CMV promoter. In some embodiments, the promoter comprises a CMV promoter. In some embodiments, the CMV promoter comprises the sequence of SEQ ID NO: 6. In some embodiments, any of the constructs described herein comprises or consists of a CMV promoter of SEQ ID NO: 6.

In some embodiments, the promoter comprises at least 90%, e.g., 90%, 91%, 92%, 93%, 94%, 95%, 96%, 97%, 98%, 99%, 99.9%, or more sequence identity to an EF1α promoter. In some embodiments, the promoter comprises an EF1α promoter. In some embodiments, the EF1α promoter comprises the sequence of SEQ ID NO: 7. In some embodiments, the EF1α promoter comprises the sequence of SEQ ID NO:7. In some embodiments, any of the constructs described herein comprises or consists of an EF1α promoter of SEQ ID NO: 7.

In some embodiments, the promoter comprises at least 90%, e.g., 90%, 91%, 92%, 93%, 94%, 95%, 96%, 97%, 98%, 99%, 99.9%, or more sequence identity to an EFS promoter. In some embodiments, the promoter comprises an EFS promoter. In some embodiments, the EFS promoter comprises the sequence of SEQ ID NO: 8. In some embodiments, the EFS promoter comprises the sequence of SEQ ID NO: 8. In some embodiments, any of the constructs described herein comprises or consists of an EFS promoter of SEQ ID NO: 8.

In some embodiments, the promoter comprises at least 90%, e.g., 90%, 91%, 92%, 93%, 94%, 95%, 96%, 97%, 98%, 99%, 99.9%, or more sequence identity to a murine stem cell virus (MSCV) promoter. In some embodiments, the promoter comprises a MSCV promoter. In some embodiments, the MSCV promoter comprises the sequence of SEQ ID NO: 9. In some embodiments, any of the constructs described herein comprises or consists of a MSCV promoter of SEQ ID NO:9.

In some embodiments, the promoter comprises at least 90%, e.g., 90%, 91%, 92%, 93%, 94%, 95%, 96%, 97%, 98%, 99%, 99.9%, or more sequence identity to a phosphoglycerate kinase (PGK) promoter. In some embodiments, the promoter comprises a PGK promoter. In some embodiments, the PGK promoter comprises the sequence of SEQ ID NO:10. In some embodiments, any of the constructs described herein comprises or consists of a PGK promoter of SEQ ID NO:10.

In some embodiments, the nucleic acid sequence encoding the suicide gene is disposed 5′ to the nucleic acid sequence encoding the second PEBL. In some embodiments, a bicistronic construct comprises or consists of from 5′ to 3′ end: a suicide gene or a kill gene, an operation linker (e.g., IRES or a ribosomal codon skipping site, which is also referred to as a sequence encoding a 2A self-cleaving peptide), a CD8 signal peptide, a CD3 PEBL, a Myc tag, a repeat of GS linker (e.g., GGGGS (SEQ ID NO: 230)) before 2 amino acids, and an ER retention sequence. In some embodiments, the suicide gene is a CD20 or derivative thereof.

In some embodiments, the suicide gene is a truncated CD20 (CD20t). In some embodiments, the ribosomal codon skipping site is based on a 2A self-cleaving peptide (see, e.g., Table 3). In some embodiments, the 2A self-cleaving peptide is selected from the group consisting of P2A, E2A, F2A, and T2A. In some embodiments, the ribosomal codon skipping site is a P2A. In some embodiments, the repeat of GS linker comprises at least 1 repeat, at least 2 repeats, at least 3 repeats, at least 4 repeats, at least 5 repeats, at least 6 repeats, at least 7 repeats, at least 8 repeats, at least 9 repeats, and at least 10 repeats before the 2 amino acids. In some embodiments, the repeat of GS linker comprises at most 1 repeat, at most 2 repeats, at most 3 repeats, at most 4 repeats, at most 5 repeats, at most 6 repeats, at most 7 repeats, at most 8 repeats, at most 9 repeats, and at most 10 repeats before the 2 amino acids. In some embodiments, the 2 amino acids located downstream of the GS linker comprises AE.

In some embodiments, the second bicistronic construct comprises the suicide gene, the second PEBL, and one or more of the components set forth in Table 12. In some embodiments, the suicide gene and the second PEBL comprise one or more of the components set forth in Table 12. In some embodiments, the suicide gene and the second PEBL comprise one or more of the components set forth in SEQ ID NO: 105. In some embodiments, the suicide gene comprises amino acid sequences derived from CD20 or derivative there of as described in SEQ ID NO: 106.

CD20t-P2A-UCHT1 PEBL22 sequence (SEQ ID NO: 105):
MTTPRNSVNGTFPAEPMKGPIAMQSGPKPLFRRMSSLVGPTQSFFMRES
KTLGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEGSGATNFSLLKQAGDVEENPGPMALPVTALL
LPLALLLHAARPDIQMTQTTSSLSASLGDRVTISCRASQDIRNYLNWYQ
QKPDGTVKLLIYYTSRLHSGVPSKFSGSGSGTDYSLTISNLEQEDIATY
FCQQGNTLPWTFAGGTKLEIKGGGGSGGGGSGGGGSGGGGSEVQLQQSG
PELVKPGASMKISCKASGYSFTGYTMNWVKQSHGKNLEWMGLINPYKGV
STYNQKFKDKATLTVDKSSSTAYMELLSLTSEDSAVYYCARSGYYGDSD
WYFDVWGQGTTLTVFSEQKLISEEDLGGGGSGGGGSGGGGSGGGGSAEK
DEL

TABLE 12
Amino acid sequence information for select components of a
suicide gene (or a kill gene) and a CD3 PEBL

| | |
|---|---|
| CD20t | MTTPRNSVNGTFPAEPMKGPIAMQSGPKPLFRRMSSLVGPTQSFFMRESKTLGAV |
| (SEQ ID | QIMNGLFHIALGGLLMIPAGIYAPICVTVWYPLWGGIMYIISGSLLAATEKNSRK |
| NO: 106) | CLVKGKMIMNSLSLFAAISGMILSIMDILNIKISHFLKMESLNFIRAHTPYINIY |
| | NCEPANPSEKNSPSTQYCYSIQSLFLGILSVMLIFAFFQELVIAGIVENEWKRTC |
| | SRPKSNIVLLSAEEKKEQTIEIKEEVVGLTETSSQPKNEEDIE |
| P2A | GSGATNFSLLKQAGDVEENPGP |
| (SEQ ID | |
| NO: 231) | |
| CD8 signal | MALPVTALLLPLALLLHAARP |
| peptide | |
| (SEQ ID | |
| NO: 80) | |
| UCHT1 | DIQMTQTTSSLSASLGDRVTISCRASQDIRNYLNWYQQKPDGTVKLLIYYTSRLH |
| scFv | SGVPSKFSGSGSGTDYSLTISNLEQEDIATYFCQQGNTLPWTFAGGTKLEIKGGG |
| (SEQ ID | GSGGGGSGGGGSGGGGSEVQLQQSGPELVKPGASMKISCKASGYSFTGYTMNWVK |
| NO: 104) | QSHGKNLEWMGLINPYKGVSTYNQKFKDKATLTVDKSSSTAYMELLSLTSEDSAV |
| | YYCARSGYYGDSDWYFDVWGQGTTLTVFS |
| Myc tag | EQKLISEEDL |
| (SEQ ID | |
| NO: 227) | |
| GS linker + | GGGGSGGGGGGGGSGGGGSAE |
| 2 more aa | |
| (SEQ ID | |
| NO: 229) | |
| ER | KDEL |
| retention | |
| (SEQ ID | |
| NO: 73) | |

In some embodiments, the suicide gene comprises an amino acid sequence having at least 85% (e.g., 85%, 86%, 87%, 88%, 89%, 90%, or more) sequence identity to SEQ ID NO:106. In some embodiments, the suicide gene or a kill gene comprises an amino acid sequence having at least 90% (e.g., 90%, 91%, 92%, 93%, 94%, 95%, 96%, 97%, 98%, 99%, or more) sequence identity to SEQ ID NO:106. In some embodiments, the suicide gene or a kill gene comprises an amino acid sequence having at least 90% (e.g., 90%, 91%, 92%, 93%, 94%, 95%, 96%, 97%, 98%, 99%, or more) sequence identity to SEQ ID NO: 106.

In some embodiments, an engineered immune cell of the present invention further comprises a suicide gene or a kill gene and a second PEBL encoded by a bicistronic construct comprising a nucleic acid sequence encoding an amino acid sequence of the suicide gene or a kill gene having at least 90% (e.g., 90%, 91%, 92%, 94%, 94%, 95%, 96%, 97%, 98%, 99%, or more) sequence identity to SEQ ID NO:106. In some embodiments, the engineered immune cell is a CD4+ T cell comprising a suicide gene or a kill gene and a second PEBL encoded by the bicistronic construct comprising a nucleic acid sequence encoding an amino acid sequence of the suicide gene having at least 90% sequence identity to SEQ ID NO:106?.

In some embodiments, the engineered immune cell is a CD8+ T cell comprising a suicide gene and a second PEBL encoded by the construct comprising a nucleic acid sequence encoding an amino acid sequence of the suicide gene or a kill gene having at least 90% sequence identity to SEQ ID NO: 106?. Also, provided herein is a population comprising such cells.

In some embodiments, the second retroviral vector or lentiviral vector further comprises a kill gene. In some embodiments, the nucleic acids encoding the second PEBL and the kill gene are located in the same vector that comprises the nucleic acids encoding the CAR and the first PEBL.

In some embodiments, the first vector encoding the CAR and the first PEBL and the second vector encoding the kill gene and the second PEBL are the same type of vector. In some embodiments, the first vector encoding the CAR and the first PEBL and the second vector encoding the kill gene and the second PEBL are both lentiviral vectors. In some embodiments, the first vector encoding the CAR and the first PEBL and the second vector encoding the kill gene and the second PEBL are both retroviral vectors.

In some embodiments, the first vector encoding the CAR and the first PEBL and the second vector encoding the kill gene and the second PEBL are different types of vectors. In some embodiments, the first vector encoding the CAR and the first PEBL is a lentiviral vector and the second vector encoding the kill gene and the second PEBL is a retroviral vector. In some embodiments, the first vector encoding the CAR and the first PEBL is a retroviral vector and the second vector encoding the kill gene and the second PEBL is a lentiviral vector.

In some aspects, the nucleic acid encoding the second PEBL and the nucleic acid encoding the kill gene are operably linked by an Internal Ribosome Entry Site (IRES) or a ribosomal codon skipping site. In some embodiments, the ribosomal codon skipping site comprises a 2A self-cleaving peptide. In some instances, the nucleic acid encoding the second PEBL and the nucleic acid encoding the kill gene are located under the same promoter. In some instances, the nucleic acid encoding the second PEBL and the nucleic acid encoding the kill gene are located under different promoters.

In some instances, wherein the nucleic acid encoding the second PEBL and the kill gene are located in a separate vector from the vector comprising the nucleic acids encoding the CAR and the first PEBL, the two vectors can be introduced simultaneously. In some instances, wherein the nucleic acid encoding the second PEBL and the kill gene are located in a separate vector from the vector comprising the nucleic acids encoding the CAR and the first PEBL, the two vectors can be introduced sequentially. In some instances, wherein the nucleic acid encoding the second PEBL and the kill gene are located in a separate vector from the vector comprising the nucleic acids encoding the CAR and the first PEBL, the vector encoding the CAR and the first PEBL is introduced before the vector encoding the second PEBL and the kill gene. In some instances, wherein the nucleic acid encoding the second PEBL and the kill gene are located in a separate vector from the vector comprising the nucleic acids encoding the CAR and the first PEBL, the vector encoding the CAR and the first PEBL is introduced after the vector encoding the second PEBL and the kill gene.

In some instances, wherein the nucleic acid encoding the CAR, the first PEBL, the kill gene, and the second PEBL are located in different vectors, the different vectors can be introduced simultaneously. In some instances, wherein the nucleic acid encoding the CAR, the first PEBL, the kill gene, and the second PEBL are located in different vectors, the different vectors can be introduced sequentially.

### Dual Promoter Retroviral Construct for a Suicide Gene (a Kill Gene) and a Second PEBL

Provided herein are recombinant retroviral constructs (or vectors) for simultaneous expression of a suicide gene and a second PEBL in a cell such as a T cell. In some embodiments, the retroviral constructs include a promoter operably linked to a polynucleotide encoding any of the suicide genes described herein and a promoter operably linked to a polynucleotide encoding any of the second PEBLs described herein. In some embodiments, the promoter for the suicide gene and the promoter for the second PEBL share less than 90% sequence identity, e.g., less than 90% identity, less than 80% identity, less than 75% sequence identity, less 70% sequence identity, less than 65% sequence identity, less than 60% sequence identity, less than 55% sequence identity, and the like. In some embodiments, the promoter for the suicide gene and the promoter for the second PEBL share 80% sequence identity or less, e.g., 80% identity, 75% sequence identity, 70% sequence identity, 65% sequence identity, 60% sequence identity, 55% sequence identity, and the like. In some embodiments, the promoter for the suicide gene and the promoter for the second PEBL share at least 50% sequence identity, e.g., 50% sequence identity, 55% sequence identity, 60% sequence identity, 65% sequence identity, 70% sequence identity, 75% sequence identity, 80% sequence identity, 85% sequence identity, 90% sequence identity, 95% sequence identity, or more sequence identity.

In some embodiments, the promoter for the suicide gene (referred to as the first promoter) is different than the promoter for the second PEBL (referred to as the second promoter). The first promoter and the second promoter can have the same sequence. In other instances, the first promoter and the second promoter have different sequences.

In some embodiments, the first promoter and/or second promoter comprises at least 90%, e.g., 90%, 91%, 92%, 93%, 94%, 95%, 96%, 97%, 98%, 99%, or more sequence identity to a CMV promoter. In some embodiments, the first promoter and/or second promoter comprises a CMV promoter. In some embodiments, the CMV promoter comprises the sequence of SEQ ID NO:6.

In some embodiments, the first promoter and/or second promoter comprises at least 90%, e.g., 90%, 91%, 92%, 93%, 94%, 95%, 96%, 97%, 98%, 99%, or more sequence identity to an EF1α promoter. In some embodiments, the first promoter and/or second promoter comprises an EF1α promoter. In some embodiments, the EF1α promoter comprises the sequence of SEQ ID NO:7.

In some embodiments, the first promoter and/or second promoter comprises at least 90%, e.g., 90%, 91%, 92%, 93%, 94%, 95%, 96%, 97%, 98%, 99%, or more sequence identity to an EFS promoter. In some embodiments, the first promoter and/or second promoter comprises an EFS promoter. In some embodiments, the EFS promoter comprises the sequence of SEQ ID NO:8.

In some embodiments, the first promoter and/or second promoter comprises at least 90%, e.g., 90%, 91%, 92%, 93%, 94%, 95%, 96%, 97%, 98%, 99%, or more sequence identity to a murine stem cell virus (MSCV) promoter. In some embodiments, the first promoter and/or second promoter comprises a MSCV promoter. In some embodiments, the MSCV promoter comprises the sequence of SEQ ID NO:9.

In some embodiments, the first promoter and/or second promoter comprises at least 90%, e.g., 90%, 91%, 92%, 93%, 94%, 95%, 96%, 97%, 98%, 99%, or more sequence identity to a phosphoglycerate kinase (PGK) promoter. In some embodiments, the first promoter and/or second promoter comprises a PGK promoter. In some embodiments, the PGK promoter comprises the sequence of SEQ ID NO: 10.

In some embodiments, the retroviral constructs from 5′ to 3′ include the first promoter operably linked to the polynucleotide encoding the suicide gene and the second promoter operably linked to the polynucleotide encoding the second PEBL. In various embodiments, the retroviral constructs from 5′ to 3′ include the second promoter operably linked to the polynucleotide encoding the second PEBL and the first promoter operably linked to the polynucleotide encoding the suicide gene.

In some embodiments, the first promoter is located upstream of the second promoter. In some embodiments, the first promoter is a CMV promoter and the second promoter is an EFS promoter. In some embodiments, the first promoter is a CMV promoter and the second promoter is an EF1α promoter. In some embodiments, the first promoter is a CMV promoter and the second promoter is a PGK promoter. In some embodiments, the first promoter is a CMV promoter and the second promoter is a MSCV promoter. In some embodiments, the first promoter is a CMV promoter and the second promoter is a CMV promoter. In some embodiments, the first promoter is a MSCV promoter and the second promoter is an EFS promoter. In some embodiments, the first promoter is a MSCV promoter and the second promoter is an EF1α promoter. In some embodiments, the first promoter is a MSCV promoter and the second promoter is a PGK promoter. In some embodiments, the first promoter is a MSCV promoter and the second promoter is a CMV promoter. In some embodiments, the first promoter is a MSCV promoter and the second promoter is a MSCV promoter. In some embodiments, the first promoter is a PGK promoter and the second promoter is an EFS promoter. In some embodiments, the first promoter is a PGK promoter and the second promoter is an EF1α promoter. In some embodiments, the first promoter is a PGK promoter and the second promoter is a MSCV promoter. In some embodiments, the first promoter is a PGK promoter and the second promoter is a CMV promoter. In some embodiments, the first promoter is a PGK promoter and the second promoter is a PGK promoter. In some embodiments, the first promoter is an EF1α promoter and the second promoter is a MSCV promoter. In some embodiments, the first promoter is an EF1α promoter and the second promoter is a PGK promoter. In some embodiments, the first promoter is an EF1α promoter and the second promoter is an EFS promoter. In some embodiments, the first promoter is an EF1α promoter and the second promoter is a CMV promoter. In some embodiments, the first promoter is an EF1α promoter and the second promoter is an EFlu promoter. In some embodiments, the first promoter is an EFS promoter and the second promoter is a MSCV promoter. In some embodiments, the first promoter is an EFS promoter and the second promoter is an EF1α promoter. In some embodiments, the first promoter is an EFS promoter and the second promoter is a PGK promoter. In some embodiments, the first promoter is an EFS promoter and the second promoter is a CMV promoter. In some embodiments, the first promoter is an EFS promoter and the second promoter is an EFS promoter.

### Engineered Immune Cells Expressing Bicistronic Vectors

In certain embodiments, provided is an engineered immune cell comprising a bicistronic construct comprising: (i) a polynucleotide encoding a chimeric antigen receptor (CAR), wherein the CAR comprises intracellular signaling domains of 4-1BB and CD3ζ, and an antigen binding domain that specifically binds CD7; (ii) a polynucleotide encoding a target-binding molecule linked to a localizing domain, wherein the target-binding molecule is an antigen binding domain that binds CD7, and the localizing domain comprises an endoplasmic reticulum retention sequence; and (iii) a nucleic acid sequence encoding a 2A self-cleaving peptide or an IRES sequence, as exemplified herein.

In certain embodiments, the antigen binding domain that binds CD7 in the context of the CAR, as well as in the context of the antigen binding domain against CD7 comprises: a VH sequence set forth in SEQ ID NO:32 and a VL sequence set forth in SEQ ID NO: 33; a VH sequence set forth in SEQ ID NO:34 and a VL sequence set forth in SEQ ID NO: 35; or a VH sequence set forth in SEQ ID NO:36 and a VL sequence set forth in SEQ ID NO:37. As described herein, in certain embodiments, the antigen binding domain comprises a VH and a VL having sequence that each comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOs:32 and 33, respectively; SEQ ID NOs:34 and 35, respectively; or SEQ ID NOs:36 and 37, respectively. In certain embodiments, the antigen binding domain that binds CD7 in the context of the CAR can be different from the antibody that binds CD7 in the context of the target-binding molecule (the protein expression blocker or PEBL), as described herein.

In some embodiments, the engineered immune cell comprising a bicistronic construct comprising a nucleic acid construct comprising from the 5′ end to 3′ end: a polynucleotide encoding a target-binding molecule linked to a localizing domain wherein the target-binding molecule binds CD7 (e.g., a CD7 PEBL), an IRES sequence, and a polynucleotide encoding a chimeric antigen receptor against CD7 (e.g., a CD7 CAR). In some instances, the engineered immune cell comprises a nucleic acid construct comprising SEQ ID NO:11. In some embodiments, provided herein is an engineered CD4+ T cell or a population thereof comprising a nucleic acid construct comprising SEQ ID NO:11. In other embodiments, provided herein is an engineered CD8+ T cell or a population thereof comprising a nucleic acid construct comprising SEQ ID NO:11. In some embodiments, provided herein is an engineered CD3+ T cell or a population thereof comprising a nucleic acid construct comprising SEQ ID NO:11.

In some embodiments, the engineered immune cell comprising a bicistronic construct comprising a nucleic acid construct comprising from the 5′ end to 3′ end: a polynucleotide encoding a chimeric antigen receptor against CD7, a IRES sequence, and a polynucleotide encoding a target-binding molecule linked to a localizing domain wherein the target-binding molecule binds CD7 (e.g., a PEBL against CD7). In some instances, the engineered immune cell comprises a nucleic acid construct comprising SEQ ID NO:12. In one embodiment, provided herein is an engineered CD4+ T cell or a population thereof comprising a nucleic acid construct comprising SEQ ID NO:12. In some embodiments, provided herein is an engineered CD8+ T cell or a population thereof comprising a nucleic acid construct comprising SEQ ID NO:12. In some embodiments, provided herein is an engineered CD3+ T cell or a population thereof comprising a nucleic acid construct comprising SEQ ID NO:12.

In some embodiments, the engineered immune cell comprising a bicistronic construct comprising a nucleic acid construct comprising from the 5′ end to 3′ end: a polynucleotide encoding a chimeric antigen receptor against CD7 (e.g., a CD7 CAR), a nucleic acid sequence encoding a 2A self-cleaving peptide, and a polynucleotide encoding a target-binding molecule linked to a localizing domain wherein the target-binding molecule binds CD7 (e.g., a CD7 PEBL). In some instances, the engineered immune cell comprises a nucleic acid construct comprising SEQ ID NO:13. In one embodiment, provided herein is an engineered CD4+ T cell or a population thereof comprising a nucleic acid construct comprising SEQ ID NO: 13. In some embodiments, provided herein is an engineered CD8+ T cell or a population thereof comprising a nucleic acid construct comprising SEQ ID NO:13. In some embodiments, provided herein is an engineered CD3+ T cell or a population thereof comprising a nucleic acid construct comprising SEQ ID NO:13.

In some embodiments, the engineered immune cell comprising a bicistronic construct comprising a nucleic acid construct comprising from the 5′ end to 3′ end: a polynucleotide encoding a target-binding molecule linked to a localizing domain wherein the target-binding molecule binds CD7, a nucleic acid sequence encoding a 2A self-cleaving peptide, and a polynucleotide encoding a chimeric antigen receptor against CD7.

In some embodiments, the engineered immune cell comprising a bicistronic construct comprising a nucleic acid construct comprising from the 5′ end to 3′ end: a promoter, a polynucleotide encoding a chimeric antigen receptor against CD7 (e.g., a CD7 CAR), a nucleic acid sequence encoding a 2A self-cleaving peptide, and a polynucleotide encoding a target-binding molecule linked to a localizing domain wherein the target-binding molecule binds CD7 (e.g., a CD7 PEBL). In some instances, the engineered immune cell comprises a nucleic acid construct comprising at least 85% sequence identity to any one of the nucleic acid sequences of SEQ ID NOS: 14-16. In some instances, the engineered immune cell comprises a nucleic acid construct comprising any one of the nucleic acid sequences of SEQ ID NOS:14-16. In some embodiments, the engineered immune cell comprises a nucleic acid construct comprising at least 85% (e.g., 85%, 86%, 87%, 88%, 89%, 90%, 91%, 92%, 93%, 94%, 95%, 96%, 97%, 98%, 99%, or more) sequence identity to SEQ ID NO:14. In some embodiments, the engineered immune cell comprises a nucleic acid construct comprising at least 85% (e.g., 85%, 86%, 87%, 88%, 89%, 90%, 91%, 92%, 93%, 94%, 95%, 96%, 97%, 98%, 99%, or more) sequence identity to SEQ ID NO:15. In some embodiments, the engineered immune cell comprises a nucleic acid construct comprising at least 85% (e.g., 85%, 86%, 87%, 88%, 89%, 90%, 91%, 92%, 93%, 94%, 95%, 96%, 97%, 98%, 99%, or more) sequence identity to SEQ ID NO:16.

In some embodiments, the engineered immune cell comprising a bicistronic construct comprising a nucleic acid construct comprising from the 5′ end to 3′ end: a promoter, a polynucleotide encoding a chimeric antigen receptor against CD7, a nucleic acid sequence encoding a 2A self-cleaving peptide, and a polynucleotide encoding a target-binding molecule linked to a localizing domain wherein the target-binding molecule binds CD7. In some instances, the promoter is selected from a MSCV promoter, PGK promoter, EF1α promoter, and EFS promoter. In some instances, the engineered immune cell comprises a polynucleotide comprising SEQ ID NO:14. In one embodiment, provided herein is an engineered CD4+ T cell or a population thereof comprising a nucleic acid construct comprising SEQ ID NO: 14. In some embodiments, provided herein is an engineered CD8+ T cell or a population thereof comprising a nucleic acid construct comprising SEQ ID NO:14. In some embodiments, provided herein is an engineered CD3+ T cell or a population thereof comprising a nucleic acid construct comprising SEQ ID NO:14.

In some instances, the engineered immune cell comprises a polynucleotide comprising SEQ ID NO:15. In one embodiment, provided herein is an engineered CD4+ T cell or a population thereof comprising a nucleic acid construct comprising SEQ ID NO:15. In some embodiments, provided herein is an engineered CD8+ T cell or a population thereof comprising a nucleic acid construct comprising SEQ ID NO:15. In one embodiment, provided herein is an engineered CD4+ T cell or a population thereof comprising a nucleic acid construct comprising SEQ ID NO:15. In some embodiments, provided herein is an engineered CD3+ T cell or a population thereof comprising a nucleic acid construct comprising SEQ ID NO:15.

In some instances, the engineered immune cell comprises a polynucleotide comprising SEQ ID NO:16. In one embodiment, provided herein is an engineered CD4+ T cell or a population thereof comprising a nucleic acid construct comprising SEQ ID NO:16.

In some embodiments, provided herein is an engineered CD8+ T cell or a population thereof comprising a nucleic acid construct comprising SEQ ID NO:16. In some embodiments, provided herein is an engineered CD3+ T cell or a population thereof comprising a nucleic acid construct comprising SEQ ID NO:16.

In some embodiments, the engineered immune cells described herein or a population thereof comprise at least 10% CD7 CAR+/CD7-negative T cells, at least 15% CD7 CAR+/CD7-negative T cells, at least 20% CD7 CAR+/CD7-negative T cells, at least 25% CD7 CAR+/CD7-negative T cells, at least 30% CD7 CAR+/CD7-negative T cells, at least 35% CD7 CAR+/CD7-negative T cells, at least 40% CD7 CAR+/CD7-negative T cells, at least 45% CD7 CAR+/CD7-negative T cells, at least 50% CD7 CAR+/CD7-negative T cells, at least 55% CD7 CAR+/CD7-negative T cells, at least 60% CD7 CAR+/CD7-negative T cells, at least 65% CD7 CAR+/CD7-negative T cells, at least 70% CD7 CAR+/CD7-negative T cells, at least 75% CD7 CAR+/CD7-negative T cells, at least 80% CD7 CAR+/CD7-negative T cells, at least 85% CD7 CAR+/CD7-negative T cells, at least 90% CD7 CAR+/CD7-negative T cells, at least 95% CD7 CAR+/CD7-negative T cells, at least 96% CD7 CAR+/CD7-negative T cells, at least 97% CD7 CAR+/CD7-negative T cells, at least 98% CD7 CAR+/CD7-negative T cells, at least 99% CD7 CAR+/CD7-negative T cells, or 100% CD7 CAR+/CD7-negative T cells. In some embodiments, the engineered immune cells outlined herein include a population of substantially purified CD7 CAR+/CD7-negative T cells wherein such cells express any one of the bicistronic constructs described.

In certain embodiments, provided is an engineered immune cell comprising two bicistronic constructs comprising: the first bicistronic construct comprises: (i) a polynucleotide encoding a chimeric antigen receptor (CAR), wherein the CAR comprises intracellular signaling domains of 4-1BB and CD3ζ, and an antigen binding domain that specifically binds CD7; (ii) a polynucleotide encoding a target-binding molecule linked to a localizing domain, wherein the target-binding molecule is an antigen binding domain that binds CD7, and the localizing domain comprises an endoplasmic reticulum retention sequence; and (iii) a nucleic acid sequence encoding a 2A self-cleaving peptide or an IRES sequence, as exemplified herein, and the second bicistronic construct comprises: (i) polynucleotide encoding a suicide gene; (ii) a polynucleotide encoding a target-binding molecule linked to a localizing domain, wherein the target-binding molecule is an antigen binding domain that binds CD3, and the localizing domain comprises an endoplasmic reticulum retention sequence; and (iii) a nucleic acid sequence encoding a 2A self-cleaving peptide or an IRES sequence, as exemplified herein.

In some instances, the engineered immune cells such as CD7 CAR+/CD7-negative T cell or PCART7-CD3PEBL T cell (allo-PCART7 cells) further comprises a fourth nucleic acid comprising a suicide gene. In some embodiments, the suicide gene comprises CD20 or a derivative thereof. In some embodiments, the suicide gene comprises CD20, a derivative thereof, or a portion thereof. In some embodiments, the suicide gene comprises a modified CD20. In some embodiments, the suicide gene comprises a truncated CD20. In some embodiments, the fourth nucleic acid is located in the second expression vector, wherein the second expression vector encode the second PEBL. In some embodiments, the fourth nucleic acid is located in the first expression vector, wherein the first expression vector encode the CAR and the first PEBL. In some embodiments, the fourth nucleic acid is located in a separate expression vector, e.g., a third expression vector.

In some instances, the nucleic acid encoding the second PEBL and the nucleic acid encoding the fourth nucleic acid comprising a suicide gene are located in a bicistronic vector (e.g., bicistronic retroviral vector, bicistronic lentiviral vector) such that the second PEBL and the fourth nucleic acid encoding the suicide gene are expressed simultaneously. In some aspects, the nucleic acid encoding the second PEBL and the nucleic acid encoding the suicide gene are the operably linked by Internal Ribosome Entry Site (IRES) or a ribosomal codon skipping site. In some embodiments, the ribosomal codon skipping site comprises a 2A self-cleaving peptide. In some instances, the nucleic acid encoding the second PEBL and the nucleic acid encoding the suicide gene is located in a second retroviral vector or a lentiviral vector.

In some instances, wherein the nucleic acid encoding the second PEBL is located in a separate vector from the vector comprising the nucleic acids encoding the CAR and the first PEBL, the two vectors can be introduced simultaneously. In some instances, wherein the nucleic acid encoding the second PEBL is located in a separate vector from the vector comprising the nucleic acids encoding the CAR and the first PEBL, the two vectors can be introduced sequentially. In some instances, wherein the nucleic acid encoding the second PEBL is located in a separate vector from the vector comprising the nucleic acids encoding the CAR and the first PEBL, the vector encoding the CAR and the first PEBL is introduced before the vector encoding the second PEBL. In some instances, wherein the nucleic acid encoding the second PEBL is located in a separate vector from the vector comprising the nucleic acids encoding the CAR and the first PEBL, the vector encoding the CAR and the first PEBL is introduced after the vector encoding the second PEBL.

In some instances, the nucleic acids encoding the CAR, the first PEBL, and the second PEBL are located in different vectors. In some instances, the nucleic acids encoding the CAR, the first PEBL, and the second PEBL are located in the same vector. In some instances, wherein the nucleic acid encoding the CAR, the first PEBL, and the second PEBL is located in different vectors, the different vectors can be introduced simultaneously. In some instances, wherein the nucleic acid encoding the CAR, the first PEBL, and the second PEBL is located in different vectors, the different vectors can be introduced sequentially.

In some instances, wherein the nucleic acids encoding the suicide gene and the second PEBL are located in a separate vector from the vector comprising the nucleic acids encoding the CAR and the first PEBL, the two vectors can be introduced simultaneously. In some instances, wherein the nucleic acids encoding the suicide gene and the second PEBL are located in a separate vector from the vector comprising the nucleic acids encoding the CAR and the first PEBL, the two vectors can be introduced sequentially. In some instances, wherein the nucleic acids encoding the suicide gene and the second PEBL are located in a separate vector from the vector comprising the nucleic acids encoding the CAR and the first PEBL, the vector encoding the CAR and the first PEBL is introduced before the vector encoding the suicide gene and the second PEBL. In some instances, wherein the nucleic acids encoding the suicide gene and the second PEBL are located in a separate vector from the vector comprising the nucleic acids encoding the CAR and the first PEBL, the vector encoding the CAR and the first PEBL is introduced after the vector encoding the suicide gene and the second PEBL.

In some instances, the nucleic acids encoding the CAR, the first PEBL, the suicide gene, and the second PEBL are located in different vectors. In some instances, the nucleic acids encoding the CAR, the first PEBL, the suicide gene, and the second PEBL are located in the same vector. In some instances, wherein the nucleic acids encoding the CAR, the first PEBL, the suicide gene, and the second PEBL are located in different vectors, the different vectors can be introduced simultaneously. In some instances, wherein the nucleic acids encoding the CAR, the first PEBL, the suicide gene, and the second PEBL are located in different vectors, the different vectors can be introduced sequentially.

In some instances, wherein the nucleic acids encoding the second PEBL and the suicide gene are located in a separate vector from the vector comprising the nucleic acids encoding the CAR and the first PEBL, the two vectors can be introduced simultaneously. In some instances, wherein the nucleic acids encoding the second PEBL and the suicide gene are located in a separate vector from the vector comprising the nucleic acids encoding the CAR and the first PEBL, the two vectors can be introduced sequentially. In some instances, wherein the nucleic acids encoding the second PEBL and the suicide gene are located in a separate vector from the vector comprising the nucleic acids encoding the CAR and the first PEBL, the vector encoding the CAR and the first PEBL is introduced before the vector encoding the second PEBL and the suicide gene. In some instances, wherein the nucleic acids encoding the second PEBL and the suicide gene are located in a separate vector from the vector comprising the nucleic acids encoding the CAR and the first PEBL, the vector encoding the CAR and the first PEBL is introduced after the vector encoding the second PEBL and the suicide gene.

In some instances, wherein the nucleic acids encoding the CAR, the first PEBL, the second PEBL, and the suicide gene are located in different vectors, the different vectors can be introduced simultaneously. In some instances, wherein the nucleic acids encoding the CAR, the first PEBL, the second PEBL, and the suicide gene are located in different vectors, the different vectors can be introduced sequentially.

### Engineered Immune Cells Expressing Dual Promoter Vectors

In some embodiments, provided is an engineered immune cell comprising a recombinant retroviral vector comprising (a) a first promoter operably linked to a first polynucleotide encoding any of the CARs described herein, and (b) a second promoter operably linked to a second polynucleotide encoding any of the PEBLs described herein. In some embodiments, the engineered immune cell comprises any of the recombinant retroviral vectors described herein containing a promoter driving CAR expression and another promoter driving PEBL expression.

In some embodiments, provided is an engineered immune cell comprising two recombinant retroviral vectors comprising: the first recombinant retroviral vector comprises (a) a first promoter operably linked to a first polynucleotide encoding any of the CARs described herein, and (b) a second promoter operably linked to a second polynucleotide encoding the first PEBL described herein; and the second recombinant retroviral vector comprises (a) a first promoter operably linked to a first polynucleotide encoding a suicide gene described herein, and (b) a second promoter operably linked to a second polynucleotide encoding the second PEBL describe herein. In some embodiments, the engineered immune cell comprises any of the recombinant retroviral vectors described herein containing a promoter driving CAR expression and another promoter driving PEBL expression. In some embodiments, the engineered immune cell comprises any of the recombinant retroviral vectors described herein containing a promoter driving suicide gene expression and another promoter driving another PEBL expression.

In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO:17. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO: 18. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO: 19. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO: 20. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO:21. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO: 22. In some embodiments, the engineered immune cell comprises a recombinant retroviral vector comprising a nucleic acid sequence having at least 85% (e.g., 85%, 86%, 87%, 88%, 89%, 90%, 91%, 92%, 93%, 94%, 95%, 96%, 97%, 98%, 99%, or more) sequence identity to SEQ ID NO: 23. In some instances, the engineered immune cell is an engineered CD4+ T cell or a population thereof or a population comprising such. In some instances, the engineered immune cell is an engineered CD8+ T cell or a population thereof or a population comprising such. In some instances, the engineered immune cell is an engineered CD3+ T cell or a population thereof or a population comprising such.

In some instances, the engineered immune cell comprises a polynucleotide comprising SEQ ID NO:17. In one embodiment, provided herein is an engineered CD4+ T cell or a population thereof comprising a nucleic acid construct comprising SEQ ID NO:17.

In some embodiments, provided herein is an engineered CD8+ T cell or a population thereof comprising a nucleic acid construct comprising SEQ ID NO:17. In some embodiments, provided herein is an engineered CD3+ T cell or a population thereof comprising a nucleic acid construct comprising SEQ ID NO:17.

In some instances, the engineered immune cell comprises a polynucleotide comprising SEQ ID NO:18. In one embodiment, provided herein is an engineered CD4+ T cell or a population thereof comprising a nucleic acid construct comprising SEQ ID NO:18.

In some embodiments, provided herein is an engineered CD8+ T cell or a population thereof comprising a nucleic acid construct comprising SEQ ID NO:18. In some embodiments, provided herein is an engineered CD3+ T cell or a population thereof comprising a nucleic acid construct comprising SEQ ID NO:18.

In some instances, the engineered immune cell comprises a polynucleotide comprising SEQ ID NO:19. In one embodiment, provided herein is an engineered CD4+ T cell or a population thereof comprising a nucleic acid construct comprising SEQ ID NO:19.

In some embodiments, provided herein is an engineered CD8+ T cell or a population thereof comprising a nucleic acid construct comprising SEQ ID NO:19. In some embodiments, provided herein is an engineered CD3+ T cell or a population thereof comprising a nucleic acid construct comprising SEQ ID NO:19.

In some instances, the engineered immune cell comprises a polynucleotide comprising SEQ ID NO:20. In one embodiment, provided herein is an engineered CD4+ T cell or a population thereof comprising a nucleic acid construct comprising SEQ ID NO:20. In some embodiments, provided herein is an engineered CD8+ T cell or a population thereof comprising a nucleic acid construct comprising SEQ ID NO:20. In some embodiments, provided herein is an engineered CD3+ T cell or a population thereof comprising a nucleic acid construct comprising SEQ ID NO:20.

In some instances, the engineered immune cell comprises a polynucleotide comprising SEQ ID NO:21. In one embodiment, provided herein is an engineered CD4+ T cell or a population thereof comprising a nucleic acid construct comprising SEQ ID NO:21. In some embodiments, provided herein is an engineered CD8+ T cell or a population thereof comprising a nucleic acid construct comprising SEQ ID NO:21. In some embodiments, provided herein is an engineered CD3+ T cell or a population thereof comprising a nucleic acid construct comprising SEQ ID NO:21.

In some instances, the engineered immune cell comprises a polynucleotide comprising SEQ ID NO:22. In one embodiment, provided herein is an engineered CD4+ T cell or a population thereof comprising a nucleic acid construct comprising SEQ ID NO:22. In some embodiments, provided herein is an engineered CD8+ T cell or a population thereof comprising a nucleic acid construct comprising SEQ ID NO:22. In some embodiments, provided herein is an engineered CD3+ T cell or a population thereof comprising a nucleic acid construct comprising SEQ ID NO:22.

In some instances, the engineered immune cell comprises a polynucleotide comprising SEQ ID NO:23. In one embodiment, provided herein is an engineered CD4+ T cell or a population thereof comprising a nucleic acid construct comprising SEQ ID NO:23. In some embodiments, provided herein is an engineered CD8+ T cell or a population thereof comprising a nucleic acid construct comprising SEQ ID NO:23. In some embodiments, provided herein is an engineered CD3+ T cell or a population thereof comprising a nucleic acid construct comprising SEQ ID NO:23.

In some embodiments, the engineered immune cells described herein or a population thereof comprise at least 10% CD7 CAR+/CD7-negative T cells, at least 15% CD7 CAR+/CD7-negative T cells, at least 20% CD7 CAR+/CD7-negative T cells, at least 25% CD7 CAR+/CD7-negative T cells, at least 30% CD7 CAR+/CD7-negative T cells, at least 35% CD7 CAR+/CD7-negative T cells, at least 40% CD7 CAR+/CD7-negative T cells, at least 45% CD7 CAR+/CD7-negative T cells, at least 50% CD7 CAR+/CD7-negative T cells, at least 55% CD7 CAR+/CD7-negative T cells, at least 60% CD7 CAR+/CD7-negative T cells, at least 65% CD7 CAR+/CD7-negative T cells, at least 70% CD7 CAR+/CD7-negative T cells, at least 75% CD7 CAR+/CD7-negative T cells, at least 80% CD7 CAR+/CD7-negative T cells, at least 85% CD7 CAR+/CD7-negative T cells, at least 90% CD7 CAR+/CD7-negative T cells, at least 95% CD7 CAR+/CD7-negative T cells, at least 96% CD7 CAR+/CD7-negative T cells, at least 97% CD7 CAR+/CD7-negative T cells, at least 98% CD7 CAR+/CD7-negative T cells, at least 99% CD7 CAR+/CD7-negative T cells, or 100% CD7 CAR+/CD7-negative T cells. In some embodiments, the engineered immune cells outlined herein include a population of substantially purified CD7 CAR+/CD7-negative T cells wherein such cells express any one of the dual promoter constructs described.

CD7 CAR+ Engineered Immune Cells with Reduced Expression of Endogenous CD7

In some embodiments, the engineered immune cells described herein express a CD7 CAR and have reduced or no endogenous CD7 expression compared to a non-engineered immune cell. Such engineered immune cells express a CD7 PEBL that minimizes or eliminates endogenous expression of CD7 on the surface of the immune cell. In some embodiments, reduced expression of CD7 refers to a downregulation or partial downregulation of surface CD7 by the cell. In some cases, reduced expression includes an at least 5% (e.g., at least 5%, 6%, 7%, 8%, 9%, 10%, 11%, 12%, 13%, 14%, 15%, 16%, 17%, 18%, 19%, 20%, 24%, 25%, 28%, 40%, 35%, 40%, 45%, 50%, 55%, 60%, 65%, 70%, 75%, 80%, 85%, 86%, 88%, 90%, 91%, 92%, 93%, 94%, 95%, 96%, 97%, 98%, 99% or 100%) reduction in expression level compared to the expression level of a comparable wild-type or non-engineered cell. In some embodiments, engineered immune cells outlined herein include a population of substantially purified CD7 CAR+/CD7-negative T cells.

In some embodiments, the engineered immune cells described herein express a CD7 PEBL having at least 90% sequence identity to SEQ ID NO:24. In some embodiments, the engineered immune cells express a CD7 PEBL having at least 90% sequence identity to SEQ ID NO:25. In some embodiments, the engineered immune cells express a CD7 PEBL having at least 90% sequence identity to SEQ ID NO:26. In some embodiments, the engineered immune cells express a CD7 PEBL having at least 90% sequence identity to SEQ ID NO:27.

In some embodiments, the engineered immune cells described herein express a CD7 CAR having at least 90% sequence identity to SEQ ID NO:28. In some embodiments, the engineered immune cells express a CD7 CAR having at least 90% sequence identity to SEQ ID NO:29. In some embodiments, the engineered immune cells express a CD7 CAR having at least 90% sequence identity to SEQ ID NO:30. In some embodiments, the engineered immune cells express a CD7 CAR having at least 90% sequence identity to SEQ ID NO:31.

In some embodiments, the engineered immune cells described herein express a CD7 PEBL having at least 90% sequence identity to SEQ ID NO:24 and a CD7 CAR having at least 90% sequence identity to SEQ ID NO:28. In some embodiments, the engineered immune cells described herein express a CD7 PEBL having at least 90% sequence identity to SEQ ID NO:24 and a CD7 CAR having at least 90% sequence identity to SEQ ID NO:30.

In some embodiments, the engineered immune cells express a CD7 PEBL having at least 90% sequence identity to SEQ ID NO:25 and express a CD7 CAR having at least 90% sequence identity to SEQ ID NO:29. In some embodiments, the engineered immune cells express a CD7 PEBL having at least 90% sequence identity to SEQ ID NO:25 and express a CD7 CAR having at least 90% sequence identity to SEQ ID NO:31.

In some embodiments, the engineered immune cells express a CD7 PEBL having at least 90% sequence identity to SEQ ID NO:26 and a CD7 CAR having at least 90% sequence identity to SEQ ID NO:28. In some embodiments, the engineered immune cells express a CD7 PEBL having at least 90% sequence identity to SEQ ID NO:26 and a CD7 CAR having at least 90% sequence identity to SEQ ID NO:30.

In some embodiments, the engineered immune cells express a CD7 PEBL having at least 90% sequence identity to SEQ ID NO:27 and express a CD7 CAR having at least 90% sequence identity to SEQ ID NO:29. In some embodiments, the engineered immune cells express a CD7 PEBL having at least 90% sequence identity to SEQ ID NO:27 and express a CD7 CAR having at least 90% sequence identity to SEQ ID NO:31.

In certain embodiments, the engineered immune cell is an engineered T cell, an engineered natural killer (NK) cell, an engineered NK/T cell, an engineered monocyte, an engineered macrophage, or an engineered dendritic cell. In some embodiments, the engineered immune cell is an engineered CD4+ T cell. In some embodiments, the engineered immune cell is an engineered CD8+ T cell. In some embodiments, the engineered immune cell is an engineered CD3+ T cell. Also provided is a population of any one of the engineered cells described herein.

In some embodiments, provided herein is a population of engineered immune cells (e.g., CD3+ T cells, CD4+ T cells, or CD8+ T cells) comprising at least about 50% (e.g., from about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative cells. In some embodiments, provided herein is a population of engineered immune cells comprising at least about 50% (e.g., from about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative CD4+ T cells. In some embodiments, provided herein is a population of engineered immune cells comprising at least about 50% (e.g., from about 50%, 55%, 58%0, 60%, 62%, 64%, 66%, 68%, 70%, 71%, 73%, 75%, 77%, 78%7, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative CD8+ T cells. Such a population of cells can be produced from peripheral blood mononuclear cells (PBMC), purified CD4+ T cells, purified CD8+ T cells, or a population comprising purified CD4+ T cells and purified CD8+ T cells.

In some embodiments, the engineered immune cells described herein are cultured to generate a highly pure population of CD7 CAR-T cells that have reduced expression of endogenous CD7. The level of purity can be at least about 75% (e.g., from about 75%, 76%, 77%, 78%, 79% 80%, 8%1%, 82%, 83%, 84% 85%, 86%, 87%, 88%, 89%, 90%, 91%, 92%, 93%, 94%, 95%, 96%, 97%, 98%, 99%, or more) CD7 CAR-T cells with no surface expression of CD7. The expression level of CD7 can be determined according to standard methods known to those in the art including, but not limited to immunocytochemistry, flow cytometry, and FACS analysis.

In some embodiments, the engineered immune cells of the present invention include CD45RO+ cells. In some embodiments, the engineered immune cells include CCR7-negative cells. In some embodiments, the engineered immune cells include central memory T cells. In some embodiments, the engineered immune cells include effector memory T cells. In some embodiments, the engineered immune cells include effector T cells. In some embodiments, the engineered immune cells include naive T cells.

In some instances, a population of engineered immune cells comprises effector memory T cells, central memory T cells, effector T cells, and naive T cells. In some embodiments, the population of engineered immune cells comprises a higher percentage of effector memory T cells and central memory T cells than effector T cells and naive T cells.

In some instances, a population of engineered immune cells comprises PD1-negative cells. In some instances, a population of engineered immune cells comprises TIM-1-negative cells.

In some embodiments, the engineered immune cells generate an immune response and secrete interferon-γ. The engineered immune cells induce T-cell mediated cytotoxicity in response to a cancer cell such as a CD7 expressing cancer cell.

In some embodiments, cells described herein comprising a bicistronic expression vector can be used to generate a population of CD7 CAR+/CD7-negative T cells. The CD7 CAR+/CD7-negative T cells can be expanded and enriched over time. The CD7 CAR+/CD7-negative T cells can be generated from cells including, but not limited to, bulk PBMCs, purified T cells comprising CD4+ and CD8+ T cells, and purified CD3+ T cells. The CD7 CAR+/CD7-negative T cells can be used to produce different subsets of T cells including effector memory T cells, central memory T cells, effector T cells, and naive T cells.

In another aspect, also provided is a method for producing the engineered immune cell (e.g., engineered CD3+ T cell, engineered CD4+ T cell, and engineered CD8+ T cell) having any of the embodiments described herein, the method comprising introducing into an immune cell any of the bicistronic constructs or dual promoter constructs of the present invention. In some embodiments, the engineered immune cells are derived from immune cells obtained from a subject that will receive the engineered immune cells as a therapy. In some embodiments, the engineered immune cells are derived from immune cells obtained from a donor and the resulting engineered immune cells are administered to a subject as a therapy.

In various aspects, also provided is a kit for producing an engineered immune cell described herein. The present kit can be used to produce, e.g., allogeneic or autologous T cells having anti-CD7 CAR-mediated cytotoxic activity. In some embodiments, the kit is useful for producing allogeneic effector T cells having anti-CD7 CAR-mediated cytotoxic activity. In certain embodiments, the kit is useful for producing autologous effector T cells having anti-CD7 CAR-mediated cytotoxic activity.

Accordingly, provided herein is a kit comprising any one of the bicistronic constructs or dual promoter constructs described herein.

In certain embodiments, the bicistronic construct further comprise sequences (e.g., plasmid or vector sequences) that allow, e.g., cloning and/or expression. For example, the nucleotide sequence can be provided as part of a plasmid for ease of cloning into other plasmids and/or vectors (expression vectors or viral expression vectors) for, e.g., transfection, transduction, or electroporation into a cell (e.g., an immune cell).

Typically, the kits are compartmentalized for ease of use and can include one or more containers with reagents. In certain embodiments, all of the kit components are packaged together. Alternatively, one or more individual components of the kit can be provided in a separate package from the other kit components. The kits can also include instructions for using the kit components.

In some aspects, provided herein are kits configured for producing the engineered immune cells described herein. In some aspects, a kit comprises (a) a first expression vector comprising a nucleotide sequence encoding (i) a domain that binds to a subunit of a T cell receptor (TCR) complex linked to (b) a synthetic localizing domain and a nucleotide sequence encoding a kill gene; and (ii) a second expression vector comprising a nucleotide sequence encoding a chimeric antigen receptor (CAR) and a nucleotide sequence encoding a surface polypeptide binding domain linked to a synthetic surface polypeptide localizing domain.

In some embodiments, the first expression vector or the second expression vector can be a retroviral vector, a lentiviral vector, an adenoviral vector, or an adeno-associated viral vector. In some embodiments, the first expression vector or the second expression vector can be a lentiviral vector. In some embodiments, the first expression vector or the second expression vector can be a retroviral vector. In some embodiments, the CAR of the kit may comprise a target binding domain. In some embodiments, the target binding domain of the CAR may bind to CD7. In some embodiments, the surface polypeptide of (ii) can be CD7. In some embodiments, the subunit of the TCR complex can be CD3ε. In some embodiments, the subunit of the TCR complex can be CD3ε, CD3γ, CD3δ, TCRα, or TCRβ.

In some embodiments, the nucleotide sequence encoding (i) the domain that binds to a subunit of a TCR complex linked to (ii) a synthetic localizing domain and the nucleotide sequence encoding the kill gene can be operably linked by an Internal Ribosome Entry Site (IRES) or a ribosomal codon skipping site. In some embodiments, the nucleotide sequence encoding the CAR and the nucleotide sequence encoding the surface polypeptide binding domain linked to the synthetic surface polypeptide localizing domain are operably linked by an Internal Ribosome Entry Site (IRES) or a ribosomal codon skipping site. In some embodiments, the ribosomal codon skipping site can be a 2A self-cleaving peptide.

In some embodiments, the first expression vector and the second expression vector of the kit described herein are mixed at a ratio of at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 1:2, at least 1:3, at least 1:4, at least 1:5, at least 1:6, at least 1:7, or at least 1:8. In some embodiments, the first expression vector and the second expression vector of the kit described herein are mixed at a ratio of at least 2:1.

CD7 CAR+ Engineered Immune Cells with Reduced Expression of Endogenous CD7 and CD3

In some aspects, the engineered immune cells described herein express a CD7 CAR and have reduced endogenous CD7 expression and CD3 expression compared to a non-engineered immune cell. Such engineered immune cells express a CD7 PEBL and a CD3 PEBL that minimize or eliminate endogenous expression of CD7 and CD3 on the surface of the immune cell, respectively. In some embodiments, reduced expression of CD7 and CD3 refers to a downregulation or partial downregulation of surface expression of CD7 and CD3 on the cell surface. In some cases, reduced expression includes an at least 5% (e.g., at least 5%, 6%, 7%, 8%, 9%, 10%, 11%, 12%, 13%, 14%, 15%, 16%, 17%, 18%, 19%, 20%, 24%, 25%, 28%, 30%, 35%, 40%, 45%, 50%, 55%, 60%, 65%, 70%, 75%, 80%, 85%, 86%, 88%, 90%, 91%, 92%, 93%, 94%, 95%, 96%, 97%, 98%, 99% or 100%) reduction in expression level compared to the expression level of a comparable wild-type or non-engineered cell. In some embodiments, engineered immune cells outlined herein include a population of substantially purified CD7 CAR+/CD7-negative/CD3-negative T cells.

In certain embodiments, the engineered immune cell is an engineered T cell, an engineered natural killer (NK) cell, an engineered NK/T cell, an engineered monocyte, an engineered macrophage, or an engineered dendritic cell. In some embodiments, the engineered immune cell is an engineered CD4+ T cell. In some embodiments, the engineered immune cell is an engineered CD8+ T cell. In some embodiments, the engineered immune cell is an engineered CD3+ T cell. Also provided is a population of any one of the engineered cells described herein.

In some embodiments, provided herein is a population of engineered immune cells (e.g., CD3+ T cells, CD4+ T cells, or CD8+ T cells) comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative, and endogenous CD3-negative cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79% 80%, 8%1%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative, and endogenous CD3-negative CD4+ T cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79% 80%, 8%1%, 82%, 83%, 84% 85%, 86%, 87%, 88%, 90%, 91%, 92%, 93%, 94%, 95%, 96%, 97%, 98%, 99%, or more) CD7 CAR-positive, endogenous CD7-negative, and endogenous CD3-negative CD8+ T cells. Such a population of cells can be produced from peripheral blood mononuclear cells (PBMC), purified CD4+ T cells, purified CD8+ T cells, or a population comprising purified CD4+ T cells and purified CD8+ T cells.

In some embodiments, the engineered immune cells described herein are cultured to generate a highly pure population of CD7 CAR-T cells that have reduced expression of endogenous CD7 and endogenous CD3. The level of purity can be at least about 75% (e.g., about 75%, 76%, 77%, 78%, 79%, 80%, 81%, 82%, 83%, 84% 85%, 86%, 87%, 88%, 89%, 90%, 91%, 92%, 93%, 94%, 95%, 96%, 97%, 98%, 99%, or more) CD7 CAR-T cells with no surface expression of CD7 and CD3. The expression level CD7 and CD3 can be determined according to standard methods known to those in the art including, but not limited to immunocytochemistry, flow cytometry, and FACS analysis.

In some embodiments, the engineered immune cells of the present invention include CD45RO+ cells. In some embodiments, the engineered immune cells include CCR7-negative cells. In some embodiments, the engineered immune cells include central memory T cells. In some embodiments, the engineered immune cells include effector memory T cells. In some embodiments, the engineered immune cells include effector T cells. In some embodiments, the engineered immune cells include naive T cells.

In some instances, a population of engineered immune cells comprises effector memory T cells, central memory T cells, effector T cells, and naive T cells. In some embodiments, the population of engineered immune cells comprises a higher percentage of effector memory T cells and central memory T cells than effector T cells and naive T cells. In some embodiments, the population of engineered immune cells comprises about 40% or more effector memory T cells.

In some instances, a population of engineered immune cells comprises PD1-negative cells. In some instances, a population of engineered immune cells comprises TIM-1-negative cells. In some embodiments, the population comprises about 60% or more PD1-negative, TIM-1-negative cells. In some embodiments, the population comprises about 4% to about 20% PD1 positive, TIM-1 positive cells.

In some embodiments, the engineered immune cells generate an immune response and secrete interferon-γ. The engineered immune cells induce T-cell mediated cytotoxicity in response to a cancer cell such as a CD7 expressing cancer cell.

In some embodiments, cells described herein comprising a bicistronic expression vector and another expression vector that can be used to generate a population of CD7 CAR+/CD7-neg/CD3-neg T cells. The CD7 CAR+/CD7-neg/CD3-neg T cells can be expanded and enriched over time. The CD7 CAR+/CD7-neg/CD3-neg T cells can be generated from cells including, but not limited to, bulk PBMCs, purified T cells comprising CD4+ and CD8+ T cells. The CD7 CAR+/CD7-neg/CD3-neg T cells can be used to produce different subsets of T cells including effector memory T cells, central memory T cells, effector T cells, and naive T cells.

In another aspect, also provided is a method for producing the engineered immune cell expressing a chimeric antigenic receptor (CAR) targeting a cell surface protein, the method comprising introducing into an immune cell a) a first nucleic acid comprising a nucleotide sequence encoding a first target binding domain linked to a first localizing domain; b) a second nucleic acid comprising a nucleotide sequence encoding a chimeric antigen receptor (CAR), wherein the CAR comprises a second target binding domain; c) a third nucleic acid comprising a nucleotide sequence encoding a third target binding domain linked to a second localizing domain, wherein the first and second target binding domain bind to a first immune cell surface receptor molecule and the third target binding domain binds to a second immune cell surface receptor molecule; and ii) culturing the engineered immune cell, thereby producing said engineered immune cell. In some embodiments, the engineered immune cells are derived from immune cells obtained from a subject that will receive the engineered immune cells as a therapy. In some embodiments, the engineered immune cells are derived from immune cells obtained from a donor and the resulting engineered immune cells are administered to a subject as a therapy.

In various aspects, also provided is a kit for producing an engineered immune cell described herein. The present kit can be used to produce, e.g., CD7 CAR+/CD7-neg/CD3-neg T cells with less risk of mediating GvHD.

Accordingly, provided herein is a kit comprising a first expression vector comprising: a) a first nucleic acid comprising a nucleotide sequence encoding a first target binding domain linked to a localizing domain; b) a second nucleic acid comprising a nucleotide sequence encoding a chimeric antigen receptor (CAR), wherein the CAR comprises a second target binding domain; a second expression vector comprising a third nucleic acid comprising a nucleotide sequence encoding a third target binding domain linked to a localizing domain; and wherein the first and second target binding domain bind to a first immune cell surface receptor molecule and the third target binding domain binds to a second immune cell surface receptor molecule.

In certain embodiments, the bicistronic construct further comprise sequences (e.g., plasmid or vector sequences) that allow, e.g., cloning and/or expression. For example, the nucleotide sequence can be provided as part of a plasmid for ease of cloning into other plasmids and/or vectors (expression vectors or viral expression vectors) for, e.g., transfection, transduction, or electroporation into a cell (e.g., an immune cell).

Typically, the kits are compartmentalized for ease of use and can include one or more containers with reagents. In certain embodiments, all of the kit components are packaged together. Alternatively, one or more individual components of the kit can be provided in a separate package from the other kit components. The kits can also include instructions for using the kit components.

CD7 CAR+ Engineered Immune Cells with Reduced Expression of Endogenous CD7 and CD3 and Induced Expression of a Suicide Gene (a Kill Gene)

In some aspects, the engineered immune cells described herein express a CD7 CAR and a suicide gene and have reduced endogenous CD7 expression and CD3 expression compared to a non-engineered immune cell. In some embodiments, the suicide gene is CD20 or derivative thereof. In some embodiments, the suicide gene is a truncated CD20. Such engineered immune cells express a CD7 PEBL and a CD3 PEBL that minimize or eliminate endogenous expression of CD7 and CD3 on the surface of the immune cell, respectively. In some embodiments, reduced expression of CD7 and CD3 refers to a downregulation or partial downregulation of surface expression of CD7 and CD3 on the cell surface. In some cases, reduced expression includes an at least 5% (e.g., at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 86%, 88%, 90%, 91%, 92%, 93%, 94%, 95%, 96%, 97%, 98%, 99% or 100%) reduction in expression level compared to the expression level of a comparable wild-type or non-engineered cell. In some embodiments, engineered immune cells outlined herein include a population of substantially purified CD7 CAR-positive/CD20-positive/CD7-negative/CD3-negative T cells.

In certain embodiments, the engineered immune cell is an engineered T cell, an engineered natural killer (NK) cell, an engineered NK/T cell, an engineered monocyte, an engineered macrophage, or an engineered dendritic cell. In some embodiments, the engineered immune cell is an engineered CD4+ T cell. In some embodiments, the engineered immune cell is an engineered CD8+ T cell. In some embodiments, the engineered immune cell is an engineered CD3+ T cell. Also provided is a population of any one of the engineered cells described herein.

In some embodiments, provided herein is a population of engineered immune cells (e.g., CD3+ T cells, CD4+ T cells, or CD8+ T cells) comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, CD20-positive, endogenous CD7-negative, and endogenous CD3-negative cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, CD20-positive, endogenous CD7-negative, and endogenous CD3-negative CD4+ T cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 93%, 94%, 95%, 96%, 97%, 98%, 99%, or more) CD7 CAR-positive, CD20-positive, endogenous CD7-negative, and endogenous CD3-negative CD8+ T cells. Such a population of cells can be produced from peripheral blood mononuclear cells (PBMC), purified CD4+ T cells, purified CD8+ T cells, or a population comprising purified CD4+ T cells and purified CD8+ T cells.

In some embodiments, the engineered immune cells described herein are cultured to generate a highly pure population of CD7 CAR-positive and CD20-positive T cells that have reduced expression of endogenous CD7 and endogenous CD3. The level of purity can be at least about 75% (e.g., about 75%, 76%, 77%, 78%, 79%, 80%, 81%, 82%, 83%, 84% 85%, 86%, 87%, 88%, 89%, 90%, 91%, 92%, 93%, 94%, 95%, 96%, 97%, 98%, 99%, or more) CD7 CAR-positive and CD20-positive T cells with no surface expression of CD7 and CD3.

The expression level of CD7 and CD3 can be determined according to standard methods known to those in the art including, but not limited to immunocytochemistry, flow cytometry, and FACS analysis.

In some embodiments, the engineered immune cells of the present invention include CD45RO+ cells. In some embodiments, the engineered immune cells include CCR7-negative cells. In some embodiments, the engineered immune cells include central memory T cells. In some embodiments, the engineered immune cells include effector memory T cells. In some embodiments, the engineered immune cells include effector T cells. In some embodiments, the engineered immune cells include naive T cells.

In some instances, a population of engineered immune cells comprises effector memory T cells, central memory T cells, effector T cells, and naive T cells. In some embodiments, the population of engineered immune cells comprises a higher percentage of effector memory T cells and central memory T cells than effector T cells and naive T cells. In some embodiments, the population of engineered immune cells comprises about 40% or more effector memory T cells.

In some instances, a population of engineered immune cells comprises PD1-negative cells. In some instances, a population of engineered immune cells comprises TIM-1-negative cells. In some embodiments, the population comprises about 60% or more PD1-negative, TIM-1-negative cells. In some embodiments, the population comprises about 4% to about 20% PD1 positive, TIM-1 positive cells.

In some embodiments, the engineered immune cells generate an immune response and secrete interferon-γ. The engineered immune cells induce T-cell mediated cytotoxicity in response to a cancer cell such as a CD7 expressing cancer cell.

In some embodiments, cells described herein comprising two bicistronic expression vectors that can be used to generate a population of CD7 CAR-positive/CD20-positive/CD7-negative/CD3-negative T cells. The CD7 CAR-positive/CD20-positive/CD7-negative/CD3-negative T cells can be expanded and enriched over time. The CD7 CAR-positive/CD20-positive/CD7-negative/CD3-negative T cells can be generated from cells including, but not limited to, bulk PBMCs, purified T cells comprising CD4+ and CD8+ T cells. The CD7 CAR-positive/CD20-positive/CD7-negative/CD3-negative T cells can be used to produce different subsets of T cells including effector memory T cells, central memory T cells, effector T cells, and naive T cells.

In another aspect, also provided is a method for i) producing the engineered immune cell expressing a chimeric antigenic receptor (CAR) targeting a cell surface protein, the method comprising introducing into an immune cell a) a first nucleic acid comprising a nucleotide sequence encoding a first target binding domain linked to a first localizing domain; b) a second nucleic acid comprising a nucleotide sequence encoding a chimeric antigen receptor (CAR), wherein the CAR comprises a second target binding domain; c) a third nucleic acid comprising a nucleotide sequence encoding a third target binding domain linked to a second localizing domain, wherein the first and second target binding domain bind to a first immune cell surface receptor molecule and the third target binding domain binds to a second immune cell surface receptor molecule; d) a fourth nucleic acid comprising a nucleotide sequence encoding a suicide gene; and ii) culturing the engineered immune cell, thereby producing said engineered immune cell. In some embodiments, the engineered immune cells are derived from immune cells obtained from a subject that will receive the engineered immune cells as a therapy. In some embodiments, the engineered immune cells are derived from immune cells obtained from a donor and the resulting engineered immune cells are administered to a subject as a therapy. In some embodiments, the engineered immune cells are autologous engineered immune cells. In some embodiments, the engineered immune cells are allogeneic engineered immune cells.

In various aspects, also provided is a kit for producing an engineered immune cell described herein. The present kit can be used to produce, e.g., CD7 CAR-positive/CD20-positive/CD7-negative/CD3-negative T cells with less risk of mediating GvHD.

Accordingly, provided herein is a kit comprising a first expression vector comprising: a) a first nucleic acid comprising a nucleotide sequence encoding a first target binding domain linked to a localizing domain; b) a second nucleic acid comprising a nucleotide sequence encoding a chimeric antigen receptor (CAR), wherein the CAR comprises a second target binding domain; a second expression vector comprising a) a third nucleic acid comprising a nucleotide sequence encoding a third target binding domain linked to a localizing domain; b) a fourth nucleic acid comprising a nucleotide sequence encoding a suicide gene; and wherein the first and second target binding domain bind to a first immune cell surface receptor molecule and the third target binding domain binds to a second immune cell surface receptor molecule.

### Methods for Producing Engineered Immune Cells

In some aspects, provided herein are methods of producing a population of engineered immune cells. The method may comprise a) introducing into immune cells a first nucleic acid sequence encoding (i) a domain that binds to a subunit of a T cell receptor (TCR) complex linked to (ii) a synthetic localizing domain; a second nucleic acid sequence encoding a chimeric antigen receptor (CAR); and a third nucleic acid sequence encoding a surface polypeptide binding domain linked to a synthetic surface polypeptide localizing domain (e.g., PEBL), thereby producing the population of engineered immune cells.

The method may further comprise b) culturing the population of engineered immune cells; thereby expressing the CAR and downregulating surface expression of the TCR complex and the surface polypeptide in the population of engineered immune cell; wherein the amount of the first nucleic acid sequence encoding (i) a domain that binds to a subunit of a T cell receptor (TCR) complex linked to (ii) a synthetic localizing domain is at least twice as high as the amount of the second nucleic acid sequence encoding a CAR or the third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times, at least about 15 times, at least about 20 times, at least about 25 times, at least about 30 times, at least about 35 times, or at least about 40 times as high as the amount of the second nucleic acid sequence encoding the CAR or the third nucleic acid sequence encoding a surface polypeptide binding domain linked to a synthetic surface polypeptide localizing domain. In some embodiments, the amount of the first nucleic acid sequence encoding (i) a domain that binds to a subunit of a T cell receptor (TCR) complex linked to (ii) a synthetic localizing domain in the engineered immune cell is at most about 40 times, at most about 35 times, at most about 30 times, at most about 25 times, at most about 20 times, at most about 15 times, at most about 10 times, at most about 9 times, at most about 8 times, at most about 7 times, at most about 6 times, at most about 5 times, at most about 4 times, at most about 3 times, or at most about 2 times as high as the amount of the second nucleic acid sequence encoding the CAR or the third nucleic acid sequence encoding a surface polypeptide binding domain linked to a synthetic surface polypeptide localizing domain.

In some embodiments, the amount of the first nucleic acid sequence encoding (i) a domain that binds to a subunit of a T cell receptor (TCR) complex linked to (ii) a synthetic localizing domain in the engineered immune cell is from about 2 times to about 20 times as high as the amount of the second nucleic acid sequence encoding the CAR or the third nucleic acid sequence encoding a surface polypeptide binding domain linked to a synthetic surface polypeptide localizing domain. In some embodiments, the amount of the first nucleic acid sequence encoding (i) a domain that binds to a subunit of a T cell receptor (TCR) complex linked to (ii) a synthetic localizing domain in the engineered immune cell is from about 2 times to about 3 times, from about 2 times to about 4 times, from about 2 times to about 5 times, from about 2 times to about 6 times, from about 2 times to about 7 times, from about 2 times to about 8 times, from about 2 times to about 9 times, from about 2 times to about 10 times, from about 2 times to about 15 times, from about 2 times to about 7 times, from about 2 times to about 20 times, from about 3 times to about 4 times, from about 3 times to about 5 times, from about 3 times to about 6 times, from about 3 times to about 7 times, from about 3 times to about 8 times, from about 3 times to about 9 times, from about 3 times to about 10 times, from about 3 times to about 15 times, from about 3 times to about 7 times, from about 3 times to about 20 times, from about 4 times to about 5 times, from about 4 times to about 6 times, from about 4 times to about 7 times, from about 4 times to about 8 times, from about 4 times to about 9 times, from about 4 times to about 10 times, from about 4 times to about 15 times, from about 4 times to about 7 times, from about 4 times to about 20 times, from about 5 times to about 6 times, from about 5 times to about 7 times, from about 5 times to about 8 times, from about 5 times to about 9 times, from about 5 times to about 10 times, from about 5 times to about 15 times, from about 5 times to about 7 times, from about 5 times to about 20 times, from about 6 times to about 7 times, from about 6 times to about 8 times, from about 6 times to about 9 times, from about 6 times to about 10 times, from about 6 times to about 15 times, from about 6 times to about 7 times, from about 6 times to about 20 times, from about 7 times to about 8 times, from about 7 times to about 9 times, from about 7 times to about 10 times, from about 7 times to about 15 times, from about 7 times to about 7 times, from about 7 times to about 20 times, from about 8 times to about 9 times, from about 8 times to about 10 times, from about 8 times to about 15 times, from about 8 times to about 7 times, from about 8 times to about 20 times, from about 9 times to about 10 times, from about 9 times to about 15 times, from about 9 times to about 7 times, from about 9 times to about 20 times, from about 10 times to about 15 times, from about 10 times to about 7 times, from about 10 times to about 20 times, from about 15 times to about 7 times, from about 15 times to about 20 times, or about 7 times to about 20 times as high as the amount of the second nucleic acid sequence encoding the CAR or the third nucleic acid sequence encoding a surface polypeptide binding domain linked to a synthetic surface polypeptide localizing domain.

The CAR may comprise a target binding domain that binds to the surface polypeptide and the surface polypeptide can be CD7. Downregulation of the surface polypeptide may prevent fratricide of the population of engineered immune cells by the CAR. The nucleic acid sequence encoding the CAR and the nucleic acid sequence encoding the PEBL may be located on the same nucleic acid molecule, which can be a bicistronic vector. The nucleic acid sequence encoding the domain that binds to a subunit of a TCR complex may be on a separate nucleic acid molecule than the nucleic acid sequence encoding the CAR and the nucleic acid sequence encoding the PEBL. The separate nucleic acid molecule may be another vector. The bicistronic vector and the vector comprising the nucleic acid sequence encoding the domain that binds to a subunit of a TCR complex may be introduced to immune cells concurrently or sequentially.

In some embodiments, in the method provided herein, step a) may comprise cotransduction of the bicistronic vector comprising the second nucleic acid sequence and the third nucleic acid sequence, and the first vector comprising the first nucleic acid sequence. In some embodiments, in the method provided herein, step a) may comprise sequential transduction of the bicistronic vector comprising the second nucleic acid sequence and the third nucleic acid sequence, and the first vector comprising the first nucleic acid sequence. When transduction occurs sequentially, the vector comprising the first nucleic acid sequence may be introduced into immune cells at least 1 hour, at least 2 hours, at least 5 hours, at least 10 hours, at least 12 hours, at least 18 hours, at least 24 hours, at least 36 hours, at least 48 hours, at least 3 days, at least 4 days, or at least 5 days prior to transduction of the bicistronic vector comprising the second nucleic acid sequence and the third nucleic acid sequence.

In some embodiments, the multiplicity of infection (MOI) of the bicistronic vector may be at least about 5, at least about 10, at least about 15, at least about 20, at least about 25, at least about 30, at least about 40, or at least about 50. In some embodiments, the multiplicity of infection (MOI) of the bicistronic vector may be at most about 50, at most about 40, at most about 30, at most about 25, at most about 20, at most about 15, at most about 10, or at most about 5. In some embodiments, the multiplicity of infection (MOI) of the bicistronic vector may be about 3 to about 60. In some embodiments, the multiplicity of infection (MOI) of the bicistronic vector may be about 3 to about 5, from about 3 to about 7, from about 3 to about 10, from about 3 to about 12, from about 3 to about 15, from about 3 to about 20, from about 3 to about 25, from about 3 to about 30, from about 3 to about 40, from about 3 to about 50, from about 3 to about 60, from about 5 to about 7, from about 5 to about 10, from about 5 to about 12, from about 5 to about 15, from about 5 to about 20, from about 5 to about 25, from about 5 to about 30, from about 5 to about 40, from about 5 to about 50, from about 5 to about 60, from about 7 to about 10, from about 7 to about 12, from about 7 to about 15, from about 7 to about 20, from about 7 to about 25, from about 7 to about 30, from about 7 to about 40, from about 7 to about 50, from about 7 to about 60, from about 10 to about 12, from about 10 to about 15, from about 10 to about 20, from about 10 to about 25, from about 10 to about 30, from about 10 to about 40, from about 10 to about 50, from about 10 to about 60, from about 12 to about 15, from about 12 to about 20, from about 12 to about 25, from about 12 to about 30, from about 12 to about 40, from about 12 to about 50, from about 12 to about 60, from about 15 to about 20, from about 15 to about 25, from about 15 to about 30, from about 15 to about 40, from about 15 to about 50, from about 15 to about 60, from about 20 to about 25, from about 20 to about 30, from about 20 to about 40, from about 20 to about 50, from about 20 to about 60, from about 25 to about 30, from about 25 to about 40, from about 25 to about 50, from about 25 to about 60, from about 30 to about 40, from about 30 to about 50, from about 30 to about 60, from about 40 to about 50, from about 40 to about 60, or about 50 to about 60.

In some embodiments, the multiplicity of infection (MOI) of the vector (e.g., a first vector) comprising a domain that binds to a subunit of a TCR complex may be at least about 10, at least about 15, at least about 20, at least about 25, at least about 30, at least about 40, at least about 50, at least about 60, or at least about 75. In some embodiments, the multiplicity of infection (MOI) of the vector comprising a domain that binds to a subunit of a TCR complex may be at most about 60, at most about 50, at most about 40, at most about 30, at most about 25, at most about 20, at most about 15, or at most about 10. In some embodiments, the multiplicity of infection (MOI) of the vector comprising a domain that binds to a subunit of a TCR complex vector may be about 10 to about 75. In some embodiments, the multiplicity of infection (MOI) of the vector comprising a domain that binds to a subunit of a TCR complex may be about 10 to about 15, from about 10 to about 20, from about 10 to about 25, from about 10 to about 30, from about 10 to about 35, from about 10 to about 40, from about 10 to about 45, from about 10 to about 50, from about 10 to about 55, from about 10 to about 60, from about 10 to about 75, from about 15 to about 20, from about 15 to about 25, from about 15 to about 30, from about 15 to about 35, from about 15 to about 40, from about 15 to about 45, from about 15 to about 50, from about 15 to about 55, from about 15 to about 60, from about 15 to about 75, from about 20 to about 25, from about 20 to about 30, from about 20 to about 35, from about 20 to about 40, from about 20 to about 45, from about 20 to about 50, from about 20 to about 55, from about 20 to about 60, from about 20 to about 75, from about 25 to about 30, from about 25 to about 35, from about 25 to about 40, from about 25 to about 45, from about 25 to about 50, from about 25 to about 55, from about 25 to about 60, from about 25 to about 75, from about 30 to about 35, from about 30 to about 40, from about 30 to about 45, from about 30 to about 50, from about 30 to about 55, from about 30 to about 60, from about 30 to about 75, from about 35 to about 40, from about 35 to about 45, from about 35 to about 50, from about 35 to about 55, from about 35 to about 60, from about 35 to about 75, from about 40 to about 45, from about 40 to about 50, from about 40 to about 55, from about 40 to about 60, from about 40 to about 75, from about 45 to about 50, from about 45 to about 55, from about 45 to about 60, from about 45 to about 75, from about 50 to about 55, from about 50 to about 60, from about 50 to about 75, from about 55 to about 60, from about 55 to about 75, or about 60 to about 75.

In some embodiments, a ratio of an MOI of the bicistronic vector and an MOI of the vector comprising a domain that binds to a subunit of a TCR complex (e.g., a first vector) may be at least about 1:2, at least about 1:3, at least about 1:4, at least about 1:5, at least about 1:6, at least about 1:7, at least about 1:8, at least about 1:10, at least about 1:12, or at least about 1:16. In some embodiments, a ratio of an MOI of the bicistronic vector and an MOI of the vector comprising a domain that binds to a subunit of a TCR complex (e.g., a first vector) may be at most about 1:16, at most about 1:12, at most about 1:10, at most about 1:8, at most about 1:7, at most about 1:6, at most about 1:5, at most about 1:4, at most about 1:3, or at most about 1:2.

In some embodiments, a ratio of a vector copy number (VCN) of a CD3 PEBL to CD7 CAR may be at least about 1:1, at least about 1.5:1, at least about 1.6:1, at least about 1.7:1, at least about 1.8:1, at least about 1.9:1, at least about 2:1, at least about 2.1:1, at least about 2.2:1, at least about 2.3:1, at least about 2.4:1, at least about 2.5:1, at least about 2.6:1, at least about 2.7:1, at least about 2.8:1, at least about 2.9:1, at least about 3:1, at least about 3.5:1, at least about 4:1, at least about 4.5:1, at least about 5:1, at least about 6:1, at least about 7:1, at least about 8:1, at least about 9:1, or at least about 10:1. In some embodiments, a ratio of a vector copy number (VCN) of a CD3 PEBL to CD7 CAR may be at most about 10:1, at most about 9:1, at most about 8:1, at most about 7:1, at most about 6:1, at most about 5:1, at most about 4.5:1, at most about 4:1, at most about 3.5:1, at most about 3:1, at most about 2.9:1, at most about 2.8:1, at most about 2.7:1, at most about 2.6:1, at most about 2.5:1, at most about 2.4:1, at most about 2.3:1, at most about 2.2:1, at most about 2.1:1, at most about 2:1, at most about 1.5:1, or at most about 1:1. In some embodiments, a ratio of a vector copy number (VCN) of a CD3 PEBL to CD7 PEBL may be at least about 1:1, at least about 1.5:1, at least about 1.6:1, at least about 1.7:1, at least about 1.8:1, at least about 1.9:1, at least about 2:1, at least about 2.1:1, at least about 2.2:1, at least about 2.3:1, at least about 2.4:1, at least about 2.5:1, at least about 2.6:1, at least about 2.7:1, at least about 2.8:1, at least about 2.9:1, at least about 3:1, at least about 3.5:1, at least about 4:1, at least about 4.5:1, at least about 5:1, at least about 6:1, at least about 7:1, at least about 8:1, at least about 9:1, or at least about 10:1. In some embodiments, a ratio of a vector copy number (VCN) of a CD3 PEBL to CD7 PEBL may be at most about 10:1, at most about 9:1, at most about 8:1, at most about 7:1, at most about 6:1, at most about 5:1, at most about 4.5:1, at most about 4:1, at most about 3.5:1, at most about 3:1, at most about 2.9:1, at most about 2.8:1, at most about 2.7:1, at most about 2.6:1, at most about 2.5:1, at most about 2.4:1, at most about 2.3:1, at most about 2.2:1, at most about 2.1:1, at most about 2:1, at most about 1.5:1, or at most about 1:1.

In some embodiments, the method of producing engineered immune cells comprises introducing to the immune cells a nucleic acid sequence encoding a kill gene. In some embodiments, the kill gene encodes a surface antigen, and the surface antigen may comprise CD20 or a fragment and/or derivative thereof. In some embodiments, the kill gene comprises a kill gene described herein.

In some embodiments, the method of producing engineered immune cells comprises depleting cells that express the TCR complex from the population of engineered immune cells. In some embodiments, the method of producing engineered immune cells comprises enriching cells that express the surface antigen, thereby depleting cells that express the TCR complex. In some embodiments, the method of producing engineered immune cells comprises, prior to depleting, enriching cells that express the surface antigen, thereby producing a population of CD20+TCR− engineered immune cells.

In some embodiments, the method of producing engineered immune cells comprises culturing the population of engineered immune cells comprising expanding the population of engineered immune cells for at least 1 day, at least 2 days, at least 3 days, at least 4 days, at least 5 days, at least 6 days, at least 7 days, at least 8 days, at least 9 days, or at least 10 days between the introducing and the depleting. In some embodiments, after the process of depleting, at least 75%, at least 80%, at least 85%, at least 90%, at least 91%, at least 92%, at least 93%, at least 94%, at least 95%, at least 96%, at least 97%, at least 98%, or at least 99% of the population of CD20+TCR− engineered immune cells do not express CD3.

In some embodiments, following the manufacturing, the produced population of engineered immune cells may comprise the engineered immune cells described herein.

### Administering Engineered Immune Cells

In other aspects, also provided is a method of treating cancer in a subject in need thereof, comprising administering a therapeutic amount of an engineered immune cell having any of the embodiments described herein to the subject, thereby treating cancer in a subject in need thereof.

In certain embodiments, the method comprises administering a therapeutic amount of an engineered immune cell comprising a bicistronic viral construct comprising a polynucleotide comprising a nucleic acid sequence encoding a CAR and a polynucleotide comprising a nucleic acid sequence encoding a PEBL. In various embodiments, the method comprises administering a therapeutic amount of any one of the engineered immune cells described herein comprising a recombinant retroviral vector comprising: (a) a first promoter operably linked to a first polynucleotide encoding a CD7 chimeric antigen receptor (CD7 CAR) as outlined herein; and (b) a second promoter operably linked to a second polynucleotide encoding a CD7 protein expression blocker (CD7 PEBL) as outlined herein.

In some embodiments, the method comprises administering a therapeutic amount of an engineered immune cell comprising two bicistronic viral constructs comprising: a first bicistronic viral construct comprises a polynucleotide comprising a nucleic acid sequence encoding a CAR and a polynucleotide comprising a nucleic acid sequence encoding a first PEBL; and a second bicistronic viral construct comprises a polynucleotide comprising a nucleic acid sequence encoding a suicide gene and a polynucleotide comprising a nucleic acid sequence encoding a second PEBL. In various embodiments, the method comprises administering a therapeutic amount of any one of the engineered immune cells described herein comprising a recombinant retroviral vector comprising: (a) a first promoter operably linked to a first polynucleotide encoding a CD7 chimeric antigen receptor (CD7 CAR) as outlined herein; (b) a second promoter operably linked to a second polynucleotide encoding a CD7 protein expression blocker (CD7 PEBL); (c) a third promoter operably linked to a third polynucleotide encoding a suicide gene, e.g., CD20; and (d) a fourth promoter operably linked to a fourth polynucleotide encoding a CD3 protein expression blocker (CD3 PEBL) as outlined herein.

In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:11 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:12 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:13 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:14 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO: 15 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:16 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:17 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:18 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:19 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:20 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:21 is administered to a subject having cancer. In some embodiments, a therapeutic amount of an engineered immune cell (e.g., engineered CD3+ T cell, engineered CD4+ T cell, or engineered CD8+ T cell) or a population thereof comprising a nucleic acid construct comprising SEQ ID NO:22 is administered to a subject having cancer. In some embodiments, a therapeutic amount of an engineered immune cell (e.g., engineered CD3+ T cell, engineered CD4+ T cell, or engineered CD8+ T cell) or a population thereof comprising a nucleic acid construct comprising SEQ ID NO:23 is administered to a subject having cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 CAR of SEQ ID NO:29 is administered to a subject with cancer. In some embodiments, a therapeutic amount of a population of engineered immune cells (e.g., engineered CD3+ T cells, engineered CD4+ T cells, or engineered CD8+ T cells) comprising a CD7 CAR of SEQ ID NO:31 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 and a CD7 CAR of SEQ ID NO:29 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 and a CD7 CAR of SEQ ID NO:29 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 and a CD7 CAR of SEQ ID NO:31 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 and a CD7 CAR of SEQ ID NO:31 is administered to a subject with cancer.

In some embodiments, a therapeutic amount of a population of engineered immune cells (e.g., engineered CD3+ T cells, engineered CD4+ T cells, or engineered CD8+ T cells) is administered to a subject with cancer, wherein the engineered immune cells comprise SEQ ID NO: 95.

In certain embodiments, the cancer is a T cell malignancy, e.g., T cell leukemia or T cell lymphoma, such a T-cell acute lymphoblastic leukemia, T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, mycosis fungoides, Sezary syndrome, primary cutaneous gamma-delta T-cell lymphoma, peripheral T-cell lymphoma not otherwise specified, angioimmunoblastic T-cell lymphoma, anaplastic large cell lymphoma. In certain embodiments, the T cell malignancy is early T-cell progenitor acute lymphoblastic leukemia (ETP-ALL).

In some embodiments, the engineered immune cell is autologous to the subject in need of treatment, e.g., cancer treatment. In other embodiments, the engineered immune cell is allogeneic to the subject in need of treatment.

In certain embodiments, the engineered immune cell is administered into the subject by intravenous infusion, intra-arterial infusion, direct injection into tumor and/or perfusion of tumor bed after surgery, implantation at a tumor site in an artificial scaffold, intrathecal administration, and intraocular administration.

In certain embodiments, the engineered immune cell is administered by infusion into the subject. Methods of infusing immune cells (e.g., allogeneic or autologous immune cells) are known in the art. A sufficient number of cells are administered to the recipient in order to ameliorate the symptoms of the disease. Typically, dosages of 10⁷ to 10¹⁰ cells are infused in a single setting, e.g., dosages of 10⁹ cells. Infusions are administered either as a single 10⁹ cell dose or divided into several 10⁹ cell dosages. The frequency of infusions can be daily, every 2 to 30 days or even longer intervals if desired or indicated. The quantity of infusions is generally at least 1 infusion per subject and preferably at least 3 infusions, as tolerated, or until the disease symptoms have been ameliorated. The cells can be infused intravenously at a rate of 50-250 ml/hr. Other suitable modes of administration include intra-arterial infusion, intraperitoneal infusion, direct injection into tumor and/or perfusion of tumor bed after surgery, implantation at the tumor site in an artificial scaffold, intrathecal administration. Methods of adapting the present invention to such modes of delivery are readily available to one skilled in the art.

In certain embodiments, the method of treating cancer according to the present invention is combined with at least one other known cancer therapy, e.g., radiotherapy, chemotherapy, or other immunotherapy.

In other aspects, also provided is use of an engineered immune cell having any of the embodiments described herein for treating cancer, comprising administering a therapeutic amount of the engineered immune cell to a subject in need thereof. In certain embodiments, the cancer is a T cell malignancy. In certain embodiments, the T cell malignancy is early T-cell progenitor acute lymphoblastic leukemia (ETP-ALL).

In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, and intrathecal administration.

In some aspects, provided herein are methods for modulating an immune system in a subject in need thereof, the method comprising: administering a therapeutically effective amount of engineered immune cells. The engineered immune cells can comprise: (a) a first nucleic acid sequence encoding (i) a domain that binds to a subunit of a T cell receptor (TCR) complex linked to (ii) a synthetic localizing domain; (b) a second nucleic acid sequence encoding a chimeric antigen receptor (CAR); and (c) a third nucleic acid sequence encoding a surface polypeptide binding domain linked to a synthetic surface polypeptide localizing domain. The domain that binds to the subunit of a TCR complex linked to the synthetic localizing domain can downregulate surface expression of the TCR complex, thereby modulating the immune system of the subject in need thereof.

In some embodiments, modulating an immune system as used herein may refer to using a therapeutic agent to modify an immune response in a subject. In some embodiments, modulating an immune system may comprise administering a therapeutically effective amount of an agent to modify an immune response in a subject. In some embodiments, modulating an immune system may comprise administering an engineered immune cell described herein to reduce the risk of a subject mounting an immune response to an agent.

In some aspects, provided herein are methods for treating a disease in a subject in need thereof, the method comprising: administering a therapeutically effective amount of engineered immune cells. The engineered immune cells can comprise: (a) a first nucleic acid sequence encoding (i) a domain that binds to a subunit of a T cell receptor (TCR) complex linked to (ii) a synthetic localizing domain; (b) a second nucleic acid sequence encoding a chimeric antigen receptor (CAR); and (c) a third nucleic acid sequence encoding a surface polypeptide binding domain linked to a synthetic surface polypeptide localizing domain. The domain that binds to the subunit of a TCR complex linked to the synthetic localizing domain can downregulate surface expression of the TCR complex, thereby treating the disease in the subject in need thereof.

In some embodiments, a subject may have symptoms of graft-versus-host disease (GvHD). In some embodiments, a subject may have a condition and/or associated with an allogeneic transplant. In some embodiments, modulating an immune system can comprise preventing, alleviating, or reducing the risk of GvHD and/or symptoms of GvHD. In some embodiments, modulating an immune system in a subject suffering from GvHD may comprise the killing of CD7+ T cells in vitro or ex vivo due to a CD7 CAR. Without wishing to be bound by theory, a PEBL described herein may downregulate a TCR/CD3 complex on a cell surface thereby reducing the risk of a subject developing graft-versus-host-disease (GvHD) in response to an administered agent. The engineered immune cells may reduce the risk of developing GvHD in a subject. In some embodiments, a subject may have a reduced risk of developing a graft-versus-host disease (GvHD) response after administering the engineered immune cells described herein, compared to a risk associated with administration of otherwise identical immune cells comprising a CAR alone or a CD7 PEBL alone.

In some embodiments, a subject may have cancer. In some embodiments, the cancer may be a T cell malignancy, e.g., T cell leukemia or T cell lymphoma, such as T-cell acute lymphoblastic leukemia, T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, mycosis fungoides, Sezary syndrome, primary cutaneous gamma-delta T-cell lymphoma, peripheral T-cell lymphoma not otherwise specified, angioimmunoblastic T-cell lymphoma, anaplastic large cell lymphoma.

In some embodiments, the cancer comprises T-cell acute lymphoblastic leukemia (T-ALL), early T-cell progenitor acute lymphoblastic leukemia (ETP-ALL), acute myeloid leukemia, or T-cell lymphoblastic lymphoma. In some embodiments, the cancer can be a CD7 positive cancer. In some embodiments, modulating the immune system can comprise treating the cancer and/or treating symptoms of the cancer.

In some embodiments, the CAR comprises a target binding domain that binds to the surface polypeptide. In some embodiments, the surface polypeptide can be CD7. In some embodiments, the subunit of the TCR complex is CD3ε, CD3γ, or CD36. In some embodiments, the subunit of the TCR complex is CD3ε. In some embodiments, the domain binding to the subunit of the TCR complex (e.g., CD3ε) can be an antibody or an antigen binding domain. In some embodiments, the antibody binds to CD3ε (e.g., an anti-CD3ε antibody). In some embodiments, the antibody can be a single chain Fv (scFv) or a single domain antibody (sdAb).

In some embodiments, the anti-CD3ε antibody comprises a heavy chain complementarity-determining region (HC CDR) 1 of SEQ ID NOs: 212 or 215, a HC CDR2 of SEQ ID NOs: 213 or 216, a HC CDR3 of SEQ ID NOs: 214 or 217, and a light chain (LC) CDR1 of SEQ ID NOs: 218 or 221, a LC CDR2 of SEQ ID NOs: 219 or 222, a LC CDR3 of SEQ ID NOs: 220 or 223. In some embodiments, the anti-CD3ε antibody comprises a heavy chain variable domain having an amino acid sequence with at least 70%, at least 75%, at least 80%, at least 85%, at least 90%, at least 95%, at least 96%, at least 97%, at least 98%, at least 99%, or 100% sequence identity to SEQ ID NO: 114. In some embodiments, the anti-CD3ε antibody comprises a light chain variable domain having an amino acid sequence with at least 70%, at least 75%, at least 80%, at least 85%, at least 90%, at least 95%, at least 96%, at least 97%, at least 98%, at least 99%, or 100% sequence identity to SEQ ID NO: 115. In some embodiments, the anti-CD3ε antibody comprises an amino acid sequence having at least 70%, at least 75%, at least 80%, at least 85%, at least 90%, at least 95%, at least 96%, at least 97%, at least 98%, at least 99%, or 100% sequence identity to SEQ ID NO: 104.

In some embodiments, the engineered immune cell can be a T cell or a natural killer (NK) cell. In some embodiments, the engineered immune cell can be a T cell, a natural killer (NK) cell, a NK/T cell, a monocyte, a macrophage, or a dendritic cell. In some embodiments, the engineered immune cell can be a CD4+ T cell or a CD8+ T cell. In some embodiments, the engineered immune cells suppress tumor growth in a subject. In some embodiments, the engineered immune cells suppress, reduce, or prevent tumor growth in a subject. In some embodiments, the engineered immune cells shrink the tumor to at least about two times, at least about three times, at least about four times, at least about five times, at least about six times, at least about seven times, at least about eight times, at least about nine times, or at least about ten times its size prior to contact with the engineered immune cells. In some embodiments, the engineered immune cells may reduce the tumor burden by at least about two times, at least about three times, at least about four times, at least about five times, at least about six times, at least about seven times, at least about eight times, at least about nine times, or at least about ten times compared to a tumor burden prior to contact with the engineered immune cells.

In some embodiments, any engineered immune cell described herein may be used to modulate an immune system in a subject in need thereof. An exemplary engineered immune cell may comprise a bicistronic vector comprising CD7 CAR and CD7 PEBL. An exemplary engineered immune cell may comprise a bicistronic vector comprising CD3 PEBL and a kill gene.

In some aspects, provided herein are methods of depleting engineered immune cells after administration in a subject in need thereof. In some aspects, the method comprises administering to the subject a population of engineered immune cells that express: (a) a chimeric antigen receptor (CAR) specific for a T cell surface antigen; (b) a first protein expression blocker (PEBL) that downregulates cell surface expression of the T cell surface antigen; (c) a second PEBL that downregulates cell surface expression of a subunit of a T cell receptor (TCR) complex; and (d) a kill protein.

In some embodiments, the kill protein comprises CD20 or a derivative thereof. In some embodiments, the kill protein comprises CD20, a derivative thereof, or a portion thereof. In some embodiments, the kill protein comprises a modified CD20. In some embodiments, the kill protein comprises a truncated CD20 (CD20t). In some embodiments, the CD20t comprises an amino acid sequence of SEQ ID NO: 106. In some embodiments, the CD20t comprises an amino acid sequence with at least 70%, at least 75%, at least 80%, at least 85%, at least 90%, at least 95%, at least 96%, at least 97%, at least 98%, at least 99%, or 100% sequence identity to SEQ ID NO: 106.

In some embodiments, a method of depleting engineered immune cells after administration in a subject in need thereof may comprise administering rituximab to the subject to induce elimination of the population of engineered immune cells, thereby depleting the population of engineered immune cells. In some embodiments, a method of depleting engineered immune cells after administration in a subject in need thereof may comprise administering ofatumumab to the subject to induce elimination of the population of engineered immune cells, thereby depleting the population of engineered immune cells. In some embodiments, the subject may have been diagnosed with an immune condition. In some embodiments, the subject may have been diagnosed with a T cell malignancy. In some embodiments, the CAR may be specific for CD7. In some embodiments, the subunit of the TCR complex downregulated by the second PEBL is CD3ε, CD3γ, or CD36. In some embodiments, the subunit of the TCR complex downregulated by the second PEBL is CD3ε.

In some embodiments, the population of engineered immune cells comprises T cells or natural killer (NK) cells. In some embodiments, the population of engineered immune cells can be T cells, natural killer (NK) cells, NK/T cells, monocytes, macrophages, or dendritic cells. In some embodiments, the population of engineered immune cells can be CD4+ T cells or CD8+ T cells.

### EMBODIMENT PARAGRAPHS

Embodiment 1. An engineered immune cell, comprising: (a) a first nucleic acid comprising a nucleotide sequence encoding a first target binding domain linked to a first localizing domain; (b) a second nucleic acid comprising a nucleotide sequence encoding a chimeric antigen receptor (CAR), wherein the CAR comprises a second target binding domain; (c) a third nucleic acid comprising a nucleotide sequence encoding a third target binding domain linked to a second localizing domain; and wherein the first and second target binding domain bind to a first immune cell surface receptor molecule and the third target binding domain binds to a second immune cell surface receptor molecule.

Embodiment 2. The engineered immune cell of embodiment 1, wherein the first immune cell surface receptor molecule is CD7.

Embodiment 3. The engineered immune cell of embodiment 1 or 2, wherein the second immune cell surface receptor molecule is CD3.

Embodiment 4. The engineered immune cell of any one of embodiments 1 to 3, wherein the first and second target binding domains are first and second antibodies, respectively, and wherein the amino acid sequence of the first antibody and the amino acid sequence of the second antibody are at least 80% identical.

Embodiment 5. The engineered immune cell of embodiment 4, wherein the amino acid sequences of CDRs of the first antibody and the amino acid sequences of CDR1-3 of the second antibody are at least 90% identical.

Embodiment 6. The engineered immune cell of any one of embodiments 4 to 5, wherein the first antibody comprises CDR sequences of TH69.

Embodiment 7. The engineered immune cell of any one of embodiments 4 to 6, wherein the second antibody comprises CDR sequences of TH69.

Embodiment 8. The engineered immune cell of any one of embodiments 3 to 7, wherein the third target binding domain comprises an anti-CD3 antibody.

Embodiment 9. The engineered immune cell of any one of embodiments 1 to 8, wherein the first and second nucleic acids are located in a first expression vector.

Embodiment 10. The engineered immune cell of embodiment 9, wherein the first expression vector is a bicistronic lentiviral expression vector.

Embodiment 11. The engineered immune cell of embodiment 9 or 10, wherein the first and second nucleic acids are operably linked by Internal Ribosome Entry Site (IRES) or a ribosomal codon skipping site.

Embodiment 12. The engineered immune cell of embodiment 11, wherein the ribosomal codon skipping site comprises a 2A self-cleaving peptide.

Embodiment 13. The engineered immune cell of embodiment 9, wherein the third nucleic acid is located in a second expression vector.

Embodiment 14. The engineered immune cell of any one of embodiments 1 to 13, further comprising a fourth nucleic acid, wherein the fourth nucleic acid comprises a suicide gene or a kill gene.

Embodiment 15. The engineered immune cell of embodiment 14, wherein the suicide gene or a kill gene comprises CD20 or a derivative thereof.

Embodiment 16. The engineered immune cell of embodiment 14 or 15, wherein the fourth nucleic acid is located in the second expression vector.

Embodiment 17. The engineered immune cell of any one of embodiments 1 to 16, wherein the second expression vector is a lentiviral vector.

Embodiment 18. The engineered immune cell of any one of embodiments 1 to 17, wherein the first and second localization domains comprise an endoplasmic reticulum (ER) retention sequence, a Golgi retention sequence, a proteasome localizing sequence, or a transmembrane domain.

Embodiment 19. The engineered immune cell of embodiment 18, wherein the ER retention sequence comprises KDEL (SEQ ID NO: 73) or KKXX.

Embodiment 20. The engineered immune cell of embodiment 18 or 19, wherein the first and second localizing domain are the same amino acid sequences.

Embodiment 21. The engineered immune cell of embodiment 18 or 19, wherein the first and second localizing domain comprises different amino acid sequences.

Embodiment 22. The engineered immune cell of any one of embodiments 1 to 21, wherein the first nucleic acid further comprises a nucleic acid sequence encoding a linker that couples the first target binding domain and the first localization domain.

Embodiment 23. The engineered immune cell of any one of embodiments 1 to 22, wherein the CAR comprises a transmembrane domain, a 4-1BB intracellular signaling domain, and a CD3ζ intracellular signaling domain.

Embodiment 24. The engineered immune cell of any one of embodiments 1 to 23, wherein the surface expression of the first and second cell surface receptor molecules are downregulated in the engineered immune cell.

Embodiment 25. The engineered immune cell of any one of embodiments 1 to 24, wherein the engineered immune cell is a T cell or an NK cell.

Embodiment 26. A method of producing an engineered immune cell expressing a chimeric antigenic receptor (CAR) targeting a cell surface protein, the method comprising: (i) introducing into an immune cell: (a) a first nucleic acid comprising a nucleotide sequence encoding a first target binding domain linked to a first localizing domain; (b) a second nucleic acid comprising a nucleotide sequence encoding a chimeric antigen receptor (CAR), wherein the CAR comprises a second target binding domain; (c) a third nucleic acid comprising a nucleotide sequence encoding a third target binding domain linked to a second localizing domain, wherein the first and second target binding domain bind to a first immune cell surface receptor molecule and the third target binding domain binds to a second immune cell surface receptor molecule; and (ii) culturing the engineered immune cell, thereby producing said engineered immune cell.

Embodiment 27. The method of embodiment 26, wherein expression of the first or third target binding domains prevents fratricide of the engineered immune cell during step (ii)

Embodiment 28. The method of embodiment 26 or 27, wherein the first immune cell surface receptor molecule is CD7.

Embodiment 29. The method of any one of embodiments 26 to 28, wherein the second immune cell surface receptor molecule is CD3.

Embodiment 30. The method of any one of embodiments 26 to 29, wherein the immune cell is a T cell or an NK cell.

Embodiment 31. The method of any one of embodiments 26 to 30, wherein the introduction of the first, second, and third nucleic acids does not affect expansion or function of the engineered immune cell.

Embodiment 32. The method of any one of embodiments 26 to 31, wherein the introduction of the third nucleic acid does not affect the cytotoxicity of the engineered immune cell compared to an engineered immune cell transduced with the first and second nucleic acids, but not with the third nucleic acid.

Embodiment 33. A method for treating a cancer in a subject in need thereof, comprising: administering a therapeutically effective amount of an engineered immune cell, wherein said engineered immune cell comprises: (a) a first nucleic acid comprising a nucleotide sequence encoding a first target binding domain linked to a first localizing domain; (b) a second nucleic acid comprising a nucleotide sequence encoding a chimeric antigen receptor (CAR), wherein the CAR comprises a second target binding domain; (c) a third nucleic acid comprising a nucleotide sequence encoding a third target binding domain linked to a second localizing domain, wherein the first and second target binding domain bind to a first immune cell surface receptor molecule and the third target binding domain binds to a second immune cell surface receptor molecule; and thereby treating the cancer.

Embodiment 34. The method of embodiment 33, wherein the first immune cell surface receptor molecule is CD7.

Embodiment 35. The method of embodiment 33 or 34, wherein the second immune cell surface receptor molecule is CD3.

Embodiment 36. The method of any one of embodiments 33 to 35, wherein the engineered immune cell is a T cell.

Embodiment 37. The method of any one of embodiments 33 to 36, wherein the cancer comprises CD7 positive cancer.

Embodiment 38. The method of any one of embodiments 33 to 37, wherein the cancer comprises acute lymphoblastic leukemia (T-ALL), early T-cell progenitor acute lymphoblastic leukemia (ETP-ALL), acute myeloid leukemia, or T-cell lymphoblastic lymphoma.

Embodiment 39. The method of any one of embodiments 33 to 38, wherein the administration of the engineered immune cell suppresses tumor cell growth in the subject.

Embodiment 40. A method for preventing, alleviating, or reducing symptoms of graft-versus-host disease (GvHD) in a subject in need thereof, comprising: administering a therapeutically effective amount of an engineered immune cell, wherein said engineered immune cell comprises: (a) a first nucleic acid comprising a nucleotide sequence encoding a first target binding domain linked to a localizing domain; (b) a second nucleic acid comprising a nucleotide sequence encoding a chimeric antigen receptor (CAR), wherein the CAR comprises a second target binding domain; (c) a third nucleic acid comprising a nucleotide sequence encoding a third target binding domain linked to a localizing domain, wherein the first and second target binding domain bind to a first immune cell surface receptor molecule and the third target binding domain binds to a second immune cell surface receptor molecule; wherein the expression of the third target binding domain downregulates surface expression of an immune cell receptor of the engineered immune cell, preventing immune cell receptor mediated antigen recognition, thereby preventing, alleviating, or reducing symptoms of GvHD.

Embodiment 41. The method of embodiment 40, wherein the first immune cell surface receptor molecule is CD7.

Embodiment 42. The method of embodiment 40 or 41, wherein the second immune cell surface receptor molecule is CD3.

Embodiment 43. The method of any one of embodiments 40 to 42, wherein the engineered immune cell is a T cell or an NK cell.

Embodiment 44. A kit comprising: a first expression vector comprising: (a) a first nucleic acid comprising a nucleotide sequence encoding a first target binding domain linked to a localizing domain; (b) a second nucleic acid comprising a nucleotide sequence encoding a chimeric antigen receptor (CAR), wherein the CAR comprises a second target binding domain; a second expression vector comprising a third nucleic acid comprising a nucleotide sequence encoding a third target binding domain linked to a localizing domain; and wherein the first and second target binding domain bind to a first immune cell surface receptor molecule and the third target binding domain binds to a second immune cell surface receptor molecule.

Embodiment 45. The kit of embodiment 44, wherein the first expression vector is a bicistronic retroviral expression vector.

Embodiment 46. The kit of embodiment 44 or 45, wherein the second expression vector is a retroviral vector.

Embodiment 47. The kit of embodiment 45 or 46, wherein the first expression vector is a bicistronic lentiviral vector.

Embodiment 48. The kit of any one of embodiments 45 to 47, wherein the second expression vector is a lentiviral vector.

Embodiment 49. The kit of any one of embodiments 44 to 48, wherein the first immune cell surface receptor molecule is CD7.

Embodiment 50. The kit of any one of embodiments 44 to 49, wherein the second immune cell surface receptor molecule is CD3.

Embodiment 51. The kit of any one of embodiments 44 to 50, wherein the first and second nucleic acids are operably linked by Internal Ribosome Entry Site (IRES) or a ribosomal codon skipping site.

Embodiment 52. The kit of embodiment 51, wherein the ribosomal codon skipping site comprises a 2A self-cleaving peptide.

### EXAMPLES

### Example 1: Generation of Anti-CD7 CAR-T Cells Lacking T-Cell Receptor without Gene Editing for Allogeneic CAR-T Cell Therapy of T-Cell Acute Lymphoblastic Leukemia (T-ALL)

CAR-T cell therapy has revolutionized the treatment of B-lineage ALL and other B-cell malignancies but the development of cell therapies for T-cell malignancies has lagged behind. In T-ALL, CD7 is an excellent CAR target because it is consistently and highly expressed in leukemic cells and is retained during chemotherapy and at relapse. However, CD7 is also expressed in normal T cells and expression of anti-CD7 CARs triggers T-cell fratricide, resulting in a marked decrease in viable and/or effective CAR-T cells. We developed an autologous anti-CD7 CAR-T cell product (PCART7) by utilizing a CD7 protein expression blocker (PEBL) to downregulate CD7 surface expression and prevent fratricide. PEBLs consist of a target binding domain fused to an intracellular retention domain that enables relocalization of surface proteins intracellularly (Png et al. Blood Adv 2017; Kamiya et al. Blood Adv 2018; Kamiya et al., JCI 2019). Autologous T cells lacking CD7 and expressing anti-CD7 CARs can be effectively produced under cGMP conditions and the technology is being tested in clinical trials.

Generation of autologous CAR-T cells for the treatment of relapsed/refractory T-ALL may be hampered by the presence of a high percentage of leukemic cells in peripheral blood and/or low T cell numbers. An allogeneic anti-CD7 CAR-T cell product available “off-the shelf” would represent a tool to reduce leukemic burden as a bridge to transplant or to autologous CAR-T cell therapy. Because allogeneic T cells can be activated upon contact of their T-cell receptor (TCR)/CD3 complex with the host tissues, their infusion carries a risk of causing graft versus host disease (GvHD). In this example, a CD3 PEBL that can effectively downregulate surface TCR/CD3 was developed. In addition, a lentiviral-based process that allows simultaneous expression of this PEBL together with anti-CD7 PEBL and anti-CD7 CAR was developed. Surface CD7 and TCR/CD3 were retained intracellularly by the PEBLs, thereby minimizing fratricide and abrogating TCR-mediated antigen recognition.

### Methods

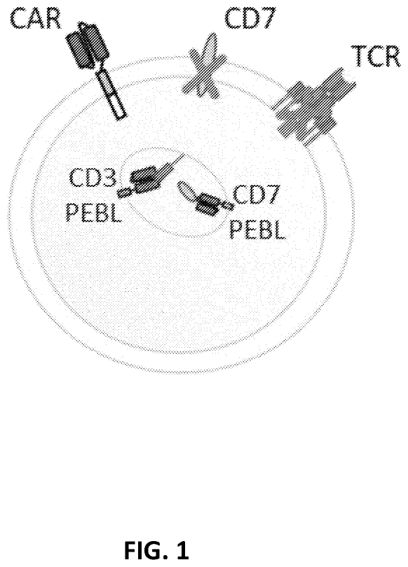

Allogeneic PCART7 (allo-PCART7) cells were manufactured from healthy donors T cells by using a double transduction with a bicistronic lentiviral vector delivering anti-CD7 PEBL and anti-CD7 CAR, and a second lentiviral vector delivering anti-CD3 PEBL. FIG. 1 shows composition of allogeneic PCART7-CD3PEBL or allo-PCART7 cells.

PBMC cells were thawed and recovered. T cells were activated with TransAct and 120 IU/ml IL-2. Activated T cells were transduced with lentivirus. On Day 3, LVV media (lentiviral media) was removed and cells were passaged. Electroporation for T cells was done by using Buffer T from Neon Transfection System. On Day 15, CD3 depletion was performed to deplete CD3+ population from all-cell product. Cells were labelled with CD3 magnetic beads, then separated by magnetic separation using LD columns.

### TCR Responsiveness Assay

This experiment was performed to test TCR responsiveness to TransAct in PCART7 cells with CD3 downregulation by CD3 PEBL (e.g., OKT3 PEBL5).

For TransAct stimulation, cells were counted and seeded at 1×10⁶ cells per ml with fresh culture media in 24-well plate. Next, 10 ul of TransAct (10 ul per 10⁶ cells) and 1201U/ml of IL-2 was added into respective wells. IL-2 containing media was added into non-treated control wells. Cells were incubated for 48 hours and collected and stained for FACS analysis.

### Chronic Stimulation Assay

This assay was performed to determine cell surface CD3 expression of PCART7-CD3 PEBL cells upon repeated stimulation with TransAct or irradiated CD3-KO Jurkat cells to assess the effectiveness of CD3 PEBL in retaining CD3.

On Day 0, cells were counted before seeding in a 6 well plate at 2×10⁶ cells per condition (5×10⁵ cells per ml). Cells were stimulated and counted every 3-4 days for the following groups:
Non-stimulated.
TransAct (10 ul per ml).
Irradiated CD3-KO Jurkat cells at 1:1 E:T ratio.

On day 21, cells were stained with CAR, CD3, and CD25 markers for FACS analysis.

### Cytotoxicity Assay—Incucyte

This experiment was performed to evaluate cytotoxic function of allo-PCART7 cells. Effector and target cells were co-cultured in a 96-well flat-bottom plate. The following conditions were plated: target cells only (Jurkat-GFP or Nalm-6 GFP), effector cells+Jurkat GFP, and effector cells+Nalm6-GFP. Untransduced T cells were used as a control.

### Results

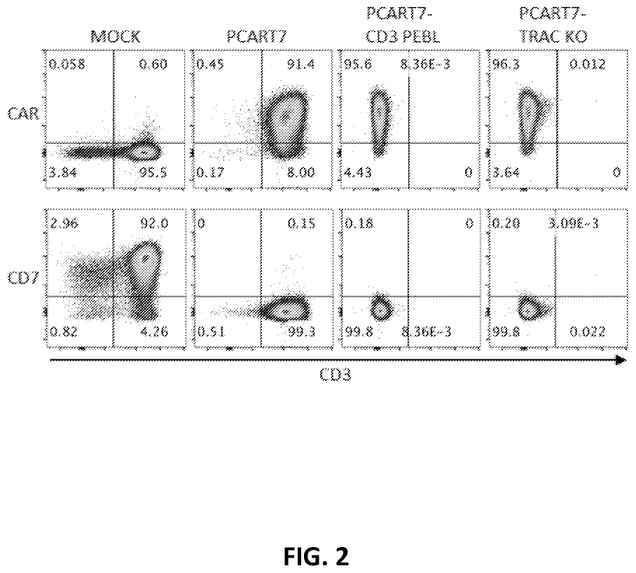

As shown in FIG. 2, more than 90% of the allo-PCART7 cells were of the desired CAR+CD7−CD3− phenotype. Following depletion of residual CD3/TCRαβ+ cells, allogeneic PCART7 cell purity was >99%. These results were reproduced across multiple donors. Furthermore, the efficient downregulation of surface TCR/CD3 by CD3 PEBL was similar to that achieved by CRISPR knockout of TRAC (PCART7-TRAC KO).

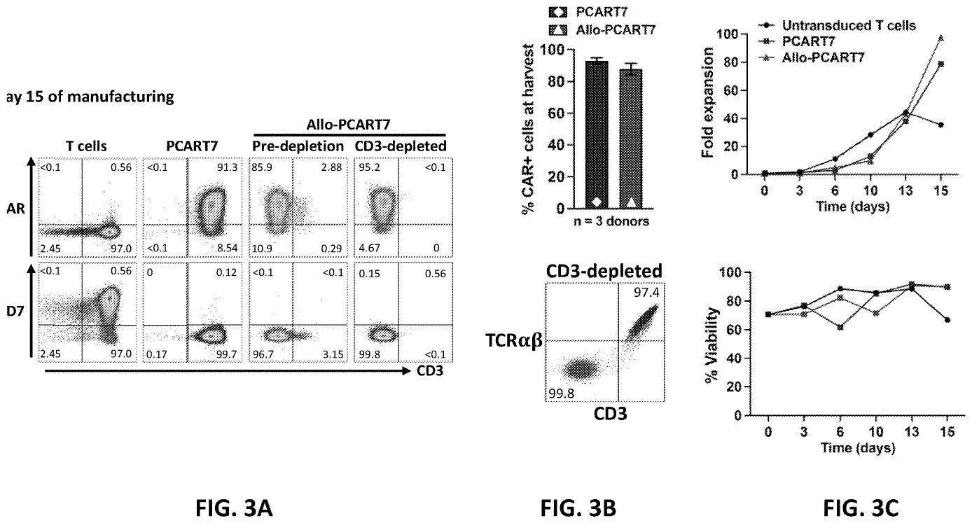

Further, results in FIGS. 3A-3C show phenotypic characteristics of allo-PCART7. FIG. 3A shows that 15 days after the start of manufacturing, engineered T cells which were stained with respective antibodies had desired surface phenotype as analyzed by flow cytometry. CD3-depletion was performed and this process was able to increase the quality of allo-PCART7 by reducing the percentage of CD3+ cells to less than 0.1%. FIG. 3B top panel shows that at the time of harvest, the percentage of CAR+ cells for PCART7 and allo-PCART7 were similar between the two groups. However, as depicted in FIG. 3B bottom panel, in contrast to PCART7, allo-PCART7 cells are TCRαβ negative and CD3 negative as analyzed by flow cytometry. FIG. 3C shows that PCART7 and allo-PCART7 have similar expansion and cell viability throughout the 15-day manufacturing process.

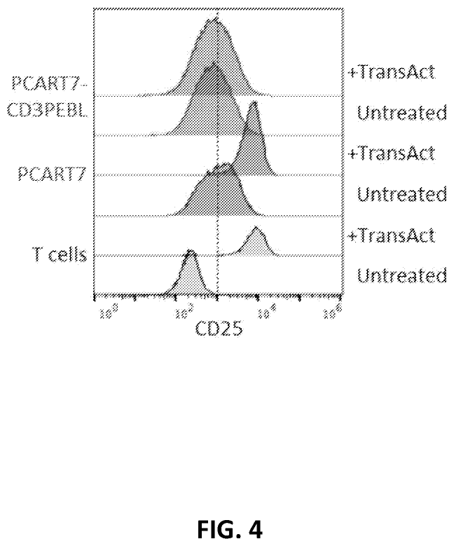

In order to investigate if allo-PCART7 can be activated through the T cell receptor/CD3 complex (TCR), cells were stimulated with TransAct, a polymeric nanomatrix covalently linked to anti-CD3 and anti-CD28 antibodies for activation of T-cells. Cells were treated for 72 hours with or without TransAct before detecting CD25 activation marker by flow cytometry. CD25 is a cell surface marker for activated lymphocytes during active immune response such as in GvHD. FIG. 4 depicts that allogeneic PCART7 were not responsive to TransAct stimulation and did not upregulate CD25. This result shows that allogeneic PCART7 cells cannot be stimulated through the TCR.

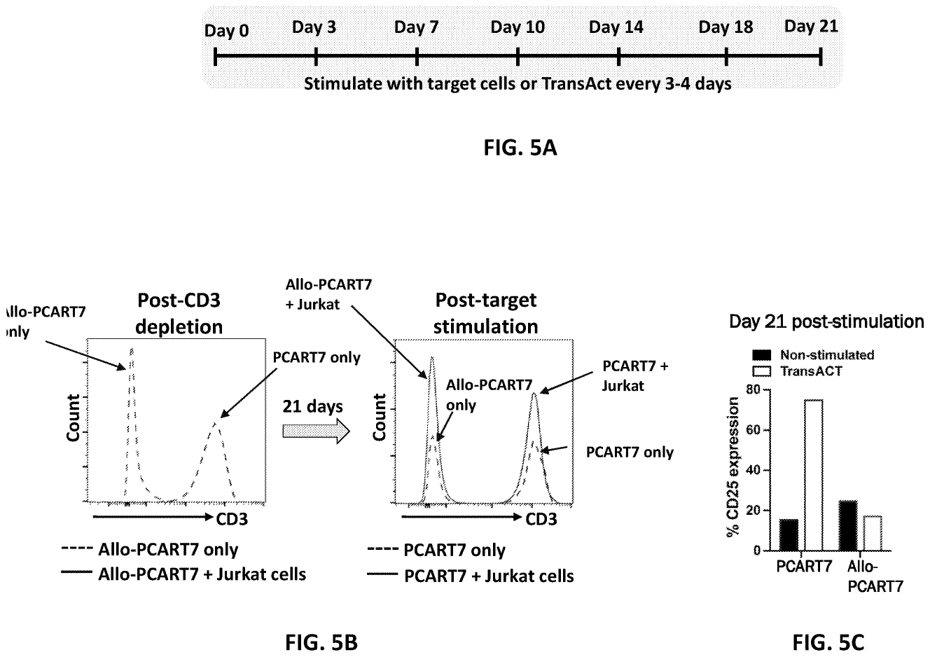

Further, results in FIGS. 5A-5C show that allo-PCART7 cells had effective CD3 downregulation. FIG. 5A shows the experimental timeline. Briefly, allo-PCART7 cells were stimulated with target cells or TransAct (CD3 and CD28 activation of T-cells) every 3-4 days over a period of 21 days to assess the long-term effectiveness of CD3 PEBL in retaining CD3 intracellularly. FIG. 5B shows that allo-PCART7 cells did not express CD3 and are distinguished from CD3-positive PCART7 cells both before and after stimulation with CD3-KO Jurkat cells (indicated by solid line). After stimulation with CD3-KO Jurkat cells, the number of allo-PCART7 cells and PCART7 cells were increased, but CD3 level remained unchanged, indicating that in allo-PCART7 cells, CD3 remain effectively retained inside the cells by CD3 PEBL. Solid line indicates co-culture of CD3-KO Jurkat cells with either allo-PCART7 cells or PCART7 cells. Dashed line indicates either allo-PCART7 cells only or PCART7 cells only. FIG. 5C shows that at Day 21 post-stimulation with TransAct, % expression level of CD25, which is a cell surface marker for activated lymphocytes during active immune response such as in GvHD, remained unchanged in allo-PCART7 cells. On the other hand, PCART7 cells showed a large increase in % CD25 expression after stimulation.

In vitro cytotoxicity experiment was performed. Briefly, non-transduced T cells, PCART7-CD3PEBL (allo-PCART7), or PCART7 cells were co-cultured with GFP expressing CD7+ Jurkat cells (left) or CD7 negative Nalm6 cells (right) at 1:1 effector-to-target (E:T) ratio. GFP intensities from target cells were monitored using IncuCyte® live cell imaging system.

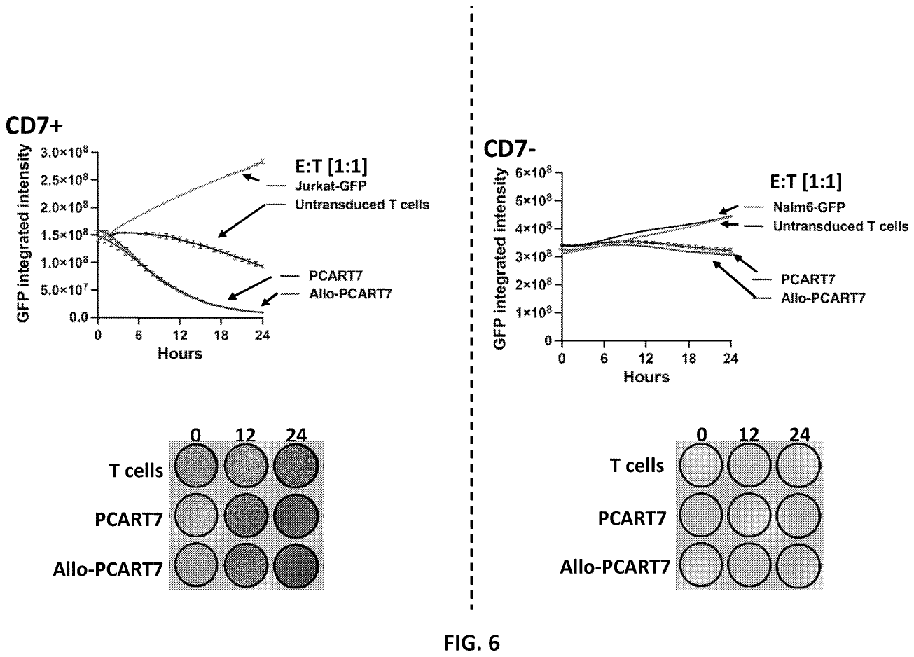

FIG. 6 depicts that allo-PCART7 cells can effectively and specifically kill T-cell acute lymphoblastic leukemia (T-ALL) cells in vitro. In this experiment, the anti-tumor potential of allo-PCART7 cells was evaluated in vitro by co-culturing allo-PCART7 cells with T-ALL cells at 1:1 effector-to-target (E:T) ratio. CD7+ and CD7 negative ALL cells were engineered to express green fluorescent protein, and the co-culture experiment was performed using an IncuCyte® system to observe the effect on target cells in real-time. The value on the y-axis correlates with the number of target cells in the well and the x-axis is the co-culture time. As shown on the left panel, CD7+T-ALL cells (Jurkat-GFP) expand over time when cultured alone. However, once these cells were co-cultured with PCART7 cells or allo-PCART7 cells, there was a decrease in CD7+ target cells, reaching an undetectable level. As shown on the right panel, CD7 negative B cell precursor leukemia cell line (Nalm6-GFP) was used as control target cells. Co-culture with engineered T cells, either PCART7 cells or allo-PCART7 cells, did not affect CD7 negative cells, indicating that there was minimal non-specific killing by these engineered T cells.

In conclusion, this study demonstrates the feasibility of using two PEBLs simultaneously for intracellular protein retention without perturbing critical CAR-T cell functions. The use of PEBL technology to generate allogeneic PCART7 cells fits seamlessly in cGMP manufacturing protocols and avoids the risk of genotoxicity arising from the use of gene-editing techniques. Because the resulting allogeneic PCART7 cells had high potency and were devoid of TCR reactivity, they represent an attractive new tool for off-the-shelf CAR-T cell therapy of T-ALL and other CD7+ malignancies.

### Example 2: Allogeneic Anti-CD7 CAR-T Cells Lacking Cell Surface CD7 and T Cell Receptors (Allo-PCART7) Exert Potent Anti-Tumor Efficacy but are not Xenoreactive

In this example, validation of allo-PCART7 product was performed in vivo.

### Methods

### Anti-Tumor Efficacy Validation in NSG Mice

This experiment was performed to validate anti-tumor efficacy of allo-PCART7 cells in NSG mice.

On Day 0, each NSG mice was injected intravenously with 1×10⁶ CCRF-CEM leukemic cells expressing firefly luciferase and green fluorescent protein. On Day 3, each mouse was injected with 150 mg/kg D-luciferin intraperitoneally to perform IVIS imaging prior to intravenous infusion of 1×10⁷ allo-PCART7 cells and intraperitoneal infusion of 20,000 IU IL-2. Additional IVIS imaging was performed on Days 7, 14, and 21. Blood samples were collected on Days 10, 17, and 24. From Day 3 onwards, 20,000 IU of IL-2 was infused three times a week.

### Xenoreactivity Assessment In Vivo (GvHD In Vivo Experiment)

This experiment was performed to assess xenoreactivity of allo-PCART7 product in NSG mice.

On Day −1, gamma irradiation of NSG mice at 2.5Gy was performed. On Day 0, allo-PCART7 cells were infused intravenously and IL-2 infusion was also performed intraperitoneally. On Day 7 and at the end of the experiment, blood samples were collected. After Day 0, weight measurement and intraperitoneal infusion of 20,000 IU of IL-2 were performed three times a week until the end of the experiment.

### Results

In order to test the in vivo efficacy of allo-PCART7 cells, an NSG tumor xenograft mouse model was used. Allogenic PCART7-CD3PEBL cells (anti-CD7 CAR-T cells lacking CD7 and TCR/CD3) were produced using the protocol described in Example 1. Briefly, NSG mice were intravenously injected with 1×10⁶ CCRF-CEM leukemic cells expressing firefly luciferase and green fluorescent protein. Three days later, tumor-bearing mice were treated with PBS (vehicle), non-transduced T cells, PCART7 cells, or PCART7-CD3PEBL (allo-PCART7) cells. Bioluminescence from tumor cells were detected using IVIS imaging.

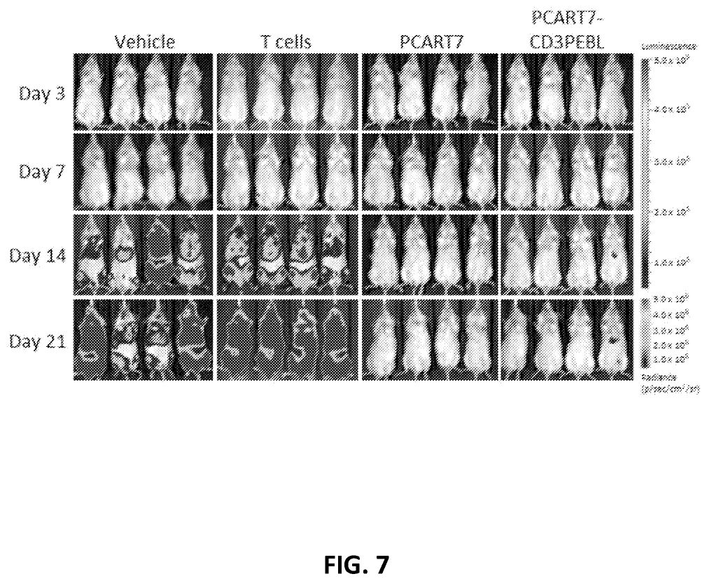

As shown in FIG. 7, in a xenograft model using the CCRF-CEM T-ALL cell line, allogeneic PCART7 cells effectively suppressed tumor growth and improved survival.

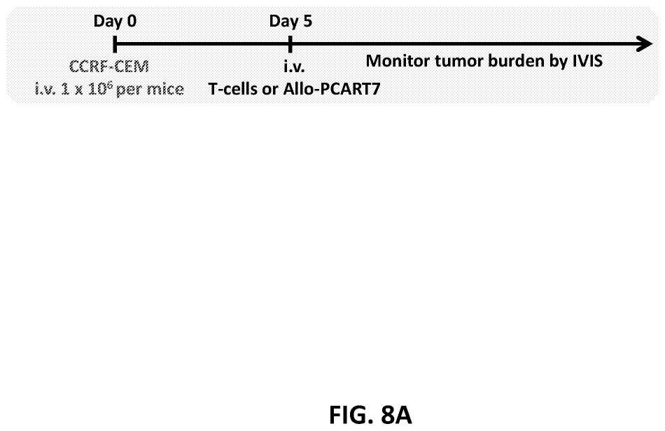

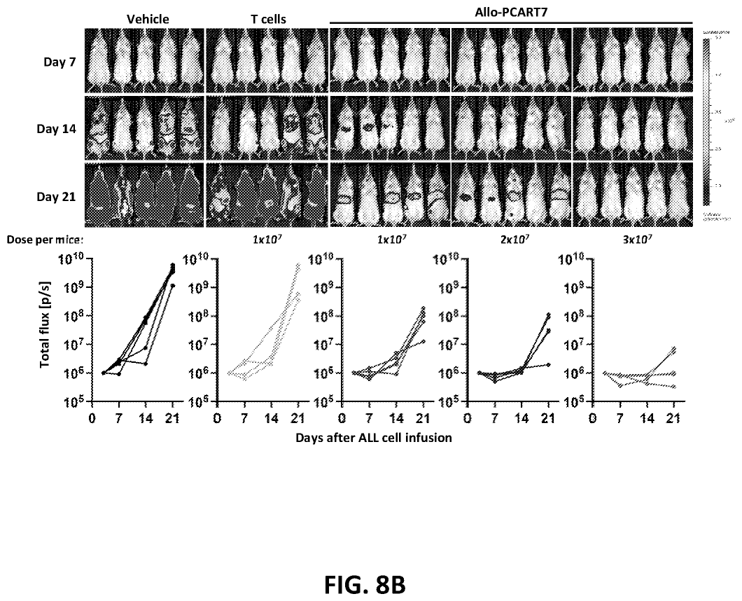

To further confirm this result, a similar experiment was performed. Briefly, as shown in FIG. 8A, on Day 0, NSG mice were infused with 1×10⁶ CCRF-CEM Luc-GFP cells, a T-ALL cell line, via intravenous injection (i.v.). On Day 5, these mice then received either PBS (vehicle), non-transduced T cells, or allo-PCART7 cells via i.v. injection. Tumor growth was then monitored by IVIS imaging. As shown in FIG. 8B, mice in the vehicle group and the non-transduced T cells group had high tumor burden at Day 21. However, mice treated with allo-PCART7 cells revealed a dose-dependent suppression of tumor cells. Total flux [p/s] over time graph is shown below each group and shows that the highest dose treatment resulted in the lowest tumor burden. These results indicate that allo-PCART7 cells are effective in killing leukemic cells in mice.

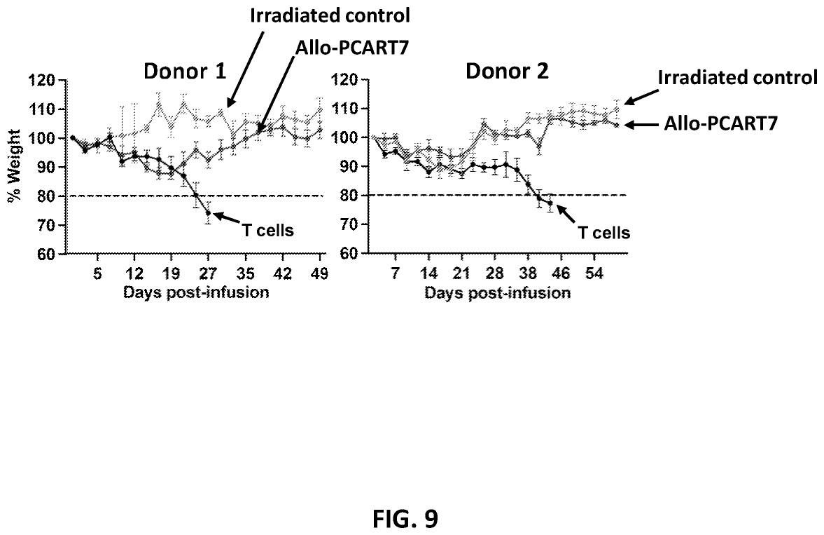

The xenoreactivity of allo-PCART7 cells was also evaluated in a mouse model of GvHD in NSG mice. In this experiment, NSG mice were irradiated with 2.5 Gy and injected with 1×10⁷ non-transduced T cells or allo-PCART7 cells intravenously (n=5 mice per group) one day after the irradiation. After the injection, body weights of the treated mice were measured for at least 7 weeks to monitor for GvHD. Mice were euthanized when mean weight reduction exceeded 20% in two consecutive measurements. As shown in FIG. 9, across 2 donors, no weight losses exceeding 20% were observed in the group of mice engrafted with allogeneic PCART7-CD3PEBL cells, whereas more than 20% body weight loss was observed in the group of mice engrafted with T cells. Such results indicate that in contrast to non-transduced T cells, allogeneic TCR/CD3 negative PCART7-CD3PEBL cells do not exert GvHD. Overall, these results showed that allo-PCART7 cells are highly effective in killing leukemic cells in mice and are not xenoreactive.

### Example 3: Generation of Allo-PCART7 Cells with a Kill Gene (Allo-PCART7^{KG} Cells)

In order to provide an additional safety mechanism in the engineered immune cells, e.g., allo-PCART7 cells, a kill gene or a suicide gene is incorporated into the engineered allo-PCART7 cells. CD20 is a non-immunogenic protein which has a dual function as a selection marker and as a suicide gene. A truncated form of CD20 (CD20t form) can be used as a kill gene. In case of adverse events, this CD20t can be used to eliminate allo-PCART7 after injection or infusion into the patient by administering a therapeutic monoclonal antibody targeting CD20, e.g., rituximab. In this example, the engineered allo-PCART7 cells with a kill gene is called “allo-PCART7^{KG}” cells. Binding of anti-CD20 antibody, e.g., rituximab to allo-PCART7^{KG}, triggers various antibody-dependent effector mechanisms such as complement-dependent cytotoxicity, thereby specifically eliminating allo-PCART7^{KG} cells.

### Methods

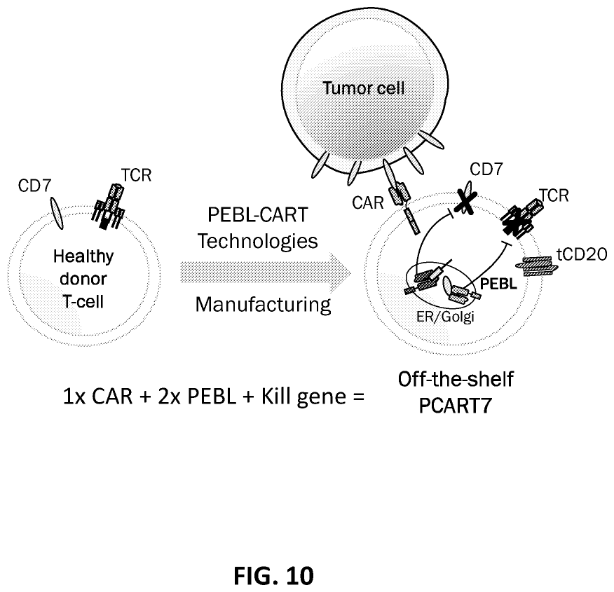

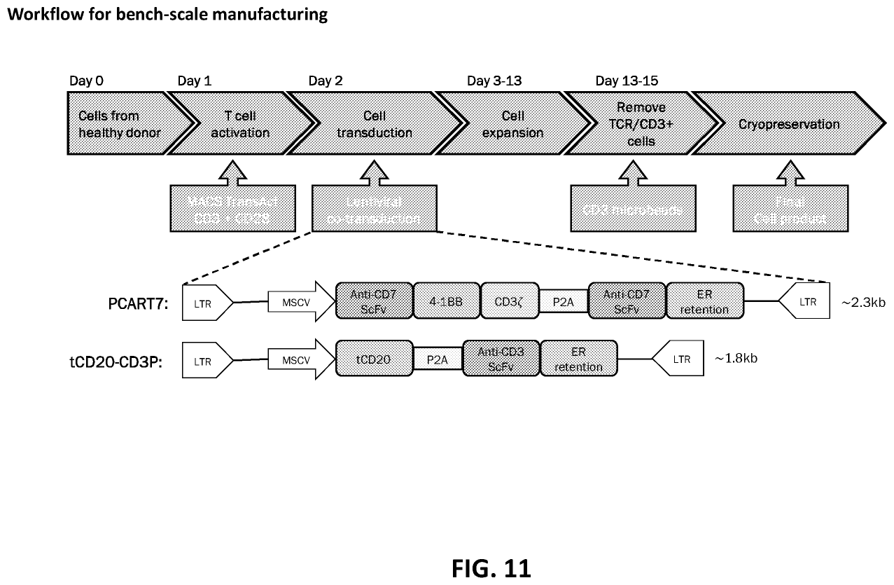

Allo-PCART7^{KG} cells were manufactured from healthy donor T cells by using a double transduction with a bicistronic lentiviral vector delivering anti-CD7 PEBL and anti-CD7 CAR, and a second bicistronic lentiviral vector delivering anti-CD3 PEBL and a kill gene. A truncated CD20 (CD20t) was used as a kill gene in this experiment. FIG. 10 shows scheme of the manufacturing process of allo-PCART7^{KG} cells using healthy donor T cells. FIG. 11 depicts the detailed workflow of the allo-PCART7^{KG} manufacturing process. Briefly, frozen PBMCs or CD4+/CD8+ enriched T cells collected from healthy donors were thawed on Day 0. On Day 1, T cells were activated using TransAct. On Day 2, T cells were transduced using lentiviral vectors for co-transduction of 2 PEBLs (CD3 PEBL and CD7 PEBL), with CD7 CAR, and optionally a suicide gene. Following the transduction, cells were expanded from Day 3 to Day 13 in vitro. Between Day 13 and Day 15, CD3 depletion of engineered T cells was performed once using CD3 microbeads. These cells were then cryopreserved as a final cell product afterward. In this example, allo-PCART7 cells were transduced to express CD20t, which was used as a suicide gene.

### Results

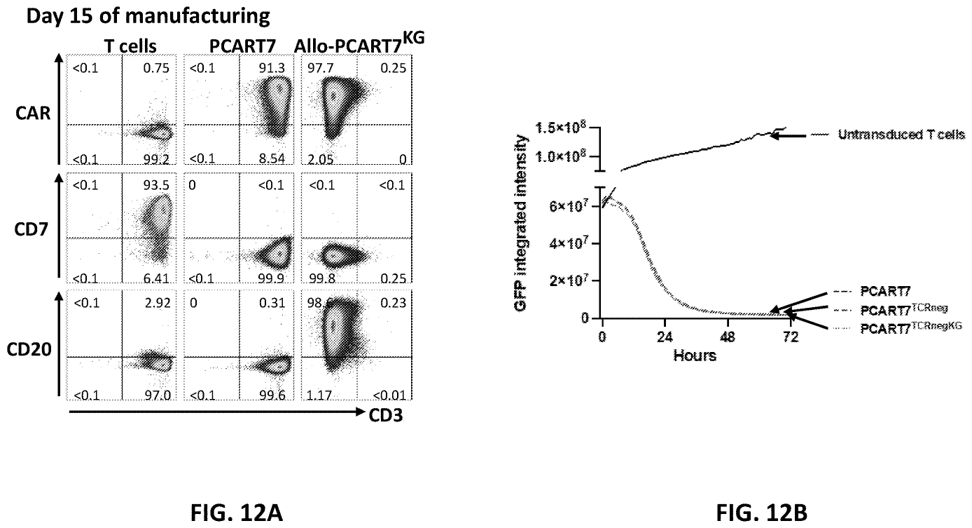

As shown in FIG. 12A, at 15 days after manufacturing began, allo-PCART7^{KG} cells were stained with respective antibodies and had the desired surface phenotype (CAR+/CD7-/CD3−/CD20+) as analyzed by flow cytometry. FIG. 12B compares the cytotoxic activity of PCART7 cells to allo-PCART7 cells (PCART7^{TCRneg}) and allo-PCART7^{KG} cells (PCART7^{TCRnegKG}). This result shows that there was no difference in the killing capacity and targeting of CD7+ cells (Jurkat cells) between PCART7 cells, allo-PCART7 cells (PCART7^{TCRneg}), and allo-PCART7^{KG} cells (PCART7^{TCRnegKG}), suggesting that a suicide gene, e.g., CD20t, does not affect the function of the allo-PCART7^{KG} cells (PCART7^{TCRnegKG}). These results showed desirable phenotypic and functional characteristics of allo-PCART7^{KG} cells (PCART7^{TCRnegKG}).

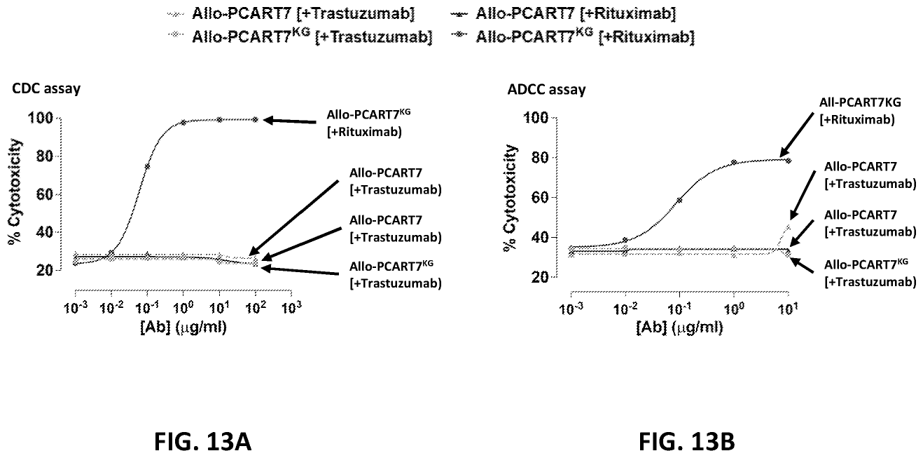

Further, in order to validate the function of the kill gene, a complement-dependent cytotoxicity (CDC) assay and an antibody-dependent cellular cytotoxicity (ADCC) assay were performed. As shown in FIGS. 13A-13B, results from the CDC assay and ADCC assay show that allo-PCART7^{KG} cells, which express CD20t, were recognized by rituximab, an anti-CD20 monoclonal antibody, resulting in initiation of the complement cascade and recognition by effector cells respectively, thereby specifically eliminating allo-PCART7^{KG} cells. Addition of rituximab did not induce cytotoxicity in allo-PCART7 cells that did not express CD20t. Trastuzumab, which is a monoclonal antibody targeting HER2, was used as a negative control. Overall, these data show that the kill gene, CD20t, is functional and enables specific depletion of allo-PCART7^{KG} cells.

### Example 4: Effects of Sequential Transduction of PCART7 and CD3 PEBL

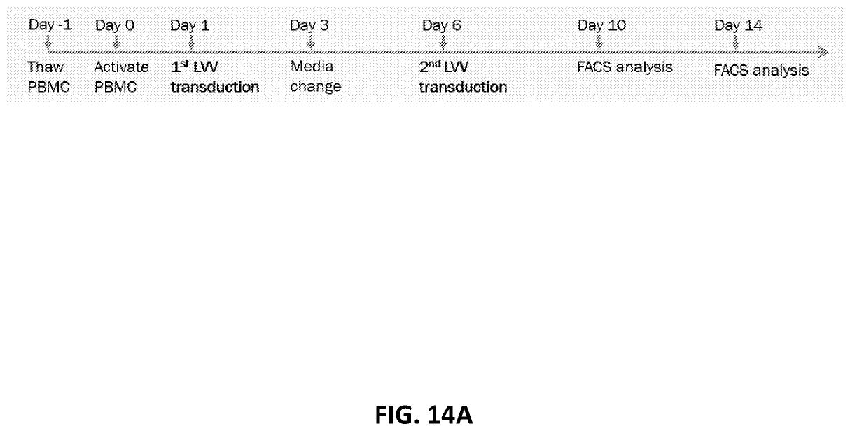

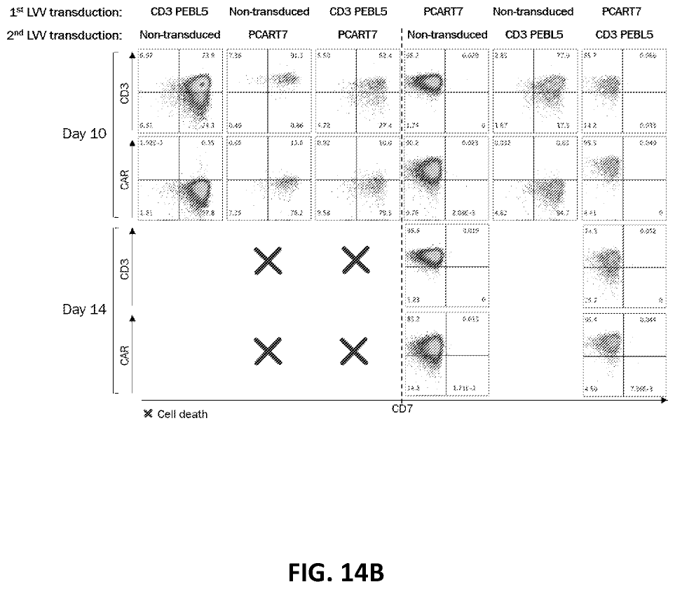

To determine the feasibility of sequential transduction to generate allo-PCART7 product, cells were transduced on Day 1 with either PCART7 or CD3 PEBL then transduced on Day 6 with the alternative construct (FIG. 14A). CAR, CD3 and CD7 expression was assessed by flow cytometry. FIG. 14B shows that for transduction of CD3 PEBL followed by PCART7, there were no viable cells during harvest potentially due to low expression of PCART7 (e.g., fratricide not prevented due to insufficient CD7 PEBL). For transduction of PCART7 followed by CD3 PEBL, poor CD3 downregulation was observed due to lower transduction efficiency for the second lentiviral vector transduction. Overall, these data show that sequential transduction with CD3 PEBL and PCART7 does not produce allo-PCART7 cells of the desired phenotype (CAR+CD7−CD3−).

### Example 5: Comparison of OKT3 PEBL and UCHT1 PEBL for CD3 Downregulation

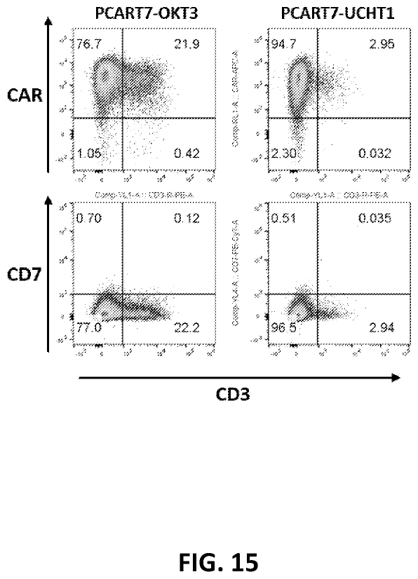

In this experiment, T cells were transduced with PCART7 (MOI 10) and either OKT3 PEBL or UCHT1 PEBL (MOI 20). CAR, CD7, and CD3 expression was examined by flow cytometry after 15 days. As shown in FIG. 15, UCHT1 PEBL showed greater downregulation of surface CD3 in allo-PCART7 cells.

### Example 6: Optimization of MOI for CD3 PEBL in Manufacturing Allo-PCART7 Cells

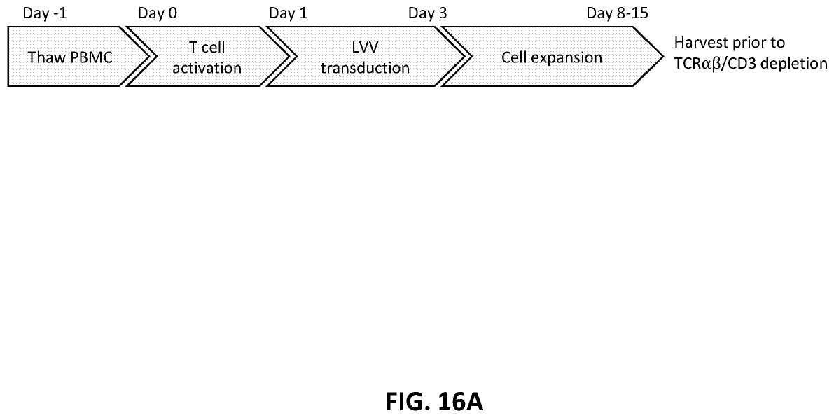

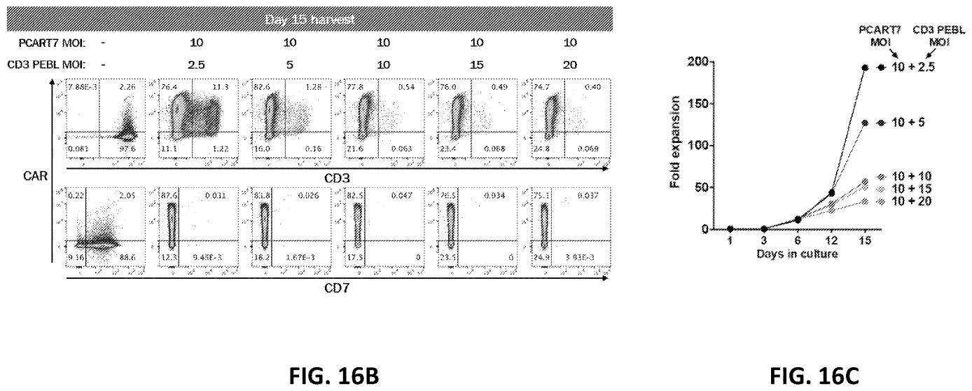

Following T cell activation, cells were cotransduced with PCART7 and CD3 PEBL, with the CD3 PEBL at varying multiplicity of infection (MOI). MOI refers to the number of viral particles per cell. An MOI of 5 indicates that there are five transducing units per cell in a well. Following lentiviral vector transduction, cells were expanded and harvested prior to TCRαβ/CD3 depletion (FIG. 16A). CAR, CD7, and CD3 expression was examined by flow cytometry. There was a MOI-dependent downregulation of CD3 by CD3 PEBL, whereby increasing MOI led to greater downregulation of CD3 (FIG. 16B). Cotransduction of PCART7 with CD3 PEBL MOI of greater than 5 resulted in greater than 95% CAR+CD7−CD3− cell population (FIG. 16C). Additionally, cotransduction of PCART7 with CD3 PEBL MOI of 5 (10+5) showed robust cell expansion. These data demonstrate that a CD3 PEBL MOI of 5 or more results in sufficient downregulation of CD3.

### Example 7: Identification of Minimum Vector Copy Number Ratio of CD3 PEBL:PCART7 for CD3 Retention

In this experiment, CD3 PEBL MOI was varied across conditions to determine a minimum VCN ratio for stable CD3 retention. T cells were transduced with PCART7 (MOI 10) and CD3 PEBL (UCHT1) with MOI of 5, 10, 20, 30, 40, or 50 (see Table in FIG. 17A). Following transduction, cells underwent CD3 depletion on day 15 of manufacturing. CD3-depleted samples were cultured over 14 days, during which CD3 expression was measured by flow cytometry (FIG. 17A). CD3-depleted samples were also examined for vector copy number quantification.

The results from flow cytometry are quantified in FIG. 17B with percentage of CD3-negative cells plotted against CD3 PEBL/PCART7 VCN ratio. A CD3 PEBL/PCART7 VCN ratio of approximately 2 was determined to be sufficient for stable CD3 retention (i.e., greater than 95% of CD3-negative cells were maintained at Day 14).

### Example 8: Optimization of Kill Gene and CD3 PEBL Vector Construct

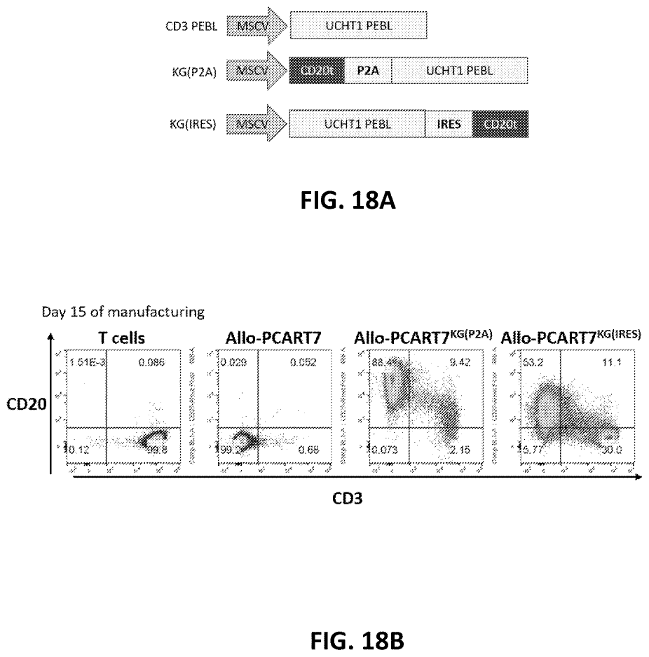

This experiment compared CD3 and CD20 expression across three constructs (FIG. 18A): CD3 PEBL with no kill gene (KG) sequence, CD3 PEBL with KG(P2A) (CD20t-P2A-UCHT1 PEBL), and CD3 PEBL with KG(IRES) (UCHT1 PEBL-IRES-CD20t). T cells were cotransduced with PCART7 and the CD3 PEBL only construct or either KG-CD3 PEBL construct. CD3 and CD20 expression were assessed by flow cytometry on day 15 of manufacturing (FIG. 18B).

The KG(P2A) construct (Allo-PCART7^{KG(P2A)}) showed superior performance compared to that of KG(IRES) (Allo-PCART7^{KG(IRES)}) when cotransduced with PCART7. The KG(P2A) demonstrated higher CD20 expression and greater CD3 downregulation. CD20t in the first position before UCHT1 PEBL allowed for the use of P2A instead of IRES. The KG(P2A) construct provided for better transduction and higher expression of CD20t.

### Example 9: Optimization of MOI for Kill Gene-CD3 PEBL in Manufacturing Allo-PCART7^{KG} Cells

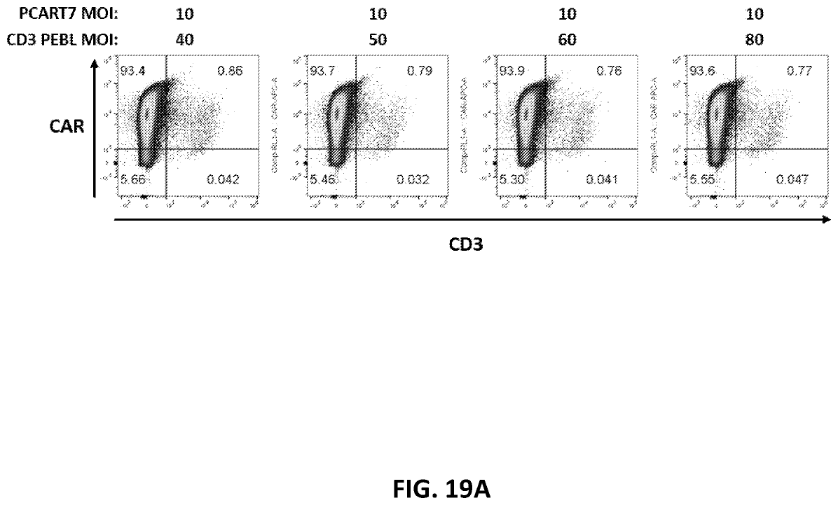

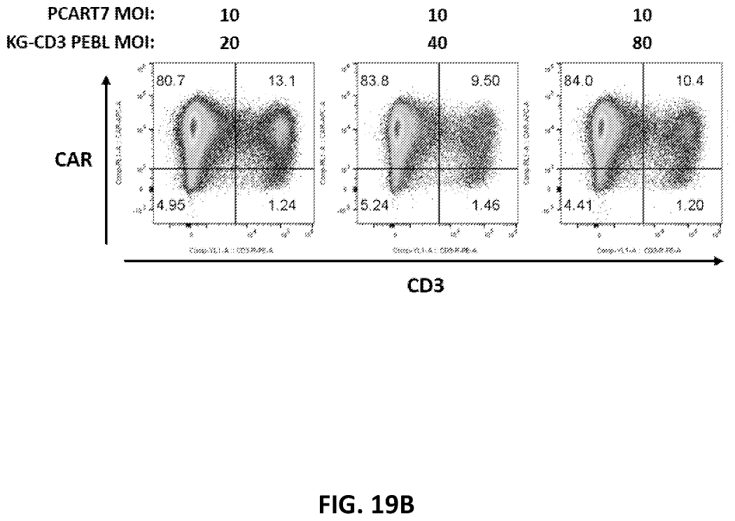

T cells were cotransduced with PCART7 lentiviral vector (MOI 10) and CD3 PEBL (UCHT1). CD3 PEBL was transduced at varying MOIs of 40, 50, 60, or 80. Following 15 days of manufacturing, CD7 and CD3 expression was analyzed by flow cytometry (FIG. 19A). In a separate experiment, T cells were cotransduced with PCART7 lentiviral vector (MOI 10) and CD3 PEBL (UCHT1) with a kill gene (KG-CD3 PEBL). KG-CD3 PEBL was transduced at varying MOIs of 20, 40, and 80. CAR and CD3 expression was analyzed by flow cytometry (FIG. 19B). PCART7 at MOI 10 and KG-CD3 PEBL at MOI 40 was shown to be the optimal ratio for cotransduction. The ratio was effective for CAR expression and downregulation of CD3.

### Example 10: Effects of CD20 Enrichment on Allo-PCART7^{KG} Phenotype

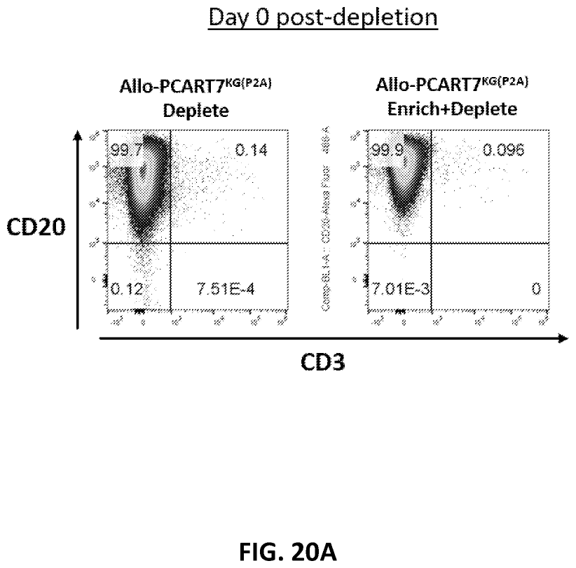

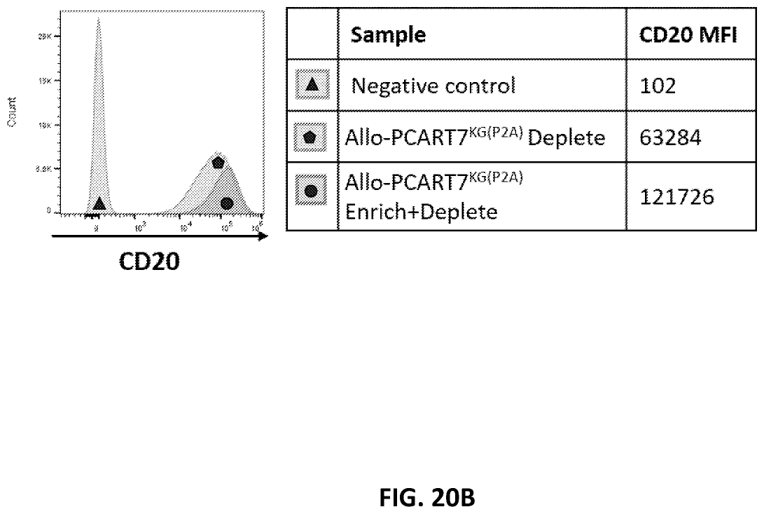

Allo-PCART7^{KG(P2A)} cells underwent CD3 depletion either with or without a prior CD20 enrichment step. Following cell manufacturing, CD3 and CD20 expression was analyzed by flow cytometry and CD20 median fluorescence intensity (MFI) was quantified. At day 0 post-depletion, cells that underwent CD20 enrichment showed greater CD20 expression (FIG. 20A). Addition of a CD20 enrichment step to the cell manufacturing process improved cell phenotype and homogeneity by recovering CD20-high expressing cells. Allo-PCART7^{KG(P2A)} cells that underwent a CD20 enrichment step prior to CD3 depletion had higher CD20 MFI and a narrower distribution of CD20 expression compared to cells that only underwent CD3 depletion (FIG. 20B).

### Example 11: Functional Evaluation of CD20 Enriched Allo-PCART7^{KG} Cells

T cells were cotransduced with PCART7 and CD3 PEBL KG(P2A) constructs. Following cell expansion, CD20 was enriched, TCR/CD3 was depleted, and cells were functionally assessed.

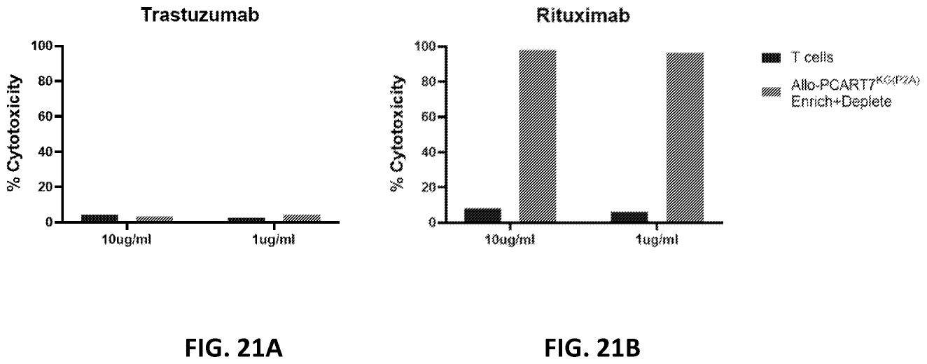

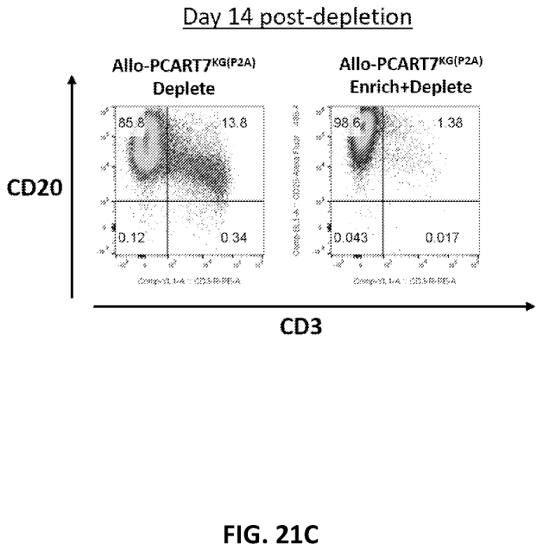

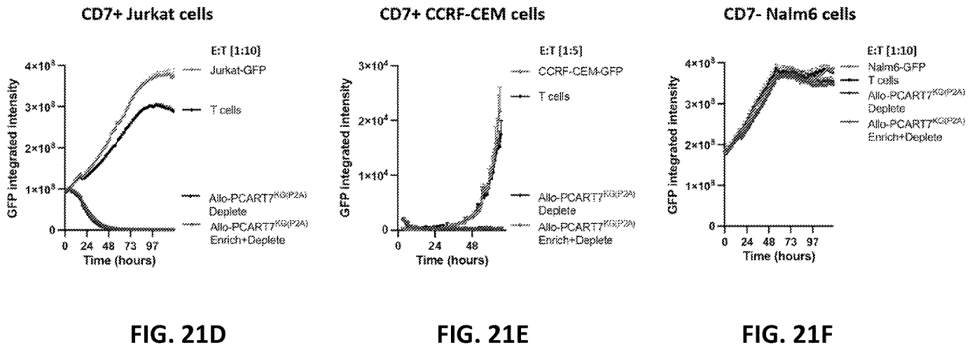

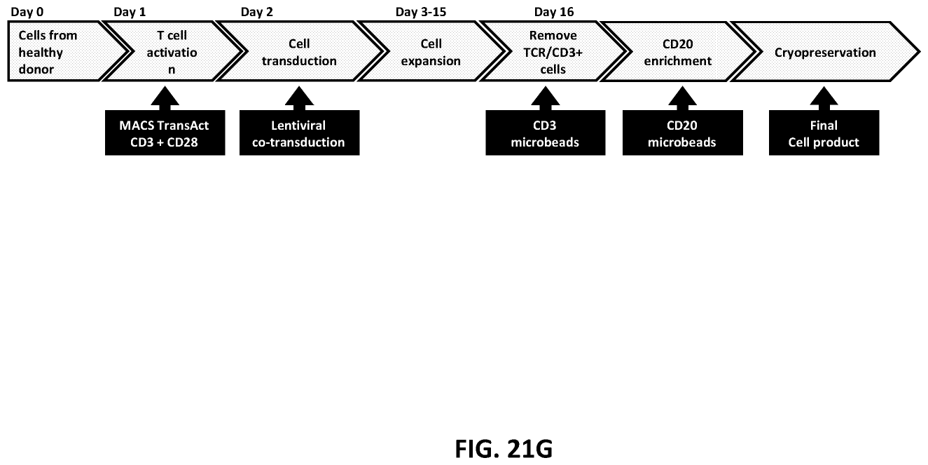

Cytotoxicity was evaluated using a complement-dependent cytotoxicity (CDC) assay. Non-transduced control T cells and CD20 enriched allo-PCART7^{KG(P2A)} cells were treated with Trastuzumab or Rituximab (FIGS. 21A-21B). CD20-enriched cells were efficiently eliminated by Rituximab but not Trastuzumab while non-transduced T cells were not killed by either. CD3 retention was also monitored over 14 days post-depletion in allo-PCART7^{KG(P2A)} cells with and without CD20 enrichment. CD20 and CD3 expression were examined by flow cytometry. (FIG. 21C). A CD3-positive population emerged 14 days post-depletion in allo-PCART7^{KG(P2A)} cells without CD20 enrichment (FIG. 21C, see left cytometry plot). CD20-enrichment enhanced CD3 retention and overcame the CD3 emergence by selecting for CD20-high, hence CD3 PEBL-high, expressing cells. CD20-enriched allo-PCART7^{KG(P2A)} cells exhibited good CD3 retention (i.e., greater than 95% CD3-negative) at 14 days post-depletion. This was comparable to allo-PCART7 cells with no kill gene.

Cytotoxic activity of allo-PCART7 and CD20-enriched allo-PCART7^{KG(P2A)} cells against GFP-expressing CD7+ and CD7− leukemic cells was assessed using an IncuCyte® live-cell analysis system. CD20-enriched allo-PCART7^{KG(P2A)} cells exhibited comparable killing efficacy to allo-PCART7^{KG(P2A)} cells that had not undergone CD20 enrichment (FIGS. 21D-20F). Cytotoxic activity was seen only against cells that were CD7+(see FIGS. 21D-21E). An experimental procedure for incorporating CD20 enrichment in production of allo-PCART7^{KG(P2A)} cells can be found in FIG. 21G.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### APPENDIX A

TABLE 13
Exemplary nucleotide sequences

| SEQ ID | | |
|---|---|---|
| NO. | Name | Sequence |
| 1 | CPPT-CMV- | ATCGATTTTAAAAGAAAAGGGGGGATTGGGGGGTACAGTGCAGGGGAAAGAATAGTAGACATA |
| | MCS-PGK- | ATAGCAACAGACATACAAACTAAAGAATTACAAAAACAAATTACAAAAATTCAAAATTTTCGG |
| | GFP-WPRE | GTTTATTAATTAACAGGGACATGCAGAGATCCAGTTTAAGCTTGGGAGTTCCGCGTTACATAA |
| | | CTTACGGTAAATGGCCCGCCTGGCTGACCGCCCAACGACCCCCGCCCATTGACGTCAATAATG |
| | | ACGTATGTTCCCATAGTAACGCCAATAGGGACTTTCCATTGACGTCAATGGGTGGAGTATTTA |
| | | CGGTAAACTGCCCACTTGGCAGTACATCAAGTGTATCATATGCCAAGTACGCCCCCTATTGAC |
| | | GTCAATGACGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTTGGCAGTAC |
| | | ATCAATGGGCGTGGATAGCGGTTTGACTCACGGGGATTTCCAAGTCTCCACCCCATTGACGTC |
| | | AATGGGAGTTTGTTTTGGCACCAAAATCAACGGGACTTTCCAAAATGTCGTAACAACTCCGCC |
| | | CCATTGACGCAAATGGGCGGTAGGCGTGTACGGTGGGAGGTCTATATAAGCAGAGCTCGTTTA |
| | | GTGAACCGTCAGATCGCCTGGAGACGCCATCCACGCTGTTTTGACCTCCATAGAAGACACCGA |
| | | CTCTACTAGAGGATCGCTAGCGCTACCGGACTCAGATCGAATTCCTCAAGCTTCCTCGAGTGC |
| | | AGTCGACGCGGGCCCGGGATCCCGCGACTCTAGATAATTCTACCGGGTAGGGGAGGCGCTTTT |
| | | CCCAAGGCAGTCTGGAGCATGCGCTTTAGCAGCCCCGCTGGGCACTTGGCGCTACACAAGTGG |
| | | CCTCTGGCCTCGCACACATTCCACATCCACCGGTAGGCGCCAACCGGCTCCGTTCTTTGGTGG |
| | | CCCCTTCGCGCCACCTTCTACTCCTCCCCTAGTCAGGAAGTTCCCCCCCGCCCCGCAGCTCGC |
| | | GTCGTGCAGGACGTGACAAATGGAAGTAGCACGTCTCACTAGTCTCGTGCAGATGGACAGCAC |
| | | CGCTGAGCAATGGAAGCGGGTAGGCCTTTGGGGCAGCGGCCAATAGCAGCTTTGCTCCTTCGC |
| | | TTTCTGGGCTCAGAGGCTGGGAAGGGGTGGGTCCGGGGGGGGCTCAGGGGGGGGCTCAGGGG |
| | | CGGGGCGGGCGCCCGAAGGTCCTCCGGAGGCCCGGCATTCTGCACGCTTCAAAAGCGCACGTC |
| | | TGCCGCGCTGTTCTCCTCTTCCTCATCTCCGGGCCTTTCGACTGGCCACCGGCTTCCACCATG |
| | | GTGAGCAAGGGCGAGGAGCTGTTCACCGGGGTGGTGCCCATCCTGGTCGAGCTGGACGGCGAC |
| | | GTAAACGGCCACAAGTTCAGCGTGTCCGGCGAGGGCGAGGGCGATGCCACCTACGGCAAGCTG |
| | | ACCCTGAAGTTCATCTGCACCACCGGCAAGCTGCCCGTGCCCTGGCCCACCCTCGTGACCACC |
| | | CTGACCTACGGCGTGCAGTGCTTCAGCCGCTACCCCGACCACATGAAGCAGCACGACTTCTTC |
| | | AAGTCCGCCATGCCCGAAGGCTACGTCCAGGAGCGCACCATCTTCTTCAAGGACGACGGCAAC |
| | | TACAAGACCCGCGCCGAGGTGAAGTTCGAGGGCGACACCCTGGTGAACCGCATCGAGCTGAAG |
| | | GGCATCGACTTCAAGGAGGACGGCAACATCCTGGGGCACAAGCTGGAGTACAACTACAACAGC |
| | | CACAACGTCTATATCATGGCCGACAAGCAGAAGAACGGCATCAAGGTGAACTTCAAGATCCGC |
| | | CACAACATCGAGGACGGCAGCGTGCAGCTCGCCGACCACTACCAGCAGAACACCCCCATCGGC |
| | | GACGGCCCCGTGCTGCTGCCCGACAACCACTACCTGAGCACCCAGTCCGCCCTGAGCAAAGAC |
| | | CCCAACGAGAAGCGCGATCACATGGTCCTGCTGGAGTTCGTGACCGCCGCCGGGATCACTCTC |
| | | GGCATGGACGAGCTGTACAAGTAACGCGTCTGGAACAATCAACCTCTGGATTACAAAATTTGT |
| | | GAAAGATTGACTGGTATTCTTAACTATGTTGCTCCTTTTACGCTATGTGGATACGCTGCTTTA |
| | | ATGCCTTTGTATCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCGTGGTGTGCACT |
| | | GTGTTTGCTGACGCAACCCCCACTGGTTGGGGCATTGCCACCACCTGTCAGCTCCTTTCCGGG |
| | | ACTTTCGCTTTCCCCCTCCCTATTGCCACGGCGGAACTCATCGCCGCCTGCCTTGCCCGCTGC |
| | | TGGACAGGGGCTCGGCTGTTGGGCACTGACAATTCCGTGGTGTTGTCGGGGAAGCTGACGTCC |
| | | TTTCCATGGCTGCTCGCCTGTGTTGCCACCTGGATTCTGCGCGGGACGTCCTTCTGCTACGTC |
| | | CCTTCGGCCCTCAATCCAGCGGACCTTCCTTCCCGCGGCCTGCTGCCGGCTCTGCGGCCTCTT |
| | | CCGCGTCTTCGCCTTCGCCCTCAGACGAGTCGGATCTCCCTTTGGGCCGCCTCCCCGCCTGG |
| 2 | Anti-human | ATGGCTCTGCCTGTGACCGCACTGCTGCTGCCCCTGGCTCTGCTGCTGCACGCCGCAAGACCT |
| | CD7 (TH69) | GCCGCATACAAGGATATTCAGATGACTCAGACCACAAGCTCCCTGAGCGCCTCCCTGGGAGAC |
| | PEBL | CGAGTGACAATCTCTTGCAGTGCATCACAGGGAATTAGCAACTACCTGAATTGGTATCAGCAG |
| | | AAGCCAGATGGCACTGTGAAACTGCTGATCTACTATACCTCTAGTCTGCACAGTGGGGTCCCC |
| | | TCACGATTCAGCGGATCCGGCTCTGGGACAGACTACAGCCTGACTATCTCCAACCTGGAGCCC |
| | | GAAGATATTGCCACCTACTATTGCCAGCAGTACTCCAAGCTGCCTTATACCTTTGGCGGGGGA |
| | | ACAAAGCTGGAGATTAAAAGGGGAGGAGGAGGAAGCGGAGGAGGAGGATCCGGAGGCGGGGGA |
| | | TCTGGAGGAGGAGGAAGTGAGGTGCAGCTGGTCGAATCTGGAGGAGGACTGGTGAAGCCAGGA |
| | | GGATCTCTGAAACTGAGTTGTGCCGCTTCAGGCCTGACCTTCTCAAGCTACGCCATGAGCTGG |
| | | GTGCGACAGACACCTGAGAAGCGGCTGGAATGGGTCGCTAGCATCTCCTCTGGCGGGTTCACA |
| | | TACTATCCAGACTCCGTGAAAGGCAGATTTACTATCTCTCGGGATAACGCAAGAAATATTCTG |
| | | TACCTGCAGATGAGTTCACTGAGGAGCGAGGACACCGCAATGTACTATTGTGCCAGGGACGAA |
| | | GTGCGCGGCTATCTGGATGTCTGGGGAGCTGGCACTACCGTCACCGTCTCCAGCGAGCAGAAA |
| | | CTGATTAGCGAAGAGGACCTGAAAGATGAACTGTAA |
| 3 | Anti-human | ATGGCTCTGCCCGTCACCGCTCTGCTGCTGCCTCTGGCTCTGCTGCTGCACGCCGCAAGACCC |
| | CD7 (3A1F) | CAGGTCCAGCTGCAGGAGTCAGGGGCAGAGCTGGTGAAACCCGGAGCCAGTGTCAAACTGTCC |
| | PEBL | TGTAAGGCCAGCGGCTATACTTTCACCAGCTACTGGATGCACTGGGTGAAACAGAGGCCAGGA |
| | | CAGGGCCTGGAGTGGATCGGCAAGATTAACCCCAGCAATGGGCGCACCAACTACAACGAAAAG |
| | | TTTAAATCCAAGGCTACACTGACTGTGGACAAGAGCTCCTCTACCGCATACATGCAGCTGAGT |
| | | TCACTGACATCTGAAGATAGTGCCGTGTACTATTGCGCCAGAGGCGGGGTCTACTATGACCTG |
| | | TACTATTACGCACTGGATTATTGGGGGCAGGGAACCACAGTGACTGTCAGCTCCGGAGGAGGA |
| | | GGATCCGGCGGAGGAGGCTCTGGGGGAGGCGGGAGTGACATCGAGCTGACACAGTCTCCAGCC |
| | | ACTCTGAGCGTGACCCCTGGCGATTCTGTCAGTCTGTCATGTAGAGCTAGCCAGTCCATCTCT |
| | | AACAATCTGCACTGGTACCAGCAGAAATCACATGAAAGCCCTCGGCTGCTGATTAAGAGTGCT |
| | | TCACAGAGCATCTCCGGGATTCCAAGCAGATTCTCTGGCAGTGGGTCAGGAACCGACTTTACA |
| | | CTGTCCATTAACTCTGTGGAGACCGAAGATTTCGGCATGTATTTTTGCCAGCAGAGCAATTCC |
| | | TGGCCTTACACATTCGGAGGCGGGACTAAACTGGAGATTAAGAGGGAGCAGAAACTGATTAGC |
| | | GAAGAGGACCTGAAGGACGAACTGTGA |
| 4 | Anti-human | ATGGCTCTGCCTGTGACTGCCCTGCTGCTGCCCCTGGCCCTGCTGCTGCACGCTGCTAGACCC |
| | CD7 (TH69) | GCCGCATACAAGGACATTCAGATGACTCAGACTACATCCAGCCTGTCAGCTTCCCTGGGCGAT |
| | CAR | CGAGTGACTATCAGCTGTTCAGCAAGCCAGGGGATCAGCAATTACCTGAACTGGTACCAGCAG |
| | | AAGCCCGACGGAACTGTCAAGCTGCTGATCTACTATACTTCCAGTCTGCATAGCGGGGTCCCT |
| | | AGCCGATTCTCTGGGAGTGGAAGCGGCACCGACTATAGCCTGACCATTTCCAACCTGGAACCA |
| | | GAGGACATTGCCACCTATTACTGTCAGCAGTATTCAAAGCTGCCCTATACCTTTGGCGGAGGG |
| | | ACTAAGCTGGAGATCAAGCGAGGAGGGGGAGGATCAGGGGGAGGGGGGAGCGGAGGCGGGGGG |
| | | AGTGGCGGCGGAGGGTCAGAAGTCCAGCTGGTCGAGAGTGGGGGCGGACTGGTGAAACCTGGG |
| | | GGCTCTCTGAAACTGAGCTGTGCAGCCAGTGGCCTGACATTCTCCTCATATGCTATGTCTTGG |
| | | GTGCGGCAGACCCCAGAAAAGAGACTGGAGTGGGTGGCTTCAATCTCTTCCGGGGGCTTCACT |
| | | TACTATCCTGACAGCGTGAAAGGCAGATTCACTATCTCCAGGGATAACGCACGGAATATCCTG |
| | | TATCTGCAGATGAGCTCACTGAGATCCGAGGACACCGCAATGTACTACTGCGCAAGAGACGAA |
| | | GTGCGGGGGTATCTGGACGTGTGGGGAGCAGGCACTACCGTCACCGTGTCTAGCACCACTACA |
| | | CCTGCACCAAGGCCTCCCACACCCGCTCCCACTATCGCTTCCCAGCCACTGTCCCTGAGGCCC |
| | | GAGGCCTGCAGGCCAGCAGCTGGCGGAGCCGTGCATACTAGGGGGCTGGACTTCGCTTGCGAC |
| | | ATCTACATCTGGGCCCCACTGGCAGGGACATGCGGAGTCCTGCTGCTGTCCCTGGTCATCACA |
| | | CTGTACTGCAAGCGGGGGCGCAAAAAACTGCTGTATATCTTTAAGCAGCCTTTCATGAGACCA |
| | | GTGCAGACAACCCAGGAGGAAGATGGGTGCTCATGCCGGTTTCCCGAGGAGGAGGAAGGCGGC |
| | | TGCGAGCTGAGGGTGAAGTTTTCCCGCTCAGCAGATGCTCCTGCCTACCAGCAGGGCCAGAAC |
| | | CAGCTGTATAATGAGCTGAACCTGGGCAGACGCGAAGAGTATGATGTGCTGGACAAAAGGCGG |
| | | GGAAGAGACCCCGAAATGGGAGGGAAGCCAAGGCGGAAAAACCCCCAGGAGGGCCTGTACAAT |
| | | GAGCTGCAGAAGGACAAAATGGCAGAGGCTTACAGTGAGATTGGGATGAAGGGAGAGAGACGG |
| | | AGGGGAAAAGGGCACGATGGCCTGTACCAGGGGCTGAGCACAGCAACCAAAGATACTTATGAC |
| | | GCACTGCACATGCAGGCACTGCCACCCAGATGA |
| 5 | Anti-human | ATGGCTCTGCCCGTCACCGCTCTGCTGCTGCCTCTGGCTCTGCTGCTGCACGCTGCTCGACCA |
| | CD7 (3A1F) | CAGGTCCAGCTGCAGGAGTCAGGAGCTGAGCTGGTGAAGCCAGGGGCAAGCGTCAAACTGTCC |
| | CAR | TGCAAGGCCTCTGGATATACATTCACTAGCTACTGGATGCACTGGGTGAAACAGAGACCCGGA |
| | | CAGGGCCTGGAGTGGATCGGAAAGATTAACCCTAGCAATGGCAGGACCAACTACAACGAAAAG |
| | | TTTAAATCCAAGGCAACCCTGACAGTGGACAAGAGCTCCTCTACAGCCTACATGCAGCTGAGT |
| | | TCACTGACTTCAGAGGATAGCGCAGTGTACTATTGCGCCAGAGGCGGGGTCTACTATGACCTG |
| | | TACTATTACGCCCTGGATTATTGGGGGCAGGGAACCACAGTGACTGTCAGCTCCGGAGGAGGA |
| | | GGAAGCGGAGGAGGAGGATCCGGAGGAGGAGGATCTGACATCGAGCTGACCCAGAGTCCTGCT |
| | | ACACTGAGCGTGACTCCAGGCGATTCTGTCAGTCTGTCATGTCGGGCAAGCCAGTCCATCTCT |
| | | AACAATCTGCACTGGTACCAGCAGAAATCCCATGAATCTCCACGACTGCTGATTAAGAGTGCC |
| | | TCACAGAGCATCTCCGGCATTCCCTCCCGGTTCTCTGGCAGTGGGTCAGGAACTGACTTTACC |
| | | CTGAGTATTAACTCAGTGGAGACAGAAGATTTCGGCATGTATTTTTGCCAGCAGAGCAATTCC |
| | | TGGCCCTACACTTTCGGAGGCGGGACCAAACTGGAGATCAAGCGGACTACCACACCAGCTCCA |
| | | AGACCACCTACCCCTGCACCAACAATTGCTAGTCAGCCACTGTCACTGAGACCAGAAGCATGT |
| | | AGGCCTGCAGCTGGAGGAGCTGTGCACACCAGAGGCCTGGACTTTGCCTGCGATATCTACATT |
| | | TGGGCTCCTCTGGCAGGAACCTGTGGCGTGCTGCTGCTGTCTCTGGTCATCACACTGTATTGC |
| | | AAACGAGGGCGGAAGAAACTGCTGTACATTTTCAAGCAGCCCTTTATGAGGCCTGTGCAGACT |
| | | ACCCAGGAGGAAGACGGCTGCAGCTGTCGCTTCCCTGAGGAAGAGGAAGGGGGATGTGAGCTG |
| | | CGCGTCAAATTTTCTCGAAGTGCAGATGCCCCAGCTTATCAGCAGGGCCAGAACCAGCTGTAC |
| | | AACGAGCTGAATCTGGGGCGGAGAGAGGAATATGACGTGCTGGATAAGAGGCGAGGAAGGGAC |
| | | CCAGAAATGGGAGGGAAACCACGACGGAAGAACCCCCAGGAGGGCCTGTATAATGAACTGCAG |
| | | AAAGACAAGATGGCAGAGGCCTACTCTGAAATCGGGATGAAAGGAGAGAGAAGGCGCGGCAAG |
| | | GGGCATGATGGCCTGTACCAGGGGCTGAGCACAGCAACCAAAGACACCTATGACGCACTGCAC |
| | | ATGCAGGCACTGCCACCACGGTAATGA |
| 6 | CMV promoter | AAGCTTGGGAGTTCCGCGTTACATAACTTACGGTAAATGGCCCGCCTGGCTGACCGCCCAACG |
| | | ACCCCCGCCCATTGACGTCAATAATGACGTATGTTCCCATAGTAACGCCAATAGGGACTTTCC |
| | | ATTGACGTCAATGGGTGGAGTATTTACGGTAAACTGCCCACTTGGCAGTACATCAAGTGTATC |
| | | ATATGCCAAGTACGCCCCCTATTGACGTCAATGACGGTAAATGGCCCGCCTGGCATTATGCCC |
| | | AGT |
| 7 | EF1α promoter | GGCTCCGGTGCCCGTCAGTGGGCAGAGCGCACATCGCCCACAGTCCCCGAGAAGTTGGGGGGA |
| | | GGGGTCGGCAATTGAACCGGTGCCTAGAGAAGGTGGCGCGGGGTAAACTGGGAAAGTGATGTC |
| | | GTGTACTGGCTCCGCCTTTTTCCCGAGGGTGGGGGAGAACCGTATATAAGTGCAGTAGTCGCC |
| | | GTGAACGTTCTTTTTCGCAACGGGTTTGCCGCCAGAACACAGGTAAGTGCCGTGTGTGGTTCC |
| | | CGCGGGCCTGGCCTCTTTACGGGTTATGGCCCTTGCGTGCCTTGAATTACTTCCACCTGGCTG |
| | | CAGTACGTGATTCTTGATCCCGAGCTTCGGGTTGGAAGTGGGTGGGAGAGTTCGAGGCCTTGC |
| | | GCTTAAGGAGCCCCTTCGCCTCGTGCTTGAGTTGAGGCCTGGCCTGGGCGCTGGGGCCGCCGC |
| | | GTGCGAATCTGGTGGCACCTTCGCGCCTGTCTCGCTGCTTTCGATAAGTCTCTAGCCATTTAA |
| | | AATTTTTGATGACCTGCTGCGACGCTTTTTTTCTGGCAAGATAGTCTTGTAAATGCGGGCCAA |
| | | GATCTGCACACTGGTATTTCGGTTTTTGGGGCCGCGGGCGGCGACGGGGCCCGTGCGTCCCAG |
| | | CGCACATGTTCGGCGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCCCGCCCTGGGCG |
| | | GCAAGGCTGGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCACCCACACAA |
| | | AGGAAAAGGGCCTTTCCGTCCTCAGCCGTCGCTTCATGTGACTCCACGGAGTACCGGGCGCCG |
| | | TCCAGGCACCTCGATTAGTTCTCGAGCTTTTGGAGTACGTCGTCTTTAGGTTGGGGGGAGGGG |
| | | TTTTATGCGATGGAGTTTCCCCACACTGAGTGGGTGGAGACTGAAGTTAGGCCAGCTTGGCAC |
| | | TTGATGTAATTCTCCTTGGAATTTGCCCTTTTTGAGTTTGGATCTTGGTTCATTCTCAAGCCT |
| | | CAGACAGTGGTTCAAAGTTTTTTTCTTCCATTTCAGGTGTCGTGA |
| 8 | EFS promoter | ATTCTACCGGGTAGGGGAGGCGCTTTTCCCAAGGCAGTCTGGAGCATGCGCTTTAGCAGCCCC |
| | | GCTGGGCACTTGGCGCTACACAAGTGGCCTCTGGCCTCGCACACATTCCACATCCACCGGTAG |
| | | GCGCCAACCGGCTCCGTTCTTTGGTGGCCCCTTCGCGCCACCTTCTACTCCTCCCCTAGTCAG |
| | | GAAGTTCCCCCCCGCCCCGCAGCTCGCGTCGTGCAGGACGTGACAAATGGAAGTAGCACGTCT |
| | | CACTAGTCTCGTGCAGATGGACAGCACCGCTGAGCAATGGAAGCGGGTAGGCCTTTGGGGCAG |
| | | CGGCCAATAGCAGCTTTGCTCCTTCGCTTTCTGGGCTCAGAGGCTGGGAAGGGGTGGGTCCGG |
| | | GGGCGGGCTCAGGGGCGGGCTCAGGGGCGGGGCGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGA |
| 9 | MSCV | GGAATGAAAGACCCCACCTGTAGGTTTGGCAAGCTAGCTTAAGTAACGCCATTTTGCAAGGCA |
| | promoter | TGGAAAATACATAACTGAGAATAGAGAAGTTCAGATCAAGGTTAGGAACAGAGAGACAGCAGA |
| | | ATATGGGCCAAACAGGATATCTGTGGTAAGCAGTTCCTGCCCCGGCTCAGGGCCAAGAACAGA |
| | | TGGTCCCCAGATGCGGTCCCGCCCTCAGCAGTTTCTAGAGAACCATCAGATGTTTCCAGGGTG |
| | | CCCCAAGGACCTGAAATGACCCTGTGCCTTATTTGAACTAACCAATCAGTTCGCTTCTCGCTT |
| | | CTGTTCGCGCGCTTCTGCTCCCCGAGCTCAATAAAAGAGCCCACAACCCCTCACTCGGCGCGC |
| | | CAGTCC |
| 10 | PGK promoter | ATTCTACCGGGTAGGGGAGGCGCTTTTCCCAAGGCAGTCTGGAGCATGCGCTTTAGCAGCCCC |
| | | GCTGGGCACTTGGCGCTACACAAGTGGCCTCTGGCCTCGCACACATTCCACATCCACCGGTAG |
| | | GCGCCAACCGGCTCCGTTCTTTGGTGGCCCCTTCGCGCCACCTTCTACTCCTCCCCTAGTCAG |
| | | GAAGTTCCCCCCCGCCCCGCAGCTCGCGTCGTGCAGGACGTGACAAATGGAAGTAGCACGTCT |
| | | CACTAGTCTCGTGCAGATGGACAGCACCGCTGAGCAATGGAAGCGGGTAGGCCTTTGGGGCAG |
| | | CGGCCAATAGCAGCTTTGCTCCTTCGCTTTCTGGGCTCAGAGGCTGGGAAGGGGTGGGTCCGG |
| | | GGGCGGGCTCAGGGGCGGGCTCAGGGGCGGGGGGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGA |
| 11 | Anti-human | ATGGCTCTGCCTGTGACCGCACTGCTGCTGCCCCTGGCTCTGCTGCTGCACGCCGCAAGACCT |
| | CD7 (TH69) | GCCGCATACAAGGATATTCAGATGACTCAGACCACAAGCTCCCTGAGCGCCTCCCTGGGAGAC |
| | PEBL-IRES- | CGAGTGACAATCTCTTGCAGTGCATCACAGGGAATTAGCAACTACCTGAATTGGTATCAGCAG |
| | Anti-human | AAGCCAGATGGCACTGTGAAACTGCTGATCTACTATACCTCTAGTCTGCACAGTGGGGTCCCC |
| | CD7 (TH69) | TCACGATTCAGCGGATCCGGCTCTGGGACAGACTACAGCCTGACTATCTCCAACCTGGAGCCC |
| | CAR | GAAGATATTGCCACCTACTATTGCCAGCAGTACTCCAAGCTGCCTTATACCTTTGGCGGGGGA |
| | | ACAAAGCTGGAGATTAAAAGGGGAGGAGGAGGAAGCGGAGGAGGAGGATCCGGAGGCGGGGGA |
| | | TCTGGAGGAGGAGGAAGTGAGGTGCAGCTGGTCGAATCTGGAGGAGGACTGGTGAAGCCAGGA |
| | | GGATCTCTGAAACTGAGTTGTGCCGCTTCAGGCCTGACCTTCTCAAGCTACGCCATGAGCTGG |
| | | GTGCGACAGACACCTGAGAAGCGGCTGGAATGGGTCGCTAGCATCTCCTCTGGCGGGTTCACA |
| | | TACTATCCAGACTCCGTGAAAGGCAGATTTACTATCTCTCGGGATAACGCAAGAAATATTCTG |
| | | TACCTGCAGATGAGTTCACTGAGGAGCGAGGACACCGCAATGTACTATTGTGCCAGGGACGAA |
| | | GTGCGCGGCTATCTGGATGTCTGGGGAGCTGGCACTACCGTCACCGTCTCCAGCGAGCAGAAA |
| | | CTGATTAGCGAAGAGGACCTGAAAGATGAACTGTAACGGGATCAATTCCGCCCCCCCCCTAAC |
| | | GTTACTGGCCGAAGCCGCTTGGAATAAGGCCGGTGTGCGTTTGTCTATATGTTATTTTCCACC |
| | | ATATTGCCGTCTTTTGGCAATGTGAGGGCCCGGAAACCTGGCCCTGTCTTCTTGACGAGCATT |
| | | CCTAGGGGTCTTTCCCCTCTCGCCAAAGGAATGCAAGGTCTGTTGAATGTCGTGAAGGAAGCA |
| | | GTTCCTCTGGAAGCTTCTTGAAGACAAACAACGTCTGTAGCGACCCTTTGCAGGCAGCGGAAC |
| | | CCCCCACCTGGCGACAGGTGCCTCTGCGGCCAAAAGCCACGTGTATAAGATACACCTGCAAAG |
| | | GCGGCACAACCCCAGTGCCACGTTGTGAGTTGGATAGTTGTGGAAAGAGTCAAATGGCTCTCC |
| | | TCAAGCGTATTCAACAAGGGGCTGAAGGATGCCCAGAAGGTACCCCATTGTATGGGATCTGAT |
| | | CTGGGGCCTCGGTGCACATGCTTTACATGTGTTTAGTCGAGGTTAAAAAAACGTCTAGGCCCC |
| | | CCGAACCACGGGGACGTGGTTTTCCTTTGAAAAACACGATAATACCATGGCTCTGCCTGTGAC |
| | | TGCCCTGCTGCTGCCCCTGGCCCTGCTGCTGCACGCTGCTAGACCCGCCGCATACAAGGACAT |
| | | TCAGATGACTCAGACTACATCCAGCCTGTCAGCTTCCCTGGGCGATCGAGTGACTATCAGCTG |
| | | TTCAGCAAGCCAGGGGATCAGCAATTACCTGAACTGGTACCAGCAGAAGCCCGACGGAACTGT |
| | | CAAGCTGCTGATCTACTATACTTCCAGTCTGCATAGCGGGGTCCCTAGCCGATTCTCTGGGAG |
| | | TGGAAGCGGCACCGACTATAGCCTGACCATTTCCAACCTGGAACCAGAGGACATTGCCACCTA |
| | | TTACTGTCAGCAGTATTCAAAGCTGCCCTATACCTTTGGCGGAGGGACTAAGCTGGAGATCAA |
| | | GCGAGGAGGGGGAGGATCAGGGGGAGGGGGGAGCGGAGGCGGGGGGAGTGGCGGCGGAGGGTC |
| | | AGAAGTCCAGCTGGTCGAGAGTGGGGGCGGACTGGTGAAACCTGGGGGCTCTCTGAAACTGAG |
| | | CTGTGCAGCCAGTGGCCTGACATTCTCCTCATATGCTATGTCTTGGGTGCGGCAGACCCCAGA |
| | | AAAGAGACTGGAGTGGGTGGCTTCAATCTCTTCCGGGGGCTTCACTTACTATCCTGACAGCGT |
| | | GAAAGGCAGATTCACTATCTCCAGGGATAACGCACGGAATATCCTGTATCTGCAGATGAGCTC |
| | | ACTGAGATCCGAGGACACCGCAATGTACTACTGCGCAAGAGACGAAGTGCGGGGGTATCTGGA |
| | | CGTGTGGGGAGCAGGCACTACCGTCACCGTGTCTAGCACCACTACACCTGCACCAAGGCCTCC |
| | | CACACCCGCTCCCACTATCGCTTCCCAGCCACTGTCCCTGAGGCCCGAGGCCTGCAGGCCAGC |
| | | AGCTGGCGGAGCCGTGCATACTAGGGGGCTGGACTTCGCTTGCGACATCTACATCTGGGCCCC |
| | | ACTGGCAGGGACATGCGGAGTCCTGCTGCTGTCCCTGGTCATCACACTGTACTGCAAGCGGGG |
| | | GCGCAAAAAACTGCTGTATATCTTTAAGCAGCCTTTCATGAGACCAGTGCAGACAACCCAGGA |
| | | GGAAGATGGGTGCTCATGCCGGTTTCCCGAGGAGGAGGAAGGCGGCTGCGAGCTGAGGGTGAA |
| | | GTTTTCCCGCTCAGCAGATGCTCCTGCCTACCAGCAGGGCCAGAACCAGCTGTATAATGAGCT |
| | | GAACCTGGGCAGACGCGAAGAGTATGATGTGCTGGACAAAAGGCGGGGAAGAGACCCCGAAAT |
| | | GGGAGGGAAGCCAAGGCGGAAAAACCCCCAGGAGGGCCTGTACAATGAGCTGCAGAAGGACAA |
| | | AATGGCAGAGGCTTACAGTGAGATTGGGATGAAGGGAGAGAGACGGAGGGGAAAAGGGCACGA |
| | | TGGCCTGTACCAGGGGCTGAGCACAGCAACCAAAGATACTTATGACGCACTGCACATGCAGGC |
| | | ACTGCCACCCAGATGACAGCCAGGGGATTTCACCACTCAAAGGCCAGACCTGCAGACGCCCAG |
| | | ATTATGAGACACA |
| 12 | Anti-human | ATGGCTCTGCCTGTGACTGCCCTGCTGCTGCCCCTGGCCCTGCTGCTGCACGCTGCTAGACCC |
| | CD7 (TH69) | GCCGCATACAAGGACATTCAGATGACTCAGACTACATCCAGCCTGTCAGCTTCCCTGGGCGAT |
| | CAR-IRES-Anti- | CGAGTGACTATCAGCTGTTCAGCAAGCCAGGGGATCAGCAATTACCTGAACTGGTACCAGCAG |
| | human CD7 | AAGCCCGACGGAACTGTCAAGCTGCTGATCTACTATACTTCCAGTCTGCATAGCGGGGTCCCT |
| | (TH69) PEBL | AGCCGATTCTCTGGGAGTGGAAGCGGCACCGACTATAGCCTGACCATTTCCAACCTGGAACCA |
| | | GAGGACATTGCCACCTATTACTGTCAGCAGTATTCAAAGCTGCCCTATACCTTTGGCGGAGGG |
| | | ACTAAGCTGGAGATCAAGCGAGGAGGGGGAGGATCAGGGGGAGGGGGGAGCGGAGGCGGGGGG |
| | | AGTGGCGGCGGAGGGTCAGAAGTCCAGCTGGTCGAGAGTGGGGGCGGACTGGTGAAACCTGGG |
| | | GGCTCTCTGAAACTGAGCTGTGCAGCCAGTGGCCTGACATTCTCCTCATATGCTATGTCTTGG |
| | | GTGCGGCAGACCCCAGAAAAGAGACTGGAGTGGGTGGCTTCAATCTCTTCCGGGGGCTTCACT |
| | | TACTATCCTGACAGCGTGAAAGGCAGATTCACTATCTCCAGGGATAACGCACGGAATATCCTG |
| | | TATCTGCAGATGAGCTCACTGAGATCCGAGGACACCGCAATGTACTACTGCGCAAGAGACGAA |
| | | GTGCGGGGGTATCTGGACGTGTGGGGAGCAGGCACTACCGTCACCGTGTCTAGCACCACTACA |
| | | CCTGCACCAAGGCCTCCCACACCCGCTCCCACTATCGCTTCCCAGCCACTGTCCCTGAGGCCC |
| | | GAGGCCTGCAGGCCAGCAGCTGGCGGAGCCGTGCATACTAGGGGGCTGGACTTCGCTTGCGAC |
| | | ATCTACATCTGGGCCCCACTGGCAGGGACATGCGGAGTCCTGCTGCTGTCCCTGGTCATCACA |
| | | CTGTACTGCAAGCGGGGGCGCAAAAAACTGCTGTATATCTTTAAGCAGCCTTTCATGAGACCA |
| | | GTGCAGACAACCCAGGAGGAAGATGGGTGCTCATGCCGGTTTCCCGAGGAGGAGGAAGGCGGC |
| | | TGCGAGCTGAGGGTGAAGTTTTCCCGCTCAGCAGATGCTCCTGCCTACCAGCAGGGCCAGAAC |
| | | CAGCTGTATAATGAGCTGAACCTGGGCAGACGCGAAGAGTATGATGTGCTGGACAAAAGGCGG |
| | | GGAAGAGACCCCGAAATGGGAGGGAAGCCAAGGCGGAAAAACCCCCAGGAGGGCCTGTACAAT |
| | | GAGCTGCAGAAGGACAAAATGGCAGAGGCTTACAGTGAGATTGGGATGAAGGGAGAGAGACGG |
| | | AGGGGAAAAGGGCACGATGGCCTGTACCAGGGGCTGAGCACAGCAACCAAAGATACTTATGAC |
| | | GCACTGCACATGCAGGCACTGCCACCCAGATGACAGCCAGGGGATTTCACCACTCAAAGGCCA |
| | | GACCTGCAGACGCCCAGATTATGAGACACACGGGATCAATTCCGCCCCCCCCCTAACGTTACT |
| | | GGCCGAAGCCGCTTGGAATAAGGCCGGTGTGCGTTTGTCTATATGTTATTTTCCACCATATTG |
| | | CCGTCTTTTGGCAATGTGAGGGCCCGGAAACCTGGCCCTGTCTTCTTGACGAGCATTCCTAGG |
| | | GGTCTTTCCCCTCTCGCCAAAGGAATGCAAGGTCTGTTGAATGTCGTGAAGGAAGCAGTTCCT |
| | | CTGGAAGCTTCTTGAAGACAAACAACGTCTGTAGCGACCCTTTGCAGGCAGCGGAACCCCCCA |
| | | CCTGGCGACAGGTGCCTCTGCGGCCAAAAGCCACGTGTATAAGATACACCTGCAAAGGCGGCA |
| | | CAACCCCAGTGCCACGTTGTGAGTTGGATAGTTGTGGAAAGAGTCAAATGGCTCTCCTCAAGC |
| | | GTATTCAACAAGGGGCTGAAGGATGCCCAGAAGGTACCCCATTGTATGGGATCTGATCTGGGG |
| | | CCTCGGTGCACATGCTTTACATGTGTTTAGTCGAGGTTAAAAAAACGTCTAGGCCCCCCGAAC |
| | | CACGGGGACGTGGTTTTCCTTTGAAAAACACGATAATACCATGGCTCTGCCTGTGACCGCACT |
| | | GCTGCTGCCCCTGGCTCTGCTGCTGCACGCCGCAAGACCTGCCGCATACAAGGATATTCAGAT |
| | | GACTCAGACCACAAGCTCCCTGAGCGCCTCCCTGGGAGACCGAGTGACAATCTCTTGCAGTGC |
| | | ATCACAGGGAATTAGCAACTACCTGAATTGGTATCAGCAGAAGCCAGATGGCACTGTGAAACT |
| | | GCTGATCTACTATACCTCTAGTCTGCACAGTGGGGTCCCCTCACGATTCAGCGGATCCGGCTC |
| | | TGGGACAGACTACAGCCTGACTATCTCCAACCTGGAGCCCGAAGATATTGCCACCTACTATTG |
| | | CCAGCAGTACTCCAAGCTGCCTTATACCTTTGGCGGGGGAACAAAGCTGGAGATTAAAAGGGG |
| | | AGGAGGAGGAAGCGGAGGAGGAGGATCCGGAGGCGGGGGATCTGGAGGAGGAGGAAGTGAGGT |
| | | GCAGCTGGTCGAATCTGGAGGAGGACTGGTGAAGCCAGGAGGATCTCTGAAACTGAGTTGTGC |
| | | CGCTTCAGGCCTGACCTTCTCAAGCTACGCCATGAGCTGGGTGCGACAGACACCTGAGAAGCG |
| | | GCTGGAATGGGTCGCTAGCATCTCCTCTGGCGGGTTCACATACTATCCAGACTCCGTGAAAGG |
| | | CAGATTTACTATCTCTCGGGATAACGCAAGAAATATTCTGTACCTGCAGATGAGTTCACTGAG |
| | | GAGCGAGGACACCGCAATGTACTATTGTGCCAGGGACGAAGTGCGCGGCTATCTGGATGTCTG |
| | | GGGAGCTGGCACTACCGTCACCGTCTCCAGCGAGCAGAAACTGATTAGCGAAGAGGACCTGAA |
| | | AGATGAACTGTAA |
| 13 | Anti-human | ATGGCTCTGCCTGTGACTGCCCTGCTGCTGCCCCTGGCCCTGCTGCTGCACGCTGCTAGACCC |
| | CD7 (TH69) | GCCGCATACAAGGACATTCAGATGACTCAGACTACATCCAGCCTGTCAGCTTCCCTGGGCGAT |
| | CAR-P2A- | CGAGTGACTATCAGCTGTTCAGCAAGCCAGGGGATCAGCAATTACCTGAACTGGTACCAGCAG |
| | Anti-human | AAGCCCGACGGAACTGTCAAGCTGCTGATCTACTATACTTCCAGTCTGCATAGCGGGGTCCCT |
| | CD7 (TH69) | AGCCGATTCTCTGGGAGTGGAAGCGGCACCGACTATAGCCTGACCATTTCCAACCTGGAACCA |
| | PEBL | GAGGACATTGCCACCTATTACTGTCAGCAGTATTCAAAGCTGCCCTATACCTTTGGCGGAGGG |
| | | ACTAAGCTGGAGATCAAGCGAGGAGGGGGAGGATCAGGGGGAGGGGGGAGCGGAGGCGGGGGG |
| | | AGTGGCGGCGGAGGGTCAGAAGTCCAGCTGGTCGAGAGTGGGGGCGGACTGGTGAAACCTGGG |
| | | GGCTCTCTGAAACTGAGCTGTGCAGCCAGTGGCCTGACATTCTCCTCATATGCTATGTCTTGG |
| | | GTGCGGCAGACCCCAGAAAAGAGACTGGAGTGGGTGGCTTCAATCTCTTCCGGGGGCTTCACT |
| | | TACTATCCTGACAGCGTGAAAGGCAGATTCACTATCTCCAGGGATAACGCACGGAATATCCTG |
| | | TATCTGCAGATGAGCTCACTGAGATCCGAGGACACCGCAATGTACTACTGCGCAAGAGACGAA |
| | | GTGCGGGGGTATCTGGACGTGTGGGGAGCAGGCACTACCGTCACCGTGTCTAGCACCACTACA |
| | | CCTGCACCAAGGCCTCCCACACCCGCTCCCACTATCGCTTCCCAGCCACTGTCCCTGAGGCCC |
| | | GAGGCCTGCAGGCCAGCAGCTGGCGGAGCCGTGCATACTAGGGGGCTGGACTTCGCTTGCGAC |
| | | ATCTACATCTGGGCCCCACTGGCAGGGACATGCGGAGTCCTGCTGCTGTCCCTGGTCATCACA |
| | | CTGTACTGCAAGCGGGGGCGCAAAAAACTGCTGTATATCTTTAAGCAGCCTTTCATGAGACCA |
| | | GTGCAGACAACCCAGGAGGAAGATGGGTGCTCATGCCGGTTTCCCGAGGAGGAGGAAGGCGGC |
| | | TGCGAGCTGAGGGTGAAGTTTTCCCGCTCAGCAGATGCTCCTGCCTACCAGCAGGGCCAGAAC |
| | | CAGCTGTATAATGAGCTGAACCTGGGCAGACGCGAAGAGTATGATGTGCTGGACAAAAGGCGG |
| | | GGAAGAGACCCCGAAATGGGAGGGAAGCCAAGGCGGAAAAACCCCCAGGAGGGCCTGTACAAT |
| | | GAGCTGCAGAAGGACAAAATGGCAGAGGCTTACAGTGAGATTGGGATGAAGGGAGAGAGACGG |
| | | AGGGGAAAAGGGCACGATGGCCTGTACCAGGGGCTGAGCACAGCAACCAAAGATACTTATGAC |
| | | GCACTGCACATGCAGGCACTGCCACCCAGAGGCAGCGGCGCCACAAACTTCTCTCTGCTAAAG |
| | | CAAGCAGGTGATGTTGAAGAAAACCCCGGGCCTATGGCTCTGCCTGTGACCGCACTGCTGCTG |
| | | CCCCTGGCTCTGCTGCTGCACGCCGCAAGACCTGCCGCATACAAGGATATTCAGATGACTCAG |
| | | ACCACAAGCTCCCTGAGCGCCTCCCTGGGAGACCGAGTGACAATCTCTTGCAGTGCATCACAG |
| | | GGAATTAGCAACTACCTGAATTGGTATCAGCAGAAGCCAGATGGCACTGTGAAACTGCTGATC |
| | | TACTATACCTCTAGTCTGCACAGTGGGGTCCCCTCACGATTCAGCGGATCCGGCTCTGGGACA |
| | | GACTACAGCCTGACTATCTCCAACCTGGAGCCCGAAGATATTGCCACCTACTATTGCCAGCAG |
| | | TACTCCAAGCTGCCTTATACCTTTGGCGGGGGAACAAAGCTGGAGATTAAAAGGGGAGGAGGA |
| | | GGAAGCGGAGGAGGAGGATCCGGAGGCGGGGGATCTGGAGGAGGAGGAAGTGAGGTGCAGCTG |
| | | GTCGAATCTGGAGGAGGACTGGTGAAGCCAGGAGGATCTCTGAAACTGAGTTGTGCCGCTTCA |
| | | GGCCTGACCTTCTCAAGCTACGCCATGAGCTGGGTGCGACAGACACCTGAGAAGCGGCTGGAA |
| | | TGGGTCGCTAGCATCTCCTCTGGCGGGTTCACATACTATCCAGACTCCGTGAAAGGCAGATTT |
| | | ACTATCTCTCGGGATAACGCAAGAAATATTCTGTACCTGCAGATGAGTTCACTGAGGAGCGAG |
| | | GACACCGCAATGTACTATTGTGCCAGGGACGAAGTGCGCGGCTATCTGGATGTCTGGGGAGCT |
| | | GGCACTACCGTCACCGTCTCCAGCGAGCAGAAAC T GAT TAGCGAAGAGGACCT |
| | | GAAAGATGAACTGTAA |
| 14 | MSCV | GGAATGAAAGACCCCACCTGTAGGTTTGGCAAGCTAGCTTAAGTAACGCCATTTTGCAAGGCA |
| | promoter-Anti- | TGGAAAATACATAACTGAGAATAGAGAAGTTCAGATCAAGGTTAGGAACAGAGAGACAGCAGA |
| | human CD7 | ATATGGGCCAAACAGGATATCTGTGGTAAGCAGTTCCTGCCCCGGCTCAGGGCCAAGAACAGA |
| | (TH69) CAR- | TGGTCCCCAGATGCGGTCCCGCCCTCAGCAGTTTCTAGAGAACCATCAGATGTTTCCAGGGTG |
| | p2A-Anti- | CCCCAAGGACCTGAAATGACCCTGTGCCTTATTTGAACTAACCAATCAGTTCGCTTCTCGCTT |
| | human CD7 | CTGTTCGCGCGCTTCTGCTCCCCGAGCTCAATAAAAGAGCCCACAACCCCTCACTCGGCGCGC |
| | (TH69) PEBL | CAGTCCGAATTCGGCTTCCACCATGGCTCTGCCTGTGACTGCCCTGCTGCTGCCCCTGGCCCT |
| | | GCTGCTGCACGCTGCTAGACCCGCCGCATACAAGGACATTCAGATGACTCAGACTACATCCAG |
| | | CCTGTCAGCTTCCCTGGGCGATCGAGTGACTATCAGCTGTTCAGCAAGCCAGGGGATCAGCAA |
| | | TTACCTGAACTGGTACCAGCAGAAGCCCGACGGAACTGTCAAGCTGCTGATCTACTATACTTC |
| | | CAGTCTGCATAGCGGGGTCCCTAGCCGATTCTCTGGGAGTGGAAGCGGCACCGACTATAGCCT |
| | | GACCATTTCCAACCTGGAACCAGAGGACATTGCCACCTATTACTGTCAGCAGTATTCAAAGCT |
| | | GCCCTATACCTTTGGCGGAGGGACTAAGCTGGAGATCAAGCGAGGAGGGGGAGGATCAGGGGG |
| | | AGGGGGGAGCGGAGGCGGGGGGAGTGGCGGCGGAGGGTCAGAAGTCCAGCTGGTCGAGAGTGG |
| | | GGGCGGACTGGTGAAACCTGGGGGCTCTCTGAAACTGAGCTGTGCAGCCAGTGGCCTGACATT |
| | | CTCCTCATATGCTATGTCTTGGGTGCGGCAGACCCCAGAAAAGAGACTGGAGTGGGTGGCTTC |
| | | AATCTCTTCCGGGGGCTTCACTTACTATCCTGACAGCGTGAAAGGCAGATTCACTATCTCCAG |
| | | GGATAACGCACGGAATATCCTGTATCTGCAGATGAGCTCACTGAGATCCGAGGACACCGCAAT |
| | | GTACTACTGCGCAAGAGACGAAGTGCGGGGGTATCTGGACGTGTGGGGAGCAGGCACTACCGT |
| | | CACCGTGTCTAGCACCACTACACCTGCACCAAGGCCTCCCACACCCGCTCCCACTATCGCTTC |
| | | CCAGCCACTGTCCCTGAGGCCCGAGGCCTGCAGGCCAGCAGCTGGCGGAGCCGTGCATACTAG |
| | | GGGGCTGGACTTCGCTTGCGACATCTACATCTGGGCCCCACTGGCAGGGACATGCGGAGTCCT |
| | | GCTGCTGTCCCTGGTCATCACACTGTACTGCAAGCGGGGGCGCAAAAAACTGCTGTATATCTT |
| | | TAAGCAGCCTTTCATGAGACCAGTGCAGACAACCCAGGAGGAAGATGGGTGCTCATGCCGGTT |
| | | TCCCGAGGAGGAGGAAGGCGGCTGCGAGCTGAGGGTGAAGTTTTCCCGCTCAGCAGATGCTCC |
| | | TGCCTACCAGCAGGGCCAGAACCAGCTGTATAATGAGCTGAACCTGGGCAGACGCGAAGAGTA |
| | | TGATGTGCTGGACAAAAGGCGGGGAAGAGACCCCGAAATGGGAGGGAAGCCAAGGCGGAAAAA |
| | | CCCCCAGGAGGGCCTGTACAATGAGCTGCAGAAGGACAAAATGGCAGAGGCTTACAGTGAGAT |
| | | TGGGATGAAGGGAGAGAGACGGAGGGGAAAAGGGCACGATGGCCTGTACCAGGGGCTGAGCAC |
| | | AGCAACCAAAGATACTTATGACGCACTGCACATGCAGGCACTGCCACCCAGATGAGGCAGCGG |
| | | CGCCACAAACTTCTCTCTGCTAAAGCAAGCAGGTGATGTTGAAGAAAACCCCGGGCCTATGGC |
| | | TCTGCCTGTGACCGCACTGCTGCTGCCCCTGGCTCTGCTGCTGCACGCCGCAAGACCTGCCGC |
| | | ATACAGCAGTATTCAGATGACTCAGACCACAAGCTCCCTGAGCGCCTCCCTGGGAGACCGAGT |
| | | GACAATCTCTTGCAGTGCATCACAGGGAATTAGCAACTACCTGAATTGGTATCAGCAGAAGCC |
| | | AGATGGCACTGTGAAACTGCTGATCTACTATACCTCTAGTCTGCACAGTGGGGTCCCCTCACG |
| | | ATTCAGCGGATCCGGCTCTGGGACAGACTACAGCCTGACTATCTCCAACCTGGAGCCCGAAGA |
| | | TATTGCCACCTACTATTGCCAGCAGTACTCCAAGCTGCCTTATACCTTTGGCGGGGGAACAAA |
| | | GCTGGAGATTAAAAGGGGAGGAGGAGGAAGCGGAGGAGGAGGATCCGGAGGCGGGGGATCTGG |
| | | AGGAGGAGGAAGTGAGGTGCAGCTGGTCGAATCTGGAGGAGGACTGGTGAAGCCAGGAGGATC |
| | | TCTGAAACTGAGTTGTGCCGCTTCAGGCCTGACCTTCTCAAGCTACGCCATGAGCTGGGTGCG |
| | | ACAGACACCTGAGAAGCGGCTGGAATGGGTCGCTAGCATCTCCTCTGGCGGGTTCACATACTA |
| | | TCCAGACTCCGTGAAAGGCAGATTTACTATCTCTCGGGATAACGCAAGAAATATTCTGTACCT |
| | | GCAGATGAGTTCACTGAGGAGCGAGGACACCGCAATGTACTATTGTGCCAGGGACGAAGTGCG |
| | | CGGCTATCTGGATGTCTGGGGAGCTGGCACTACCGTCACCGTCTCCAGCGAGCAGAAACTGAT |
| | | TAGCGAAGAGGACCTGAAAGATGAACTGTAA |
| 15 | EF1α promoter- | GGCTCCGGTGCCCGTCAGTGGGCAGAGCGCACATCGCCCACAGTCCCCGAGAAGTTGGGGGGA |
| | Anti-human | GGGGTCGGCAATTGAACCGGTGCCTAGAGAAGGTGGCGCGGGGTAAACTGGGAAAGTGATGTC |
| | CD7 (TH69) | GTGTACTGGCTCCGCCTTTTTCCCGAGGGTGGGGGAGAACCGTATATAAGTGCAGTAGTCGCC |
| | CAR-P2A- | GTGAACGTTCTTTTTCGCAACGGGTTTGCCGCCAGAACACAGGTAAGTGCCGTGTGTGGTTCC |
| | Anti-human | CGCGGGCCTGGCCTCTTTACGGGTTATGGCCCTTGCGTGCCTTGAATTACTTCCACCTGGCTG |
| | CD7 (TH69) | CAGTACGTGATTCTTGATCCCGAGCTTCGGGTTGGAAGTGGGTGGGAGAGTTCGAGGCCTTGC |
| | PEBL | GCTTAAGGAGCCCCTTCGCCTCGTGCTTGAGTTGAGGCCTGGCCTGGGCGCTGGGGCCGCCGC |
| | | GTGCGAATCTGGTGGCACCTTCGCGCCTGTCTCGCTGCTTTCGATAAGTCTCTAGCCATTTAA |
| | | AATTTTTGATGACCTGCTGCGACGCTTTTTTTCTGGCAAGATAGTCTTGTAAATGCGGGCCAA |
| | | GATCTGCACACTGGTATTTCGGTTTTTGGGGCCGCGGGCGGCGACGGGGCCCGTGCGTCCCAG |
| | | CGCACATGTTCGGCGAGGCGGGGCCTGCGAGCGCGGCCACCGAGAATCGGACGGGGGTAGTCT |
| | | CAAGCTGGCCGGCCTGCTCTGGTGCCTGGCCTCGCGCCGCCGTGTATCGCCCCGCCCTGGGCG |
| | | GCAAGGCTGGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCACCCACACAA |
| | | AGGAAAAGGGCCTTTCCGTCCTCAGCCGTCGCTTCATGTGACTCCACGGAGTACCGGGCGCCG |
| | | TCCAGGCACCTCGATTAGTTCTCGAGCTTTTGGAGTACGTCGTCTTTAGGTTGGGGGGAGGGG |
| | | TTTTATGCGATGGAGTTTCCCCACACTGAGTGGGTGGAGACTGAAGTTAGGCCAGCTTGGCAC |
| | | TTGATGTAATTCTCCTTGGAATTTGCCCTTTTTGAGTTTGGATCTTGGTTCATTCTCAAGCCT |
| | | CAGACAGTGGTTCAAAGTTTTTTTCTTCCATTTCAGGTGTCGTGAGAATTCGGCTTCCACCAT |
| | | GGCTCTGCCTGTGACTGCCCTGCTGCTGCCCCTGGCCCTGCTGCTGCACGCTGCTAGACCCGC |
| | | CGCATACAAGGACATTCAGATGACTCAGACTACATCCAGCCTGTCAGCTTCCCTGGGCGATCG |
| | | AGTGACTATCAGCTGTTCAGCAAGCCAGGGGATCAGCAATTACCTGAACTGGTACCAGCAGAA |
| | | GCCCGACGGAACTGTCAAGCTGCTGATCTACTATACTTCCAGTCTGCATAGCGGGGTCCCTAG |
| | | CCGATTCTCTGGGAGTGGAAGCGGCACCGACTATAGCCTGACCATTTCCAACCTGGAACCAGA |
| | | GGACATTGCCACCTATTACTGTCAGCAGTATTCAAAGCTGCCCTATACCTTTGGCGGAGGGAC |
| | | TAAGCTGGAGATCAAGCGAGGAGGGGGAGGATCAGGGGGAGGGGGGAGCGGAGGCGGGGGGAG |
| | | TGGCGGCGGAGGGTCAGAAGTCCAGCTGGTCGAGAGTGGGGGGGGACTGGTGAAACCTGGGGG |
| | | CTCTCTGAAACTGAGCTGTGCAGCCAGTGGCCTGACATTCTCCTCATATGCTATGTCTTGGGT |
| | | GCGGCAGACCCCAGAAAAGAGACTGGAGTGGGTGGCTTCAATCTCTTCCGGGGGCTTCACTTA |
| | | CTATCCTGACAGCGTGAAAGGCAGATTCACTATCTCCAGGGATAACGCACGGAATATCCTGTA |
| | | TCTGCAGATGAGCTCACTGAGATCCGAGGACACCGCAATGTACTACTGCGCAAGAGACGAAGT |
| | | GCGGGGGTATCTGGACGTGTGGGGAGCAGGCACTACCGTCACCGTGTCTAGCACCACTACACC |
| | | TGCACCAAGGCCTCCCACACCCGCTCCCACTATCGCTTCCCAGCCACTGTCCCTGAGGCCCGA |
| | | GGCCTGCAGGCCAGCAGCTGGCGGAGCCGTGCATACTAGGGGGCTGGACTTCGCTTGCGACAT |
| | | CTACATCTGGGCCCCACTGGCAGGGACATGCGGAGTCCTGCTGCTGTCCCTGGTCATCACACT |
| | | GTACTGCAAGCGGGGGCGCAAAAAACTGCTGTATATCTTTAAGCAGCCTTTCATGAGACCAGT |
| | | GCAGACAACCCAGGAGGAAGATGGGTGCTCATGCCGGTTTCCCGAGGAGGAGGAAGGCGGCTG |
| | | CGAGCTGAGGGTGAAGTTTTCCCGCTCAGCAGATGCTCCTGCCTACCAGCAGGGCCAGAACCA |
| | | GCTGTATAATGAGCTGAACCTGGGCAGACGCGAAGAGTATGATGTGCTGGACAAAAGGCGGGG |
| | | AAGAGACCCCGAAATGGGAGGGAAGCCAAGGCGGAAAAACCCCCAGGAGGGCCTGTACAATGA |
| | | GCTGCAGAAGGACAAAATGGCAGAGGCTTACAGTGAGATTGGGATGAAGGGAGAGAGACGGAG |
| | | GGGAAAAGGGCACGATGGCCTGTACCAGGGGCTGAGCACAGCAACCAAAGATACTTATGACGC |
| | | ACTGCACATGCAGGCACTGCCACCCAGATGAGGCAGCGGCGCCACAAACTTCTCTCTGCTAAA |
| | | GCAAGCAGGTGATGTTGAAGAAAACCCCGGGCCTATGGCTCTGCCTGTGACCGCACTGCTGCT |
| | | GCCCCTGGCTCTGCTGCTGCACGCCGCAAGACCTGCCGCATACAAGGATATTCAGATGACTCA |
| | | GACCACAAGCTCCCTGAGCGCCTCCCTGGGAGACCGAGTGACAATCTCTTGCAGTGCATCACA |
| | | GGGAATTAGCAACTACCTGAATTGGTATCAGCAGAAGCCAGATGGCACTGTGAAACTGCTGAT |
| | | CTACTATACCTCTAGTCTGCACAGTGGGGTCCCCTCACGATTCAGCGGATCCGGCTCTGGGAC |
| | | AGACTACAGCCTGACTATCTCCAACCTGGAGCCCGAAGATATTGCCACCTACTATTGCCAGCA |
| | | GTACTCCAAGCTGCCTTATACCTTTGGCGGGGGAACAAAGCTGGAGATTAAAAGGGGAGGAGG |
| | | AGAAGCCAGAGGAGGAGGATCCGGAGGCGGGGGATCTGGAGGAGGAGGAAGTGAGGTGCAGCT |
| | | GGTCGAATCTGGAGGAGGACTGGTGAAGCCAGGAGGATCTCTGAAACTGAGTTGTGCCGCTTC |
| | | AGGCCTGACCTTCTCAAGCTACGCCATGAGCTGGGTGCGACAGACACCTGAGAAGCGGCTGGA |
| | | ATGGGTCGCTAGCATCTCCTCTGGCGGGTTCACATACTATCCAGACTCCGTGAAAGGCAGATT |
| | | TACTATCTCTCGGGATAACGCAAGAAATATTCTGTACCTGCAGATGAGTTCACTGAGGAGCGA |
| | | GGACACCGCAATGTACTATTGTGCCAGGGACGAAGTGCGCGGCTATCTGGATGTCTGGGGAGC |
| | | TGGCACTACCGTCACCGTCTCCAGCGAGCAGAAACTGATTAGCGAAGAGGACCTGAAAGATGA |
| | | ACTGTAA |
| 16 | EFS promoter- | GGGCAGAGCGCACATCGCCCACAGTCCCCGAGAAGTTGGGGGGAGGGGTCGGCAATTGAACCG |
| | Anti-human | GTGCCTAGAGAAGGTGGCGCGGGGTAAACTGGGAAAGTGATGTCGTGTACTGGCTCCGCCTTT |
| | CD7 (TH69) | TTCCCGAGGGTGGGGGAGAACCGTATATAAGTGCAGTAGTCGCCGTGAACGTTCTTTTTCGCA |
| | CAR-P2A- | ACGGGTTTGCCGCCAGAACACAGGTGTCGTGACCGCGGGTCTAGAGGATCCGGTACTCGAGGA |
| | Anti-human | ACTGAAAAACCAGAAAGTTAACTGGTAAGTTTAGTCTTTTTGTCTTTTATTTCAGGTCCCGGA |
| | CD7 (TH69) | TCCGGTGGTGGTGCAAATCAAAGAACTGCTCCTCAGTGGATGTTGCCTTTACTTCTAGGAATT |
| | PEBL | CGGCTTCCACCATGGCTCTGCCTGTGACTGCCCTGCTGCTGCCCCTGGCCCTGCTGCTGCACG |
| | | CTGCTAGACCCGCCGCATACAAGGACATTCAGATGACTCAGACTACATCCAGCCTGTCAGCTT |
| | | CCCTGGGCGATCGAGTGACTATCAGCTGTTCAGCAAGCCAGGGGATCAGCAATTACCTGAACT |
| | | GGTACCAGCAGAAGCCCGACGGAACTGTCAAGCTGCTGATCTACTATACTTCCAGTCTGCATA |
| | | GCGGGGTCCCTAGCCGATTCTCTGGGAGTGGAAGCGGCACCGACTATAGCCTGACCATTTCCA |
| | | ACCTGGAACCAGAGGACATTGCCACCTATTACTGTCAGCAGTATTCAAAGCTGCCCTATACCT |
| | | TTGGCGGAGGGACTAAGCTGGAGATCAAGCGAGGAGGGGGAGGATCAGGGGGAGGGGGGAGCG |
| | | GAGGCGGGGGGAGTGGCGGCGGAGGGTCAGAAGTCCAGCTGGTCGAGAGTGGGGGCGGACTGG |
| | | TGAAACCTGGGGGCTCTCTGAAACTGAGCTGTGCAGCCAGTGGCCTGACATTCTCCTCATATG |
| | | CTATGTCTTGGGTGCGGCAGACCCCAGAAAAGAGACTGGAGTGGGTGGCTTCAATCTCTTCCG |
| | | GGGGCTTCACTTACTATCCTGACAGCGTGAAAGGCAGATTCACTATCTCCAGGGATAACGCAC |
| | | GGAATATCCTGTATCTGCAGATGAGCTCACTGAGATCCGAGGACACCGCAATGTACTACTGCG |
| | | CAAGAGACGAAGTGCGGGGGTATCTGGACGTGTGGGGAGCAGGCACTACCGTCACCGTGTCTA |
| | | GCACCACTACACCTGCACCAAGGCCTCCCACACCCGCTCCCACTATCGCTTCCCAGCCACTGT |
| | | CCCTGAGGCCCGAGGCCTGCAGGCCAGCAGCTGGCGGAGCCGTGCATACTAGGGGGCTGGACT |
| | | TCGCTTGCGACATCTACATCTGGGCCCCACTGGCAGGGACATGCGGAGTCCTGCTGCTGTCCC |
| | | TGGTCATCACACTGTACTGCAAGCGGGGGCGCAAAAAACTGCTGTATATCTTTAAGCAGCCTT |
| | | TCATGAGACCAGTGCAGACAACCCAGGAGGAAGATGGGTGCTCATGCCGGTTTCCCGAGGAGG |
| | | AGGAAGGCGGCTGCGAGCTGAGGGTGAAGTTTTCCCGCTCAGCAGATGCTCCTGCCTACCAGC |
| | | AGGGCCAGAACCAGCTGTATAATGAGCTGAACCTGGGCAGACGCGAAGAGTATGATGTGCTGG |
| | | ACAAAAGGCGGGGAAGAGACCCCGAAATGGGAGGGAAGCCAAGGCGGAAAAACCCCCAGGAGG |
| | | GCCTGTACAATGAGCTGCAGAAGGACAAAATGGCAGAGGCTTACAGTGAGATTGGGATGAAGG |
| | | GAGAGAGACGGAGGGGAAAAGGGCACGATGGCCTGTACCAGGGGCTGAGCACAGCAACCAAAG |
| | | ATACTTATGACGCACTGCACATGCAGGCACTGCCACCCAGATGAGGCAGCGGCGCCACAAACT |
| | | TCTCTCTGCTAAAGCAAGCAGGTGATGTTGAAGAAAACCCCGGGCCTATGGCTCTGCCTGTGA |
| | | CCGCACTGCTGCTGCCCCTGGCTCTGCTGCTGCACGCCGCAAGACCTGCCGCATACAAGGATA |
| | | TTCAGATGACTCAGACCACAAGCTCCCTGAGCGCCTCCCTGGGAGACCGAGTGACAATCTCTT |
| | | GCAGTGCATCACAGGGAATTAGCAACTACCTGAATTGGTATCAGCAGAAGCCAGATGACTCTG |
| | | TGAAACTGCTGATCTACTATACCTCTAGTCTGCACAGTGGGGTCCCCTCACGATTCAGCGGAT |
| | | CCGGCTCTGGGACAGACTACAGCCTGACTATCTCCAACCTGGAGCCCGAAGATATTGCCACCT |
| | | ACTATTGCCAGCAGTACTCCAAGCTGCCTTATACCTTTGGCGGGGGAACAAAGCTGGAGATTA |
| | | AAAGGGGAGGAGGAGGAAGCGGAGGAGGAGGATCCGGAGGCGGGGGATCTGGAGGAGGAGGAA |
| | | GTGAGGTGCAGCTGGTCGAATCTGGAGGAGGACTGGTGAAGCCAGGAGGATCTCTGAAACTGA |
| | | GTTGTGCCGCTTCAGGCCTGACCTTCTCAAGCTACGCCATGAGCTGGGTGCGACAGACACCTG |
| | | AGAAGCGGCTGGAATGGGTCGCTAGCATCTCCTCTGGCGGGTTCACATACTATCCAGACTCCG |
| | | TGAAAGGCAGATTTACTATCTCTCGGGATAACGCAAGAAATATTCTGTACCTGCAGATGAGTT |
| | | CACTGAGGAGCGAGGACACCGCAATGTACTATTGTGCCAGGGACGAAGTGCGCGGCTATCTGG |
| | | ATGTCTGGGGAGCTGGCACTACCGTCACCGTCTCCAGCGAGCAGAAACT |
| | | GATTAGCGAAGAGGACCTGAAAGATGAACTGTAA |
| 17 | MSCV | TTAATTAATTTATTTAGTCTCCAGAAAAAGGGGGGAATGAAAGACCCCACCTGTAGGTTTGGC |
| | promoter-Anti- | AAGCTAGCTTAAGTAACGCCATTTTGCAAGGCATGGAAAATACATAACTGAGAATAGAGAAGT |
| | human CD7 | TCAGATCAAGGTTAGGAACAGAGAGACAGCAGAATATGGGCCAAACAGGATATCTGTGGTAAG |
| | (TH69) CAR- | CAGTTCCTGCCCCGGCTCAGGGCCAAGAACAGATGGTCCCCAGATGCGGTCCCGCCCTCAGCA |
| | PGK promoter- | GTTTCTAGAGAACCATCAGATGTTTCCAGGGTGCCCCAAGGACCTGAAATGACCCTGTGCCTT |
| | Anti-human | ATTTGAACTAACCAATCAGTTCGCTTCTCGCTTCTGTTCGCGCGCTTCTGCTCCCCGAGCTCA |
| | CD7 (TH69) | ATAAAAGAGCCCACAACCCCTCACTCGGCGCGCCAGTCCGAATTCGGCTTCCACCATGGCTCT |
| | PEBL | GCCTGTGACTGCCCTGCTGCTGCCCCTGGCCCTGCTGCTGCACGCTGCTAGACCCGCCGCATA |
| | | CAAGGACATTCAGATGACTCAGACTACATCCAGCCTGTCAGCTTCCCTGGGCGATCGAGTGAC |
| | | TATCAGCTGTTCAGCAAGCCAGGGGATCAGCAATTACCTGAACTGGTACCAGCAGAAGCCCGA |
| | | CGGAACTGTCAAGCTGCTGATCTACTATACTTCCAGTCTGCATAGCGGGGTCCCTAGCCGATT |
| | | CTCTGGGAGTGGAAGCGGCACCGACTATAGCCTGACCATTTCCAACCTGGAACCAGAGGACAT |
| | | TGCCACCTATTACTGTCAGCAGTATTCAAAGCTGCCCTATACCTTTGGCGGAGGGACTAAGCT |
| | | GGAGATCAAGCGAGGAGGGGGAGGATCAGGGGGAGGGGGGAGCGGAGGCGGGGGGAGTGGCGG |
| | | CGGAGGGTCAGAAGTCCAGCTGGTCGAGAGTGGGGGCGGACTGGTGAAACCTGGGGGCTCTCT |
| | | GAAACTGAGCTGTGCAGCCAGTGGCCTGACATTCTCCTCATATGCTATGTCTTGGGTGCGGCA |
| | | GACCCCAGAAAAGAGACTGGAGTGGGTGGCTTCAATCTCTTCCGGGGGCTTCACTTACTATCC |
| | | TGACAGCGTGAAAGGCAGATTCACTATCTCCAGGGATAACGCACGGAATATCCTGTATCTGCA |
| | | GATGAGCTCACTGAGATCCGAGGACACCGCAATGTACTACTGCGCAAGAGACGAAGTGCGGGG |
| | | GTATCTGGACGTGTGGGGAGCAGGCACTACCGTCACCGTGTCTAGCACCACTACACCTGCACC |
| | | AAGGCCTCCCACACCCGCTCCCACTATCGCTTCCCAGCCACTGTCCCTGAGGCCCGAGGCCTG |
| | | CAGGCCAGCAGCTGGCGGAGCCGTGCATACTAGGGGGCTGGACTTCGCTTGCGACATCTACAT |
| | | CTGGGCCCCACTGGCAGGGACATGCGGAGTCCTGCTGCTGTCCCTGGTCATCACACTGTACTG |
| | | CAAGCGGGGGCGCAAAAAACTGCTGTATATCTTTAAGCAGCCTTTCATGAGACCAGTGCAGAC |
| | | AACCCAGGAGGAAGATGGGTGCTCATGCCGGTTTCCCGAGGAGGAGGAAGGCGGCTGCGAGCT |
| | | GAGGGTGAAGTTTTCCCGCTCAGCAGATGCTCCTGCCTACCAGCAGGGCCAGAACCAGCTGTA |
| | | TAATGAGCTGAACCTGGGCAGACGCGAAGAGTATGATGTGCTGGACAAAAGGCGGGGAAGAGA |
| | | CCCCGAAATGGGAGGGAAGCCAAGGCGGAAAAACCCCCAGGAGGGCCTGTACAATGAGCTGCA |
| | | GAAGGACAAAATGGCAGAGGCTTACAGTGAGATTGGGATGAAGGGAGAGAGACGGAGGGGAAA |
| | | AGGGCACGATGGCCTGTACCAGGGGCTGAGCACAGCAACCAAAGATACTTATGACGCACTGCA |
| | | CATGCAGGCACTGCCACCCAGATGACTCGAGTGCAGTCGACGCGGGCCCGGGATCCCGCGACT |
| | | CTAGATAATTCTACCGGGTAGGGGAGGCGCTTTTCCCAAGGCAGTCTGGAGCATGCGCTTTAG |
| | | CAGCCCCGCTGGGCACTTGGCGCTACACAAGTGGCCTCTGGCCTCGCACACATTCCACATCCA |
| | | CCGGTAGGCGCCAACCGGCTCCGTTCTTTGGTGGCCCCTTCGCGCCACCTTCTACTCCTCCCC |
| | | TAGTCAGGAAGTTCCCCCCCGCCCCGCAGCTCGCGTCGTGCAGGACGTGACAAATGGAAGTAG |
| | | CACGTCTCACTAGTCTCGTGCAGATGGACAGCACCGCTGAGCAATGGAAGCGGGTAGGCCTTT |
| | | GGGGCAGCGGCCAATAGCAGCTTTGCTCCTTCGCTTTCTGGGCTCAGAGGCTGGGAAGGGGTG |
| | | GGTCCGGGGGCGGGCTCAGGGGGGGGCTCAGGGGCGGGGGGGGCGCCCGAAGGTCCTCCGGAG |
| | | GCCCGGCATTCTGCACGCTTCAAAAGCGCACGTCTGCCGCGCTGTTCTCCTCTTCCTCATCTC |
| | | CGGGCCTTTCGACTGGCCAGGCTTCCACCATGGCTCTGCCTGTGACCGCACTGCTGCTGCCCC |
| | | TGGCTCTGCTGCTGCACGCCGCAAGACCTGCCGCATACAAGGATATTCAGATGACTCAGACCA |
| | | CAAGCTCCCTGAGCGCCTCCCTGGGAGACCGAGTGACAATCTCTTGCAGTGCATCACAGGGAA |
| | | TTAGCAACTACCTGAATTGGTATCAGCAGAAGCCAGATGGCACTGTGAAACTGCTGATCTACT |
| | | ATACCTCTAGTCTGCACAGTGGGGTCCCCTCACGATTCAGCGGATCCGGCTCTGGGACAGACT |
| | | ACAGCCTGACTATCTCCAACCTGGAGCCCGAAGATATTGCCACCTACTATTGCCAGCAGTACT |
| | | CCAAGCTGCCTTATACCTTTGGCGGGGGAACAAAGCTGGAGATTAAAAGGGGAGGAGGAGGAA |
| | | GCGGAGGAGGAGGATCCGGAGGCGGGGGATCTGGAGGAGGAGGAAGTGAGGTGCAGCTGGTCG |
| | | AATCTGGAGGAGGACTGGTGAAGCCAGGAGGATCTCTGAAACTGAGTTGTGCCGCTTCAGGCC |
| | | TGACCTTCTCAAGCTACGCCATGAGCTGGGTGCGACAGACACCTGAGAAGCGGCTGGAATGGG |
| | | TCGCTAGCATCTCCTCTGGCGGGTTCACATACTATCCAGACTCCGTGAAAGGCAGATTTACTA |
| | | TCTCTCGGGATAACGCAAGAAATATTCTGTACCTGCAGATGAGTTCACTGAGGAGCGAGGACA |
| | | CCGCAATGTACTATTGTGCCAGGGACGAAGTGCGCGGCTATCTGGATGTCTGGGGAGCTGGCA |
| | | CTACCGTCACCGTCTCCAGCGAGCAGAAACTGATTAGCGAAGAGGACCTGAAAGATGAACTGT |
| | | AACGCGT |
| 18 | MSCV | GGAATGAAAGACCCCACCTGTAGGTTTGGCAAGCTAGCTTAAGTAACGCCATTTTGCAAGGCA |
| | promoter-Anti- | TGGAAAATACATAACTGAGAATAGAGAAGTTCAGATCAAGGTTAGGAACAGAGAGACAGCAGA |
| | human CD7 | ATATGGGCCAAACAGGATATCTGTGGTAAGCAGTTCCTGCCCCGGCTCAGGGCCAAGAACAGA |
| | (TH69) CAR- | TGGTCCCCAGATGCGGTCCCGCCCTCAGCAGTTTCTAGAGAACCATCAGATGTTTCCAGGGTG |
| | EF1a pro | CCCCAAGGACCTGAAATGACCCTGTGCCTTATTTGAACTAACCAATCAGTTCGCTTCTCGCTT |
| | rooter-Anti- | CTGTTCGCGCGCTTCTGCTCCCCGAGCTCAATAAAAGAGCCCACAACCCCTCACTCGGCGCGC |
| | human CD7 | CAGTCCGAATTCGGCTTCCACCATGGCTCTGCCTGTGACTGCCCTGCTGCTGCCCCTGGCCCT |
| | (TH69) PEBL | GCTGCTGCACGCTGCTAGACCCGCCGCATACAAGGACATTCAGATGACTCAGACTACATCCAG |
| | | CCTGTCAGCTTCCCTGGGCGATCGAGTGACTATCAGCTGTTCAGCAAGCCAGGGGATCAGCAA |
| | | TTACCTGAACTGGTACCAGCAGAAGCCCGACGGAACTGTCAAGCTGCTGATCTACTATACTTC |
| | | CAGTCTGCATAGCGGGGTCCCTAGCCGATTCTCTGGGAGTGGAAGCGGCACCGACTATAGCCT |
| | | GACCATTTCCAACCTGGAACCAGAGGACATTGCCACCTATTACTGTCAGCAGTATTCAAAGCT |
| | | GCCCTATACCTTTGGCGGAGGGACTAAGCTGGAGATCAAGCGAGGAGGGGGAGGATCAGGGGG |
| | | AGGGGGGAGCGGAGGCGGGGGGAGTGGCGGCGGAGGGTCAGAAGTCCAGCTGGTCGAGAGTGG |
| | | GGGCGGACTGGTGAAACCTGGGGGCTCTCTGAAACTGAGCTGTGCAGCCAGTGGCCTGACATT |
| | | CTCCTCATATGCTATGTCTTGGGTGCGGCAGACCCCAGAAAAGAGACTGGAGTGGGTGGCTTC |
| | | AATCTCTTCCGGGGGCTTCACTTACTATCCTGACAGCGTGAAAGGCAGATTCACTATCTCCAG |
| | | GGATAACGCACGGAATATCCTGTATCTGCAGATGAGCTCACTGAGATCCGAGGACACCGCAAT |
| | | GTACTACTGCGCAAGAGACGAAGTGCGGGGGTATCTGGACGTGTGGGGAGCAGGCACTACCGT |
| | | CACCGTGTCTAGCACCACTACACCTGCACCAAGGCCTCCCACACCCGCTCCCACTATCGCTTC |
| | | CCAGCCACTGTCCCTGAGGCCCGAGGCCTGCAGGCCAGCAGCTGGCGGAGCCGTGCATACTAG |
| | | GGGGCTGGACTTCGCTTGCGACATCTACATCTGGGCCCCACTGGCAGGGACATGCGGAGTCCT |
| | | GCTGCTGTCCCTGGTCATCACACTGTACTGCAAGCGGGGGCGCAAAAAACTGCTGTATATCTT |
| | | TAAGCAGCCTTTCATGAGACCAGTGCAGACAACCCAGGAGGAAGATGGGTGCTCATGCCGGTT |
| | | TCCCGAGGAGGAGGAAGGCGGCTGCGAGCTGAGGGTGAAGTTTTCCCGCTCAGCAGATGCTCC |
| | | TGCCTACCAGCAGGGCCAGAACCAGCTGTATAATGAGCTGAACCTGGGCAGACGCGAAGAGTA |
| | | TGATGTGCTGGACAAAAGGCGGGGAAGAGACCCCGAAATGGGAGGGAAGCCAAGGCGGAAAAA |
| | | CCCCCAGGAGGGCCTGTACAATGAGCTGCAGAAGGACAAAATGGCAGAGGCTTACAGTGAGAT |
| | | TGGGATGAAGGGAGAGAGACGGAGGGGAAAAGGGCACGATGGCCTGTACCAGGGGCTGAGCAC |
| | | AGCAACCAAAGATACTTATGACGCACTGCACATGCAGGCACTGCCACCCAGATGAGTCGACGG |
| | | CTCCGGTGCCCGTCAGTGGGCAGAGCGCACATCGCCCACAGTCCCCGAGAAGTTGGGGGGAGG |
| | | GGTCGGCAATTGAACCGGTGCCTAGAGAAGGTGGCGCGGGGTAAACTGGGAAAGTGATGTCGT |
| | | GTACTGGCTCCGCCTTTTTCCCGAGGGTGGGGGAGAACCGTATATAAGTGCAGTAGTCGCCGT |
| | | GAACGTTCTTTTTCGCAACGGGTTTGCCGCCAGAACACAGGTAAGTGCCGTGTGTGGTTCCCG |
| | | CGGGCCTGGCCTCTTTACGGGTTATGGCCCTTGCGTGCCTTGAATTACTTCCACCTGGCTGCA |
| | | GTACGTGATTCTTGATCCCGAGCTTCGGGTTGGAAGTGGGTGGGAGAGTTCGAGGCCTTGCGC |
| | | TTAAGGAGCCCCTTCGCCTCGTGCTTGAGTTGAGGCCTGGCCTGGGCGCTGGGGCCGCCGCGT |
| | | GCGAATCTGGTGGCACCTTCGCGCCTGTCTCGCTGCTTTCGATAAGTCTCTAGCCATTTAAAA |
| | | TTTTTGATGACCTGCTGCGACGCTTTTTTTCTGGCAAGATAGTCTTGTAAATGCGGGCCAAGA |
| | | TCTGCACACTGGTATTTCGGTTTTTGGGGCCGCGGGCGGCGACGGGGCCCGTGCGTCCCAGCG |
| | | CACATGTTCGGCGAGGCGGGGCCTGCGAGCGCGGCCACCGAGAATCGGACGGGGGTAGTCTCA |
| | | AGCTGGCCGGCCTGCTCTGGTGCCTGGCCTCGCGCCGCCGTGTATCGCCCCGCCCTGGGCGGC |
| | | AAGGCTGGCCCGGTCGGCACCAGTTGCGTGAGCGGAAAGATGGCCGCTTCCCGGCCCTGCTGC |
| | | AGGGAGCTCAAAATGGAGGACGCGGCGCTCGGGAGAGCGGGCGGGTGAGTCACCCACACAAAG |
| | | GAAAAGGGCCTTTCCGTCCTCAGCCGTCGCTTCATGTGACTCCACGGAGTACCGGGCGCCGTC |
| | | CAGGCACCTCGATTAGTTCTCGAGCTTTTGGAGTACGTCGTCTTTAGGTTGGGGGGAGGGGTT |
| | | TTATGCGATGGAGTTTCCCCACACTGAGTGGGTGGAGACTGAAGTTAGGCCAGCTTGGCACTT |
| | | GATGTAATTCTCCTTGGAATTTGCCCTTTTTGAGTTTGGATCTTGGTTCATTCTCAAGCCTCA |
| | | GACAGTGGTTCAAAGTTTTTTTCTTCCATTTCAGGTGTCGTGACCCGGGGGCTTCCACCATGG |
| | | CTCTGCCTGTGACCGCACTGCTGCTGCCCCTGGCTCTGCTGCTGCACGCCGCAAGACCTGCCG |
| | | CATACAAGGATATTCAGATGACTCAGACCACAAGCTCCCTGAGCGCCTCCCTGGGAGACCGAG |
| | | TGACAATCTCTTGCAGTGCATCACAGGGAATTAGCAACTACCTGAATTGGTATCAGCAGAAGC |
| | | CAGATGGCACTGTGAAACTGCTGATCTACTATACCTCTAGTCTGCACAGTGGGGTCCCCTCAC |
| | | GATTCAGCGGATCCGGCTCTGGGACAGACTACAGCCTGACTATCTCCAACCTGGAGCCCGAAG |
| | | ATATTGCCACCTACTATTGCCAGCAGTACTCCAAGCTGCCTTATACCTTTGGCGGGGGAACAA |
| | | AGCTGGAGATTAAAAGGGGAGGAGGAGGAAGCGGAGGAGGAGGATCCGGAGGCGGGGGATCTG |
| | | GAGGAGGAGGAAGTGAGGTGCAGCTGGTCGAATCTGGAGGAGGACTGGTGAAGCCAGGAGGAT |
| | | CTCTGAAACTGAGTTGTGCCGCTTCAGGCCTGACCTTCTCAAGCTACGCCATGAGCTGGGTGC |
| | | GACAGACACCTGAGAAGCGGCTGGAATGGGTCGCTAGCATCTCCTCTGGCGGGTTCACATACT |
| | | ATCCAGACTCCGTGAAAGGCAGATTTACTATCTCTCGGGATAACGCAAGAAATATTCTGTACC |
| | | TGCAGATGAGTTCACTGAGGAGCGAGGACACCGCAATGTACTATTGTGCCAGGGACGAAGTGC |
| | | GCGGCTATCTGGATGTCTGGGGAGCTGGCACTACCGTCACCGTCTCCAGCGAGCAGAAACTGA |
| | | TTAGCGAAGAGGACCTGAAAGATGAACTGTAA |
| 19 | MSCV | GGAATGAAAGACCCCACCTGTAGGTTTGGCAAGCTAGCTTAAGTAACGCCATTTTGCAAGGCA |
| | promoter-anti- | TGGAAAATACATAACTGAGAATAGAGAAGTTCAGATCAAGGTTAGGAACAGAGAGACAGCAGA |
| | human CD7 | ATATGGGCCAAACAGGATATCTGTGGTAAGCAGTTCCTGCCCCGGCTCAGGGCCAAGAACAGA |
| | (TH69) CAR- | TGGTCCCCAGATGCGGTCCCGCCCTCAGCAGTTTCTAGAGAACCATCAGATGTTTCCAGGGTG |
| | EFS promoter- | CCCCAAGGACCTGAAATGACCCTGTGCCTTATTTGAACTAACCAATCAGTTCGCTTCTCGCTT |
| | Anti-human | CTGTTCGCGCGCTTCTGCTCCCCGAGCTCAATAAAAGAGCCCACAACCCCTCACTCGGCGCGC |
| | CD7 (TH69) | CAGTCC/GAATTCGGCTTCCACC/ATGGCTCTGCCTGTGACTGCCCTGCTGCTGCCCCTGGCC |
| | PEBL | CTGCTGCTGCACGCTGCTAGACCCGCCGCATACAAGGACATTCAGATGACTCAGACTACATCC |
| | | AGCCTGTCAGCTTCCCTGGGCGATCGAGTGACTATCAGCTGTTCAGCAAGCCAGGGGATCAGC |
| | | AATTACCTGAACTGGTACCAGCAGAAGCCCGACGGAACTGTCAAGCTGCTGATCTACTATACT |
| | | TCCAGTCTGCATAGCGGGGTCCCTAGCCGATTCTCTGGGAGTGGAAGCGGCACCGACTATAGC |
| | | CTGACCATTTCCAACCTGGAACCAGAGGACATTGCCACCTATTACTGTCAGCAGTATTCAAAG |
| | | CTGCCCTATACCTTTGGCGGAGGGACTAAGCTGGAGATCAAGCGAGGAGGGGGAGGATCAGGG |
| | | GGAGGGGGGAGCGGAGGCGGGGGGAGTGGCGGCGGAGGGTCAGAAGTCCAGCTGGTCGAGAGT |
| | | GGGGGCGGACTGGTGAAACCTGGGGGCTCTCTGAAACTGAGCTGTGCAGCCAGTGGCCTGACA |
| | | TTCTCCTCATATGCTATGTCTTGGGTGCGGCAGACCCCAGAAAAGAGACTGGAGTGGGTGGCT |
| | | TCAATCTCTTCCGGGGGCTTCACTTACTATCCTGACAGCGTGAAAGGCAGATTCACTATCTCC |
| | | AGGGATAACGCACGGAATATCCTGTATCTGCAGATGAGCTCACTGAGATCCGAGGACACCGCA |
| | | ATGTACTACTGCGCAAGAGACGAAGTGCGGGGGTATCTGGACGTGTGGGGAGCAGGCACTACC |
| | | GTCACCGTGTCTAGCACCACTACACCTGCACCAAGGCCTCCCACACCCGCTCCCACTATCGCT |
| | | TCCCAGCCACTGTCCCTGAGGCCCGAGGCCTGCAGGCCAGCAGCTGGCGGAGCCGTGCATACT |
| | | AGGGGGCTGGACTTCGCTTGCGACATCTACATCTGGGCCCCACTGGCAGGGACATGCGGAGTC |
| | | CTGCTGCTGTCCCTGGTCATCACACTGTACTGCAAGCGGGGGCGCAAAAAACTGCTGTATATC |
| | | TTTAAGCAGCCTTTCATGAGACCAGTGCAGACAACCCAGGAGGAAGATGGGTGCTCATGCCGG |
| | | TTTCCCGAGGAGGAGGAAGGCGGCTGCGAGCTGAGGGTGAAGTTTTCCCGCTCAGCAGATGCT |
| | | CCTGCCTACCAGCAGGGCCAGAACCAGCTGTATAATGAGCTGAACCTGGGCAGACGCGAAGAG |
| | | TATGATGTGCTGGACAAAAGGCGGGGAAGAGACCCCGAAATGGGAGGGAAGCCAAGGCGGAAA |
| | | AACCCCCAGGAGGGCCTGTACAATGAGCTGCAGAAGGACAAAATGGCAGAGGCTTACAGTGAG |
| | | ATTGGGATGAAGGGAGAGAGACGGAGGGGAAAAGGGCACGATGGCCTGTACCAGGGGCTGAGC |
| | | ACAGCAACCAAAGATACTTATGACGCACTGCACATGCAGGCACTGCCACCCAGATGAGTCGAC |
| | | GGGCAGAGCGCACATCGCCCACAGTCCCCGAGAAGTTGGGGGGAGGGGTCGGCAATTGAACCG |
| | | GTGCCTAGAGAAGGTGGCGCGGGGTAAACTGGGAAAGTGATGTCGTGTACTGGCTCCGCCTTT |
| | | TTCCCGAGGGTGGGGGAGAACCGTATATAAGTGCAGTAGTCGCCGTGAACGTTCTTTTTCGCA |
| | | ACGGGTTTGCCGCCAGAACACAGGTGTCGTGACCGCGGGTCTAGAGGATCCGGTACTCGAGGA |
| | | ACTGAAAAACCAGAAAGTTAACTGGTAAGTTTAGTCTTTTTGTCTTTTATTTCAGGTCCCGGA |
| | | TCCGGTGGTGGTGCAAATCAAAGAACTGCTCCTCAGTGGATGTTGCCTTTACTTCTAGCCCGG |
| | | GGGCTTCCACCATGGCTCTGCCTGTGACCGCACTGCTGCTGCCCCTGGCTCTGCTGCTGCACG |
| | | CCGCAAGACCTGCCGCATACAAGGATATTCAGATGACTCAGACCACAAGCTCCCTGAGCGCCT |
| | | CCCTGGGAGACCGAGTGACAATCTCTTGCAGTGCATCACAGGGAATTAGCAACTACCTGAATT |
| | | GGTATCAGCAGAAGCCAGATGGCACTGTGAAACTGCTGATCTACTATACCTCTAGTCTGCACA |
| | | GTGGGGTCCCCTCACGATTCAGCGGATCCGGCTCTGGGACAGACTACAGCCTGACTATCTCCA |
| | | ACCTGGAGCCCGAAGATATTGCCACCTACTATTGCCAGCAGTACTCCAAGCTGCCTTATACCT |
| | | TTGGCGGGGGAACAAAGCTGGAGATTAAAAGGGGAGGAGGAGGAAGCGGAGGAGGAGGATCCG |
| | | GAGGCGGGGGATCTGGAGGAGGAGGAAGTGAGGTGCAGCTGGTCGAATCTGGAGGAGGACTGG |
| | | TGAAGCCAGGAGGATCTCTGAAACTGAGTTGTGCCGCTTCAGGCCTGACCTTCTCAAGCTACG |
| | | CCATGAGCTGGGTGCGACAGACACCTGAGAAGCGGCTGGAATGGGTCGCTAGCATCTCCTCTG |
| | | GCGGGTTCACATACTATCCAGACTCCGTGAAAGGCAGATTTACTATCTCTCGGGATAACGCAA |
| | | GAAATATTCTGTACCTGCAGATGAGTTCACTGAGGAGCGAGGACACCGCAATGTACTATTGTG |
| | | CCAGGGACGAAGTGCGCGGCTATCTGGATGTCTGGGGAGCTGGCACTACCGTCACCGTCTCCA |
| | | GCGAGCAGAAACTGATTAGCGAAGAGGACCTGAAAGATGAACTGTAA |
| 20 | PGK promoter- | ATTCTACCGGGTAGGGGAGGCGCTTTTCCCAAGGCAGTCTGGAGCATGCGCTTTAGCAGCCCC |
| | anti-human | GCTGGGCACTTGGCGCTACACAAGTGGCCTCTGGCCTCGCACACATTCCACATCCACCGGTAG |
| | CD7 (3A1F) | GCGCCAACCGGCTCCGTTCTTTGGTGGCCCCTTCGCGCCACCTTCTACTCCTCCCCTAGTCAG |
| | CAR-EF1a | GAAGTTCCCCCCCGCCCCGCAGCTCGCGTCGTGCAGGACGTGACAAATGGAAGTAGCACGTCT |
| | promoter-Anti- | CACTAGTCTCGTGCAGATGGACAGCACCGCTGAGCAATGGAAGCGGGTAGGCCTTTGGGGCAG |
| | human CD7 | CGGCCAATAGCAGCTTTGCTCCTTCGCTTTCTGGGCTCAGAGGCTGGGAAGGGGTGGGTCCGG |
| | (TH69) PEBL | GGGCGGGCTCAGGGGCGGGCTCAGGGGCGGGGGGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGAGAATTCGGCTTCCACCATGGCTCTGCCCGTCACCGCTCTGCTGCTGCCTCTGGCTCTG |
| | | CTGCTGCACGCTGCTCGACCACAGGTCCAGCTGCAGGAGTCAGGAGCTGAGCTGGTGAAGCCA |
| | | GGGGCAAGCGTCAAACTGTCCTGCAAGGCCTCTGGATATACATTCACTAGCTACTGGATGCAC |
| | | TGGGTGAAACAGAGACCCGGACAGGGCCTGGAGTGGATCGGAAAGATTAACCCTAGCAATGGC |
| | | AGGACCAACTACAACGAAAAGTTTAAATCCAAGGCAACCCTGACAGTGGACAAGAGCTCCTCT |
| | | ACAGCCTACATGCAGCTGAGTTCACTGACTTCAGAGGATAGCGCAGTGTACTATTGCGCCAGA |
| | | GGCGGGGTCTACTATGACCTGTACTATTACGCCCTGGATTATTGGGGGCAGGGAACCACAGTG |
| | | ACTGTCAGCTCCGGAGGAGGAGGAAGCGGAGGAGGAGGATCCGGAGGAGGAGGATCTGACATC |
| | | GAGCTGACCCAGAGTCCTGCTACACTGAGCGTGACTCCAGGCGATTCTGTCAGTCTGTCATGT |
| | | CGGGCAAGCCAGTCCATCTCTAACAATCTGCACTGGTACCAGCAGAAATCCCATGAATCTCCA |
| | | CGACTGCTGATTAAGAGTGCCTCACAGAGCATCTCCGGCATTCCCTCCCGGTTCTCTGGCAGT |
| | | GGGTCAGGAACTGACTTTACCCTGAGTATTAACTCAGTGGAGACAGAAGATTTCGGCATGTAT |
| | | TTTTGCCAGCAGAGCAATTCCTGGCCCTACACTTTCGGAGGCGGGACCAAACTGGAGATCAAG |
| | | CGGACTACCACACCAGCTCCAAGACCACCTACCCCTGCACCAACAATTGCTAGTCAGCCACTG |
| | | TCACTGAGACCAGAAGCATGTAGGCCTGCAGCTGGAGGAGCTGTGCACACCAGAGGCCTGGAC |
| | | TTTGCCTGCGATATCTACATTTGGGCTCCTCTGGCAGGAACCTGTGGCGTGCTGCTGCTGTCT |
| | | CTGGTCATCACACTGTATTGCAAACGAGGGCGGAAGAAACTGCTGTACATTTTCAAGCAGCCC |
| | | TTTATGAGGCCTGTGCAGACTACCCAGGAGGAAGACGGCTGCAGCTGTCGCTTCCCTGAGGAA |
| | | GAGGAAGGGGGATGTGAGCTGCGCGTCAAATTTTCTCGAAGTGCAGATGCCCCAGCTTATCAG |
| | | CAGGGCCAGAACCAGCTGTACAACGAGCTGAATCTGGGGCGGAGAGAGGAATATGACGTGCTG |
| | | GATAAGAGGCGAGGAAGGGACCCAGAAATGGGAGGGAAACCACGACGGAAGAACCCCCAGGAG |
| | | GGCCTGTATAATGAACTGCAGAAAGACAAGATGGCAGAGGCCTACTCTGAAATCGGGATGAAA |
| | | GGAGAGAGAAGGCGCGGCAAGGGGCATGATGGCCTGTACCAGGGGCTGAGCACAGCAACCAAA |
| | | GACACCTATGACGCACTGCACATGCAGGCACTGCCACCACGGTAATGAGTCGACGGCTCCGGT |
| | | GCCCGTCAGTGGGCAGAGCGCACATCGCCCACAGTCCCCGAGAAGTTGGGGGGAGGGGTCGGC |
| | | AATTGAACCGGTGCCTAGAGAAGGTGGCGCGGGGTAAACTGGGAAAGTGATGTCGTGTACTGG |
| | | CTCCGCCTTTTTCCCGAGGGTGGGGGAGAACCGTATATAAGTGCAGTAGTCGCCGTGAACGTT |
| | | CTTTTTCGCAACGGGTTTGCCGCCAGAACACAGGTAAGTGCCGTGTGTGGTTCCCGCGGGCCT |
| | | GGCCTCTTTACGGGTTATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGAGGCCTTGCGCTTAAGGA |
| | | GCCCCTTCGCCTCGTGCTTGAGTTGAGGCCTGGCCTGGGCGCTGGGGCCGCCGCGTGCGAATC |
| | | TGGTGGCACCTTCGCGCCTGTCTCGCTGCTTTCGATAAGTCTCTAGCCATTTAAAATTTTTGA |
| | | TGACCTGCTGCGACGCTTTTTTTCTGGCAAGATAGTCTTGTAAATGCGGGCCAAGATCTGCAC |
| | | ACTGGTATTTCGGTTTTTGGGGCCGCGGGCGGCGACGGGGCCCGTGCGTCCCAGCGCACATGT |
| | | TCGGCGAGGCGGGGCCTGCGAGCGCGGCCACCGAGAATCGGACGGGGGTAGTCTCAAGCTGGC |
| | | CGGCCTGCTCTGGTGCCTGGCCTCGCGCCGCCGTGTATCGCCCCGCCCTGGGCGGCAAGGCTG |
| | | GCCCGGTCGGCACCAGTTGCGTGAGCGGAAAGATGGCCGCTTCCCGGCCCTGCTGCAGGGAGC |
| | | TCAAAATGGAGGACGCGGCGCTCGGGAGAGCGGGCGGGTGAGTCACCCACACAAAGGAAAAGG |
| | | GCCTTTCCGTCCTCAGCCGTCGCTTCATGTGACTCCACGGAGTACCGGGCGCCGTCCAGGCAC |
| | | CTCGATTAGTTCTCGAGCTTTTGGAGTACGTCGTCTTTAGGTTGGGGGGAGGGGTTTTATGCG |
| | | ATGGAGTTTCCCCACACTGAGTGGGTGGAGACTGAAGTTAGGCCAGCTTGGCACTTGATGTAA |
| | | TTCTCCTTGGAATTTGCCCTTTTTGAGTTTGGATCTTGGTTCATTCTCAAGCCTCAGACAGTG |
| | | GTTCAAAGTTTTTTTCTTCCATTTCAGGTGTCGTGACCCGGGGGCTTCCACCATGGCTCTGCC |
| | | TGTGACCGCACTGCTGCTGCCCCTGGCTCTGCTGCTGCACGCCGCAAGACCTGCCGCATACAA |
| | | GGATATTCAGATGACTCAGACCACAAGCTCCCTGAGCGCCTCCCTGGGAGACCGAGTGACAAT |
| | | CTCTTGCAGTGCATCACAGGGAATTAGCAACTACCTGAATTGGTATCAGCAGAAGCCAGATGG |
| | | CACTGTGAAACTGCTGATCTACTATACCTCTAGTCTGCACAGTGGGGTCCCCTCACGATTCAG |
| | | CGGATCCGGCTCTGGGACAGACTACAGCCTGACTATCTCCAACCTGGAGCCCGAAGATATTGC |
| | | CACCTACTATTGCCAGCAGTACTCCAAGCTGCCTTATACCTTTGGCGGGGGAACAAAGCTGGA |
| | | GATTAAAAGGGGAGGAGGAGGAAGCGGAGGAGGAGGATCCGGAGGCGGGGGATCTGGAGGAGG |
| | | AGGAAGTGAGGTGCAGCTGGTCGAATCTGGAGGAGGACTGGTGAAGCCAGGAGGATCTCTGAA |
| | | ACTGAGTTGTGCCGCTTCAGGCCTGACCTTCTCAAGCTACGCCATGAGCTGGGTGCGACAGAC |
| | | ACCTGAGAAGCGGCTGGAATGGGTCGCTAGCATCTCCTCTGGCGGGTTCACATACTATCCAGA |
| | | CTCCGTGAAAGGCAGATTTACTATCTCTCGGGATAACGCAAGAAATATTCTGTACCTGCAGAT |
| | | GAGTTCACTGAGGAGCGAGGACACCGCAATGTACTATTGTGCCAGGGACGAAGTGCGCGGCTA |
| | | TCTGGATGTCTGGGGAGCTGGCACTACCGTCACCGTCTCCAGCGAGCAGAAACTGATTAGCGA |
| | | AGAGGACCTGAAAGATGAACTGTAA |
| 21 | PGK promoter- | ATTCTACCGGGTAGGGGAGGCGCTTTTCCCAAGGCAGTCTGGAGCATGCGCTTTAGCAGCCCC |
| | anti-human | GCTGGGCACTTGGCGCTACACAAGTGGCCTCTGGCCTCGCACACATTCCACATCCACCGGTAG |
| | CD7 (TH69) | GCGCCAACCGGCTCCGTTCTTTGGTGGCCCCTTCGCGCCACCTTCTACTCCTCCCCTAGTCAG |
| | CAR-EF1a | GAAGTTCCCCCCCGCCCCGCAGCTCGCGTCGTGCAGGACGTGACAAATGGAAGTAGCACGTCT |
| | promoter-Anti- | CACTAGTCTCGTGCAGATGGACAGCACCGCTGAGCAATGGAAGCGGGTAGGCCTTTGGGGCAG |
| | human CD7 | CGGCCAATAGCAGCTTTGCTCCTTCGCTTTCTGGGCTCAGAGGCTGGGAAGGGGTGGGTCCGG |
| | (3A1F) PEBL | GGGCGGGCTCAGGGGGGGGCTCAGGGGCGGGGGGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGAGAATTCGGCTTCCACCATGGCTCTGCCTGTGACTGCCCTGCTGCTGCCCCTGGCCCTG |
| | | CTGCTGCACGCTGCTAGACCCGCCGCATACAAGGACATTCAGATGACTCAGACTACATCCAGC |
| | | CTGTCAGCTTCCCTGGGCGATCGAGTGACTATCAGCTGTTCAGCAAGCCAGGGGATCAGCAAT |
| | | TACCTGAACTGGTACCAGCAGAAGCCCGACGGAACTGTCAAGCTGCTGATCTACTATACTTCC |
| | | AGTCTGCATAGCGGGGTCCCTAGCCGATTCTCTGGGAGTGGAAGCGGCACCGACTATAGCCTG |
| | | ACCATTTCCAACCTGGAACCAGAGGACATTGCCACCTATTACTGTCAGCAGTATTCAAAGCTG |
| | | CCCTATACCTTTGGCGGAGGGACTAAGCTGGAGATCAAGCGAGGAGGGGGAGGATCAGGGGGA |
| | | GGGGGGAGCGGAGGCGGGGGGAGTGGCGGCGGAGGGTCAGAAGTCCAGCTGGTCGAGAGTGGG |
| | | GGCGGACTGGTGAAACCTGGGGGCTCTCTGAAACTGAGCTGTGCAGCCAGTGGCCTGACATTC |
| | | TCCTCATATGCTATGTCTTGGGTGCGGCAGACCCCAGAAAAGAGACTGGAGTGGGTGGCTTCA |
| | | ATCTCTTCCGGGGGCTTCACTTACTATCCTGACAGCGTGAAAGGCAGATTCACTATCTCCAGG |
| | | GATAACGCACGGAATATCCTGTATCTGCAGATGAGCTCACTGAGATCCGAGGACACCGCAATG |
| | | TACTACTGCGCAAGAGACGAAGTGCGGGGGTATCTGGACGTGTGGGGAGCAGGCACTACCGTC |
| | | ACCGTGTCTAGCACCACTACACCTGCACCAAGGCCTCCCACACCCGCTCCCACTATCGCTTCC |
| | | CAGCCACTGTCCCTGAGGCCCGAGGCCTGCAGGCCAGCAGCTGGCGGAGCCGTGCATACTAGG |
| | | GGGCTGGACTTCGCTTGCGACATCTACATCTGGGCCCCACTGGCAGGGACATGCGGAGTCCTG |
| | | CTGCTGTCCCTGGTCATCACACTGTACTGCAAGCGGGGGCGCAAAAAACTGCTGTATATCTTT |
| | | AAGCAGCCTTTCATGAGACCAGTGCAGACAACCCAGGAGGAAGATGGGTGCTCATGCCGGTTT |
| | | CCCGAGGAGGAGGAAGGCGGCTGCGAGCTGAGGGTGAAGTTTTCCCGCTCAGCAGATGCTCCT |
| | | GCCTACCAGCAGGGCCAGAACCAGCTGTATAATGAGCTGAACCTGGGCAGACGCGAAGAGTAT |
| | | GATGTGCTGGACAAAAGGCGGGGAAGAGACCCCGAAATGGGAGGGAAGCCAAGGCGGAAAAAC |
| | | CCCCAGGAGGGCCTGTACAATGAGCTGCAGAAGGACAAAATGGCAGAGGCTTACAGTGAGATT |
| | | GGGATGAAGGGAGAGAGACGGAGGGGAAAAGGGCACGATGGCCTGTACCAGGGGCTGAGCACA |
| | | GCAACCAAAGATACTTATGACGCACTGCACATGCAGGCACTGCCACCCAGATGAGTCGACGGC |
| | | TCCGGTGCCCGTCAGTGGGCAGAGCGCACATCGCCCACAGTCCCCGAGAAGTTGGGGGGAGGG |
| | | GTCGGCAATTGAACCGGTGCCTAGAGAAGGTGGCGCGGGGTAAACTGGGAAAGTGATGTCGTG |
| | | TACTGGCTCCGCCTTTTTCCCGAGGGTGGGGGAGAACCGTATATAAGTGCAGTAGTCGCCGTG |
| | | AACGTTCTTTTTCGCAACGGGTTTGCCGCCAGAACACAGGTAAGTGCCGTGTGTGGTTCCCGC |
| | | GGGCCTGGCCTCTTTACGGGTTATGGCCCTTGCGTGCCTTGAATTACTTCCACCTGGCTGCAG |
| | | TACGTGATTCTTGATCCCGAGCTTCGGGTTGGAAGTGGGTGGGAGAGTTCGAGGCCTTGCGCT |
| | | TAAGGAGCCCCTTCGCCTCGTGCTTGAGTTGAGGCCTGGCCTGGGCGCTGGGGCCGCCGCGTG |
| | | CGAATCTGGTGGCACCTTCGCGCCTGTCTCGCTGCTTTCGATAAGTCTCTAGCCATTTAAAAT |
| | | TTTTGATGACCTGCTGCGACGCTTTTTTTCTGGCAAGATAGTCTTGTAAATGCGGGCCAAGAT |
| | | CTGCACACTGGTATTTCGGTTTTTGGGGCCGCGGGCGGCGACGGGGCCCGTGCGTCCCAGCGC |
| | | ACATGTTCGGCGAGGCGGGGCCTGCGAGCGCGGCCACCGAGAATCGGACGGGGGTAGTCTCAA |
| | | GCTGGCCGGCCTGCTCTGGTGCCTGGCCTCGCGCCGCCGTGTATCGCCCCGCCCTGGGCGGCA |
| | | AGGCTGGCCCGGTCGGCACCAGTTGCGTGAGCGGAAAGATGGCCGCTTCCCGGCCCTGCTGCA |
| | | GGGAGCTCAAAATGGAGGACGCGGCGCTCGGGAGAGCGGGCGGGTGAGTCACCCACACAAAGG |
| | | AAAAGGGCCTTTCCGTCCTCAGCCGTCGCTTCATGTGACTCCACGGAGTACCGGGCGCCGTCC |
| | | AGGCACCTCGATTAGTTCTCGAGCTTTTGGAGTACGTCGTCTTTAGGTTGGGGGGAGGGGTTT |
| | | TATGCGATGGAGTTTCCCCACACTGAGTGGGTGGAGACTGAAGTTAGGCCAGCTTGGCACTTG |
| | | ATGTAATTCTCCTTGGAATTTGCCCTTTTTGAGTTTGGATCTTGGTTCATTCTCAAGCCTCAG |
| | | ACAGTGGTTCAAAGTTTTTTTCTTCCATTTCAGGTGTCGTGACCCGGGGGCTTCCACCATGGC |
| | | TCTGCCCGTCACCGCTCTGCTGCTGCCTCTGGCTCTGCTGCTGCACGCCGCAAGACCCCAGGT |
| | | CCAGCTGCAGGAGTCAGGGGCAGAGCTGGTGAAACCCGGAGCCAGTGTCAAACTGTCCTGTAA |
| | | GGCCAGCGGCTATACTTTCACCAGCTACTGGATGCACTGGGTGAAACAGAGGCCAGGACAGGG |
| | | CCTGGAGTGGATCGGCAAGATTAACCCCAGCAATGGGCGCACCAACTACAACGAAAAGTTTAA |
| | | ATCCAAGGCTACACTGACTGTGGACAAGAGCTCCTCTACCGCATACATGCAGCTGAGTTCACT |
| | | GACATCTGAAGATAGTGCCGTGTACTATTGCGCCAGAGGGGGGGTCTACTATGACCTGTACTA |
| | | TTACGCACTGGATTATTGGGGGCAGGGAACCACAGTGACTGTCAGCTCCGGAGGAGGAGGATC |
| | | CGGCGGAGGAGGCTCTGGGGGAGGCGGGAGTGACATCGAGCTGACACAGTCTCCAGCCACTCT |
| | | GAGCGTGACCCCTGGCGATTCTGTCAGTCTGTCATGTAGAGCTAGCCAGTCCATCTCTAACAA |
| | | TCTGCACTGGTACCAGCAGAAATCACATGAAAGCCCTCGGCTGCTGATTAAGAGTGCTTCACA |
| | | GAGCATCTCCGGGATTCCAAGCAGATTCTCTGGCAGTGGGTCAGGAACCGACTTTACACTGTC |
| | | CATTAACTCTGTGGAGACCGAAGATTTCGGCATGTATTTTTGCCAGCAGAGCAATTCCTGGCC |
| | | TTACACATTCGGAGGCGGGACTAAACTGGAGATTAAGAGGGAGCAGAAACTGATTAGCGAAGA |
| | | GGACCTGAAGGACGAACTGTGA |
| 22 | PGK promoter- | ATTCTACCGGGTAGGGGAGGCGCTTTTCCCAAGGCAGTCTGGAGCATGCGCTTTAGCAGCCCC |
| | anti-human | GCTGGGCACTTGGCGCTACACAAGTGGCCTCTGGCCTCGCACACATTCCACATCCACCGGTAG |
| | CD7 (TH69) | GCGCCAACCGGCTCCGTTCTTTGGTGGCCCCTTCGCGCCACCTTCTACTCCTCCCCTAGTCAG |
| | CAR-EFla | GAAGTTCCCCCCCGCCCCGCAGCTCGCGTCGTGCAGGACGTGACAAATGGAAGTAGCACGTCT |
| | promoter-Anti- | CACTAGTCTCGTGCAGATGGACAGCACCGCTGAGCAATGGAAGCGGGTAGGCCTTTGGGGCAG |
| | human CD7 | CGGCCAATAGCAGCTTTGCTCCTTCGCTTTCTGGGCTCAGAGGCTGGGAAGGGGTGGGTCCGG |
| | (TH69) PEBL | GGGCGGGCTCAGGGGCGGGCTCAGGGGCGGGGCGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGAGAATTCGGCTTCCACCATGGCTCTGCCTGTGACTGCCCTGCTGCTGCCCCTGGCCCTG |
| | | CTGCTGCACGCTGCTAGACCCGCCGCATACAAGGACATTCAGATGACTCAGACTACATCCAGC |
| | | CTGTCAGCTTCCCTGGGCGATCGAGTGACTATCAGCTGTTCAGCAAGCCAGGGGATCAGCAAT |
| | | TACCTGAACTGGTACCAGCAGAAGCCCGACGGAACTGTCAAGCTGCTGATCTACTATACTTCC |
| | | AGTCTGCATAGCGGGGTCCCTAGCCGATTCTCTGGGAGTGGAAGCGGCACCGACTATAGCCTG |
| | | ACCATTTCCAACCTGGAACCAGAGGACATTGCCACCTATTACTGTCAGCAGTATTCAAAGCTG |
| | | CCCTATACCTTTGGCGGAGGGACTAAGCTGGAGATCAAGCGAGGAGGGGGAGGATCAGGGGGA |
| | | GGGGGGAGCGGAGGCGGGGGGAGTGGCGGCGGAGGGTCAGAAGTCCAGCTGGTCGAGAGTGGG |
| | | GGCGGACTGGTGAAACCTGGGGGCTCTCTGAAACTGAGCTGTGCAGCCAGTGGCCTGACATTC |
| | | TCCTCATATGCTATGTCTTGGGTGCGGCAGACCCCAGAAAAGAGACTGGAGTGGGTGGCTTCA |
| | | ATCTCTTCCGGGGGCTTCACTTACTATCCTGACAGCGTGAAAGGCAGATTCACTATCTCCAGG |
| | | GATAACGCACGGAATATCCTGTATCTGCAGATGAGCTCACTGAGATCCGAGGACACCGCAATG |
| | | TACTACTGCGCAAGAGACGAAGTGCGGGGGTATCTGGACGTGTGGGGAGCAGGCACTACCGTC |
| | | ACCGTGTCTAGCACCACTACACCTGCACCAAGGCCTCCCACACCCGCTCCCACTATCGCTTCC |
| | | CAGCCACTGTCCCTGAGGCCCGAGGCCTGCAGGCCAGCAGCTGGCGGAGCCGTGCATACTAGG |
| | | GGGCTGGACTTCGCTTGCGACATCTACATCTGGGCCCCACTGGCAGGGACATGCGGAGTCCTG |
| | | CTGCTGTCCCTGGTCATCACACTGTACTGCAAGCGGGGGCGCAAAAAACTGCTGTATATCTTT |
| | | AAGCAGCCTTTCATGAGACCAGTGCAGACAACCCAGGAGGAAGATGGGTGCTCATGCCGGTTT |
| | | CCCGAGGAGGAGGAAGGCGGCTGCGAGCTGAGGGTGAAGTTTTCCCGCTCAGCAGATGCTCCT |
| | | GCCTACCAGCAGGGCCAGAACCAGCTGTATAATGAGCTGAACCTGGGCAGACGCGAAGAGTAT |
| | | GATGTGCTGGACAAAAGGCGGGGAAGAGACCCCGAAATGGGAGGGAAGCCAAGGCGGAAAAAC |
| | | CCCCAGGAGGGCCTGTACAATGAGCTGCAGAAGGACAAAATGGCAGAGGCTTACAGTGAGATT |
| | | GGGATGAAGGGAGAGAGACGGAGGGGAAAAGGGCACGATGGCCTGTACCAGGGGCTGAGCACA |
| | | GCAACCAAAGATACTTATGACGCACTGCACATGCAGGCACTGCCACCCAGATGAGTCGACGGC |
| | | TCCGGTGCCCGTCAGTGGGCAGAGCGCACATCGCCCACAGTCCCCGAGAAGTTGGGGGGAGGG |
| | | GTCGGCAATTGAACCGGTGCCTAGAGAAGGTGGCGCGGGGTAAACTGGGAAAGTGATGTCGTG |
| | | TACTGGCTCCGCCTTTTTCCCGAGGGTGGGGGAGAACCGTATATAAGTGCAGTAGTCGCCGTG |
| | | AACGTTCTTTTTCGCAACGGGTTTGCCGCCAGAACACAGGTAAGTGCCGTGTGTGGTTCCCGC |
| | | GGGCCTGGCCTCTTTACGGGTTATGGCCCTTGCGTGCCTTGAATTACTTCCACCTGGCTGCAG |
| | | TACGTGATTCTTGATCCCGAGCTTCGGGTTGGAAGTGGGTGGGAGAGTTCGAGGCCTTGCGCT |
| | | TAAGGAGCCCCTTCGCCTCGTGCTTGAGTTGAGGCCTGGCCTGGGCGCTGGGGCCGCCGCGTG |
| | | CGAATCTGGTGGCACCTTCGCGCCTGTCTCGCTGCTTTCGATAAGTCTCTAGCCATTTAAAAT |
| | | TTTTGATGACCTGCTGCGACGCTTTTTTTCTGGCAAGATAGTCTTGTAAATGCGGGCCAAGAT |
| | | CTGCACACTGGTATTTCGGTTTTTGGGGCCGCGGGCGGCGACGGGGCCCGTGCGTCCCAGCGC |
| | | ACATGTTCGGCGAGGCGGGGCCTGCGAGCGCGGCCACCGAGAATCGGACGGGGGTAGTCTCAA |
| | | GCTGGCCGGCCTGCTCTGGTGCCTGGCCTCGCGCCGCCGTGTATCGCCCCGCCCTGGGCGGCA |
| | | AGGCTGGCCCGGTCGGCACCAGTTGCGTGAGCGGAAAGATGGCCGCTTCCCGGCCCTGCTGCA |
| | | GGGAGCTCAAAATGGAGGACGCGGCGCTCGGGAGAGCGGGCGGGTGAGTCACCCACACAAAGG |
| | | AAAAGGGCCTTTCCGTCCTCAGCCGTCGCTTCATGTGACTCCACGGAGTACCGGGCGCCGTCC |
| | | AGGCACCTCGATTAGTTCTCGAGCTTTTGGAGTACGTCGTCTTTAGGTTGGGGGGAGGGGTTT |
| | | TATGCGATGGAGTTTCCCCACACTGAGTGGGTGGAGACTGAAGTTAGGCCAGCTTGGCACTTG |
| | | ATGTAATTCTCCTTGGAATTTGCCCTTTTTGAGTTTGGATCTTGGTTCATTCTCAAGCCTCAG |
| | | ACAGTGGTTCAAAGTTTTTTTCTTCCATTTCAGGTGTCGTGACCCGGGGGCTTCCACCATGGC |
| | | TCTGCCTGTGACCGCACTGCTGCTGCCCCTGGCTCTGCTGCTGCACGCCGCAAGACCTGCCGC |
| | | ATACAAGGATATTCAGATGACTCAGACCACAAGCTCCCTGAGCGCCTCCCTGGGAGACCGAGT |
| | | GACAATCTCTTGCAGTGCATCACAGGGAATTAGCAACTACCTGAATTGGTATCAGCAGAAGCC |
| | | AGATGGCACTGTGAAACTGCTGATCTACTATACCTCTAGTCTGCACAGTGGGGTCCCCTCACG |
| | | ATTCAGCGGATCCGGCTCTGGGACAGACTACAGCCTGACTATCTCCAACCTGGAGCCCGAAGA |
| | | TATTGCCACCTACTATTGCCAGCAGTACTCCAAGCTGCCTTATACCTTTGGCGGGGGAACAAA |
| | | GCTGGAGATTAAAAGGGGAGGAGGAGGAAGCGGAGGAGGAGGATCCGGAGGCGGGGGATCTGG |
| | | AGGAGGAGGAAGTGAGGTGCAGCTGGTCGAATCTGGAGGAGGACTGGTGAAGCCAGGAGGATC |
| | | TCTGAAACTGAGTTGTGCCGCTTCAGGCCTGACCTTCTCAAGCTACGCCATGAGCTGGGTGCG |
| | | ACAGACACCTGAGAAGCGGCTGGAATGGGTCGCTAGCATCTCCTCTGGCGGGTTCACATACTA |
| | | TCCAGACTCCGTGAAAGGCAGATTTACTATCTCTCGGGATAACGCAAGAAATATTCTGTACCT |
| | | GCAGATGAGTTCACTGAGGAGCGAGGACACCGCAATGTACTATTGTGCCAGGGACGAAGTGCG |
| | | CGGCTATCTGGATGTCTGGGGAGCTGGCACTACCGTCACCGTCTCCAGCGAGCAGAAACTGAT |
| | | TAGCGAAGAGGACCTGAAAGATGAACTGTAA |
| 23 | PGK promoter- | ATTCTACCGGGTAGGGGAGGCGCTTTTCCCAAGGCAGTCTGGAGCATGCGCTTTAGCAGCCCC |
| | anti-human | GCTGGGCACTTGGCGCTACACAAGTGGCCTCTGGCCTCGCACACATTCCACATCCACCGGTAG |
| | CD7 (TH69) | GCGCCAACCGGCTCCGTTCTTTGGTGGCCCCTTCGCGCCACCTTCTACTCCTCCCCTAGTCAG |
| | CAR-EFS | GAAGTTCCCCCCCGCCCCGCAGCTCGCGTCGTGCAGGACGTGACAAATGGAAGTAGCACGTCT |
| | promoter-Anti- | CACTAGTCTCGTGCAGATGGACAGCACCGCTGAGCAATGGAAGCGGGTAGGCCTTTGGGGCAG |
| | human CD7 | CGGCCAATAGCAGCTTTGCTCCTTCGCTTTCTGGGCTCAGAGGCTGGGAAGGGGTGGGTCCGG |
| | (TH69) PEBL | GGGCGGGCTCAGGGGGGGGCTCAGGGGCGGGGGGGGCGCCCGAAGGTCCTCCGGAGGCCCGGC |
| | | ATTCTGCACGCTTCAAAAGCGCACGTCTGCCGCGCTGTTCTCCTCTTCCTCATCTCCGGGCCT |
| | | TTCGAGAATTCGGCTTCCACCATGGCTCTGCCTGTGACTGCCCTGCTGCTGCCCCTGGCCCTG |
| | | CTGCTGCACGCTGCTAGACCCGCCGCATACAAGGACATTCAGATGACTCAGACTACATCCAGC |
| | | CTGTCAGCTTCCCTGGGCGATCGAGTGACTATCAGCTGTTCAGCAAGCCAGGGGATCAGCAAT |
| | | TACCTGAACTGGTACCAGCAGAAGCCCGACGGAACTGTCAAGCTGCTGATCTACTATACTTCC |
| | | AGTCTGCATAGCGGGGTCCCTAGCCGATTCTCTGGGAGTGGAAGCGGCACCGACTATAGCCTG |
| | | ACCATTTCCAACCTGGAACCAGAGGACATTGCCACCTATTACTGTCAGCAGTATTCAAAGCTG |
| | | CCCTATACCTTTGGCGGAGGGACTAAGCTGGAGATCAAGCGAGGAGGGGGAGGATCAGGGGGA |
| | | GGGGGGAGCGGAGGCGGGGGGAGTGGCGGCGGAGGGTCAGAAGTCCAGCTGGTCGAGAGTGGG |
| | | GGCGGACTGGTGAAACCTGGGGGCTCTCTGAAACTGAGCTGTGCAGCCAGTGGCCTGACATTC |
| | | TCCTCATATGCTATGTCTTGGGTGCGGCAGACCCCAGAAAAGAGACTGGAGTGGGTGGCTTCA |
| | | ATCTCTTCCGGGGGCTTCACTTACTATCCTGACAGCGTGAAAGGCAGATTCACTATCTCCAGG |
| | | GATAACGCACGGAATATCCTGTATCTGCAGATGAGCTCACTGAGATCCGAGGACACCGCAATG |
| | | TACTACTGCGCAAGAGACGAAGTGCGGGGGTATCTGGACGTGTGGGGAGCAGGCACTACCGTC |
| | | ACCGTGTCTAGCACCACTACACCTGCACCAAGGCCTCCCACACCCGCTCCCACTATCGCTTCC |
| | | CAGCCACTGTCCCTGAGGCCCGAGGCCTGCAGGCCAGCAGCTGGCGGAGCCGTGCATACTAGG |
| | | GGGCTGGACTTCGCTTGCGACATCTACATCTGGGCCCCACTGGCAGGGACATGCGGAGTCCTG |
| | | CTGCTGTCCCTGGTCATCACACTGTACTGCAAGCGGGGGCGCAAAAAACTGCTGTATATCTTT |
| | | AAGCAGCCTTTCATGAGACCAGTGCAGACAACCCAGGAGGAAGATGGGTGCTCATGCCGGTTT |
| | | CCCGAGGAGGAGGAAGGCGGCTGCGAGCTGAGGGTGAAGTTTTCCCGCTCAGCAGATGCTCCT |
| | | GCCTACCAGCAGGGCCAGAACCAGCTGTATAATGAGCTGAACCTGGGCAGACGCGAAGAGTAT |
| | | GATGTGCTGGACAAAAGGCGGGGAAGAGACCCCGAAATGGGAGGGAAGCCAAGGCGGAAAAAC |
| | | CCCCAGGAGGGCCTGTACAATGAGCTGCAGAAGGACAAAATGGCAGAGGCTTACAGTGAGATT |
| | | GGGATGAAGGGAGAGAGACGGAGGGGAAAAGGGCACGATGGCCTGTACCAGGGGCTGAGCACA |
| | | GCAACCAAAGATACTTATGACGCACTGCACATGCAGGCACTGCCACCCAGATGAGTCGACGGG |
| | | CAGAGCGCACATCGCCCACAGTCCCCGAGAAGTTGGGGGGAGGGGTCGGCAATTGAACCGGTG |
| | | CCTAGAGAAGGTGGCGCGGGGTAAACTGGGAAAGTGATGTCGTGTACTGGCTCCGCCTTTTTC |
| | | CCGAGGGTGGGGGAGAACCGTATATAAGTGCAGTAGTCGCCGTGAACGTTCTTTTTCGCAACG |
| | | GGTTTGCCGCCAGAACACAGGTGTCGTGACCGCGGGTCTAGAGGATCCGGTACTCGAGGAACT |
| | | GAAAAACCAGAAAGTTAACTGGTAAGTTTAGTCTTTTTGTCTTTTATTTCAGGTCCCGGATCC |
| | | GGTGGTGGTGCAAATCAAAGAACTGCTCCTCAGTGGATGTTGCCTTTACTTCTAGCCCGGGGG |
| | | CTTCCACCATGGCTCTGCCTGTGACCGCACTGCTGCTGCCCCTGGCTCTGCTGCTGCACGCCG |
| | | CAAGACCTGCCGCATACAAGGATATTCAGATGACTCAGACCACAAGCTCCCTGAGCGCCTCCC |
| | | TGGGAGACCGAGTGACAATCTCTTGCAGTGCATCACAGGGAATTAGCAACTACCTGAATTGGT |
| | | ATCAGCAGAAGCCAGATGGCACTGTGAAACTGCTGATCTACTATACCTCTAGTCTGCACAGTG |
| | | GGGTCCCCTCACGATTCAGCGGATCCGGCTCTGGGACAGACTACAGCCTGACTATCTCCAACC |
| | | TGGAGCCCGAAGATATTGCCACCTACTATTGCCAGCAGTACTCCAAGCTGCCTTATACCTTTG |
| | | GCGGGGGAACAAAGCTGGAGATTAAAAGGGGAGGAGGAGGAAGCGGAGGAGGAGGATCCGGAG |
| | | GCGGGGGATCTGGAGGAGGAGGAAGTGAGGTGCAGCTGGTCGAATCTGGAGGAGGACTGGTGA |
| | | AGCCAGGAGGATCTCTGAAACTGAGTTGTGCCGCTTCAGGCCTGACCTTCTCAAGCTACGCCA |
| | | TGAGCTGGGTGCGACAGACACCTGAGAAGCGGCTGGAATGGGTCGCTAGCATCTCCTCTGGCG |
| | | GGTTCACATACTATCCAGACTCCGTGAAAGGCAGATTTACTATCTCTCGGGATAACGCAAGAA |
| | | ATATTCTGTACCTGCAGATGAGTTCACTGAGGAGCGAGGACACCGCAATGTACTATTGTGCCA |
| | | GGGACGAAGTGCGCGGCTATCTGGATGTCTGGGGAGCTGGCACTACCGTCACCGTCTCCAGCG |
| | | AGCAGAAACTGATTAGCGAAGAGGACCTGAAAGATGAACTGTAA |

TABLE 14
Exemplary amino acid sequences for anti-CD7 PEBL and anti-CD7 CAR

| SEQ ID | | |
|---|---|---|
| NO | Name | Sequence |
| 24 | Anti CD7 | MALPVTALLLPLALLLHAARPAAYKDIQMTQTTSSLSASLGDRVTISCSASQGISNYLNWYQQ |
| | (TH69) PEBL | KPDGTVKLLIYYTSSLHSGVPSRFSGSGSGTDYSLTISNLEPEDIATYYCQQYSKLPYTFGGG |
| | | TKLEIKR//GGGGSGGGGSGGGGSGGGGSEVQLVESGGGLVKPGGSLKLSCAASGLTFSSYAM |
| | | SWVRQTPEKRLEWVASISSGGFTYYPDSVKGRFTISRDNARNILYLQMSSLRSEDTAMYYCAR |
| | | DEVRGYLDVWGAGTTVTVSSEQKLISEEDLKDEL |
| 25 | Anti CD7 | PMALPVTALLLPLALLLHAARPAAYKDIQMTQTTSSLSASLGDRVTISCSASQGISNYLNWYQ |
| | (TH69) PEBL | QKPDGTVKLLIYYTSSLHSGVPSRFSGSGSGTDYSLTISNLEPEDIATYYCQQYSKLPYTFGG |
| | variant | GTKLEIKRGGGGSGGGGSGGGGSGGGGSEVQLVESGGGLVKPGGSLKLSCAASGLTFSSYAMS |
| | | WVRQTPEKRLEWVASISSGGFTYYPDSVKGRFTISRDNARNILYLQMSSLRSEDTAMYYCARD |
| | | EVRGYLDVWGAGTTVTVSSEQKLISEEDLKDEL |
| 26 | Anti CD7 | MALPVTALLLPLALLLHAARPQVQLQESGAELVKPGASVKLSCKASGYTFTSYWMHWVKQRPG |
| | (3A1F) PEBL | QGLEWIGKINPSNGRTNYNEKFKSKATLTVDKSSSTAYMQLSSLTSEDSAVYYCARGGVYYDL |
| | | YYYALDYWGQGTTVTVSSGGGGSGGGGSGGGGSDIELTQSPATLSVTPGDSVSLSCRASQSIS |
| | | NNLHWYQQKSHESPRLLIKSASQSISGIPSRFSGSGSGTDFTLSINSVETEDFGMYFCQQSNS |
| | | WPYTFGGGTKLEIKREQKLISEEDLKDEL |
| 27 | Anti CD7 | PMALPVTALLLPLALLLHAARPQVQLQESGAELVKPGASVKLSCKASGYTFTSYWMHWVKQRP |
| | (3A1F) PEBL | GQGLEWIGKINPSNGRTNYNEKFKSKATLTVDKSSSTAYMQLSSLTSEDSAVYYCARGGVYYD |
| | variant | LYYYALDYWGQGTTVTVSSGGGGSGGGGSGGGGSDIELTQSPATLSVTPGDSVSLSCRASQSI |
| | | SNNLHWYQQKSHESPRLLIKSASQSISGIPSRFSGSGSGTDFTLSINSVETEDFGMYFCQQSN |
| | | SWPYTFGGGTKLEIKREQKLISEEDLKDEL |
| 28 | Anti CD7 | MALPVTALLLPLALLLHAARPAAYKDIQMTQTTSSLSASLGDRVTISCSASQGISNYLNWYQQ |
| | (TH69) CAR | KPDGTVKLLIYYTSSLHSGVPSRFSGSGSGTDYSLTISNLEPEDTATYYCQQYSKLPYTFGGG |
| | | TKLEIKRGGGGSGGGGSGGGGSGGGGSEVQLVESGGGLVKPGGSLKLSCAASGLTFSSYAMSW |
| | | VRQTPEKRLEWVASISSGGFTYYPDSVKGRFTISRDNARNILYLQMSSLRSEDTAMYYCARDE |
| | | VRGYLDVWGAGTTVTVSSTTTPAPRPPTPAPTIASQPLSLRPEACRPAAGGAVHTRGLDFACD |
| | | IYIWAPLAGTCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 29 | Anti CD7 | MALPVTALLLPLALLLHAARPAAYKDIQMTQTTSSLSASLGDRVTISCSASQGISNYLNWYQQ |
| | (TH69) CAR | KPDGTVKLLIYYTSSLHSGVPSRFSGSGSGTDYSLTISNLEPEDIATYYCQQYSKLPYTFGGG |
| | variant | TKLEIKRGGGGSGGGGSGGGGSGGGGSEVQLVESGGGLVKPGGSLKLSCAASGLTFSSYAMSW |
| | | VRQTPEKRLEWVASISSGGFTYYPDSVKGRFTISRDNARNILYLQMSSLRSEDTAMYYCARDE |
| | | VRGYLDVWGAGTTVTVSSTTTPAPRPPTPAPTIASQPLSLRPEACRPAAGGAVHTRGLDFACD |
| | | IYIWAPLAGTCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPRGSGATNFSLLK |
| | | QAGDVEENPG |
| 30 | Anti CD7 | MALPVTALLLPLALLLHAARPQVQLQESGAELVKPGASVKLSCKASGYTFTSYWMHWVKQRPG |
| | (3A1F) CAR | QGLEWIGKINPSNGRTNYNEKFKSKATLTVDKSSSTAYMQLSSLTSEDSAVYYCARGGVYYDL |
| | | YYYALDYWGQGTTVTVSSGGGGSGGGGSGGGGSDIELTQSPATLSVTPGDSVSLSCRASQSIS |
| | | NNLHWYQQKSHESPRLLIKSASQSISGIPSRFSGSGSGTDFTLSINSVETEDFGMYFCQQSNS |
| | | WPYTFGGGTKLEIKRTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCRFPEEEEGGCEL |
| | | RVKFSRSADAPAYQQGQNQLYNELNLGRREEYDVLDKRRGRDPEMGGKPRRKNPQEGLYNELQ |
| | | KDKMA.EAYSEIGMKGERRRGKGHDGLYQGLSTATKDTYDALHMQALPPR |
| 31 | Anti CD7 | MALPVTALLLPLALLLHAARPQVQLQESGAELVKPGASVKLSCKASGYTFTSYWMHWVKQRPG |
| | (3A1F) CAR | QGLEWIGKINPSNGRTNYNEKFKSKATLTVDKSSSTAYMQLSSLTSEDSAVYYCARGGVYYDL |
| | variant | YYYALDYWGQGTTVTVSSGGGGSGGGGSGGGGSDIELTQSPATLSVTPGDSVSLSCRASQSIS |
| | | NNLHWYQQKSHESPRLLIKSASQSISGIPSRFSGSGSGTDFTLSINSVETEDFGMYFCQQSNS |
| | | WPYTFGGGTKLEIKRTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSCRFPEEEEGGCEL |
| | | RVKFSRSADAPAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPRGSGATNFSLLKQAG |
| | | DVEENPG |
| 100 | Anti-human | MALPVTALLLPLALLLHAARPAAYKDIQMTQTTSSLSASLGDRVTISCSASQGISNYLNWYQQ |
| | CD7 (TH69) | KPDGTVKLLIYYTSSLHSGVPSRFSGSGSGTDYSLTISNLEPEDIATYYCQQYSKLPYTFGGG |
| | CAR-p2A- | TKLEIKRGGGGSGGGGSGGGGSGGGGSEVQLVESGGGLVKPGGSLKLSCAASGLTFSSYAMSW |
| | Anti-human | VRQTPEKRLEWVASISSGGFTYYPDSVKGRFTISRDNARNILYLQMSSLRSEDTAMYYCARDE |
| | CD7 (TH69) | VRGYLDVWGAGTTVTVSSTTTPAPRPPTPAPTIASQPLSLRPEACRPAAGGAVHTRGLDFACD |
| | PEBL | IYIWAPLAGTCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPRGSGATNFSLLK |
| | | QAGDVEENPGPMALPVTALLLPLALLLHAARPAAYKDIQMTQTTSSLSASLGDRVTISCSASQ |
| | | GISNYLNWYQQKPDGTVKLLIYYTSSLHSGVPSRFSGSGSGTDYSLTISNLEPEDIATYYCQQ |
| | | YSKLPYTFGGGTKLEIKRGGGGSGGGGSGGGGSGGGGSEVQLVESGGGLVKPGGSLKLSCAAS |
| | | GLTFSSYAMSWVRQTPEKRLEWVASISSGGFTYYPDSVKGRFTISRDNARNILYLQMSSLRSE |
| | | DTAMYYCARDEVRGYLDVWGAGTTVTVSSEQKLISEEDLKDEL |

1.-274. (canceled)

275. An engineered immune cell, comprising:
(a) a first nucleic acid sequence encoding (i) a domain that binds to a subunit of a T cell receptor (TCR) complex linked to (ii) a synthetic localizing domain;
(b) a second nucleic acid sequence encoding a chimeric antigen receptor (CAR);
(c) a third nucleic acid sequence encoding a surface polypeptide binding domain linked to a synthetic surface polypeptide localizing domain; and
wherein the engineered immune cell comprises at least twice as much of the first nucleic acid sequence as the second nucleic acid sequence or the third nucleic acid sequence.

276. The engineered immune cell of claim 275, wherein the subunit of the TCR complex is CD3ε.

277. The engineered immune cell of claim 275, wherein the synthetic localizing domain comprises an ER retention signal.

278. The engineered immune cell of claim 277, wherein the synthetic localizing domain further comprises a Myc tag.

279. The engineered immune cell of claim 278, wherein the first nucleic acid sequence comprises, in 5′ to 3′ direction, a sequence encoding the domain that binds to the subunit of the TCR complex, a sequence encoding the Myc tag, and a sequence encoding the ER retention signal.

280. The engineered immune cell of claim 275, wherein the second nucleic acid sequence and the third nucleic acid sequence are on the same nucleic acid molecule.

281. The engineered immune cell of claim 280, wherein the same nucleic acid molecule is a vector.

282. The engineered immune cell of claim 281, wherein the first nucleic acid sequence is on a nucleic acid molecule separate from the second nucleic acid sequence or the third nucleic acid sequence, and wherein the nucleic acid molecule having the first nucleic acid sequence is a first expression vector.

283. The engineered immune cell of claim 275, further comprising a fourth nucleic acid sequence encoding a kill gene.

284. The engineered immune cell of claim 283, wherein the kill gene comprises CD20 or a derivative thereof.

285. The engineered immune cell of claim 275, wherein the CAR comprises a target binding domain that binds to the surface polypeptide.

286. The engineered immune cell of claim 285, wherein the surface polypeptide binding domain is a first antibody or antigen binding domain thereof, and the target binding domain is a second antibody or antigen binding domain thereof.

287. The engineered immune cell of claim 286, wherein an amino acid sequence of the first antibody or antigen binding domain thereof and an amino acid sequence of the second antibody or antigen binding domain thereof are at least 80% identical.

288. The engineered immune cell of claim 275, wherein the surface polypeptide is CD7.

289. The engineered immune cell of claim 275, wherein the synthetic localizing domain or the synthetic surface polypeptide localizing domain comprises an endoplasmic reticulum (ER) retention sequence, a Golgi retention sequence, a proteasome localizing sequence, or a transmembrane domain.

290. The engineered immune cell of claim 289, wherein the synthetic localizing domain and the synthetic surface polypeptide localizing domain comprise a same ER retention sequence.

291. The engineered immune cell of claim 275, wherein a surface expression of the TCR complex and a surface expression of the surface polypeptide are downregulated in the engineered immune cell.

292. The engineered immune cell of claim 275, wherein the engineered immune cell is a T cell or a natural killer (NK) cell.

293. The engineered immune cell of claim 275, wherein the engineered immune cell is an allogeneic cell.

294. A method of treating a disease in a subject in need thereof, the method comprising: administering a therapeutically effective amount of engineered immune cells, wherein an engineered immune cell of the engineered immune cells comprises the engineered immune cell of claim 275.


SEQUENCE LISTING (233 sequences)

SEQ ID NO: 1 - DNA, 2708 bp, synthetic construct
  source 1..2708: mol_type = other DNA
atcgattttaaaagaaaaggggggattggggggtacagtgcaggggaaagaatagtagacataatagcaacagacatacaaactaaagaattacaaaaacaaattacaaaaattcaaaattttcgggtttattaattaacagggacatgcagagatccagtttaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatcgctagcgctaccggactcagatcgaattcctcaagcttcctcgagtgcagtcgacgcgggcccgggatcccgcgactctagataattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactggccaccggcttccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgg

SEQ ID NO: 2 - DNA, 855 bp, synthetic construct
  source 1..855: mol_type = other DNA
atggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 3 - DNA, 846 bp, synthetic construct
  source 1..846: mol_type = other DNA
atggctctgcccgtcaccgctctgctgctgcctctggctctgctgctgcacgccgcaagaccccaggtccagctgcaggagtcaggggcagagctggtgaaacccggagccagtgtcaaactgtcctgtaaggccagcggctatactttcaccagctactggatgcactgggtgaaacagaggccaggacagggcctggagtggatcggcaagattaaccccagcaatgggcgcaccaactacaacgaaaagtttaaatccaaggctacactgactgtggacaagagctcctctaccgcatacatgcagctgagttcactgacatctgaagatagtgccgtgtactattgcgccagaggcggggtctactatgacctgtactattacgcactggattattgggggcagggaaccacagtgactgtcagctccggaggaggaggatccggcggaggaggctctgggggaggcgggagtgacatcgagctgacacagtctccagccactctgagcgtgacccctggcgattctgtcagtctgtcatgtagagctagccagtccatctctaacaatctgcactggtaccagcagaaatcacatgaaagccctcggctgctgattaagagtgcttcacagagcatctccgggattccaagcagattctctggcagtgggtcaggaaccgactttacactgtccattaactctgtggagaccgaagatttcggcatgtatttttgccagcagagcaattcctggccttacacattcggaggcgggactaaactggagattaagagggagcagaaactgattagcgaagaggacctgaaggacgaactgtga

SEQ ID NO: 4 - DNA, 1482 bp, synthetic construct
  source 1..1482: mol_type = other DNA
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatga

SEQ ID NO: 5 - DNA, 1476 bp, synthetic construct
  source 1..1476: mol_type = other DNA
atggctctgcccgtcaccgctctgctgctgcctctggctctgctgctgcacgctgctcgaccacaggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctccggaggaggaggaagcggaggaggaggatccggaggaggaggatctgacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcggactaccacaccagctccaagaccacctacccctgcaccaacaattgctagtcagccactgtcactgagaccagaagcatgtaggcctgcagctggaggagctgtgcacaccagaggcctggactttgcctgcgatatctacatttgggctcctctggcaggaacctgtggcgtgctgctgctgtctctggtcatcacactgtattgcaaacgagggcggaagaaactgctgtacattttcaagcagccctttatgaggcctgtgcagactacccaggaggaagacggctgcagctgtcgcttccctgaggaagaggaagggggatgtgagctgcgcgtcaaattttctcgaagtgcagatgccccagcttatcagcagggccagaaccagctgtacaacgagctgaatctggggcggagagaggaatatgacgtgctggataagaggcgaggaagggacccagaaatgggagggaaaccacgacggaagaacccccaggagggcctgtataatgaactgcagaaagacaagatggcagaggcctactctgaaatcgggatgaaaggagagagaaggcgcggcaaggggcatgatggcctgtaccaggggctgagcacagcaaccaaagacacctatgacgcactgcacatgcaggcactgccaccacggtaatga

SEQ ID NO: 6 - DNA, 255 bp, synthetic construct
  source 1..255: mol_type = other DNA
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagt

SEQ ID NO: 7 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 509 bp, synthetic construct
  source 1..509: mol_type = other DNA
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcga

SEQ ID NO: 9 - DNA, 384 bp, synthetic construct
  source 1..384: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcc

SEQ ID NO: 10 - DNA, 509 bp, synthetic construct; identical to SEQ ID NO: 8
  source 1..509: mol_type = other DNA

SEQ ID NO: 11 - DNA, 2974 bp, synthetic construct
  source 1..2974: mol_type = other DNA
atggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaacgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgacagccaggggatttcaccactcaaaggccagacctgcagacgcccagattatgagacaca

SEQ ID NO: 12 - DNA, 2974 bp, synthetic construct
  source 1..2974: mol_type = other DNA
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgacagccaggggatttcaccactcaaaggccagacctgcagacgcccagattatgagacacacgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 13 - DNA, 2400 bp, synthetic construct
  source 1..2400: mol_type = other DNA
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 14 - DNA, 2803 bp, synthetic construct
  source 1..2803: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacagcagtattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 15 - DNA, 3598 bp, synthetic construct
  source 1..3598: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggagaagccagaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 16 - DNA, 2792 bp, synthetic construct
  source 1..2792: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatgactctgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 17 - DNA, 3346 bp, synthetic construct
  source 1..3346: mol_type = other DNA
ttaattaatttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgactcgagtgcagtcgacgcgggcccgggatcccgcgactctagataattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactggccaggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaacgcgt

SEQ ID NO: 18 - DNA, 3938 bp, synthetic construct
  source 1..3938: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 19 - DNA, 3132 bp, synthetic construct
  source 1..3132: mol_type = other DNA
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctagcccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 20 - DNA, 4057 bp, synthetic construct
  source 1..4057: mol_type = other DNA
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcccgtcaccgctctgctgctgcctctggctctgctgctgcacgctgctcgaccacaggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctccggaggaggaggaagcggaggaggaggatccggaggaggaggatctgacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcggactaccacaccagctccaagaccacctacccctgcaccaacaattgctagtcagccactgtcactgagaccagaagcatgtaggcctgcagctggaggagctgtgcacaccagaggcctggactttgcctgcgatatctacatttgggctcctctggcaggaacctgtggcgtgctgctgctgtctctggtcatcacactgtattgcaaacgagggcggaagaaactgctgtacattttcaagcagccctttatgaggcctgtgcagactacccaggaggaagacggctgcagctgtcgcttccctgaggaagaggaagggggatgtgagctgcgcgtcaaattttctcgaagtgcagatgccccagcttatcagcagggccagaaccagctgtacaacgagctgaatctggggcggagagaggaatatgacgtgctggataagaggcgaggaagggacccagaaatgggagggaaaccacgacggaagaacccccaggagggcctgtataatgaactgcagaaagacaagatggcagaggcctactctgaaatcgggatgaaaggagagagaaggcgcggcaaggggcatgatggcctgtaccaggggctgagcacagcaaccaaagacacctatgacgcactgcacatgcaggcactgccaccacggtaatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 21 - DNA, 4054 bp, synthetic construct
  source 1..4054: mol_type = other DNA
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcccgtcaccgctctgctgctgcctctggctctgctgctgcacgccgcaagaccccaggtccagctgcaggagtcaggggcagagctggtgaaacccggagccagtgtcaaactgtcctgtaaggccagcggctatactttcaccagctactggatgcactgggtgaaacagaggccaggacagggcctggagtggatcggcaagattaaccccagcaatgggcgcaccaactacaacgaaaagtttaaatccaaggctacactgactgtggacaagagctcctctaccgcatacatgcagctgagttcactgacatctgaagatagtgccgtgtactattgcgccagaggcggggtctactatgacctgtactattacgcactggattattgggggcagggaaccacagtgactgtcagctccggaggaggaggatccggcggaggaggctctgggggaggcgggagtgacatcgagctgacacagtctccagccactctgagcgtgacccctggcgattctgtcagtctgtcatgtagagctagccagtccatctctaacaatctgcactggtaccagcagaaatcacatgaaagccctcggctgctgattaagagtgcttcacagagcatctccgggattccaagcagattctctggcagtgggtcaggaaccgactttacactgtccattaactctgtggagaccgaagatttcggcatgtatttttgccagcagagcaattcctggccttacacattcggaggcgggactaaactggagattaagagggagcagaaactgattagcgaagaggacctgaaggacgaactgtga

SEQ ID NO: 22 - DNA, 4063 bp, synthetic construct
  source 1..4063: mol_type = other DNA
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 23 - DNA, 3257 bp, synthetic construct
  source 1..3257: mol_type = other DNA
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctagcccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 24 - protein, 284 aa, synthetic construct
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 25 - protein, 285 aa, synthetic construct
PMALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 26 - protein, 281 aa, synthetic construct
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKREQKLISEEDLKDEL

SEQ ID NO: 27 - protein, 282 aa, synthetic construct
PMALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKREQKLISEEDLKDEL

SEQ ID NO: 28 - protein, 493 aa, synthetic construct
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDTATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 514 aa, synthetic construct
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 30 - protein, 490 aa, synthetic construct
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 511 aa, synthetic construct
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 32 - protein, 117 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSS

SEQ ID NO: 33 - protein, 112 aa, synthetic construct
AAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 34 - protein, 123 aa, synthetic construct
QVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 35 - protein, 108 aa, synthetic construct
DIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKR

SEQ ID NO: 36 - protein, 120 aa, synthetic construct
DVQLVESGGGLVQPGGSRKLSCAASGFTFSSFGMHWVRQAPEKGLEWVAYISSGSSTLHYADTVKGRFTISRDNPKNTLFLQMTSLRSEDTAMYYCARWGNYPHYAMDYWGQGTSVTVSS

SEQ ID NO: 37 - protein, 111 aa, synthetic construct
DIVMTQSPASLAVSLGQRATISCRASKSVSASGYSYMHWYQQKPGQPPKLLIYLASNLESGVPARFSGSGSGTDFTLNIHPVEEEDAVTYYCQHSRELPYTFGGGTKLEIK

SEQ ID NO: 38 - DNA, 351 bp, synthetic construct
  source 1..351: mol_type = other DNA
gaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagc

SEQ ID NO: 39 - DNA, 336 bp, synthetic construct
  source 1..336: mol_type = other DNA
gccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaagg

SEQ ID NO: 40 - DNA, 369 bp, synthetic construct
  source 1..369: mol_type = other DNA
caggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctcc

SEQ ID NO: 41 - DNA, 324 bp, synthetic construct
  source 1..324: mol_type = other DNA
gacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcgg

SEQ ID NO: 42 - DNA, 360 bp, synthetic construct
  source 1..360: mol_type = other DNA
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaccagtctaaggtctgaggacacggccatgtattactgtgcaagatggggtaactaccctcactatgctatggactactggggtcaaggaacctcagtcaccgtctcctca

SEQ ID NO: 43 - DNA, 333 bp, synthetic construct
  source 1..333: mol_type = other DNA
gacattgtgatgacccagtctcctgcttccttagctgtatctctggggcagagggccaccatctcatgcagggccagcaaaagtgtcagtgcatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgtaacctattactgtcagcacagtagggagcttccgtacacgttcggaggggggaccaagctggaaataaaa

SEQ ID NO: 44 - protein, 11 aa, synthetic construct
SASQGISNYLN

SEQ ID NO: 45 - protein, 7 aa, synthetic construct
YTSSLHS

SEQ ID NO: 46 - protein, 9 aa, synthetic construct
QQYSKLPYT

SEQ ID NO: 47 - protein, 5 aa, synthetic construct
SYAMS

SEQ ID NO: 48 - protein, 16 aa, synthetic construct
SISSGGFTYYPDSVKG

SEQ ID NO: 49 - protein, 9 aa, synthetic construct
DEVRGYLDV

SEQ ID NO: 50 - protein, 11 aa, synthetic construct
RASQSISNNLH

SEQ ID NO: 51 - protein, 7 aa, synthetic construct
SASQSIS

SEQ ID NO: 52 - protein, 9 aa, synthetic construct
QQSNSWPYT

SEQ ID NO: 53 - protein, 5 aa, synthetic construct
SYWMH

SEQ ID NO: 54 - protein, 17 aa, synthetic construct
KINPSNGRTNYNEKFKS

SEQ ID NO: 55 - protein, 14 aa, synthetic construct
GGVYYDLYYYALDY

SEQ ID NO: 56 - protein, 15 aa, synthetic construct
RASKSVSASGYSYMH

SEQ ID NO: 57 - protein, 7 aa, synthetic construct
LASNLES

SEQ ID NO: 58 - protein, 9 aa, synthetic construct
QHSRELPYT

SEQ ID NO: 59 - protein, 5 aa, synthetic construct
SFGMH

SEQ ID NO: 60 - protein, 17 aa, synthetic construct
YISSGSSTLHYADTVKG

SEQ ID NO: 61 - protein, 11 aa, synthetic construct
WGNYPHYAMDY

SEQ ID NO: 62 - DNA, 577 bp, unidentified
  source 1..577: mol_type = unassigned DNA
cgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatacc

SEQ ID NO: 63 - DNA, 57 bp, Teschovirus A
  source 1..57: mol_type = genomic DNA
gccacaaacttctctctgctaaagcaagcaggtgatgttgaagaaaaccccgggcct

SEQ ID NO: 64 - DNA, 60 bp, Equine rhinitis A virus
  source 1..60: mol_type = genomic DNA
cagtgtactaattatgctctcttgaaattggctggagatgttgagagcaacggaggtccc

SEQ ID NO: 65 - DNA, 66 bp, Foot-and-mouth disease virus
  source 1..66: mol_type = genomic DNA
gtgaaacagactttgaattttgaccttctcaagttggcgggagacgtggagtccaaccctggacct

SEQ ID NO: 66 - DNA, 54 bp, Thosea asigna virus
  source 1..54: mol_type = genomic DNA
gagggcaggggaagtcttctaacatgcggggacgtggaggaaaatcccggccca

SEQ ID NO: 67 - protein, 19 aa, Teschovirus A
ATNFSLLKQAGDVEENPGP

SEQ ID NO: 68 - protein, 20 aa, Equine rhinitis A virus
QCTNYALLKLAGDVESNPGP

SEQ ID NO: 69 - protein, 22 aa, Foot-and-mouth disease virus
VKQTLNFDLLKLAGDVESNPGP

SEQ ID NO: 70 - protein, 18 aa, Thosea asigna virus
EGRGSLLTCGDVEENPGP

SEQ ID NO: 71 - protein, 14 aa, synthetic construct
EQKLISEEDLKDEL

SEQ ID NO: 72 - protein, 26 aa, synthetic construct
GGGGSGGGGSGGGGSGGGGSAEKDEL

SEQ ID NO: 73 - protein, 4 aa, synthetic construct
KDEL

SEQ ID NO: 74 - skipped

SEQ ID NO: 75 - protein, 4 aa, synthetic construct
YQRL

SEQ ID NO: 76 - protein, 4 aa, synthetic construct
PEST

SEQ ID NO: 77 - protein, 83 aa, synthetic construct
TTTPAPRPPTPAPTIASQPLSLRPEACRPAAGGAVHTRGLDFACDIYIWAPLAGTCGVLLLSLVITLYKYKSRRSFIDEKKMP

SEQ ID NO: 78 - protein, 68 aa, synthetic construct
TTTPAPRPPTPAPTIASQPLSLRPEACRPAAGGAVHTRGLDFACDIYIWAPLAGTCGVLLLSLVITLY

SEQ ID NO: 79 - protein, 20 aa, synthetic construct
GGGGSGGGGSGGGGSGGGGS

SEQ ID NO: 80 - protein, 21 aa, synthetic construct
MALPVTALLLPLALLLHAARP

SEQ ID NO: 81 - DNA, 63 bp, synthetic construct
  source 1..63: mol_type = other DNA
atggctctgcctgtgaccgcactgctgctgcccctggctctgctgctgcacgccgcaagacct

SEQ ID NO: 82 - DNA, 60 bp, synthetic construct
  source 1..60: mol_type = other DNA
ggaggaggaggaagcggaggaggaggatccggaggcgggggatctggaggaggaggaagt

SEQ ID NO: 83 - DNA, 42 bp, synthetic construct
  source 1..42: mol_type = other DNA
gagcagaaactgattagcgaagaggacctgaaagatgaactg

SEQ ID NO: 84 - protein, 68 aa, synthetic construct; identical to SEQ ID NO: 78

SEQ ID NO: 85 - protein, 42 aa, synthetic construct
KRGRKKLLYIFKQPFMRPVQTTQEEDGCSCRFPEEEEGGCEL

SEQ ID NO: 86 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87-95 - skipped (9 entries)

SEQ ID NO: 96 - DNA, 207 bp, synthetic construct
  source 1..207: mol_type = other DNA
accactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgc

SEQ ID NO: 97 - DNA, 126 bp, synthetic construct
  source 1..126: mol_type = other DNA
aagcgggggcgcaaaaaactgctgtatatctttaagcagcctttcatgagaccagtgcagacaacccaggaggaagatgggtgctcatgccggtttcccgaggaggaggaaggcggctgcgagctg

SEQ ID NO: 98 - DNA, 335 bp, synthetic construct
  source 1..335: mol_type = other DNA
gggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccaga

SEQ ID NO: 99 - protein, 36 aa, synthetic construct
EQKLISEEDLGGGGSGGGGSGGGGSGGGGSAEKDEL

SEQ ID NO: 100 - protein, 799 aa, synthetic construct
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 101 - protein, 294 aa, synthetic construct
MALPVTALLLPLALLLHAARPQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAGGGGSGGGGSGGGGSGGGGSAEKDEL

SEQ ID NO: 102 - protein, 247 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSGGGGSGGGGSEVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 103 - protein, 306 aa, synthetic construct
MALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSEQKLISEEDLGGGGSGGGGSGGGGSGGGGSAEKDEL

SEQ ID NO: 104 - protein, 249 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 105 - protein, 591 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEGSGATNFSLLKQAGDVEENPGPMALPVTALLLPLALLLHAARPDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSEQKLISEEDLGGGGSGGGGSGGGGSGGGGSAEKDEL

SEQ ID NO: 106 - protein, 263 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 107-109 - skipped (3 entries)

SEQ ID NO: 110 - protein, 120 aa, synthetic construct
EVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 111 - protein, 107 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 112 - DNA, 360 bp, synthetic construct
  source 1..360: mol_type = other DNA
gaggtccagctgcagcagtctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagcc

SEQ ID NO: 113 - DNA, 321 bp, synthetic construct
  source 1..321: mol_type = other DNA
caaattgttctcacccagtctccagcaatcatgtctgcatctccaggggagaaggtcaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggacaaagttggaaataaaccgg

SEQ ID NO: 114 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 115 - protein, 107 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 116 - DNA, 366 bp, synthetic construct
  source 1..366: mol_type = other DNA
gaggtgcagctgcagcagagcggccccgagctggtgaagcccggcgccagcatgaagatcagctgcaaggccagcggctacagcttcaccggctacaccatgaactgggtgaagcagagccacggcaagaacctggagtggatgggcctgatcaacccctacaagggcgtgagcacctacaaccagaagttcaaggacaaggccaccctgaccgtggacaagagcagcagcaccgcctacatggagctgctgagcctgaccagcgaggacagcgccgtgtactactgcgccaggagcggctactacggcgacagcgactggtacttcgacgtgtggggccagggcaccaccctgaccgtgttcagc

SEQ ID NO: 117 - DNA, 321 bp, synthetic construct
  source 1..321: mol_type = other DNA
gacatccagatgacccagaccaccagcagcctgagcgccagcctgggcgacagggtgaccatcagctgcagggccagccaggacatcaggaactacctgaactggtaccagcagaagcccgacggcaccgtgaagctgctgatctactacaccagcaggctgcacagcggcgtgcccagcaagttcagcggcagcggcagcggcaccgactacagcctgaccatcagcaacctggagcaggaggacatcgccacctacttctgccagcagggcaacaccctgccctggaccttcgccggcggcaccaagctggagatcaag

SEQ ID NO: 118-199 - skipped (82 entries)

SEQ ID NO: 200 - protein, 5 aa, synthetic construct
RYTMH

SEQ ID NO: 201 - protein, 17 aa, synthetic construct
YINPSRGYTNYNQKFKD

SEQ ID NO: 202 - protein, 10 aa, synthetic construct
YYDDHYCLDY

SEQ ID NO: 203 - protein, 7 aa, synthetic construct
GYTFTRY

SEQ ID NO: 204 - protein, 6 aa, synthetic construct
NPSRGY

SEQ ID NO: 205 - protein, 10 aa, synthetic construct
YYDDHYCLDY

SEQ ID NO: 206 - protein, 10 aa, synthetic construct
SASSSVSYMN

SEQ ID NO: 207 - protein, 7 aa, synthetic construct
DTSKLAS

SEQ ID NO: 208 - protein, 9 aa, synthetic construct
QQWSSNPFT

SEQ ID NO: 209 - protein, 10 aa, synthetic construct
SASSSVSYMN

SEQ ID NO: 210 - protein, 7 aa, synthetic construct
DTSKLAS

SEQ ID NO: 211 - protein, 9 aa, synthetic construct
QQWSSNPFT

SEQ ID NO: 212 - protein, 5 aa, synthetic construct
GYTMN

SEQ ID NO: 213 - protein, 17 aa, synthetic construct
LINPYKGVSTYNQKFKD

SEQ ID NO: 214 - protein, 13 aa, synthetic construct
SGYYGDSDWYFDV

SEQ ID NO: 215 - protein, 7 aa, synthetic construct
GYSFTGY

SEQ ID NO: 216 - protein, 6 aa, synthetic construct
NPYKGV

SEQ ID NO: 217 - protein, 13 aa, synthetic construct
SGYYGDSDWYFDV

SEQ ID NO: 218 - protein, 11 aa, synthetic construct
RASQDIRNYLN

SEQ ID NO: 219 - protein, 7 aa, synthetic construct
YTSRLHS

SEQ ID NO: 220 - protein, 9 aa, synthetic construct
QQGNTLPWT

SEQ ID NO: 221 - protein, 11 aa, synthetic construct
RASQDIRNYLN

SEQ ID NO: 222 - protein, 7 aa, synthetic construct
YTSRLHS

SEQ ID NO: 223 - protein, 9 aa, synthetic construct
QQGNTLPWT

SEQ ID NO: 224 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 225 - protein, 4 aa, synthetic construct
KKMP

SEQ ID NO: 226 - protein, 4 aa, synthetic construct
KKTN

SEQ ID NO: 227 - protein, 10 aa, synthetic construct
EQKLISEEDL

SEQ ID NO: 228 - protein, 30 aa, synthetic construct
  VARIANT 1..30: note = This sequence may encompass 1-6 GGGGS repeating units
GGGGSGGGGSGGGGSGGGGSGGGGSGGGGS

SEQ ID NO: 229 - protein, 22 aa, synthetic construct
GGGGSGGGGSGGGGSGGGGSAE

SEQ ID NO: 230 - protein, 5 aa, synthetic construct
GGGGS

SEQ ID NO: 231 - protein, 22 aa, Teschovirus A
GSGATNFSLLKQAGDVEENPGP

SEQ ID NO: 232 - protein, 60 aa, synthetic construct
  VARIANT 1..60: note = This sequence may encompass 1-12 GGGGS repeating units
GGGGSGGGGSGGGGSGGGGSGGGGSGGGGSGGGGSGGGGSGGGGSGGGGSGGGGSGGGGS

SEQ ID NO: 233 - protein, 25 aa, synthetic construct
  VARIANT 1..25: note = This sequence may encompass 1-5 GGGGS repeating units
GGGGSGGGGSGGGGSGGGGSGGGGS